(19) United States
(12) Patent Application Publication
(10) Pub. No.: US 2025/0206790 A1
(43) Pub. Date: Jun. 26, 2025

(54) COMPOSITIONS AND METHODS FOR DEREPRESSING RE1 SILENCING TRANSCRIPTION FACTOR TARGET GENES

(71) Applicant: Alcamena Stem Cell Therapeutics, LLC, Halethorpe, MD (US)

(72) Inventors: Edmund NESTI, Spencerville, MD (US); Alexander PISARCHIK, Marriottsville, MD (US)

(21) Appl. No.: 19/004,790

(22) Filed: Dec. 30, 2024

Related U.S. Application Data
(62) Division of application No. 17/778,738, filed on May 20, 2022, now Pat. No. 12,180,255.
(60) Provisional application No. 62/939,149, filed on Nov. 22, 2019.
(60) Provisional application No. 63/086,248, filed on Oct. 1, 2020.

(51) Int. Cl. C07K 14/47; A61P 25/00

(52) U.S. Cl. CPC C07K 14/4703; A61P 25/00; C07K 2319/06

(57) ABSTRACT

The invention relates to compounds, compositions, and methods for derepressing RE1 silencing transcription factor (REST) target genes are provided. In particular, a peptide having the sequence TEDLEPPEPPLPKEN (SEQ. ID NO: 1) and EDLEPPEPPLPK (SEQ. ID NO: 15), or the reversed sequences made of D-amino acids (retro inverted, RI) nekplppeppeldet (SEQ ID NO: 16) and kplppeppelde (SEQ ID NO: 17), are disclosed for inhibiting REST activity. The peptides are useful to treat, prevent, or ameliorate conditions such as traumatic brain injury, epilepsy, dementia, Huntington's Disease (HD), chronic pain, brain cancer (including glioblastoma multiforme), pancreatic cancer; diabetes, and peripheral nerve injury.

### CROSS-REFERENCE TO RELATED APPLICATIONS AND CLAIM TO PRIORITY

This application is a divisional of U.S. application Ser. No. 17/778,738 filed May 20, 2022, which is a national stage application of International Application No. PCT/US2020/061632 filed Nov. 20, 2020, which claims priority to U.S. Provisional Patent Application Nos. 62/939,149, filed Nov. 22, 2019, and 63/086,248, filed Oct. 1, 2020, the disclosures of which are incorporated herein by reference and to which priority is claimed.

### SEQUENCE LISTING

The instant application contains a Sequence Listing which has been submitted electronically in XML file format and is hereby incorporated by reference in its entirety. Said XML copy, created on Mar. 14, 2025, is named 9097_001_DIV_SL.xml and is 559,113 bytes in size.

### FIELD OF THE INVENTION

Methods, compounds, and compositions for derepressing RE1 silencing transcription factor (REST) target genes are provided. In particular, a peptide having the sequence TEDLEPPEPPLPKEN (SEQ. ID NO: 1) and EDLEPPEPPLPK (SEQ. ID NO: 15), or the reversed sequences made of D-amino acids (retro inverted, RI) nekplppeppeldet (SEQ ID NO: 16) and kplppeppelde (SEQ ID NO: 17), are disclosed for inhibiting REST activity. The peptides are useful to treat, prevent, or ameliorate conditions such as traumatic brain injury, epilepsy, dementia, Huntington's Disease (HD), chronic pain, brain cancer (including glioblastoma multiforme), pancreatic cancer; diabetes, and peripheral nerve injury.

### BACKGROUND

The repressor element 1 (RE1) silencing transcription factor (REST) is a repressor of hundreds of neuronal genes¹. Its targets represent genes required for the terminally differentiated neuronal cell phenotype, including genes encoding voltage and ligand dependent ion channels, their receptors, growth factors, and axonal-guidance proteins (Bruce A W et al, Proc Natl Acad Sci USA 101, 10458-10463 (2004); Conaco C et al, Proc Natl Acad Sci USA 103, 2422-2427 (2006); Mortazavi A et al, Genome Res 16, 1208-1221 (2006); and Otto S J et al, J Neurosci 27, 6729-6739 (2007); all of which are incorporated by reference herein). Thus, during neurogenesis, REST is progressively down regulated to allow elaboration of the mature neuronal phenotype (Ballas et al, 2005 supra). The importance of this event is demonstrated by gain-of-function studies that indicate the persistence of REST impedes terminal neuronal differentiation (Mandel G et al, Proc Natl Acad Sci USA 108, 16789-16794 (2011) and Gao Z ei a I, J Neurosci 31, 9772-9786 (2011); both of which are incorporated by reference herein).

Relatively little is known either about the transcriptional or post-transcriptional regulation of REST (Ballas N et al 2005 supra; Ballas N et al, Neuron 31, 353-365 (2001); and Kojima T et al, Brain Res Mol Brain Res 90, 174-186 (2001); all of which are incorporated by reference herein). However, two phosphorylation sites on REST (serines 861 and 864) regulate neuronal differentiation by their interaction with C-terminal domain small phosphatase-1 (CTDSP1)². When CTDSP1 removes phosphates from serine 861 and 864, REST protein is stabilized and neuronal differentiation is inhibited²⁻⁴.

### SUMMARY OF THE INVENTION

Disclosed herein are methods, compounds, and compositions for developing peptides with high affinity to CTDSP1.

Disclosed herein are methods, compounds, and compositions for binding C-terminal domain small phosphatase 1 (CTDSP1).

Disclosed herein are REST phosphomimetic peptides, TEDLEPPEPPLPKEN (SEQ ID NO: 1), EDLEPPEPPLPK (SEQ. ID NO: 15), nekplppeppeldet (SEQ ID NO: 16) and kplppeppelde (SEQ ID NO: 17), which bind CTDSP1 to inhibit REST activity. The lowercase letters represent D-amino acids, which are resistant to degradation and therefore increase peptide half-life, without compromising binding affinity⁵.

Disclosed herein are ninety eight (98) REST phosphomimetic peptide variants (RPPv) (SEQ IDS: 18 through 117), which inhibit CTDSP1 activity on REST to varying degrees (FIG. 25).

Disclosed herein are intracellular transport peptides, such as cell penetrating peptides (CPP) and/or endosomal release sequences, (SEQ ID NOS: 118 through 137 and 140 through 159) that may be fussed to an RPP (SEQ ID NOS: 1 and 15 through 17) or an RPPv (SEQ ID NOS: 18 through 117) at the N- or C-terminus to improve intracellular transport. Also disclosed are linkers (SEQ ID NOS: 138, 139, 160 and 161) that may be inserted between an RPP or RPPv and one of the peptides listed in Table 7, to further improve intracellular transport.

Disclosed herein is that RPP (SEQ ID NO: 1 and 15 through 17) or RPPv (SEQ ID NOS: 18 through 117) fused at the N- or C-terminus to an intracellular transport peptide (SEQ ID NOS: 118 through 137 and 140 through 159) and cyclized to further improve binding affinity and stability (increase peptide half-life). Examples of cyclized fusion proteins are SEQ ID NOS: 2, 5, 12, 13, and 14.

Disclosed herein is that RPP (SEQ ID NO: 1 and 15 through 17) or RPPv (SEQ ID NOS: 18 through 137 and 140 through 159) fused at the N- or C-terminus to an intracellular transport peptide (SEQ ID NOS: 118 through 137 and 140 through 159), promotes degradation of REST protein. Examples of the fusion protein are SEQ ID NOS: 2, and 4 through 14.

Disclosed herein is that REST phosphomimetic peptide of SEQ ID NO: 1 and 15-17 or RPPv SEQ ID NOS: 18 through 117 fused at the N- or C-terminus to an intracellular transport sequence (SEQ ID NOS: 118 through 137 and 140 through 159), promotes expression of REST target genes. Examples of the fusion protein are SEQ ID NOS: 2, 4 through 14.

Disclosed herein are RPP SEQ ID NOS: 1 and 15 through 17 or RPPv (SEQ ID NOS: 18 through 117) fused at the N- or C-terminus to an intracellular transport peptide (SEQ ID NOS: 118 through 137 and 140 through 159), is useful for treating animals having a disease or condition associated with REST or CTDSP1 by administering to the animal a therapeutically effective amount of the compound so that expression of REST target genes are increased or that expression of BDNF is increased. Examples of the fusion protein are SEQ ID NOS: 2 and 4 through 14.

Disclosed herein are RPP SEQ ID NOS: 1 and 15 through 17 or RPPv (SEQ ID NOS: 18 through 117) fused to an intracellular transport peptide (SEQ ID NOS: 118 through 137 and 140 through 159), is useful for treating traumatic brain injury, chronic pain, peripheral nerve injury, epilepsy, diabetes, Alzheimer's disease, Huntington's disease, brain tumors (including glioblastoma multiforme), or pancreatic cancer in an animal. Examples of the fusion protein are SEQ ID NOS: 2 and 4 through 14.

### BRIEF DESCRIPTION OF THE DRAWINGS

The accompanying drawings are incorporated in and constitute a part of the specification. The drawings, together with the general description given above and the detailed description of the exemplary embodiments and methods given below, serve to explain the principles of the invention. In such drawings:

FIG. 1. (A) Full length REST protein showing the location of the CTDSP1 binding site. Figure discloses SEQ ID NO: 347. (B) The retro inverted (RI) REST phosphomimetic peptide integrated with a cell penetrating peptide in a cyclic format. Figure discloses SEQ ID NO: 348.

FIG. 2. Binding affinity of the REST phosphomimetic peptide (RPP) for CTDSP1 was measured using the Monolith (NanoTemper). The affinity of RPP (Table 4; SEQ ID NO: 165) for CTDSP1 (Table 2; SEQ ID NO:163) is approximately 130 pM. Binding was assessed by fluorescently labelling the His tag on CTDSP1 (40 nM) with RED-tris NTA dye (20 nM) and measuring the change in fluorescents when RPP (up to 0.5 μM) is bound.

FIG. 3. Binding affinity of the RPP for CTDSP1 was measured using the Biacore (GE Healthcare Life Sciences). CTDSP1 (Table 2; SEQ ID NOS: 163) was immobilized on a CM5 sensor chip (GE Healthcare Life Sciences) and binding affinity of (A) GST control or (B) RPP (Table 4; SEQ ID NO: 165) was measured.

FIG. 4. RPP is cell permeable and accumulates in the nucleus. (A-D) 100 nM

FLAG-RPP-CPP
(CAQKDYKDDDDKTEDLEPPEPPLPKENGRKKRRQRRRG (SEQ. ID
NO: 4))

was added to mesenchymal progenitor cells (MPCs) and incubated for 4 hours. After 6 days in culture, the RPP concentrated in the nucleus. (E, F). Over a 6-day period, the peptide translocates from the cytoplasm to the nucleus (standard deviation, n=3). (A) cytoskeleton (phalloidin); (B) nucleus (hoechst dye); (C) RPP (FLAG Ab); (D) composite image.

FIG. 5. RPP accumulates in the nuclei of the sciatic nerve in spinal cord. A 0.7 cm coronal spinal cord section from the L3 and L4 region of a Sprague Dawley (SD) male rat showing the surrounding nuclei of the sciatic nerve, harvested 48 hours after 1 mg (1 mg/mL) of peptide was injected into the sight of a mid-thigh sciatic nerve defect injury. A. Hoechst stain shows nuclei. B. FLAG antibody shows FLAG-RPP-CPP (SEQ. ID NO: 4) localization.

FIG. 6. RPP reduces REST protein levels. (Left) Western blot showing exogenously expressed REST protein levels in HEK293 cells after treatment with vehicle or 100 nM FLAG-RPP-CPP (SEQ. ID NO: 4) for 4 hours. The blot was probed with anti-REST (Millipore-Sigma, 07-579) and anti-GAPDH. (Right) Bar graph showing 100 nM FLAG-RPP-CPP decreased REST protein levels 58%. Band intensity was calculated by measuring the area of gel peaks using ImageJ.

FIG. 7. RPP increases BDNF expression. mRNA levels in HEK 293 cells measured with qPCR after a 4-hour incubation with FLAG-RPP-CPP (SEQ. ID NO: 4, 100 nM) or vehicle (PBS) 24 hours in culture. The mean fold change in expression of BDNF (2^{-DDC}_{T}) was 2.401±0.885 (standard deviation, n=3). A mean fold change of 1=no change. 2^{-DDCT} calculated as described by Livak et al., 2001.

FIG. 8. mRNA levels of Kv4.3 in NBFL cells 48 h after CTDSP1 inhibition. Cells were transfected with a plasmid expressing RPP (SEQ ID NO: 1) with GFP (+) or a control peptide with GFP (−). 24 hours after transfection, cells were sorted based on fluorescence intensity. Error bars represent standard deviation, n=2.

FIG. 9. Comparison of linear and cyclic RPP. NBFL cells were dosed with (1 μM) RPP (linear:_SEQ ID NO: 4; cyclic:_SEQ ID NO: 2, Table 8) or control peptide (SEQ ID NO: 3, Table 9) for 24 or 48 hours. Efficacy on was assessed by measuring changes in (A) BDNF, (B) NGF, and (C) Kv4.3 transcript levels. N=1

FIG. 10. BDNF and NGF mRNA expression (normalized to actin) in mesenchymal progenitor cells (MPCs), from two patients, after a 48 hour incubation with water (Ctrl) or 3 μM RPP (SEQ. ID NO: 9). mRNA levels are shown relative to control (standard deviation, n=2).

FIG. 11. iPSCs (neural stem cells (NSC)-NL5) were dosed with one of the RPP sequences (3 μM) or water control shown below. After 7 days in culture, cells were assessed for neuronal differentiation using the Microtubule Associated Protein 2 (MAP2) neuronal marker (normalized to DAPI). Media and variants were replaced on Day 3. MAP2 levels are relative to control in the bar graph (standard deviation n=4).

| Sequence | |
|---|---|
| ID number | |
| (SEQ ID | Peptide |
| NO) Control | Water |
| 5 | CTEDLEPPEPPLPKENSGDIMGEWGNEIFGAIA |
| | GFLGYGRKKRRQRRRG^{cylic} |
| 6 | TEDLEPPEPPLPKENRRWWRRWRRRRWWRr |
| 7 | EDLEPPEPPLPKRWWRRRRWRRWWRr |
| 8 | AGDLEQPEPPVAKKKKKNRRWWRRWRr |
| 9 | rrrrwrrwwrrwrrnekplppeppeldet |
| 10 | TEDLEPPEPPLPKENrrrrwrrwwrrwrr |
| 11 | TEDLEPPEPPLPKENRRRRRRC₁₄RRWWRRr |

Lowercase = D-amino acids
C₁₄ = 2-amino-tetradecanoic acid

FIG. 12. iPSCs (neural stem cells (NSC)-NL5) were with 1 μM RPP (SEQ ID NO. 9) or water control. After 7 days in culture, cells were assessed for neuronal differentiation using neuronal markers TUJ1 (class III beta-tubulin) and MAP2 (both normalized to DAPI). Media and RPP were replaced on Day 3. (standard deviation, n=6).

FIG. 13. iPSCs (neural stem cells (NSC)-NL5) were with 1 μM RPP (SEQ ID NO. 13) or water control. After 7 days in culture, cells were assessed for neuronal differentiation using neuronal markers TUJ1 and MAP2 (both normalized to DAPI). Media and RPP were replaced on Day 3. (standard deviation, n=6).

FIG. 14. RPP (SEQ. ID NO: 12) increases expression of Kv4.3, Kv7.2, Nav1.8 and OPRM1. qRT-PCR analysis of mRNA expression (normalized to β-actin), relative to water control, after incubation of adult Sprague Dawley (SD) rat L5 DRGs with various concentrations of rpp for 48 h. *=p<0.05, **=p<0.01, ***=p<0.001, one-way ANOVA and Dunnett's test, error bars=standard error of the mean.

FIG. 15. 3 μM RPP for 48 hours (SEQ ID NOS: 13 and 14) increased expression of Nav1.8. qRT-PCR analysis of mRNA expression (normalized to β-actin), relative to water control, after incubation of RPP with adult Sprague Dawley (SD) rat L5 DRGs. Error bars=standard error of the mean.

FIG. 16. RPP (SEQ. ID NO: 12) increases expression of BDNF and NGF. qRT-PCR analysis of mRNA expression (normalized to β-actin), relative to water control, after incubation of adult Sprague Dawley (SD) rat L5 DRG neurons with various concentrations of RPP for 48 h. *=p<0.05, one-way ANOVA and Dunnett's test, error bars=SEM.

FIG. 17. RPP (SEQ. ID NO: 12) does not cause necrosis in DRG neurons. LDH toxicity assay in SD rat incubated with water, RPP (1, 3, or 10 μM), or triton for 48 hours. ***=p<0.001, one-way ANOVA and Dunnett's test, error bars=SD.

FIG. 18. RNA Levels of Chronic Pain Genes After SNI. RNA fold change relative to β-actin on Day 0 (sham, n=3), 7 (n=5), or 28 (n=2) after SNI (mean±SD, ** p<0.01, *** p<0.001, one-way ANOVA with Dunnett's multiple comparison test. RNA was isolated from sciatic nerve DRG (L5).

FIG. 19. REST mRNA levels increase after TBI. (A) Normal levels of REST mRNA expression in the ipsilateral cortex of uninjured mouse. (B) REST levels increase dramatically in the mouse ipsilateral cortex 7 days after cortical controlled impact injury⁶. mRNA visualized by RNAscope with DAPI counterstain.

FIG. 20. (a) Schematic of AAVS1-Nanoluc-Halotag Knock-in (KI) construct used to engineer NCRM-1 iPSCs. The CMV-driven construct was inserted into the safe harbor AAVS1 locus of Chr. 19q by transcription activator-like effector nucleases (TALENS). AAVS1 KI was confirmed by sequencing and junction PCR (not shown). (b) Schematic of MAP2-Nanoluc-Halotag Knock-in (KI) construct used to engineer NCRM-1 iPSCs. Nanoluciferase-Halotag was knocked-in to the MAP2 transcriptional start site (TSS) (Chr. 2) using zinc finger nucleases (ZFN) and MAP2-in frame KI confirmed by sequencing and junction PCR (not shown). CMV-NLHT and MAP2-NLHT iPSCs express pluripotency markers OCT4/NANOG/TRA 1-81/TRA 1-60 (not shown). (c) Luciferase assay of CMV-NLHT iPSCs and MAP2-NLHT neural stem progenitor cells (NSPC) and neurons. Note the increasing luciferase activity with neuronal differentiation of MAP2-NLHT cells consistent with increased MAP2 expression with differentiation.

FIG. 21. Rat sciatic nerve injury model: (Left) Surgical approach of sciatic nerve transection. (Right) Immunohistochemistry-Immunofluorescence staining from section of sciatic nerve. Tissues were stained for BIII Tubulin, Laminin I, and DAPI. The scale bars=100 μm.

FIG. 22. Rat sciatic nerve harvest: (Left) En Bloc section of rat spinal column and associated peripheral nerves. (Right) Image of sciatic nerve injury site with associated lumbar nerve roots

FIG. 23. A. Compared to baseline (BASE), SNI induces sensitization of mechanical pain threshold (MT) 4-days post-injury (n=16); paired t-test, *p<0.05, SHAM shows no significant difference in mechanical threshold 4-days post-surgery. B. Analgesic effect of single S.C. injection of morphine on MT post-injury (SNI+MOR, n=4). Mean±SEM; F=4.62, *p<0.05 by one-way ANOVA before and after treatment; mean difference and 95% CI=−7.3, −14.5 to −0.135, p<0.05 by Bonferroni's Multiple Comparison test. 20 mg/Kg of MOR (n=4; F=19.03, ** p<0.001 by one-way ANOVA).

FIG. 24. Conditioned Place Preference (CPP) after conditioning with saline (control) and 4 doses of morphine (MOR) in SNI mice. MOR induced CPP in a dose-dependent manner (one-way ANOVA, F=4.167, p<0.05); 10 mg vs control (p<0.001) and 20 mg (p<0.05) by Tukey's multiple comparisons test.

FIG. 25. In vitro CTDSP1 phosphatase activity (relative to GST control) on the REST peptide (TEDpSPPpSPPLPKEN (SEQ ID NO: 329)) as a substrate after exposure to 1 μM of RPP (SEQ ID NO: 1) or one of its variants (Table 3).

FIG. 26. In vitro on target (CTDSP1) and off target phosphatase activity (1=PPA1, 2=PPM1H, 3=PPM1A, 4=PP3CA, 5=PPP1CA, 6=PP5CA), relative to water control, on the phosphorylated REST peptide (TEDpSPPpSPPLPKEN (SEQ ID NO: 329)) as a substrate after exposure to RPP (SEQ ID NO: 12). N=5, error bars are standard deviation.

FIG. 27. Building RPP libraries. A) Scheme of the experiment; B) Nucleotide Sequence of the REST construct (before mutagenesis) (SEQ ID NO: 349); C) Protein Sequence of the REST construct (before mutagenesis) (SEQ ID NO: 350). RPP, CPP and His tag are underlined.

FIG. 28. RPP (SEQ ID NO: 13) structure. Along the bottom of the structure is the CPP containing a poly arginine sequence with two 2-aminotetradecanoic acids that are integrated with the REST RI phosphomimetic (starting with the lysine at the bottom right of the structure).

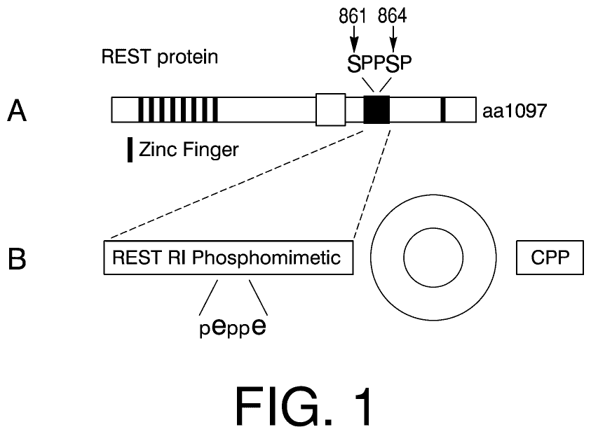

### DETAILED DESCRIPTION OF THE EMBODIMENTS

In an embodiment, the present invention provides the methods (described in examples 1 through 3) for developing peptides with high affinity to CTDSP1. Importantly, the method uses peptide evolution techniques to generate peptides with high affinity to CTDSP1.

In an embodiment, the present invention provides a peptide of SEQ ID NOS: 1 and 15 through 17 or a peptide having at least 50%, more preferably at least 60%, yet more preferably at least 70%, still more preferably at least 80%, even more preferably at least 90%, or even yet more preferably at least 95% similarity to SEQ ID NO: 1 and 15 through 17. These peptides or similar peptides thereof are referred to herein as REST phosphomimetic peptides (RPP). Importantly, however, the glutamic acids in the RPP must be maintained, particularly the glutamic acid at positions 5 and 8 of SEQ ID NO: 1, positions 4 and 7 of SEQ ID NO: 15, positions 8 and 11 of SEQ ID NO: 16, and positions 6 and 9 of SEQ ID NO: 17.

In an embodiment, the present invention provides RPPv (SEQ ID NOS: 18 through 117), or a peptide having at least 50%, more preferably at least 60%, yet more preferably at least 70%, still more preferably at least 80%, even more preferably at least 90%, or even yet more preferably at least 95% similarity to an RPPv (SEQ ID NOS: 18 through 117). The peptide or similar peptide thereof is referred to herein as the REST phosphomimetic peptide variant (RPPv) (SEQ ID NOS: 18 through 117). Importantly, however, the glutamic acids in SEQ ID NOS: 18 through 67 must be maintained, particularly the glutamic acid at positions equivalent to 5 and 8 of SEQ ID NO: 1. For the retro inverted sequences (RPPv^{RI}) (SEQ ID NOS: 68 through 117), positions 8 and 11 of SEQ ID NO: 16 must be maintained.

In an embodiment, the present invention a fusion protein between RPP (SEQ ID NOS: 1 and 15 through 17) or RPPv (SEQ ID NOS: 18 through 117) and an intracellular transport peptide (SEQ ID NOS: 118 through 137 and 140 through 159) or a peptide having at least 50%, more preferably at least 60%, yet more preferably at least 70%, still more preferably at least 80%, even more preferably at least 90%, or even yet more preferably at least 95% similarity.

The identity/similarity between two or more nucleic acid sequences, or two or more amino acid sequences, is expressed in terms of the identity or similarity between the sequences. Sequence identity can be measured in terms of percentage identity; the higher the percentage, the more identical the sequences are. Sequence similarity can be measured in terms of percentage similarity (which takes into account conservative amino acid substitutions); the higher the percentage, the more similar the sequences are.

Methods of alignment of sequences for comparison are well known in the art. Various programs and alignment algorithms are described in: Smith & Waterman, Adv. Appl. Math. 2:482, 1981; Needleman & Wunsch, J. Mol. Biol. 48:443, 1970; Pearson & Lipman, Proc. Natl. Acad. Sci. USA 85:2444, 1988; Higgins & Sharp, Gene, 73:237-44, 1988; Higgins & Sharp, CABIOS 5:151-3, 1989; Corpet et al., Nuc. Acids Res. 16:10881-90, 1988; Huang et al. Computer Appls. in the Biosciences 8, 155-65, 1992; and Pearson et al., Meth. Mol. Bio. 24:307-31, 1994. Altschul et al., J. Mol. Biol. 215:403-10, 1990, presents a detailed consideration of sequence alignment methods and homology calculations.

The NCBI Basic Local Alignment Search Tool (BLAST) (Altschul et al., J. Mol. Biol. 215:403-10, 1990) is available from several sources, including the National Center for Biological Information (NCBI, National Library of Medicine, Building 38A, Room 8N805, Bethesda, MD 20894) and on the Internet, for use in connection with the sequence analysis programs BLASTP, BLASTN, BLASTX, TBLASTN AND TBLASTX. Additional information can be found at the NCBI web site (www.ncbi.nlm.nih.gov).

BLASTN is used to compare nucleic acid sequences, while BLASTP is used to compare amino acid sequences. If the two compared sequences share homology, then the designated output file will present those regions of homology as aligned sequences. If the two compared sequences do not share homology, then the designated output file will not present aligned sequences.

Once aligned, the number of matches is determined by counting the number of positions where an identical nucleotide or amino acid residue is presented in both sequences. The percent sequence identity is determined by dividing the number of matches either by the length of the sequence set forth in the identified sequence, or by an articulated length (such as 100 consecutive nucleotides or amino acid residues from a sequence set forth in an identified sequence), followed by multiplying the resulting value by 100. For example, a nucleic acid sequence that has 1166 matches when aligned with a test sequence having 1154 nucleotides is 75.0 percent identical to the test sequence (1166/1554*100=75.0). The percent sequence identity value is rounded to the nearest tenth. For example, 75.11, 75.12, 75.13, and 75.14 are rounded down to 75.1, while 75.15, 75.16, 75.17, 75.18, and 75.19 are rounded up to 75.2. The length value will always be an integer. In another example, a target sequence containing a 15-nucleotide region that aligns with 20 consecutive nucleotides from an identified sequence as follows contains a region that shares 75 percent sequence identity to that identified sequence (that is, 15/20*100=75).

For comparisons of amino acid sequences of greater than about 30 amino acids, the Blast 2 sequences function is employed using the default BLOSUM62 matrix set to default parameters, (gap existence cost of 11, and a per residue gap cost 5 of 1). Homologs are typically characterized by possession of at least 70% sequence identity counted over the full-length alignment with an amino acid sequence using the NCBI Basic Blast 2.0, gapped blastp with databases such as the nr or swissprot database. Queries searched with the blastn program are filtered with DUST (Hancock and Armstrong, 1994, Comput. Appl. Biosci. 10:67-70). Other programs use SEG. In addition, a manual alignment can be performed. Proteins with even greater similarity will show increasing percentage identities when assessed by this method, such as at least about 75%, 80%, 85%, 90%, 95%, 98%, or 99% sequence identity to a protein. When aligning short peptides (fewer than around 30 amino acids), the alignment is performed using the Blast 2 sequences function, employing the PAM30 matrix set to default parameters (open gap 9, extension gap 1 penalties). Proteins with even greater similarity to the reference sequence will show increasing percentage identities when assessed by this method, such as at least about 60%, 70%, 75%, 80%, 85%, 90%, 95%, 98%, or 99% sequence identity to a protein. When less than the entire sequence is being compared for sequence identity, homologs will typically possess at least 75% sequence identity over short windows of 10-20 amino acids, and can possess sequence identities of at least 85%, 90%, 95% or 98% depending on their identity to the reference sequence. Methods for determining sequence identity over such short windows are described at the NCBI web site.

One indication that two nucleic acid molecules are closely related is that the two molecules hybridize to each other under stringent conditions, as described above. Nucleic acid sequences that do not show a high degree of identity may nevertheless encode identical or similar (conserved) amino acid sequences, due to the degeneracy of the genetic code. Changes in a nucleic acid sequence can be made using this degeneracy to produce multiple nucleic acid molecules that all encode substantially the same protein. An alternative (and not necessarily cumulative) indication that two nucleic acid sequences are substantially identical is that the polypeptide which the first nucleic acid encodes is immunologically cross reactive with the polypeptide encoded by the second nucleic acid.

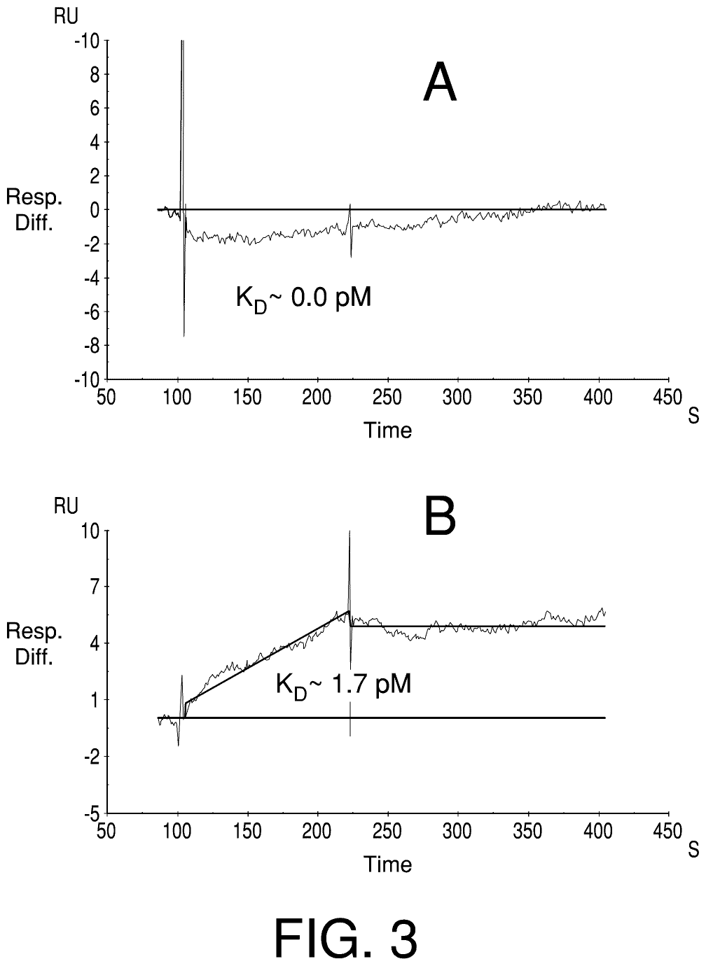

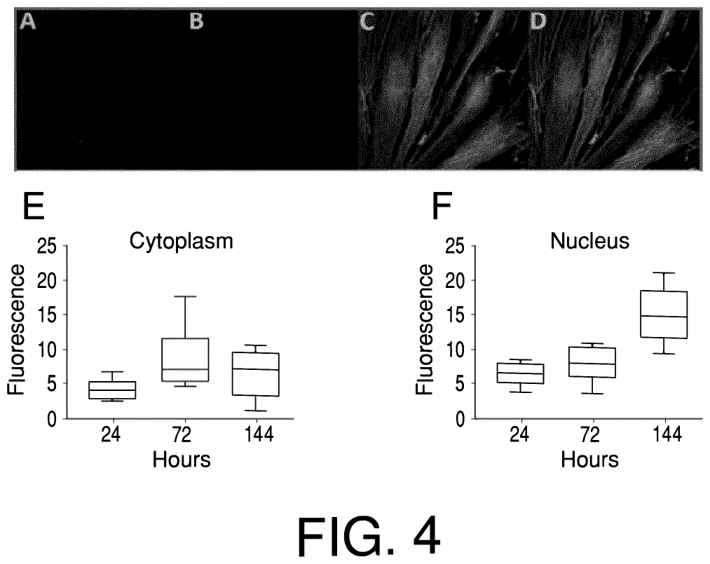

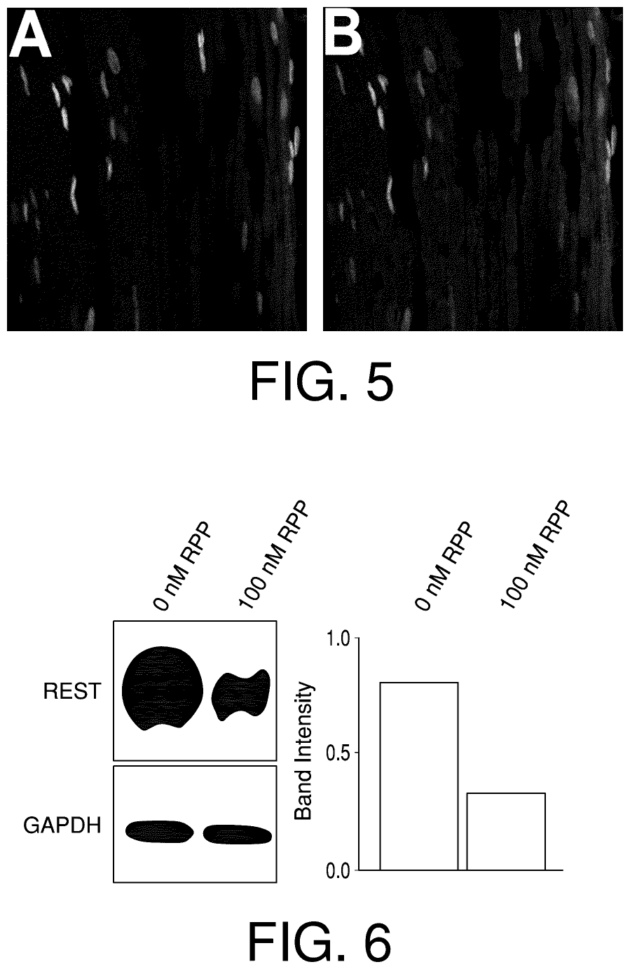

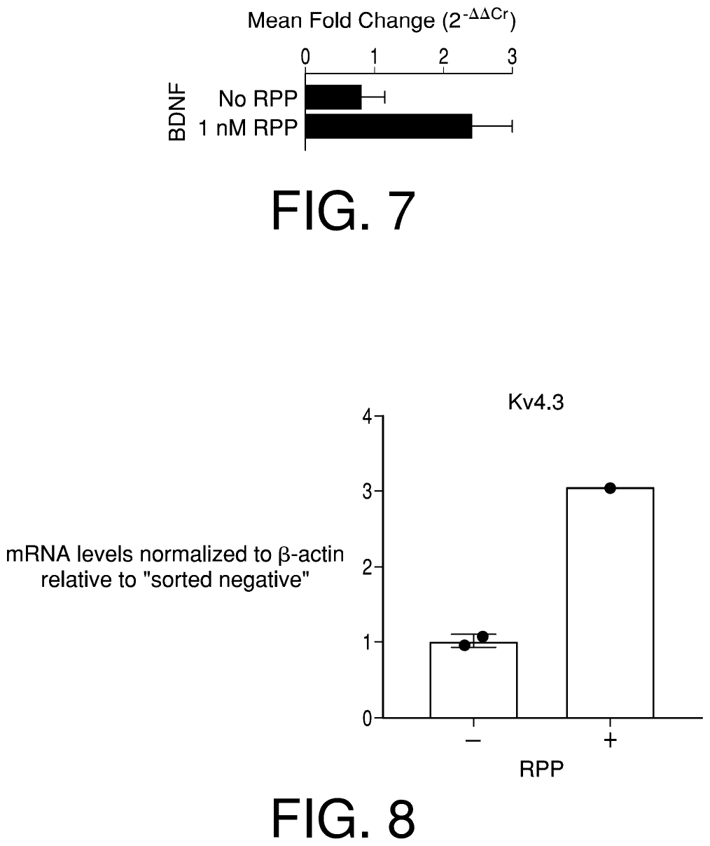

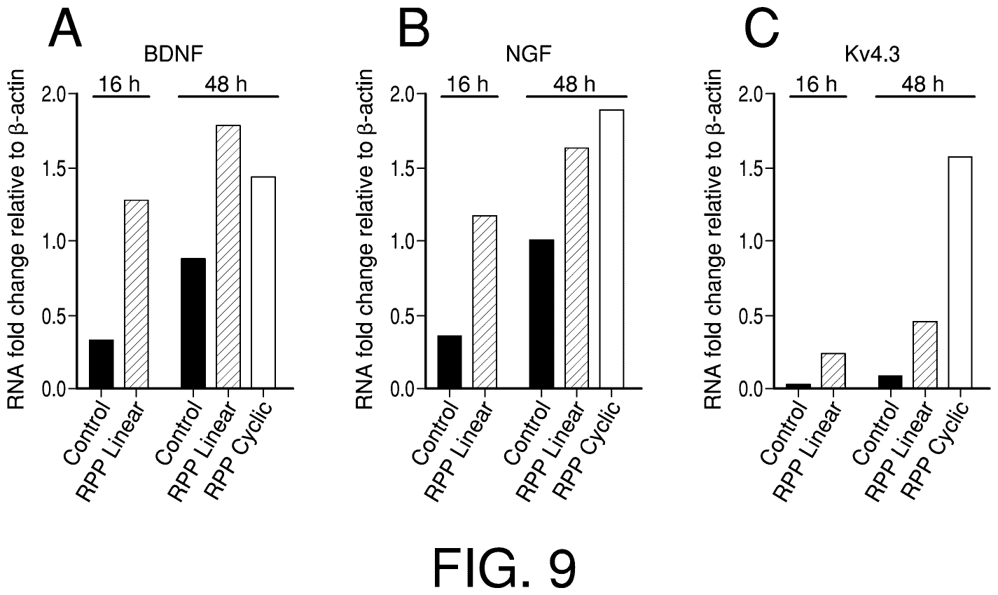

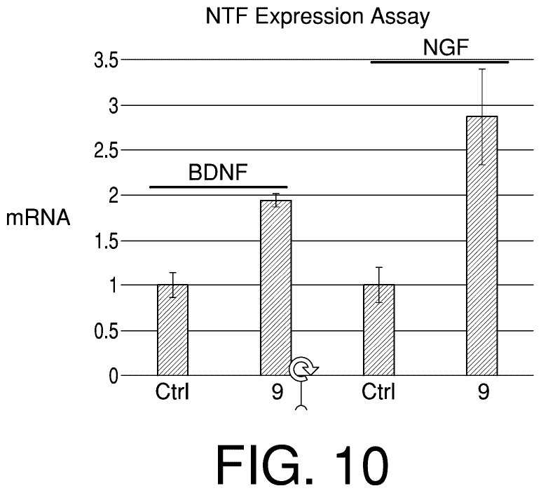

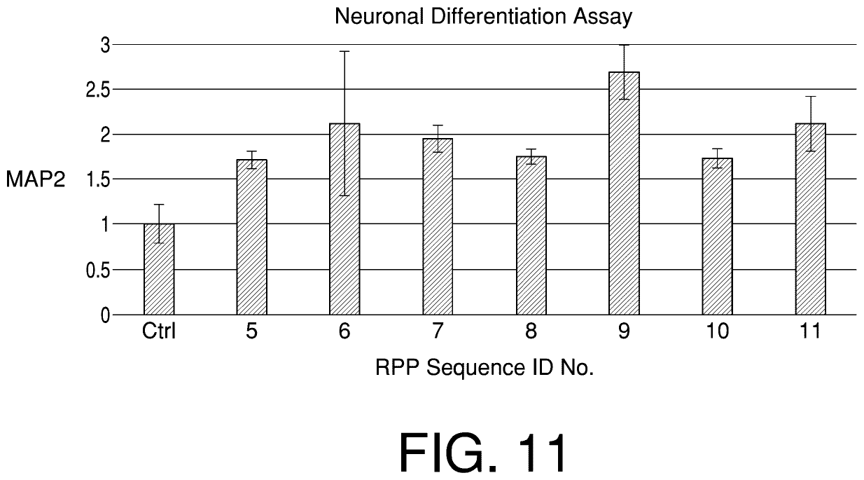

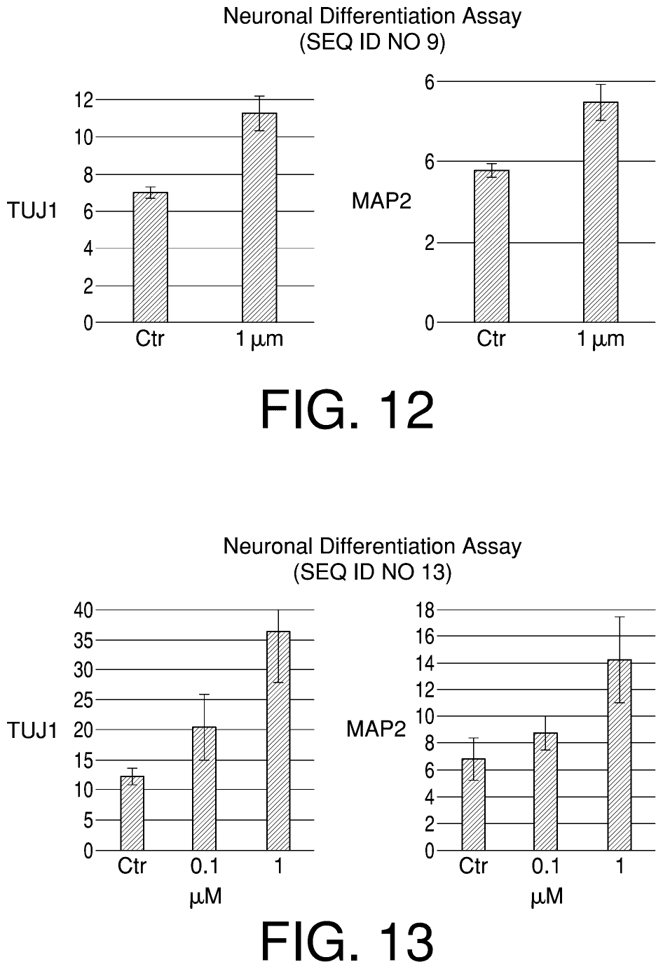

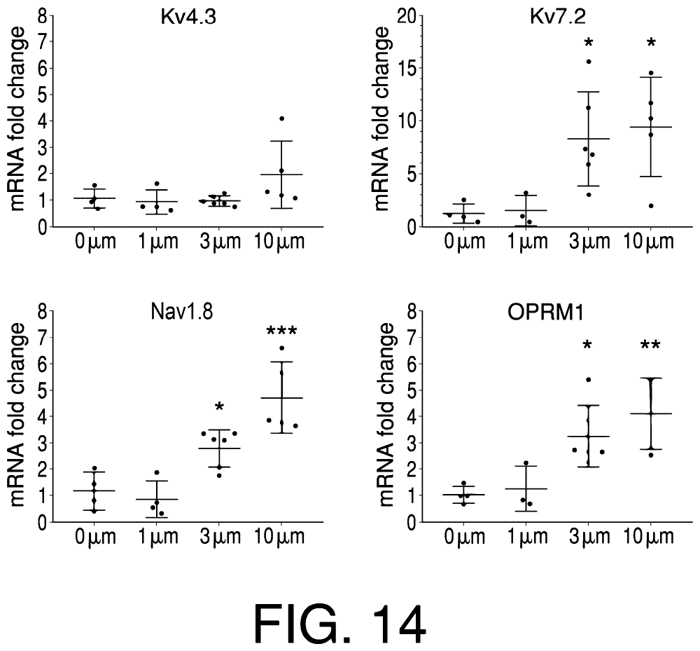

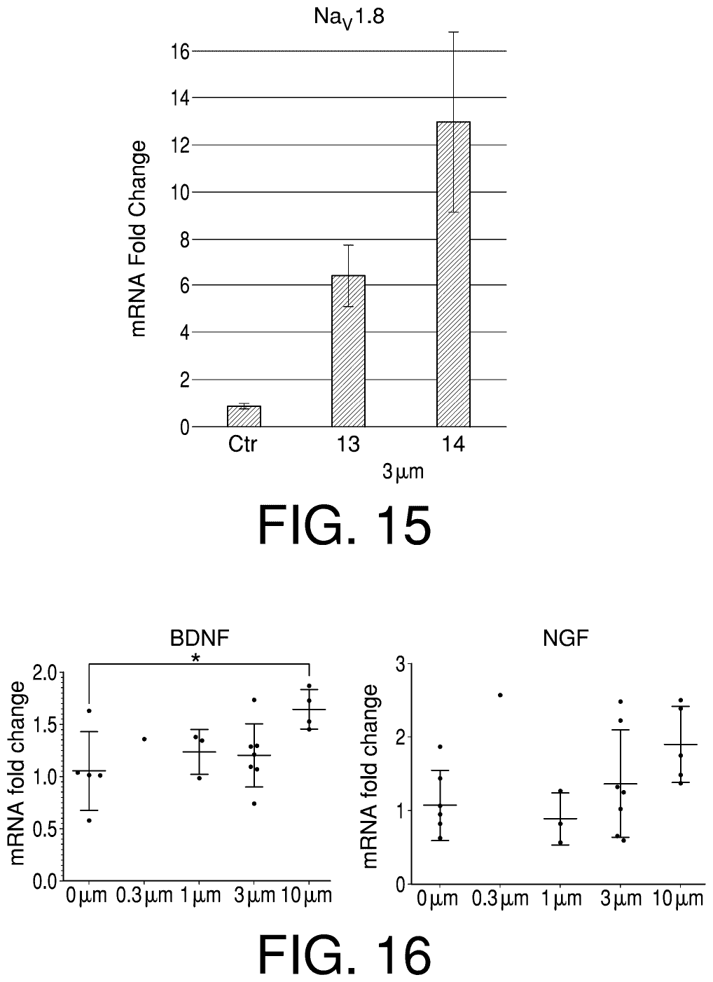

In certain embodiments, the RPP (SEQ ID NOS: 1 and 15 through 17) or RPPv (SEQ ID NOS: 18 through 117) may be fused to another peptide, e.g., to allow for cell penetration. Preferably, the RPP (SEQ ID NOS: 1 and 15 through 17) or RPPv (SEQ ID NOS: 18 through 117) is fused to a peptide sequence that allows cell penetration and escape from endosomes. More preferably, the cell penetration and escape peptide sequence is one SEQ ID NOS: 118 through 137 and 140 through 159. RPP (SEQ ID NOS: 1 and 15 through 17) or RPPv (SEQ ID NOS: 18 through 117) and cell penetration peptide (CPP) (SEQ ID NOS: 118 through 137 and 140 through 159) may be fused directly or contain a linker (e.g., SEQ ID NOS: 138, 139, 160, or 161) sequence connecting them. It has been shown that when cells or tissues are dosed with RPP (SEQ ID NOS: 1 and 15 through 17) or RPPv (SEQ ID NOS: 18 through 117) peptide fused to a CPP (SEQ ID NOS: 118 through 137 and 140 through 159), it localizes in the nucleus (FIG. 3, SEQ ID NO: 4 and FIG. 4_SEQ ID NO: 4), causes REST degradation (FIG. 5, SEQ ID NO: 4), increases the expression of genes targeted by REST (FIG. 7, SEQ. ID NO: 4, FIG. 8, SEQ ID NO: 1, FIG. 9, linear_SEQ ID NO: 4 and cyclic_SEQ ID NO: 2, FIG. 10, SEQ ID NO: 9, FIG. 14, SEQ ID NO: 12, FIG. 15, SEQ ID NOS: 13 & 14, and FIG. 16, SEQ ID NO: 12), and causes neural differentiation (FIG. 11, SEQ ID NOS: 5 through 11, FIG. 12, SEQ ID NO: 9, and FIG. 13, SEQ ID NO: 13).

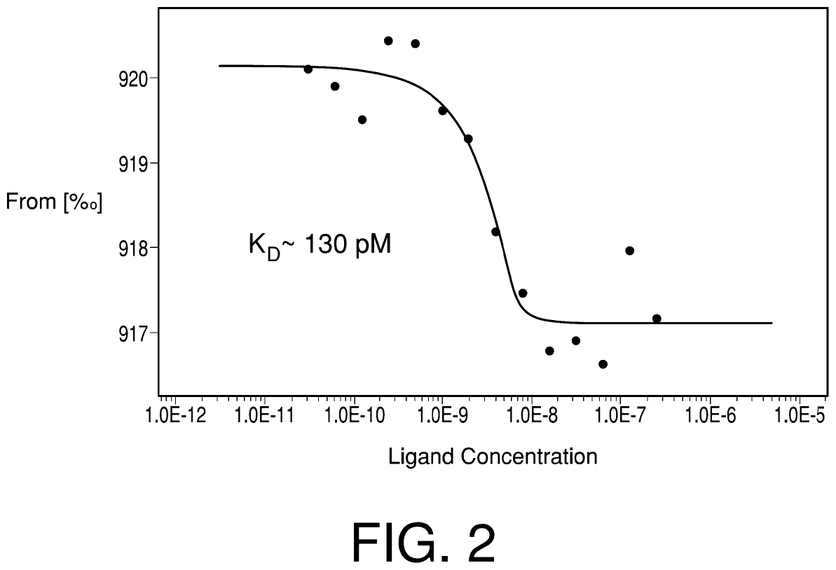

The present inventor has discovered that the RPP (SEQ ID NOS: 1 and 15 through 17) binds CTDSP1 to inhibit REST activity. The RPP has low pM affinity for CTDSP1, as demonstrated by the binding curves shown in FIGS. 2 and 3. In cells and animals, the RPP binds to CTDSP1, which prevents CTDSP1 from binding to REST.

Increased REST and associated neural gene repression underlie the pathology of the following diseases or disorders:

Traumatic Brain Injury—Traumatic brain injury (TBI) results in loss of cognitive ability due to neuronal death. Two issues should be addressed to improve cognitive recovery after TBI, 1) reduce the neuronal death that continues after injury and 2) regenerate lost neurons.

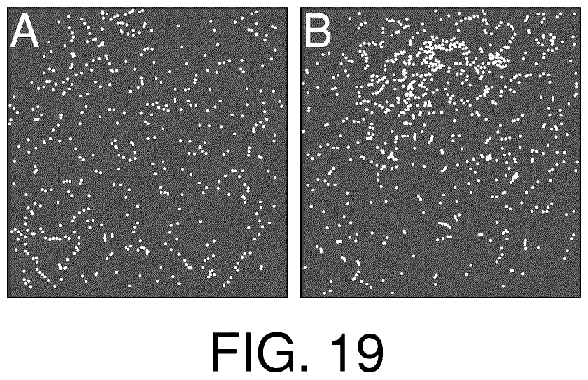

REST levels increase dramatically in the brain after TBI (FIG. 19). Consistently, several studies have shown that brain injury caused by acute ischemia induces REST expression in neurons resulting in their death⁷⁻⁹. These studies demonstrate that neuron survival improves when REST is removed⁷⁻⁹. The clinical implications are profound. For example, elevated levels of REST are correlated with an increased frequency of seizures, which commonly occur after brain injury¹⁰⁻¹². In animal models of brain injury, the rate of seizure decreases significantly when REST is inhibited^{13,14}. Also, chronically elevated levels of REST after TBI are likely to play a role in neurodegenerative diseases attributed to a history of brain injury¹⁵⁻¹⁷. Both in vitro and in vivo studies show that removing REST improves neuronal surviva¹⁷⁻⁹, function^{14,18}, and regeneration^{2,19-24}. Therefore, targeting REST after TBI should mitigate the effects of brain damage and offset the risks of developing associated age-related neurodegenerative diseases, such as Alzheimer's^{17,25}.

The basis for neurogenesis improving cognitive function in TBI patients are studies associating increased neurogenesis with improved learning, memory, and other cognitive functions²⁶⁻³⁰ and studies showing the converse, that inhibition of neurogenesis via antimitotic agents, radiation, or genetic manipulations impairs hippocampal dependent forms of memory²⁹⁻³³. Defects in neurogenesis are also linked with many diseases with cognitive etiologies including developmental disorders (e.g., microcephaly³⁴, megalencephaly³⁵, and autism³⁵) and neurodegenerative diseases (e.g., dementia and Alzheimer disease)³⁶. Experimental treatments (including blood transfusions, growth and neurotrophic factors, and stem cell transplantation^{10,37-49} have focused on regenerating neurons. These attempts have failed to stimulate the neurogenesis required to restore cognitive function, at least in part because they are not capable of terminal differentiation of neural progenitor cells in the context of TBI^{26,28,50-57} Terminal differentiation of neural progenitors into neurons is blocked at a single checkpoint by the RE1 silencing transcription factor known as REST.

Peripheral Nerve Injury-Neuronal regeneration in peripheral nerve injury (PNI) patients is inhibited in at least two ways. First, there is a reduction in neurotrophic factors (NTFs) that support the growth, survival, and differentiation of both developing and mature neurons⁵⁸. Second, there is a decrease in the expression of genes required for synaptic plasticity including axonal growth, vesicular transport, and ionic conductance⁵⁹. Both phenomena occur coincident with injury-induced expression of REST⁶⁰. Therefore, eliminating REST accelerates recovery from a PNI by reversing a block on nerve regeneration. Inhibition of CTDSP1 with the RPP promotes REST degradation (FIG. 6), increases neuronal differentiation (FIGS. 11, 12, and 13), and increases NTF expression (FIGS. 7, 9, 10, and 16), which demonstrates the potential of RPP (SEQ ID NOS: 1 and 15 through 17) or RPPv (SEQ ID NOS: 18 through 117) fused to a CPP (SEQ ID NOS: 118 through 137 and 140 through 159) to stimulate nerve regeneration.

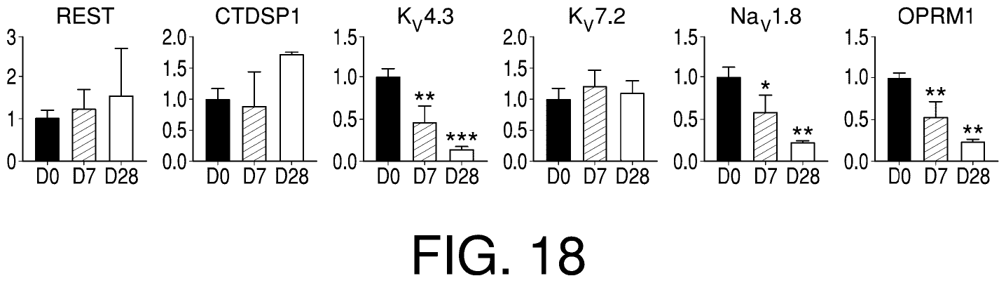

Chronic Pain-Many of the genes that are repressed in the central and peripheral circuits during chronic pain are either direct or indirect targets of the repressor element-1 (RE1)-silencing transcription factor (REST), an inhibitor of neuronal gene expression in stem cells, neural progenitors, and non-neuronal cells⁶¹⁻⁶³. REST levels are normally kept low in healthy neurons through active degradation to keep the chromatin clear of this powerful neuronal gene repressor⁶⁴⁻⁶⁹ Before the transition to neuropathic pain, REST levels significantly increase in the neurons of the peripheral nerves of mice and rats after peripheral nerve injury followed by a spike in neurons in the central nervous system where pain stimuli are processed and experienced^{60,70,71}. The activation of REST after nerve injury results in decreased expression of several genes required for normal excitability of sensory neurons, including the potassium channels Kv4.3 (Kcnd3) and Kv7.2 (Kcnq2), the sodium channel Nav1.8 (Scn10a), and the mu opioid receptor Oprm1 (FIG. 18)^{60,72-74} The basis for blocking REST to alleviate chronic pain are published studies using mouse and rat peripheral nerve injury (PNI) models^{60,72-74}. In mice, genetic knockdown of REST in sensory neurons after PNI restores gene expression of the mu opioid receptor^{60,72,73}, Nav1.8^{60,72,73}, Kv4.3^{72,73,75}, and Kv7.2^{72,73} in the dorsal root ganglion, restores normal K+M-type current in C nerve fibers⁶⁰, reduces hyperalgesia and allodynia⁷²⁻⁷⁴, and restores morphine analgesia^{60,74} in chronic pain models (Table 12). Disruption of the REST repressor complex, using an optimized REST mimetic of the mSin3 binding site, an adaptor protein which contributes to transcriptional repression of many genes including REST targets, restored C-fiber function, reduced hyperalgesia and allodynia, and restored morphine analgesia in mouse models of chronic pain (Table 12)⁷⁴. In rats, knockdown of REST controls the transition from acute to chronic pain, demonstrated by a reduction in hyperalgesia and allodynia, and restored muscarine analgesia (Table 12)^{72,73}

TABLE 12
Summary of chronic pain studies using REST blocking methods

| | Day | | | | | | |
|---|---|---|---|---|---|---|---|
| | post | | | | | | |
| REST block | injury | PNI | Assessment | Result | Species | Number | Ref. |
| Antisense | 1, 3, 5, | pSNL | Kv4.3 | Recovered [+2x | Mouse | 5 M | 75 |
| Oligo | & 6 | | | (mRNA) compared to | | | |
| | | | | vehicle control on Day | | | |
| | | | | 7] | | | |
| Antisense | 1, 3, 5, | pSNL | Oprml | Recovered [+3x | Mouse | 3 M | 60 |
| | | | | (mRNA) compared to | | | |
| | | | | Vehicle control on Day | | | |
| | | | | 7] | | | |
| Oligo | &6 | | Nav1.8 | 85% recovered [+2x | | | |
| | | | | (mRNA) compared to | | | |
| | | | | control on Day 7] | | | |
| | | | C-fiber | Recovery of function, | | | |
| | | | | compared to sham | | | |
| | | | | [PWTonDay 7] | | | |
| | | | Morphine analgesia | 85% recovery, | | | |
| | | | | compared to sham | | | |
| | | | | [PWTonDay 71 | | | |
| mSin3 decoy | 5-11 | pSNL | C-fiber | 75% reversal of | Mouse | 2-6/sex | 74 |
| | | | | hyposensitivity, | | | |
| | | | | compared to Sham | | | |
| | | | | [PWT on Day 12] | | | |
| | | | Morphine analgesia | Reversal of loss [PWT | | | |
| | | | | (ICS, IPS) on Day 12 | | | |
| | | | | & 19] | | | |
| | | Fibromyalgia | Hyperalgesia/allodynia | Reverse (Day 12 & 19) | | | |
| | | (IPS, ICS) | | | | | |
| Over | — | — | Hyperalgesia | Induction (PWT) on | Mouse | 3-5/sex | 72,73 |
| expression | | | (mechanical, hot, & cold) | Day 20, 25, 30 | | | |
| AAV-REST | | | | | | | |
| (DRG) | | | | | | | |
| cKO | Pre | CFA, SNI, | Hyperalgesia | None (PWT, Day 5, | | | |
| | | pSNL- | (mechanical, hot, & cold) | 10, 15) | | | |
| | | SNI | Kv7.2, Navi.8, Oprml, | Prevent down | | | |
| | | | Kv4.3 | regulation | | | |
| siRNA | 21-26 | SNL | Allodynia (tactile) | Reduce (up to 45%, | Rat | 6-9 M | 72,73 |
| | | | | Day 21 to 26) | | | |
| | | | Hyperalgesia | Reduce (up to 65%, | | | |
| | | | (mechanical) | Day 21 to 26) | | | |
| | | | Charm2 | Restores mRNA [2x | | | |
| | | | | compared to control | | | |
| | | | | siRNA on Day 51 | | | |
| | | | Muscarine analgesia | Restores (PWT) | | | |
| | | | (tactile & mechanical) | | | | |

Intermediate psycological stress (IPS); Intermittent cold stress (ICS); Paw withdrawal threshold (PWT)

We recognize the studies presented in Table 12 reveil a discrepancy in the reduction in hypersensitivity between the mouse conditional knockout (cKO) study (complete knockout), which showed no hypersensitivity and no downregulation of REST target genes after injury (Table 12), and the rat siRNA knockdown study, which only partially reduced allodynia (up to 45%) and hyperalgesia (up to 65%) after injury. We believe the knockout effect is more potent than siRNA knockdown, because the bioavailability of siRNAs is limited by their size (>13,000 kD) and poor cellular trafficking, due to endosomal entrapment.

We are aware that the studies in Table 12 lack rigor in that power analysis was not done to establish the number of animals per group and the animal numbers were low which makes statistical analysis questionable. Some of the studies used the less predictive partial sciatic nerve ligation (pSNL) model, versus the preferred spared nerve injury (SNI) chronic pain model. The majority of these studies only assess males. However, despite these shortcomings, the outcomes of the studies are consistent and, therefore, support our proposed in vivo efficacy assessment of drug for the treatment of chronic pain. Furthermore, because REST activity is highly conserved in mammals, we believe these studies provide strong proof-of-concept support to test modulation of REST in humans.

We discovered that REST is targeted for degradation through phosphorylation of its serines 861 and 864. REST is stabilized by dephosphorylation of these sites by the C-terminal domain small phosphatase 1 (CTDSP1) 2-4. Like REST, CTDSP1 is expressed in nonneuronal tissues where it contributes to neural gene silencing via its interaction with REST. We concluded that inhibition of CTDSP1 would be sufficient to promote REST degradation and stimulate gene expression. In support of this conclusion, we have demonstrated that knockdown of CTDSP1 in mesenchymal progenitor cells (MPCs) and nerves causes expression of REST target genes and axonal regeneration⁷⁶. We leveraged our mechanistic understanding of the REST-CTDSP1 interaction to develop a cyclic phosphomimetic peptide encompassing the relevant regulatory region of REST⁷⁷. The REST phosphomimetic peptide (RPP) has several pharmacologic attributes. It is highly stable due to its cyclic structure and D-amino acid composition (e.g., SEQ ID NO: 9, 12, 13, 14, 16, 17, and 68-117). It has low pM binding affinity exceeding that of most antibodies (FIGS. 2 & 3), suggesting a low risk of off-target effects. Its small size (<3.5 kD) indicates it should have bioavailability approaching that of a small molecule drug. Our preliminary data shows that our mimetic inhibits the activity of CTDSP1, reduces REST protein levels, and results in the expression of neural genes needed for proper neuronal activity⁷⁷.

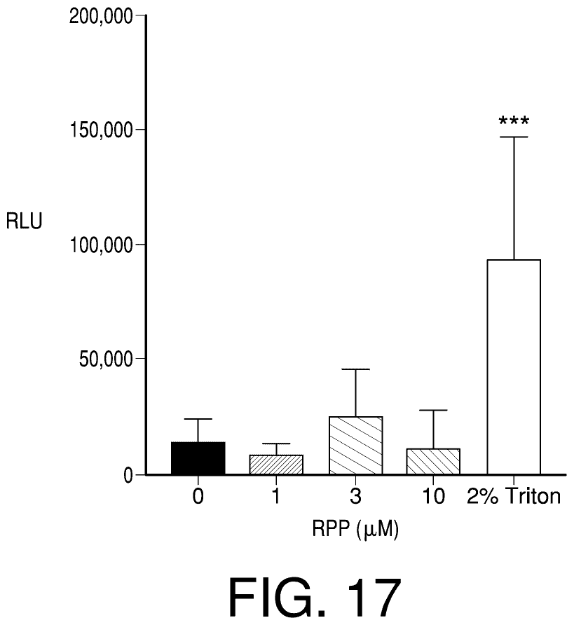

Most importantly, our approach is predicted to be safe and effective for the following reasons: 1) Our drug is selective for nuclear targets primed for regenerative response, such as after nerve injury. The basal organization of chromatin does not permit the epigenetic “writing” of a new set of instructions; nerve injury however, triggers a concerted, widespread, and transient nucleosome reorganization, a Genomic Transient Intermediate State (GTIS): a temporary nucleosomal architecture favorable for epigenetic reprograming for the necessary response⁷⁸⁻⁸¹. 2) Our drug has very high binding affinity (low pM) and therefor will not have off target affects (FIGS. 2&3). 3) We are proposing a brief treatment period of less than 1 month duration. The basis for our short treatment period is that REST is the gate keeper for terminal differentiation of neurons^{64,82}, a process which irreversibly commits a cell to that lineage²³. Neural progenitors, which have epigenetic similarities to neurons in a chronic pain state⁶³, because they are also in a GTIS, terminally differentiate after a few days of induction. Consistently, we have shown the RPP can induce neuronal differentiation (measured by MAP2 positive cells, a marker of neuronal differentiation) within 7 days (FIGS. 11, 12, and 13). Once the process has been started and the cell has been committed, there is no need for further treatment. Therefore, we expect the pain relief achieved through this early treatment period to be durable. 4) We have shown our drug is not toxic to neurons in an ex vivo DRG neuron toxicity assay measuring necrosis (FIG. 17). 5) REST and CTDSP1 are primarily involved in neurodevelopment, therefore, we predict inhibiting their activity in injured, adult cells will be well tolerated. 6) The safety of peptide drugs has made them an attractive therapeutic strategy. Currently, there are more than 68 marketed peptide drugs with global sales exceeding $14.7 billion, and 140 are in clinical development^{84,85}.

### Innovation

First-in-class drug candidate. We have developed the first drug to target a transcriptional repressor of neuronal genes, REST. The published literature show that REST expression contributes to chronic pain (Table 12). After PNI, REST and CTDSP1 expression is increased in both the peripheral (FIG. 18) and central nervous systems resulting in dysfunction⁷⁻⁹ Several published studies in rodents have shown that blocking REST alleviates chronic pain^{60,72-74}.

Innovative drug design. We have developed a cyclic phosphomimetic cell penetrating peptide that destabilizes REST by inhibiting CTDSP1³, the phosphatase that protects REST from degradation, allowing for the expression of neural-specific genes. Like REST, expression of CTDSP1 is restricted to nonneuronal cell types, except after neuronal injury. Our inhibitor is novel in that it directly targets a transcriptional checkpoint that immediately regulates the genes necessary to induce regeneration. It overcomes challenges to targeting transcription factors and to creating therapeutics against serine phosphatases. Regarding the latter, other approaches have focused on phosphoprotein phosphatases (PPPs), and the metal-dependent protein phosphatases (PPMs)⁸⁶. However, CTDSP1 is a haloacid dehalogenase (HAD) which uses two catalytic aspartic acids instead of relying solely on a metal ion for activity⁸⁷. This catalytic site is amenable to specific inhibition as demonstrated by the success in developing therapeutics against similar enzymes, such as HIV-1 protease⁸⁸⁻⁹¹. Finally, CTDSP1 has a proline dependent substrate preference, not found in other serine phosphatases, which we have exploited to improve the binding and stability of our drug.

PRELIMINARY DATA. REST represses gene expression by binding to the chromatin at repressor element-1 (RE-1) sites near the regulatory regions of neuronal genes, including ion channels, growth factors, and axonal-guidance proteins¹. Therefore, before stem cells, such as neural progenitor cells (NPCs), can terminally differentiate, they must first target REST for degradation to express the required neuronal genes⁶⁴. REST is protected from degradation by CTDSP1, which is both necessary and sufficient to prevent neuronal gene expression². Dominant-negative CTDSP1, which can bind phosphorylated targets but cannot catalyze dephosphorylation, induces terminal differentiation of P19 stem cells⁹².

We have previously shown that the interaction between CTDSP1 and REST is dependent on phosphorylation of REST serines 861 and 864 by the MAP kinase, ERK2, and that mutation of these serines to alanine increases REST stability²⁻⁴. We surmised that a non-hydrolysable phosphomimetic of this REST regulatory region could slow down CTDSP1 activity and promote REST degradation. To test our hypothesis, we developed a REST Phosphomimetic Peptide or RPP⁷⁷ to assess dose-dependent effects on REST and REST-targets. The RPP contains amino acids 858 to 870 from REST with serine 861 and 864 mutated to glutamates, which can mimic both the shape and overall charge of phosphoserine. To the C-terminus, we fused an arginine-rich cell-penetrating peptide and endosomal exit sequence derived from HIV-Tat and HA2, respectively, which are used to deliver peptides and proteins into cultured mammalian cells and live organisms and facilitate crossing of the blood-brain barrier (BBB)⁹³⁻⁹⁶.

We investigated if RPP could decrease REST protein (FIG. 6). In the left panel, the Western blot (WB) shows that RPP decreased REST (exogenous+endogenous) levels in HEK cells. In the right panel, quantification of total REST protein (exogenous+endogenous) levels by WB analysis shows an overall decrease of 58.3% in the RPP dosed HEK cells (FIG. 6).

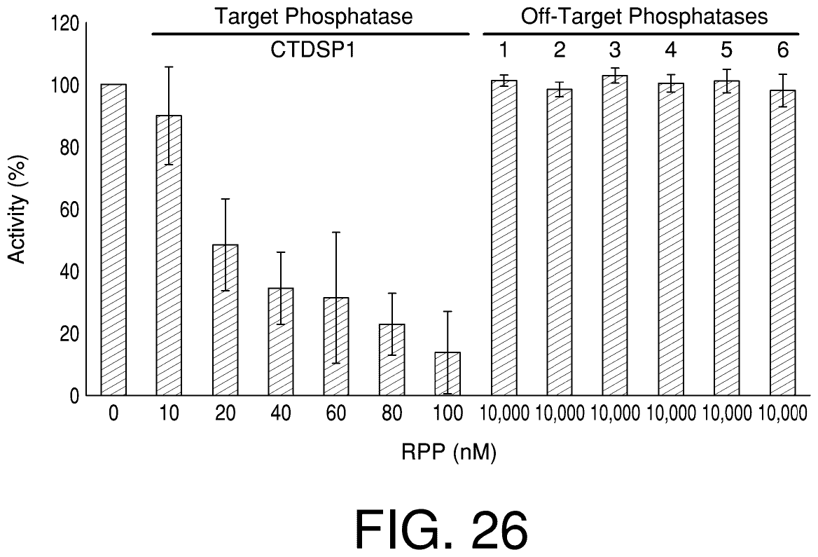

In an in vitro binding assay, RPP had low pM affinity for CTDSP1, with a slow off rate (FIGS. 2 &3). In an in vitro assay of phosphatase inhibition, RPP inhibited its target, CTDSP1, at low nM concentrations, but did not inhibit off target PP5, PP1, PPM1H, PPM1A, or PP3CA activity at 10 μM (FIG. 26). In a cell permeability and stability assay in MPCs, RPP translocated into the nucleus (FIG. 4 and FIG. 5), which is consistent with the localization profile of both REST and CTDSP1^{92,97}. RPP levels remained stable after 6 days in culture.

It has been established by that blocking REST in rodent chronic pain models, prevents downregulation of Nav1.8, Kv4.3, Kv7.2, and Oprm1 which are implicated as the cause of hyperalgesia and allodynia (Table 12). Based on these studies, we tested whether blocking CTDSP1 could stimulate the expression of REST target genes associated with chronic pain. We have also shown that NBFL cells transfected with RPP (without HIVTAT-HA2) or dosed with linear or cyclic RPP evoke an increase (2-fold and up to 4-fold, respectively) in Kv4.3 mRNA (FIGS. 8&9c).

We found that 3 μM RPP (SEQ ID NOS: 5-11) increased human neural progenitor cell (iPSC) differentiation by 2- to 2.7-times as measured by MAP2 (mature neuron marker) expression normalized to DAPI (nucleus), compared to control (FIG. 11), and 1 μM RPP (SEQ ID NO: 9 and 13) increased human neural progenitor cell (iPSC) differentiation as measured by TUJ1 (up to 36% and 3-times respectively) and MAP2 (up to 33% and 2-times respectively) (neuronal markers) expression normalized to DAPI (nucleus) (FIGS. 12 and 13). There was no increase in cell death compared to control (data not shown). The basis for using this screen is that eliminating REST in neural progenitor cells has been shown to induce neuronal differentiation²², which is a readout for ensemble derepression of neuronal genes.

In a preliminary assessment of efficacy and toxicity, RPP was assessed at 0, 1, 3, or 10 μM for 48 hours in an ex vivo culture of whole DRG neurons for its potential to induce expression of Kv4.3 (FIG. 14), Kv7.2 (FIG. 14), Nav1.8 (FIGS. 14 and 15) and OPRM1 (FIG. 14) and cause neurotoxicity (FIG. 17). At 3 μM SEQ ID NO: 12 induced Kv4.3, Kv7.2, Nav1.8, and OPRM1 approximately 0-, 6-, 3-, and 3-times, respectively, compared to control (FIG. 14). At 3 μM SEQ ID NOS: 13 and 14 induced Nav1.8 approximately 6- and 13-times, respectively (FIG. 15). At 10 μM SEQ ID NO: 12 induced Kv4.3, Kv7.2, Nav1.8, and OPRM1 approximately 2-, 7-, 5-, and 4-times, respectively (FIG. 14). In a lactate dehydrogenase LDH cytotoxicity assay, RPP sequence ID NO: 12 showed no toxicity at 0, 1, 3, or 10 μM (FIG. 16). As expected, the positive control (triton, n=6)) was toxic (FIG. 17).

Epilepsy-Epilepsy is uncontrolled electrical activity in the brain which causes confusion, loss of consciousness, and uncontrolled movements⁹⁸. It is the result of dysregulation of ion channels and receptors including SCN1A (Nav1.1), SCN2A (Nav1.2), SCN1B (Nav beta subunit 1), KCNQ2 (Kv7.2), and KCNQ3 (Kv7.3), which are targets of REST repression^{14,99}. The underlying cause of epilepsy is an increase in REST levels in the nucleus of neurons¹⁰⁰.

Diabetes-Pancreatic beta cells and neurons share similar transcriptional pathways during their differentiation program, which involve the elimination of REST¹⁰¹. Down regulation of REST target genes by over expression of REST in beta cells decreases insulin secretion^{102,103}.

Alzheimer's disease—In Alzheimer's disease, acetylcholine and choline acetyltransferase (ChAT), a transferase enzyme necessary for the synthesis of acetylcholine, are very low. The decrease in concentration of this enzymes contributes to the memory and cognition deficits associated with Alzheimer's. Increased REST levels in the brain suppress ChAT¹⁶.

Huntington's disease—In Huntington's disease, the translocation of REST from the cytoplasum to the nucleus of neurons is thought to be the cause of neuronal degeneration associated with the disease¹⁰⁴⁻¹⁰⁶.

Brain cancers, including glioblastoma multiforme—Brain cancers, including glioblastoma multiforme (GBM), originate from brain tumor initiating cells (BTICs), which are cancerous stem cells that display a robust capacity for self-renewal and can become cancerous tumors. These cells are resistant to radiation and chemotherapy and become proliferative months after treatment¹⁰⁷⁻¹⁰⁹. The RPP (SEQ ID NOS: 1 and 15 through 17) or RPPv (SEQ ID NOS: 18 through 117) peptide fused to a CPP (SEQ ID NOS: 118 through 137 and 140 through 159) has the potential to prevent tumors from forming by terminally differentiating BTICs. Once cells have terminally differentiated they can no longer proliferate and seed new tumors¹¹⁰. It is important to state that terminal differentiation is not the same as the differentiation achieved by other methods, such as by targeting chromatin remodeling with inhibitors of histone deacetylases (HDACs). Inhibition of these enzymes alters gene expression indiscriminately and cannot achieve the permanent terminal differentiation that is required to prevent recurrent GBM. Terminal differentiation of BTIC into neurons is blocked at a single checkpoint by REST. The importance of this repressor in maintaining oncogenicity is highlighted by the observation that increased levels of REST correlate with both recurrence of GBM¹¹¹⁻¹¹³ and shorter periods of disease free survival^{112,114}. The reliance of BTICs on REST to maintain their oncogenicity creates an opportunity for therapeutic intervention. REST is targeted for degradation through phosphorylation of serines 861 and 864, and that these serines are kept in a dephosphorylated state by the C-terminal domain small phosphatase, CTDSP1². Therefore, inhibition of CTDSP1 with the RPP (SEQ ID NOS: 1 and 15-17) or RPPv (SEQ ID NOS: 18 through 117) that is fused to a CPP (SEQ ID NOS: 118 through 137 and 140 through 159) should release the REST-brake on terminal differentiation and prevent the recurrence of GBM and other brain tumors.

Pancreatic cancer-REST levels are high in advance stage, metastatic positive pancreatic cancer cells¹¹⁵. Pancreatic cancer patients with high REST levels in their tumors have worse survival rates¹¹⁵. In vitro functional experiments show that knockdown of REST suppressed proliferation, migration, invasion and epithelial-mesenchymal transition of pancreatic cancer cells (AsPC-1 and PANC-1)¹¹⁵. In vivo experiments (a subcutaneous BALB/c nude mouse model and a superior mesenteric vein injection BALB/c nude mouse model) show that knockdown of REST suppressed growth and metastasis of xenografted tumors¹¹⁵. Therefore, inhibiting REST improves patient outcomes for those disorders. Accordingly, the present invention also relates to methods for inhibiting REST in a cell. In general, the method comprises contacting a cell with the RPP (SEQ ID NOS: 1 and 15 through 17) or RPPv (SEQ ID NOS: 18 through 117) peptide fused to a CPP (SEQ ID NOS: 118 through 137 and 140 through 159). For example, it can be exposing a cell for a sufficient amount of time for the RPP− (SEQ ID NOS: 1 and 15 through 17) or RPPv (SEQ ID NOS: 18 through 117) fused to a CPP (SEQ ID NOS: 118 through 137 and 140 through 159) to enter the cell and have an effect on REST activity. The method can be practiced either in vitro or in vivo. Where practiced in vitro, the method can be used to study the activity of REST, to test other compounds for the ability to supplement or antagonize the effects of the RPP− (SEQ ID NOS: 1 and 15 through 17) or RPPv (SEQ ID NOS: 18 through 117) fused to a CPP (SEQ ID NOS: 118 through 137 and 140 through 159) on REST, or for any other reason of importance to a researcher. When practiced in vivo, the method can be used as a method of treating a subject for one or more diseases or disorders associated with REST. According to the method of this aspect of the invention, preferably, activity of REST is decreased. The step of contacting a cell can be any action that causes the agent to physically contact one or more target cells. Thus, it can be by way of adding the agent directly to an in vitro culture of cells to be contacted, and allowing the agent sufficient time to diffuse through the media and contact at least one cell. Likewise, it can be through addition of the agent to cells in an aqueous environment. Alternatively, it can be by way of administering the agent to a subject via any acceptable administration route, and allowing the body of the subject to distribute the agent to the target cell through natural processes. Thus, the in vivo methods can be methods of localized or systemic delivery of the agent to a cell in animals, including all mammals and humans in particular. According to this aspect, the RPP (SEQ ID NOS: 1 and 15 through 17) or RPPv (SEQ ID NOS: 18 through 117) fused to a CPP (SEQ ID NOS: 118 through 137 and 140 through 159) can be used to treat a subject therapeutically or prophylactically, and to prepare a composition for use in treating.

In yet another embodiment, the invention provides a method of treating a subject suffering from or at risk of suffering from a disease or disorder involving REST. In general, the method comprises administering the RPP (SEQ ID NOS: 1 and 15 through 17) or RPPv (SEQ ID NOS: 18 through 117) fused to a CPP (SEQ ID NOS: 118 through 137 and 140 through 159), in an amount sufficient to affect the amount or activity of REST in the subject. In certain aspects, the binding of the RPP (SEQ ID NOS: 1 and 15 through 17) or RPPv (SEQ ID NOS: 18 through 117) fused to a CPP (SEQ ID NOS: 118 through 137 and 140 through 159) to CTDSP1 results in inhibition of REST activity in a cell. In general, the method comprises administering a sufficient amount of the RPP (SEQ ID NOS: 1 and 15 through 17) or RPPv (SEQ ID NOS: 18 through 117) fused to a CPP (SEQ ID NOS: 118 through 137 and 140 through 159) for a sufficient time to inhibit REST activity. Often, the amount administered and the amount of time is adequate to see a change in one or more clinical symptoms of a disease or disorder, or to stop progression of a disease or disorder from reaching a stage where one or more clinical symptoms are seen. According to this aspect, agent can be used to treat a subject therapeutically or prophylactically, and to prepare a composition for use in treating.

In one embodiment, the present invention provides methods for treating, alleviating, or ameliorating traumatic brain injury, chronic pain, peripheral nerve injury, epilepsy, diabetes, Alzheimer's disease, Huntington's disease, brain tumors (including glioblastoma multiforme), or pancreatic cancer in a subject. The terms “treating” or “alleviating” or “ameliorating” and similar terms used herein, include prophylaxis and full or partial treatment. The terms may also include reducing symptoms, ameliorating symptoms, reducing the severity of symptoms, reducing the incidence of the disease, or any other change in the condition of the patient, which improves the therapeutic outcome. The methods involve administering to a subject suffering from traumatic brain injury, chronic pain, peripheral nerve injury, epilepsy, diabetes, Alzheimer's disease, Huntington's disease, brain tumors (including glioblastoma multiforme), or pancreatic cancer in an animal, or a subject in need of treatment for traumatic brain injury, chronic pain, peripheral nerve injury, epilepsy, diabetes, Alzheimer's disease, Huntington's disease, brain tumors (including glioblastoma multiforme), or pancreatic cancer in an animal, the RPP (SEQ ID NOS: 1 and 15 through 17) or RPPv (SEQ ID NOS: 18 through 117) fused to a CPP (SEQ ID NOS: 118 through 137 and 140 through 159).

The administration of the RPP (SEQ ID NOS: 1 and 15 through 17) or RPPv (SEQ ID NOS: 18 through 117) fused to a CPP (SEQ ID NOS: 118 through 137 and 140 through 159) to the subject can be through any known and acceptable route. Such routes include, but are not necessarily limited to, oral, via a mucosal membrane (e.g., nasally, via inhalation, rectally, intrauterally or intravaginally, sublingually), intravenously (e.g., intravenous bolus injection, intravenous infusion), intraperitoneally, and subcutaneously. Administering can likewise be by direct injection to a site (e.g., organ, tissue) containing a target cell (i.e., a cell to be treated). Furthermore, administering can follow any number of regimens. It thus can comprise a single dose or dosing of the drug, or multiple doses or dosings over a period of time. Accordingly, treatment can involve repeating the administering step one or more times until a desired result is achieved. In embodiments, treating can continue for extended periods of time, such as weeks, months, or years. Those of skill in the art are fully capable of easily developing suitable dosing regimens for individuals based on known parameters in the art. The methods thus also contemplate controlling, but not necessarily eliminating, the disease or disorder. The preferred routes of administration in accordance with the present invention are oral and via a mucosal membrane.

The amount to be administered varies depending on the subject, stage of the disease, age of the subject, general health of the subject, and various other parameters known and routinely taken into consideration by those of skill in the medical arts. As a general matter, a sufficient amount of the agent will be administered in order to make a detectable change in the symptom of the subject. Suitable amounts are disclosed herein, and additional suitable amounts can be identified by those of skill in the art without undue or excessive experimentation.

The RPP (SEQ ID NOS: 1 and 15 through 17) or RPPv (SEQ ID NOS: 18 through 117) fused to a CPP (SEQ ID NOS: 118 through 137 and 140 through 159) is administered in a form that is acceptable, tolerable, and effective for the subject. Numerous pharmaceutical forms and formulations for biologically active agents are known in the art, and any and all of these are contemplated by the present invention. Thus, for example, the agent can be formulated in oral solution, a caplet, a capsule, an injectable, an infusible, a suppository, a lozenge, a tablet, a cream or salve, an inhalant, and the like.

Those of ordinary skill in the art will readily optimize effective dosages and administration regimens as determined by good medical practice and the clinical condition of the individual subject.

The frequency of dosing will depend on the pharmacokinetic parameters of the compounds and the routes of administration. The optimal pharmaceutical formulation will be determined by one of skill in the art depending on the route of administration and the desired dosage. Such formulations may influence the physical state, stability, rate of in vivo release and rate of in vivo clearance of the administered agents. Depending on the route of administration, a suitable dose is calculated according to body weight, body surface areas or organ size. The availability of animal models is particularly useful in facilitating a determination of appropriate dosages of a given therapeutic. Further refinement of the calculations necessary to determine the appropriate treatment dose is routinely made by those of ordinary skill in the art without undue experimentation, especially in light of the dosage information and assays disclosed herein as well as the pharmacokinetic data observed in animals or human clinical trials.

Typically, appropriate dosages are ascertained through the use of established assays for determining blood levels in conjunction with relevant dose response data. The final dosage regimen will be determined by the attending physician, considering factors which modify the action of drugs, e.g., the drug's specific activity, severity of the damage and the responsiveness of the patient, the age, condition, body weight, sex and diet of the patient, the severity of any infection, time of administration and other clinical factors. As studies are conducted, further information will emerge regarding appropriate dosage levels and duration of treatment for specific diseases and conditions. Those studies, however, are routine and within the level of skilled persons in the art. Typical dosage may be about 0.6 mg (0.01 mg/kg) to about 60 g (1 g/kg) weekly (in human), more preferably monthly.

It will be appreciated that the peptides, compositions and treatment methods of the invention are useful in fields of human medicine and veterinary medicine. Thus, the subject to be treated is a mammal, such as a human or other mammalian animal. For veterinary purposes, subjects include for example, farm animals including cows, sheep, pigs, horses and goats, companion animals such as dogs and cats, exotic and/or zoo animals, and laboratory animals including mice, rats, rabbits, guinea pigs and hamsters.

The RPP (SEQ ID NOS: 1 and 15 through 17) or RPPv (SEQ ID NOS: 18 through 117) fused to a CPP (SEQ ID NOS: 118 through 137 and 140 through 159) may be administered to a subject animal, preferably mammals, such as humans, in need thereof as a pharmaceutical or veterinary composition, such as tablets, capsules, solutions, or emulsions. The RPP (SEQ ID NOS: 1 and 15 through 17) or RPPv (SEQ ID NOS: 18 through 117) fused to a CPP (SEQ ID NOS: 118 through 137 and 140 through 159) can be present in compositions containing other ingredients. Non-limiting examples of compositions appropriate for the present invention are pharmaceutical compositions, such as in the form of tablets, pills, capsules, caplets, multiparticulates (including granules, beads, pellets and micro-encapsulated particles); powders, elixirs, syrups, suspensions, and solutions. Pharmaceutical compositions will typically comprise a pharmaceutically acceptable diluent or carrier. Pharmaceutical compositions are preferably adapted for administration parenterally (e.g., orally). Orally administrable compositions may be in solid or liquid form and may take the form of tablets, powders, suspensions, and syrups, among other things. Optionally, the compositions comprise one or more flavoring and/or coloring agents. In general, therapeutic and nutritional compositions may comprise any substance that does not significantly interfere with the action of the agent on the subject.

Pharmaceutically acceptable excipients or carriers suitable for use in such compositions are well known in the art of pharmacy. The compositions of the invention may contain 0.01-99% by weight of the agent. The compositions of the invention are generally prepared in unit dosage form. The excipients used in the preparation of these compositions are well-known in the art.

Further examples of product forms for the composition are food supplements, such as in the form of a soft gel or a hard capsule comprising an encapsulating material selected from the group consisting of gelatin, starch, modified starch, starch derivatives such as glucose, sucrose, lactose, and fructose. The encapsulating material may optionally contain cross-linking or polymerizing agents, stabilizers, antioxidants, light absorbing agents for protecting light-sensitive fills, preservatives, and the like.

In general, the term carrier may be used throughout this application to represent a composition with which the agent may be mixed, be it a pharmaceutical carrier, foodstuff, nutritional supplement or dietary aid. The materials described above may be considered carriers of the agent for the purposes of the invention. In certain embodiments of the invention, the carrier has little to no biological activity, particularly on REST.

Without further description, it is believed that one of ordinary skill in the art can, using the preceding description and the following illustrative examples, make and utilize the compounds of the present invention and practice the claimed methods. The following examples are given to illustrate the present invention. It should be understood that the invention is not to be limited to the specific conditions or details described in the examples.

### Example 1—his-CTDSP1 Plasmid Construction

The codon optimized (using IDT codon optimization tool) CTDSP1 gene was cloned into the pBAD-HisA plasmid (Thermo Fisher Scientific). First, pBAD-HisA plasmid was amplified with primers P33 and P34 (Table 1) introducing HindIII and XhoI restriction sites. The PCR reaction (20 μL) was initially heated at 95° C. for 2.5 min followed by 30 cycles of denaturation at 94° C. for 15 sec., annealing at 55° C. for 15 sec. and extension at 72° C. for 6 min. Following amplification, the PCR fragment was gel-purified by the QIAGEN gel-band purification kit and digested with HindIII and XhoI restriction enzymes. All digested fragments were purified with a QIAGEN kit and ligated in appropriate combination using T4 DNA ligase (NEB) according to recommendations of the manufacturer.

TABLE 1
Primers used for plasmid construction

| Primers | | SEQ |
|---|---|---|
| Name | Sequence | ID NO: |
| P12 | AAATAAGCTTGTATATCTCCTTCTTAAAGTTAAACA | 269 |
| P14 | AATAACTCGAGAGATCCGGCTGCTAACAAAGC | 270 |
| P17 | ATAAGCTTTAATACGACTCACTATAGGGTTAACTTTAGTAAGGAGGACAGCT | 271 |
| | AA | |
| P19 | GTGGTGATGGTGATGGTGACCTA | 272 |
| P20 | ATTATTCTCGAGTTAATAGCCGGTGCCGTGGTGATGGTGATGGTGACCTA | 273 |
| P21 | AAATACTCGAGTCGTTTTATCTGTTGTTTGTCGGT | 274 |
| P22 | AAATAAAGCTTCTCTGAATGGCGGGAGTATGAAAA | 275 |
| P23 | CCGCGAATGGTGAGATTGAGAA | 276 |
| P24 | ACGCAAAAAGGCCATCCGTCAG | 277 |
| P28 | ATTATTCTCGAGTTAATAGCCGGTGCCTAAGCCGCTACCACCACGCCGACG | 278 |
| | CTGACGG | |
| P31 | AAATAAGCTTATGGATAGTAGTGCGGTGATCA | 279 |
| P32 | ATTATTCTCGAGCTAGTGGTGATGGTGATGGT | 280 |
| P33 | AACTAAGCTTTTCCTCCTGTTAGCCCAAAAAAC | 281 |
| P34 | AATACTCGAGGCTGTTTTGGCGGATGAGAGAA | 282 |
| P35 | AAATCCATGGATAGTAGTGCGGTGATCA | 283 |
| P36 | AAATCATATGGATAGTAGTGCGGTGATCA | 284 |
| P37 | AATACTCGAGCTAGTGGTGATGGTGATGGTGAG | 285 |
| P38 | AATACTCGAGAGAGCCAGAACCAGATCCCGGA | 286 |
| P39 | TAAGCCGCTACCACCACGCCGAC | 287 |
| P40 | GGTGCAATTCCTAAAACTCCAGTACAATACTTACTGC | 288 |
| P41 | GTATTGTACTGGAGTTTTAGGAATTGCACCATTTTCCTC | 289 |
| P108 | AATACTCGAGTTATTTTGGAGGATGGTCGCCACCA | 290 |
| P109 | AATAAAGCTTATGGGATCCGGTTCTGGCTCAGGTTCTTCC | 291 |
| Degenerate Primers | | |
| Name | Sequence | |
| 11F | NAANCANCANTGNCANAGNAANATTGTGGTTCTGGCTCTGGTCGTAAAA | 292 |
| 12F | GNACNACNACNGCNAANGGNAANTTGTGGTTCTGGCTCTGGTCGTAAAA | 293 |
| 13F | GNANCACNANTGCNANAGGNANATTGTGGTTCTGGCTCTGGTCGTAAAA | 294 |
| 14F | NAACNANCACNGNCAANGNAAANTTGTGGTTCTGGCTCTGGTCGTAAAA | 295 |
| 15F | GAANCANCANTGNCANAGNAANATTGTGGTTCTGGCTCTGGTCGTAAAA | 296 |
| 16F | GAACNACNACNGCNAANGGNAANTTGTGGTTCTGGCTCTGGTCGTAAAA | 297 |
| 17F | GAANCACNANTGCNANAGGNANATTGTGGTTCTGGCTCTGGTCGTAAAA | 298 |
| 18F | GAACNANCACNGNCAANGNAAANTTGTGGTTCTGGCTCTGGTCGTAAAA | 299 |
| 19F | GAANCANCACTGCCANAGNAAAATTGTGGTTCTGGCTCTGGTCGTAAAA | 300 |
| 20F | NAACCACCANTGNCAAAGGAANATTGTGGTTCTGGCTCTGGTCGTAAAA | 301 |
| 21F | GAANCACNACTGCCANAGGNAAATTGTGGTTCTGGCTCTGGTCGTAAAA | 302 |
| 22F | GNACCACCANTGCNAAAGGAANATTGTGGTTCTGGCTCTGGTCGTAAAA | 303 |
| 11R | TGNTGNTTNCANATNTTNGGNACACATTTAGCTGTCCTCCTTACTAAAGTT | 304 |
| 12R | TNGTNGTNCCNGANCTNCGNTACACATTTAGCTGTCCTCCTTACTAAAGTT | 305 |
| 13R | TGNTNGTTNCNGATNTNCGGNACACATTTAGCTGTCCTCCTTACTAAAGTT | 306 |
| 14R | TNGTGNTNCCANANCTTNGNTACACATTTAGCTGTCCTCCTTACTAAAGTT | 307 |
| 15R | TGNTGNTTCCANATNTTNGGNACACATTTAGCTGTCCTCCTTACTAAAGTT | 308 |
| 16R | TNGTNGTTCCNGANCTNCGNTACACATTTAGCTGTCCTCCTTACTAAAGTT | 309 |
| 17R | TGNTNGTTCCNGATNTNCGGNACACATTTAGCTGTCCTCCTTACTAAAGTT | 310 |
| 18R | TNGTGNTTCCANANCTTNGNTACACATTTAGCTGTCCTCCTTACTAAAGTT | 311 |
| 19R | TGGTGNTTNCAGATCTTNGGNACACATTTAGCTGTCCTCCTTACTAAAGTT | 312 |
| 20R | TGNTGGTTCCANATNTTCGGTACACATTTAGCTGTCCTCCTTACTAAAGTT | 313 |
| 21R | TGGTNGTTNCAGATCTNCGGNACACATTTAGCTGTCCTCCTTACTAAAGTT | 314 |
| 22R | TGNTGGTTCCNGATNTTCGGTACACATTTAGCTGTCCTCCTTACTAAAGTT | 315 |

N = any nucleotide

Ligated fragments were transformed in 10G chemically competent cells (Lucigen) according to manufacturer's protocol. Transformed cells were plated on LB plates containing 50 μg/mL ampicillin and incubated overnight at 37° C. Colonies were tested for the presence of the insert by colony PCR. Colonies were picked and resuspended in 20 μl of sterile 0.9% sodium chloride solution. One μl of this solution was transferred to the PCR tube and amplified with Taq polymerase (New England Biolabs, cat #M0482S) and 30 PM of the flanking primers. Each PCR reaction (20 μL) was initially heated to 95° C. for 2.5 min followed by 30 cycles of denaturation at 94° C. for 15 sec., annealed at 55° C. for 15 sec., and extended at 72° C. for 1 min. Amplification products were visualized by agarose electrophoresis. Clones with the correct inserts were inoculated into the culture tubes containing 5 mL of LB with the appropriate antibiotic and incubated overnight at 37° C. Constructs were then purified using the Monarch Plasmid miniprep kit (NEB).

### Example 2—Expression and Purification Methods for his-CTDSP1

His-CTDSP1 (Table 2; SEQ ID NO: 162): E. coli (10G strain Lucigen) with pBAD-CTDSP1 construct were incubated overnight at 37° C. with vigorous shaking. Then 4 mL of the night culture was added to 500 mL of LB media with 50 μg/mL ampicillin in a 1 L flask and incubated at 37° C. with shaking. When the culture's OD600 reached 0.4, we added arabinose to the final concentration of 0.02% and incubated 16 hours at 30° C. with shaking. Next morning, cells were spun down (Eppendorf centrifuge 5810R) at maximum speed and frozen at −80° C. When needed, cell pellets were removed from the freezer, incubated at room temperature, lysed with 4 mL of BPER protein lysis reagent (ThermoFischer) and purified protein with HisPur Cobalt Purification kit (ThermoFischer, Cat #90091) as described by the manufacturer.

TABLE 2
CTDSP1 Nucleotide and Protein Sequences

Nucleotide Sequence (SEQ ID NO: 162)
ATGGATAGTAGTGCGGTGATCACACAAATCTCCAAGGAGGAAGCCCGTGG
GCCGCTGCGGGGGAAGGGTGATCAAAAATCGGCAGCTAGTCAAAAACCTC
GCTCTCGTGGGATACTTCATTCGCTGTTTTGCTGCGTCTGCCGCGATGAC
GGAGAAGCATTGCCTGCGCATTCAGGGGCGCCTTTACTTGTTGAGGAAAA
TGGTGCAATTCCTAAACAAACTCCAGTACAATACTTACTGCCGGAGGCAA
AGGCACAAGACAGTGATAAGATATGTGTAGTAATAGACTTAGATGAAACA
CTGGTACATTCGTCATTCAAACCTGTTAATAATGCGGATTTCATCATACC
TGTAGAAATCGACGGGGTTGTCCATCAGGTTTACGTCCTGAAGCGGCCTC
ATGTAGATGAATTTTTACAGCGGATGGGCGAGTTATTTGAATGTGTGCTG
TTTACAGCTAGTCTTGCCAAGTACGCGGATCCTGTCGCGGATTTGCTTGA
TAAGTGGGGTGCGTTTCGGGCGAGATTATTTCGCGAATCTTGCGTTTTTC
ACAGAGGTAACTACGTGAAGGACCTTAGTCGTCTGGGTAGAGATCTTAGA
AGAGTGCTGATCCTTGACAACAGCCCAGCCAGCTATGTCTTTCATCCGGA
TAACGCAGTACCCGTGGCGTCTTGGTTCGACAATATGTCGGACACGGAGC
TGCATGACCTGTTGCCGTTCTTTGAGCAGTTGAGTCGCGTTGATGACGTT
TACTCGGTTTTGCGTCAACCCCGTCCGGGATCTGGTTCTGGCTCTCACCA
TCACCATCACCACTAG
Protein Sequence (SEQ ID NO: 163)
MDSSAVITQISKEEARGPLRGKGDQKSAASQKPRSRGILHSLFCCVCRDD
GEALPAHSGAPLLVEENGAIPKQTPVQYLLPEAKAQDSDKICVVIDLDET
LVHSSFKPVNNADFIIPVEIDGVVHQVYVLKRPHVDEFLQRMGELFECVL
FTASLAKYADPVADLLDKWGAFRARLFRESCVFHRGNYVKDLSRLGRDLR
RVLILDNSPASYVFHPDNAVPVASWFDNMSDTELHDLLPFFEQLSRVDDV
YSVLRQPRPGSGSGSHHHHHH
Linker and His tag are underlined

### Example 3—Developing Peptides with High Affinity to CTDSP1

Peptide candidates were generated using RNA display and protein evolution methods.

Construction of RNA display libraries. We've built 3 different libraries of RPP variants. In all libraries serines 861 and 864 were replaced by glutamates. Library 1 was built using primers P11 through P14 (Table 1). In library 1 each position of the REST peptide was mutated. It was estimated to have 1×10⁹ variants. In library 2 (primers P14 through P18, Table 1), glutamates were not touched by the degenerate oligos. This library was expected to have 65×10⁶ variants. In library 3, only one of the glutamates and every second codon was mutated. This library was expected to generate only 65000 variants.

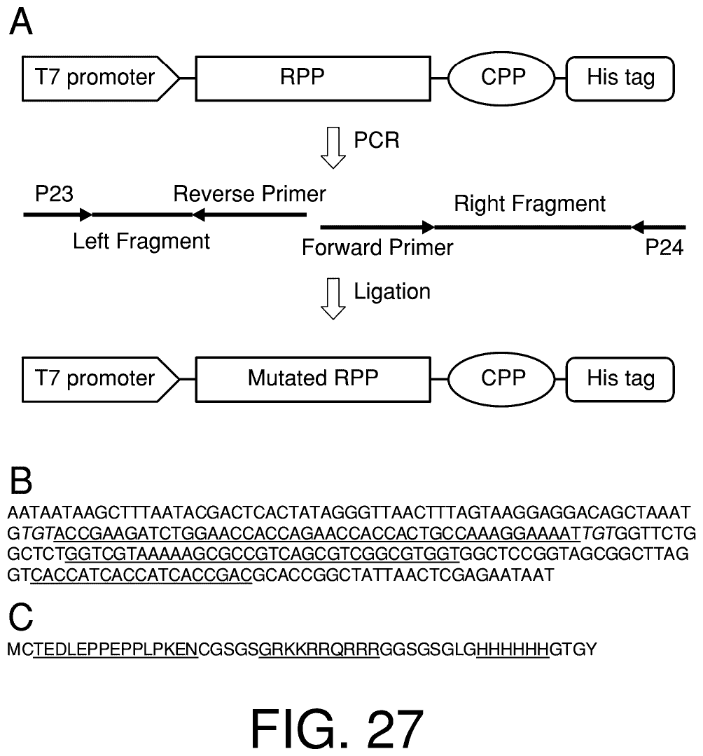

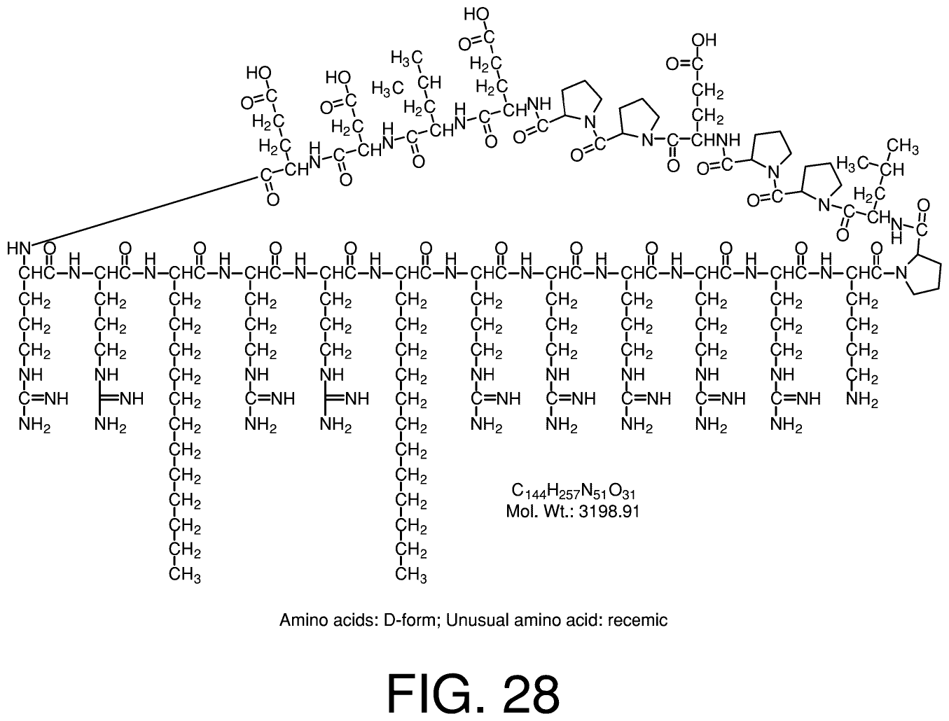

In all 3 libraries, the RPP sequence (SEQ. ID NO: 1) was diversified by synthetic degenerate oligoes. Only one degenerate base was introduced per codon: either first or the second position which gave a choice or 4 amino acids. REST cassettes were amplified from pBAD constructs as two fragments: left and right, which were reunited by ligation (FIG. 27). The left fragment was amplified with flanking forward primer P23 (Table 1) and one of the reverse primers (11R trough 22R, Table 1).

The right fragment was amplified with one of the forward primers (11F through 22F, Table 1) and the reverse primer P24 (Table 1). The PCR reaction (20 μL) was initially heated at 95° C. for 2.5 min followed by 30 cycles of denaturation at 94° C. for 15 sec., annealing at 55° C. for 15 sec. and extension at 72° C. for 40 sec. Following amplification, PCR fragments were gel-purified by the QIAGEN gel-band purification kit, mixed and ligated with T4 DNA ligase. The ligation reaction contained 20 μl of 10× ligation buffer, 100 ng of fragment mix, 0.5 μl of 100 mM ATP, 1 μl of T4 DNA ligase (NEB cat #M0202S) and 1 μl of T4 polynucleotide kinase. The reaction mix was incubated at room temperature and used as a template for PCR with flanking primers P17 and P20 (Table 1) using program described above. PCR fragment was gel-purified by the QIAGEN gel-band purification kit and used as a template in the mRNA display experiments.

For quality control purposes, we cloned and sequenced a fraction of this library. An aliquot of this library was digested by HindIII and XhoI restriction enzymes, purified by QIAGEN kit and cloned in pBAD vector as described above for CTDSP1 gene. Individual clones were sequenced by GeneWiz to confirm that REST cassettes were successfully mutated and that majority of clones were ligated without frame-shift mutations.

In vitro transcription. RNA was translated from amplified libraries using RiboMAX Large Scale RNA Production System T7 (Promega, Cat #P1300) according to the manufacturers protocol and purified by RNeasy Mini Kit (Qiagen, Cat #74104)

Ligation of mRNA to DNA linker with puromycin. XL-PSO oligonucleotide was synthesized by IDT. The sequence of the oligonucleotide was: 5′-PsoC6-(uagccggug) 2′-OMc-AAAAAAAAAAAAAAA-Spacer9-Spaser9-ACC-Puro-3′ (SEQ ID NO: 330). For ligating this oligonucleotide to mRNA we mixed the following reagents in a PCR tube: 29.5 μl of RNAse-free water. 1 μl of 1 M HEPES-KOH, pH 7.6, 5 μl of 1 M KCl, 2 μl of 25 mM spermidine, 0.5 μl of 125 mM EDTA, 8 μl of mRNA from previous step and 4 μl of 100 mM of XL-PSO oligonucleotide. PCR tube was placed in the PCR machine, heated to 70° C. for 5 min and cooled to 25° C. at 0.1° C./s speed. Then we transferred the mixture to the 96-well plate on ice, put the 365-nm handheld UV lamp on top and irradiated the plate for 20 min. After that cross-linked RNA was purified by RNeasy Mini Kit (Qiagen, Cat #74104).

In vitro translation. Translation was performed using PUREexpress in vitro Protein Synthesis Kit (NEB, Cat #E6800S). We mixed the following reagents in a 1.5 ml tube: 20 μl of Solution A, 15 μl of solution B, 0.5 μl of RNAsin Plus, 4.5 μl of water and 10 μl of cross-lined RNA (1 μg/μl). The mixture was incubated at 37° C. for 2 h.

Purification of Peptides with His-tag. We purified RNA-peptide complexes using Ni-NTA magnetic beads (Qiagen, Cat #36111). 100 μl of beads was washed with 300 μl of wash buffer (50 mM NaH₂PO₄, 300 mM NaCl, 10 mM imidazole, 0.005% Tween 20), separated on the magnetic stand and suspended in 300 μl of wash buffer. 25 μl of RNA-peptide complexes from previous step was added to the washed beads and incubated on the end-over-end shaker for 30 min at room temperature. Beads were washed 3 times with the wash buffer followed by separation on the magnetic stand and eluted with 50 μl of the elution buffer (50 mM NaH₂PO₄, 300 mM NaCl, 500 mM imidazole, 0.005% Tween 20).

Purification with Oligo-d(T)₂₅ (SEQ ID NO: 331) magnetic beads. Oligo-d(T)₂₅ (SEQ ID NO: 331) magnetic beads were purchased from New England Biolabs (Cat #S1419S). 100 μl of the bead suspension was washed with 500 μl of wash buffer I (20 mM Tris-HCl, pH 7.5, 500 mM NaCl, 1 mM EDTA) by spending them in the buffer and separating on the magnetic stand followed by suspension in 50 μl of wash buffer I. 50 μl of the RNA-peptide complexes from the previous step were mixed with 50 μl of binding buffer (100 mM Tris-HCl, pH 7.5, 1 M NaCl, 2 mM EDTA), heated at 65° C. for 2 min, placed on ice for 1 min and mixed with the washed beads. This mixture was incubated at room temperature for 5 min, then washed twice with 500 μl of wash buffer I (20 mM Tris-HCl, pH 7.8, 500 mM NaCl, 1 mM EDTA) and once with 500 μl of wash buffer II (20 mM Tris-HCl, pH 7.8, 200 mM NaCl, 1 mM EDTA).

Peptide Cyclization. RNA-peptide complexes bound by the Oligo-d(T)₂₅ (SEQ ID NO: 331) magnetic beads were incubated for 30 min in the cyclization buffer (20 mM Tris-HCl, pH 7.8, 0.00 M NaCl, 3 mM α,α″-dibromo-m-xylene (Sigma-Aldrich), 33% acetonitrile) with periodic shaking. Following incubation, beads were washed with the washing buffer III (20 mM Tris-HCl, pH 7.8, 0.3 M NaCl, 5 mM 2-mercaptoethanol), then with washing buffer IV (20 mM Tris-HCl, pH 7.8, 0.3 M NaCl, 0.5 mM TCEP) followed by washes with wash buffer I (20 mM Tris-HCl, pH 7.8, 500 mM NaCl, 1 mM EDTA) and wash buffer II (20 mM Tris-HCl, pH 7.8, 200 mM NaCl, 1 mM EDTA). Purified products were eluted from the beads by adding 30 ul of elution buffer (20 mM Tris-HCl, pH 7.8) and incubating at 65° C. for 2 min followed by immediate separation on the magnetic bead stand.

Affinity Selection. Affinity selection was performed in NUNC Maxisorp plates (Thermo Fischer Scientific). His-CTDSP1 (Table 2; SEQ ID NOS: 163) was dissolved in 100 μl of PBS, transferred to Maxisorp plate well and incubated on orbital shaker for 2 h at room temperature. Plate wells were washed twice with PBS and blocked with casein (PBSC buffer or PBS with 1% casein) at room temperature for 1 h with shaking and then washed 3 times with PBS. Negative selection wells were coated with casein only by incubating with 300 μl of the PBSC solution and washed 3 times with PBS. Purified RNA-peptide complexes were first added to these negative selection wells containing 100 μl of PBS and incubated with shaking at room temperature for 20 min. Next, this solution was transferred to the positive selection wells (covered by His-CTDSP1) containing 125 μl of PBSC and incubated with shaking at room temperature for 1 h. Off-target selection was performed by adding 25 μl of purified His-CTDSP1 to each well and incubating for 3 min. Following incubation wells were washed 3 times with PBS and used for cDNA synthesis.

cDNA Synthesis and PCR. We used SuperScript III First-Strand Synthesis System (Invitrogen, Cat #18080-051) for the cDNA synthesis. First, 16 μl of water was mixed with 2 μl of 50 mM primer P19 (Table 1) and 1 μl of the dNTP solution and added this mix to the well in the Maxisorp plate. Next, this plate was incubated at 65° C. for 5 min followed by cooling down at 4° C. for 1 min. Then we transferred 20 μl mixture from the plate to the PCR tube and added 20 μl of the reaction mixture containing 4 μl of 10× buffer, 8 μl of 25 mM MgCl2, 4 μl of 0.1M DTT, 2 μl of RNaseOUT and 2 μl of Superscript III reverse transcriptase. This mixture was incubated at 50° C. for 50 min. Then we added 2 μl of RNAse H and incubated tubes at 37° C. for 20 min.

DNA corresponding to the strong binders surviving the selection was amplified with primers P17 and P20 (Table 1). Amplifications was carried out using Vent DNA polymerase. The PCR reaction (20 μL) was initially heated at 95° C. for 2.5 min followed by 20 or 25 cycles of denaturation at 94° C. for 15 sec., annealing at 55° C. for 15 sec. and extension at 72° C. for 30 sec. Following amplification, PCR fragment was run on the agarose gel, purified by the QIAGEN gel-band purification kit according to manufacturer's protocol.

Analysis of the NGS Data. PCR reactions from each cycle were submitted for Next Generation Sequencing. We used Amplicon-EZ service GeneWiz. Unique sequences were quantified and the most abundant sequences were selected for further testing (Tables 3 and 3a)

TABLE 3
Protein and Nucleotide Sequences of RPP Variants
(SEQ ID NOS are identified in Table 19)

| RPP | | SEQ ID |
|---|---|---|
| Variant | Protein Sequence | NO: |
| v1 | MCTEDLEPPEPPLPKENC | 18 |
| v2 | MCTEAPAPPEPALPKKKKKNC | 19 |
| v3 | MCTEDLQPPTAVPQENC | 20 |
| v4 | MCTEAPAPPEPALPKKKKNC | 21 |
| v5 | MCTADLEPPEPRMEKKKVDC | 22 |
| v6 | MCTGDLQPPKTTVSKKDC | 23 |
| v7 | MCTEDLQSPKTTMTKENC | 24 |
| v8 | MCTEDLEPPEPPLPKEDC | 25 |
| v9 | MCTEDQEQQEEQLPEENC | 26 |
| v10 | MCTADLKPPKTTMTKQNC | 27 |
| v11 | MCPGDLKQPEPPMPKEYC | 28 |
| v12 | MCTEDLEPPKATMTKKDC | 29 |
| v13 | MCTEDQERPPVTKEDC | 30 |
| v14 | MCIADPEPPEAQLPEGNC | 31 |
| v15 | MCTGVQEPPEATLPKKNC | 32 |
| v16 | MCSEAQEPPESRLPQVNC | 33 |
| v17 | MCTKHLEPPGPPLPQENC | 34 |
| v18 | MCTAAPEPPEPPVSKEYC | 35 |
| v19 | MCTEDLQLPKTTMTKEYC | 36 |
| v20 | MCSVDLQPPARLRPMVNC | 37 |
| v21 | MCTGDLQPPESRQPQVNC | 38 |
| v22 | MCTGDLQPPEAQVIEVNC | 39 |
| v23 | MCTEDLQPPEPQLPEVNC | 40 |
| v25 | MCTEDMEPRKTTMTKKYC | 41 |
| v26 | MCTEAPAPPEPALPKKKKKKNC | 42 |
| v27 | MCTEAPAPPEPALPKKKKKNC | 19 |
| v28 | MCTEDLQSPKTTMTKENC | 24 |
| v29 | MCTGDLKLPEPPMSKKKKKNC | 45 |
| v30 | MCTEDLQPPKTTMAEKYC | 46 |
| v31 | MCSEDPEPPKTTMTKKNC | 47 |
| v32 | MCTEDLKPPEASLPEENC | 48 |
| v33 | MCAGDLEQPEPPVAKKKKKNC | 49 |
| v34 | MCNGDLERPEPPVAKEYC | 50 |
| v35 | MCTEDLKPPEPPLPKENC | 51 |
| v36 | MCNEALEPPPLRKEHC | 52 |
| v37 | MCPEDLERPPLTKEHC | 53 |
| v38 | MCTEDLEPPERPLPREIC | 54 |
| v39 | MCAGDLKPPETTMSKKNC | 55 |
| v40 | MCTEDLQQPERSQPMESC | 56 |
| v41 | MCPEDLQPPEPALPEKKKKKIVVLAL | 57 |
| | VVKSAVSVGVV | |
| v42 | MCTEVLVPRTTSGKGRLWFWLWSQKG | 58 |
| | HPSASACGS | |
| v43 | MCAEDLQPPPLLEAHCGSDSGRKKRR | 59 |
| | QC | |
| v44 | MCTAAPEPPEPQLPQANC | 60 |
| v45 | MCPADLQQPETSLPEENC | 61 |
| v46 | MCSVDLQPPARLRPMVNC | 62 |
| v47 | MCTEALEPPEPPLTKENC | 63 |
| v48 | MCTEAMEPPEPPLARESC | 64 |
| v49 | MCTADLQPPEASLPQQNC | 65 |
| v50 | MCTAAPEPPEPRLPEGNC | 66 |
| v51 | MCTKDLAPQAPPLLKENC | 67 |

TABLE 3b
Protein Sequences of RPP^{RI} Variants* (SEQ
ID NOS are identified in Table 19)

| RPP^{RI} | Retroverted (RI) Protein | SEQ |
|---|---|---|
| Variant | Sequence | ID NO |
| v1 | cnekplppeppeldetcm | 68 |
| v2 | cnkkkkkplapeppapaetcm | 69 |
| v3 | cneqpvatppqldetcm | 70 |
| v4 | cnkkkkplapeppapaetcm | 71 |
| v5 | cdkksvttkppqldgtcm | 72 |
| v6 | cdkksvttkppqldgtcm | 73 |
| v7 | cnektmttkpsqldetcm | 74 |
| v8 | cdekplppeppeldetcm | 75 |
| v9 | cneeplqeelleldetcm | 76 |
| v10 | cnqktmttkppkldgpcm | 77 |
| v11 | cyekpmppepqldgpcm | 78 |
| v12 | cdekktmtakppeldetcm | 79 |
| v13 | cdektvppreqdetcm | 80 |
| v14 | cngeplqaeppepdaicm | 81 |
| v15 | cnkkpltaeppeqvgtcm | 82 |
| v16 | cnvqplppgppelhktcm | 83 |
| v17 | cneqplppgppepaatcm | 84 |
| v18 | cyeksvppeppepaatcm | 85 |
| v19 | cyektmttkplqldetcm | 86 |
| v20 | cnvmprlrappqldvscm | 87 |
| v21 | cnvqpqrseppqldgtcm | 88 |
| v22 | cnveivqaeppqldgtcm | 89 |
| v23 | cnveplqpeppqldetcm | 90 |
| v25 | cykktmttkppemdetcm | 91 |
| v26 | cnkkkkkkpklapeppapaetcm | 92 |
| v27 | cnkkkkkplapeppapaetcm | 69 |
| v28 | cnektmttkpsqldetcm | 74 |
| v29 | cnkkkkksmppepplkldgtcm | 95 |
| v30 | cykeamttkppqldetcm | 96 |
| v31 | cnkktmttkppepdescm | 97 |
| v32 | cneeplsaeppkldetcm | 98 |
| v33 | cnkkkkkavppepqeldgacm | 99 |
| v34 | cyekavppepreldgncm | 100 |
| v35 | cnekplppeppkldetcm | 101 |
| v36 | chekrlpppelaencm | 102 |
| v37 | chektlppreldepcm | 103 |
| v38 | cierplpreppeldetcm | 104 |
| v39 | cnkksmtteppkldgacm | 105 |
| v40 | csmpqsrepqqledetcm | 106 |
| v41 | cvvgvsvaskvvlalvvikkkkkeplapeppqldepcm | 107 |
| v42 | csgcasasphgkqswlwfwlrgkgsttrpvlvetcm | 108 |
| v43 | cqrrkkrgsdsgchaellpppqldeacm | 109 |
| v44 | cnaqplqpeppepaatcm | 110 |
| v45 | cneeplstepqqldapcm | 111 |
| v46 | cnvmprlrappqldvscm | 87 |
| v47 | cnektlppeppelaetcm | 113 |
| v48 | cseralppeppemaetcm | 114 |
| v49 | cnqqplsaeppqldatcm | 115 |
| v50 | cngeplrpeppepaatcm | 116 |
| v51 | cnekllppaqpaldktcm | 117 |

*the lowercase letter depicts D-amino acids.

### Example 4—Construction of the Peptide-GST Fusions and their Expression

As a first step we cloned GST protein in pET29 vector. GST was codon optimized, flanked by HindIII and XhoI sites and synthesized by IDT. It was amplified by Phusion DNA polymerase (NEB, cat #M0530S) with primers P109 and P108 (Table 1). The PCR reaction (20 μL) was initially heated at 95° C. for 2.5 min followed by 30 cycles of denaturation at 94° C. for 15 sec., annealing at 55° C. for 15 sec. and extension at 72° C. for 1 min. Following amplification, PCR fragment was gel-purified by the QIAGEN gel-band purification kit according to manufacturer's protocol.

pET29 plasmid was amplified with primers P12 and P14 (Table 1) introducing HindIII and XhoI restriction sites. The PCR reaction (20 μL) was initially heated at 95° C. for 2.5 min followed by 30 cycles of denaturation at 94° C. for 15 sec., annealing at 55° C. for 15 sec. and extension at 72° C. for 6 min. Following amplification, PCR fragment was gel-purified by the QIAGEN gel-band purification kit and digested with HindIII and XhoI restriction enzymes All digested fragments were purified with QUIAGEN kit and ligated in appropriate combination using T4 DNA ligase (NEB) according to recommendations of the manufacturer. Ligated fragments were transformed in 10G chemically competent and sequenced as described above. Expression construct for GST-RPP is shown in Table 4 (SEQ ID NO: 164)

TABLE 4
Nucleotide and Protein Sequences of RPP-GST fusion

Nucleotide Sequence (SEQ ID NO: 164)
ATGTGTACCGAAGATCTGGAACCACCAGAACCACCACTGCCAAAGGAAAA
TTGTggatccGGTTCTGGCTCAGGTTCTTCCCCTATACTAGGTTATTGGA
AAATTAAGGGCCTTGTGCAACCCACTCGACTTCTTTTGGAATATCTTGAA
GAAAAATATGAAGAGCATTTGTATGAGCGCGATGAAGGTGATAAATGGCG
AAACAAAAAGTTTGAATTGGGTTTGGAGTTTCCCAATCTTCCTTATTATA
TTGATGGTGATGTTAAATTAACACAGTCTATGGCCATCATACGTTATATA
GCTGACAAGCACAACATGTTGGGTGGTTGTCCAAAAGAGCGTGCAGAGAT
TTCAATGCTTGAAGGAGCGGTTTTGGATATTAGATACGGTGTTTCGAGAA
TTGCATATAGTAAAGACTTTGAAACTCTCAAAGTTGATTTTCTTAGCAAG
CTACCTGAAATGCTGAAAATGTTCGAAGATCGTTTATGTCATAAAACATA
TTTAAATGGTGATCATGTAACCCATCCTGACTTCATGTTGTATGACGCTC
TTGATGTTGTTTTATACATGGACCCAATGTGCCTGGATGCGTTCCCAAAA
TTAGTTTGTTTTAAAAAACGTATTGAAGCTATCCCACAAATTGATAAGTA
CTTGAAATCCAGCAAGTATATAGCATGGCCTTTGCAGGGCTGGCAAGCCA
CGTTTGGTGGTGGCGACCATCCTCCAAAATAA
Protein Sequence (SEQ ID NO: 165)
MCTEDLEPPEPPLPKENCGSGSGSGSSPILGYWKIKGLVQPTRLLLEYLE
EKYEEHLYERDEGDKWRNKKFELGLEFPNLPYYIDGDVKLTQSMAIIRYI
ADKHNMLGGCPKERAEISMLEGAVLDIRYGVSRIAYSKDFETLKVDFLSK
LPEMLKMFEDRLCHKTYLNGDHVTHPDFMLYDALDVVLYMDPMCLDAFPK
LVCFKKRIEAIPQIDKYLKSSKYIAWPLQGWQATFGGGDHPPK
RPP Sequence is underlined

Selected sequences were re-created by PCR using primers shown in Table 5, gel-purified by the QIAGEN gel-band purification kit, digested by HindIII and BamHI restriction enzymes and cloned in pET vector described above in-frame with the GST tag. Ligated fragments were transformed in 10G chemically competent cells (Lucigen) according to manufacturer's protocol. Transformed cells were plated on LB plates containing 25 μg/mL kanamycin and incubated overnight at 37° C. Next morning colonies were tested for the presence of the insert by colony PCR. Colonies were picked and resuspended in 20 μl of sterile 0.9% sodium chloride solution. One μl of this solution was transferred to the PCR tube and amplified with Taq polymerase (New England Biolabs, cat #M0482S) and 30 pmoles of the flanking primers Each PCR reaction (20 μL) was initially heated at 95° C. for 2.5 min followed by 30 cycles of denaturation at 94° C. for 15 sec., annealing at 55° C. for 15 sec. and extension at 72° C. for 1 min. Amplification products were visualized by agarose electrophoresis. Clones with the correct inserts were inoculated into the culture tubes containing 25 μg/mL kanamycin and incubated overnight at 37° C. Next morning constructs were purified by Monarch Plasmid miniprep kit (NEB). Constructs were transformed into BL21 (DE3) competent cells (Lucigen) as described above.

Purification for Peptide-GST fusions. E. coli cells (BL21 strain) with the selected constructs were incubated overnight at 37° C. with vigorous shaking. Next morning, we added 1 ml of the night culture to 100 ml of LB media with 25 μg/mL kanamycin in 1 L flask and incubated it at 37° C. with shaking. When culture's OD600 reached 0.4 we added IPTG to the final concentration of 1 mM and incubated 16 h at 30° C. with shaking. Next morning, cells were spun down in the Eppendorf centrifuge 5810R at maximum speed and frozen at −80° C. When needed, cell pellets were removed from the freezer, incubated at room temperature and lysed with 3 ml of BPER protein lysis reagent (ThermoFischer). Peptide-GST fusions were purified using glutathione agarose (ThermoScientific cat #16100) as described by the manufacturer.

TABLE 5
Primers used for cloning RPP variants in-frame with GST

| RPP | | | |
|---|---|---|---|
| Variant | Primer | Primer Sequence | SEQ ID NO: |
| v1 | forward primer | AATAAAGCTTATGTGTACCGAAGATCTGGAACCACCAGAACCA | 166 |
| (RPP) | | CCACTGCCAA | |
| | reverse primer | AATAGGATCCACAATTTTCCTTTGGCAGTGGTGGTTCTGGTGGT | 167 |
| | | TCCA | |
| v2 | forward primer | AATAAAGCTTATGTGTACCGAAGCTCCGGCACCACCAGAACCA | 168 |
| | | GCACTGCCAAAG | |
| | reverse primer | AATAGGATCCACAATTTTTTTTTTTTTTCTTTGGCAGTGCTGGTT | 169 |
| | | CTGGTGGTGC | |
| v3 | forward primer | AATAAAGCTTATGTGTACCGAAGATCTGCAACCACCAACAGCA | 170 |
| | | GTGCCAC | |
| | reverse primer | AATAGGATCCACAATTTTCCTGTGGCACTGCTGTTGGTGGTTGC | 171 |
| | | AGATCTTC | |
| v4 | forward primer | AATAAAGCTTATGTGTACCGAAGCTCCGGCACCACCAGAACCA | 172 |
| | | GCACTGCCAAAG | |
| | reverse primer | AATAGGATCCACAATTTTTTTTTTTCTTTGGCAGTGCTGGTTCTG | 173 |
| | | GTGGTGC | |
| v5 | forward primer | ATGTGTACCGCAGATCTGGAACCACCAGAACCACGAATGGAA | 174 |
| | reverse primer | AATAGGATCCACAATCTACCTTTTTTTTTTCCATTCGTGGTTCTG | 175 |
| | | GTGGTTCCA | |
| v6 | forward primer | AATAAAGCTTATGTGTACCGGAGATCTGCAACCACCAAAAACA | 176 |
| | | ACAGTGTCAA | |
| | reverse primer | AATAGGATCCACAATCTTTCTTTGACACTGTTGTTTTTGGTGGT | 177 |
| | | TGCAGA | |
| v7 | forward primer | AATAAAGCTTATGTGTACCGAAGATCTGCAATCACCAAAAACA | 178 |
| | | ACAATGACAA | |
| | reverse primer | AATAGGATCCACAATTTTCCTTTGTCATTGTTGTTTTTGGTGATT | 179 |
| | | GCAGA | |
| v8 | forward primer | AATAAAGCTTATGTGTACCGAAGATCTGGAACCACCAGAACCA | 180 |
| | | CCACTGCCAA | |
| | reverse primer | AATAGGATCCCACAATCTTCCTTTGGCAGTGGTGGTTCTGGTGG | 181 |
| | | TTCCAGA | |
| v9 | forward primer | AATAAAGCTTATGTGTACCGAAGATCAGGAACAACAAGAAGA | 182 |
| | | ACAACTG | |
| | reverse primer | AATAGGATCCACAATTTTCCTCTGGCAGTTGTTCTTCTTGTTGTT | 183 |
| | | CCTGATC | |
| v10 | forward primer | AATAAAGCTTATGTGTACCGCAGATCTGAAACCACCAAAAACA | 184 |
| | | ACAATGACAA | |
| | reverse primer | AATAGGATCCACAATTTTGCTTTGTCATTGTTGTTTTTGGTGGTT | 185 |
| | | TCAGA | |
| V11 | forward primer | AATAAAGCTTATGTGTCCCGGAGATCTGAAACAACCAGAACCA | 186 |
| | | CCAATGCCAA | |
| | reverse primer | AATAGGATCCACAATATTCCTTTGGCATTGGTGGTTCTGGTTGT | 187 |
| | | TTCAGA | |
| V12 | forward primer | AATAAAGCTTATGTGTACCGAAGATCTGGAACCACCAAAAGCA | 188 |
| | | ACAATGACAA | |
| | reverse primer | AATAGGATCCACAATCTTTCTTTGTCATTGTTGCTTTTGGTGGTT | 189 |
| | | CCAGA | |
| V13 | forward primer | AATAAAGCTTATGTGTACCGAAGATCAGGAACGACCACCAGTG | 190 |
| | | ACAAAG | |
| | reverse primer | AATAGGATCCACAATCTTCCTTTGTCACTGGTGGTCGTTCCTGA | 191 |
| | | TCTTC | |
| V14 | forward primer | AATAAAGCTTATGTGTATCGCAGATCCGGAACCACCAGAAGCA | 192 |
| | | CAACTG | |
| | reverse primer | AATAGGATCCACAATTTCCCTCTGGCAGTTGTGCTTCTGGTGGT | 193 |
| | | TCCGGATCTG | |
| V15 | forward primer | AATAAAGCTTATGTGTACCGGAGTTCAGGAACCACCAGAAGCA | 194 |
| | | ACACTG | |
| | reverse primer | AATAGGATCCACAATTTTTCTTTGGCAGTGTTGCTTCTGGTGGT | 195 |
| | | TCCTGAAC | |
| v16 | forward primer | AATAAAGCTTATGTGTAGCGAAGCTCAGGAACCACCAGAATCA | 196 |
| | | CGACTG | |
| | reverse primer | AATAGGATCCACAATTTACCTGTGGCAGTCGTGATTCTGGTGGT | 197 |
| | | TCCTGAG | |
| V17 | forward primer | AATAAAGCTTATGTGTACCAAACATCTGGAACCACCAGGACCA | 198 |
| | | CCATTGC | |
| | reverse primer | AATAGGATCCACAATTTTCCTGTGGCAATGGTGGTCCTGGTGGT | 199 |
| | | TCCAGATG | |
| v18 | forward primer | AATAAAGCTTATGTGTACCGCAGCTCCGGAACCACCAGAACCA | 200 |
| | | CCAGTGT | |
| | reverse primer | AATAGGATCCACAATATTCCTTTGACACTGGTGGTTCTGGTGGT | 201 |
| | | TCCGGAG | |
| v19 | forward primer | AATAAAGCTTATGTGTACCGAAGATCTGCAACTACCAAAAACA | 202 |
| | | ACAATGACA | |
| | reverse primer | AATAGGATCCACAATATTCCTTTGTCATTGTTGTTTTTGGTAGT | 203 |
| | | TGCAGA | |
| v20 | forward primer | AATAAAGCTTATGTGTTCCGTAGATCTGCAACCACCAGCACGA | 204 |
| | | CTACGGCCAA | |
| | reverse primer | AATAGGATCCACAATTTACCATTGGCCGTAGTCGTGCTGGTGG | 205 |
| | | TTGCAGAT | |
| v21 | forward primer | AATAAAGCTTATGTGTACCGGAGATCTGCAACCACCAGAATCA | 206 |
| | | CGACAGCCA | |
| | reverse primer | AATAGGATCCACAATTTACCTGTGGCTGTCGTGATTCTGGTGGT | 207 |
| | | TGCAGAT | |
| v22 | forward primer | AATAAAGCTTATGTGTACCGGAGATCTGCAACCACCAGAAGCA | 208 |
| | | CAAGTGA | |
| | reverse primer | AATAGGATCCACAATTTACCTCTATCACTTGTGCTTCTGGTGGT | 209 |
| | | TGCAGAT | |
| v23 | forward primer | AATAAAGCTTATGTGTACCGAAGATCTGCAACCACCAGAACCA | 210 |
| | | CAACTGCCA | |
| | reverse primer | AATAGGATCCACAATTTACCTCTGGCAGTTGTGGTTCTGGTGGT | 211 |
| | | TGCAGAT | |
| v25 | forward primer | AATAAAGCTTATGTGTACCGAAGATATGGAACCACGAAAAACA | 212 |
| | | ACAATGA | |
| | reverse primer | AATAGGATCCACAATATTTCTTTGTCATTGTTGTTTTTCGTGGTT | 213 |
| | | CCATAT | |
| v26 | forward primer | AATAAAGCTTATGTGTACCGAAGCTCCGGCACCACCAGAACCA | 214 |
| | | GCACTGCCAAAG | |
| | reverse primer | AATAGGATCCACAATTTTTTTTTTTTTTTTTCTTTGGCAGTGCTG | 215 |
| | | GTTCTGGTGGT | |
| v27 | forward primer | AATAAAGCTTATGTGTACCGAAGCTCCGGCACCACCAGAACCA | 216 |
| | | GCACTGCCAAAG | |
| | reverse primer | AATAGGATCCACAATTTTTTTTTTTTTTCTTTGGCAGTGCTGGTT | 217 |
| | | CTGGTGGTG | |
| v28 | forward primer | AATAAAGCTTATGTGTACCGAAGATCTGCAATCACCAAAAACA | 218 |
| | | ACAATG | |
| | reverse primer | AATAGGATCCACAATTTTCCTTTGTCATTGTTGTTTTTGGTGATT | 219 |
| | | GCAGA | |
| v29 | forward primer | AATAAAGCTTATGTGTACCGGAGATCTGAAACTACCAGAACCA | 220 |
| | | CCAATGTCAAAG | |
| | reverse primer | AATAGGATCCACAATTTTTTTTTTTTTTCTTTGACATTGGTGGTT | 221 |
| | | CTGGTAG | |
| v30 | forward primer | AATAAAGCTTATGTGTACCGAAGATCTGCAACCACCAAAAACA | 222 |
| | | ACAATG | |
| | reverse primer | AATAGGATCCACAATATTTCTCTGCCATTGTTGTTTTTGGTGGT | 223 |
| | | TGCAGAT | |
| v31 | forward primer | AATAAAGCTTATGTGTAGCGAAGATCCGGAACCACCAAAAACA | 224 |
| | | ACAATGAC | |
| | reverse primer | AATAGGATCCACAATTTTTCTTTGTCATTGTTGTTTTTGGTGGTT | 225 |
| | | CCGGAT | |
| v32 | forward primer | AATAAAGCTTATGTGTACCGAAGATCTGAAACCACCAGAAGCA | 226 |
| | | TCACTGC | |
| | reverse primer | AATAGGATCCACAATTTTCCTCTGGCAGTGATGCTTCTGGTGGT | 227 |
| | | TTCAGAT | |
| v33 | forward primer | AATAAAGCTTATGTGTGCCGGAGATCTGGAACAACCAGAACCA | 228 |
| | | CCAGTGGCAAA | |
| | reverse primer | AATAGGATCCACAATTTTTTTTTTTTTTCTTTGCCACTGGTGGTT | 229 |
| | | CTGGTTGTTC | |
| v34 | forward primer | AATAAAGCTTATGTGTAACGGAGATCTGGAACGACCAGAACCA | 230 |
| | | CCAGTG | |
| | reverse primer | AATAGGATCCACAATATTCCTTTGCCACTGGTGGTTCTGGTCGT | 231 |
| | | TCCAGAT | |
| v35 | forward primer | AATAAAGCTTATGTGTACCGAAGATCTGAAACCACCAGAACCA | 232 |
| | | CCACTGC | |
| | reverse primer | AATAGGATCCACAATTTTCCTTTGGCAGTGGTGGTTCTGGTGGT | 233 |
| | | TTCAGAT | |
| v36 | forward primer | AATAAAGCTTATGTGTAACGAAGCTCTGGAACCACCACCACTG | 234 |
| | | CGAAAG | |
| | reverse primer | AATAGGATCCACAATGTTCCTTTCGCAGTGGTGGTGGTTCCAG | 235 |
| | | AGCTTC | |
| v37 | forward primer | AATAAAGCTTATGTGTCCCGAAGATCTGGAACGACCACCATTG | 236 |
| | | ACAAAG | |
| | reverse primer | AATAGGATCCACAATGTTCCTTTGTCAATGGTGGTCGTTCCAGA | 237 |
| | | T | |
| v38 | forward primer | AATAAAGCTTATGTGTACCGAAGATCTGGAACCACCAGAACGA | 238 |
| | | CCACTGC | |
| | reverse primer | AATAGGATCCACAAATTTCCCTTGGCAGTGGTCGTTCTGGTGGT | 239 |
| | | TCCAGA | |
| v39 | forward primer | AATAAAGCTTATGTGTGCCGGAGATCTGAAACCACCAGAAACA | 240 |
| | | ACAATGTC | |
| | reverse primer | AATAGGATCCACAATTTTTCTTTGACATTGTTGTTTCTGGTGGT | 241 |
| | | TTCAGA | |
| v40 | forward primer | AATAAAGCTTATGTGCACCGAAGATCTGCAACAACCAGAACGA | 242 |
| | | TCACAGC | |
| | reverse primer | AATAGGATCCACAACTTTCCATTGGCTGTGATCGTTCTGGTTGT | 243 |
| | | TGCAGA | |
| v41 | forward primer | AATAAAGCTTATGTGTCCCGAAGATCTGCAACCACCAGAACCA | 244 |
| | | GCACTGCCAGAGAAAAA | |
| | reverse primer | AATAGGATCCGACCACGCCGACGCTGACGGCGCTTTTTACGAC | 245 |
| | | CAGAGCCAGAACCACAA | |
| | additional | ACCAGAACCAGCACTGCCAGAGAAAAAAAAAAAAAAAATTGT | 246 |
| | primer | GGTTCTGGCTCTGGTCGT | |
| v42 | forward primer | AATAAAGCTTATGTGTACCGAAGTTCTGGTACCACGAACCACC | 247 |
| | | AGTGGCAAAGGAAG | |
| | reverse primer | AATAGGATCCGGAGCCACACGCCGATGCTGACGGATGGCCTTT | 248 |
| | | TTGCGACCAGAGCCAGAA | |
| | additional | ACCACGAACCACCAGTGGCAAAGGAAGATTGTGGTTCTGGCTC | 249 |
| | primer | TGGTCGCAAAAAGGCCA | |
| v43 | forward primer | AATAAAGCTTATGTGTGCCGAAGATCTGCAACCACCACCACTG | 250 |
| | | CTAGAGGCA | |
| | reverse primer | AATAGGATCCACACTGACGGCGCTTTTTACGACCAGAGTCAGA | 251 |
| | | ACCACAATGTGCCTCT | |
| | additional | ATCTGCAACCACCACCACTGCTAGAGGCACATTGTGGTTCTGA | 252 |
| | primer | CTCTGGTCGTAAAAAG | |
| v44 | forward primer | AATAAAGCTTATGTGTACCGCAGCTCCGGAACCACCAGAACCA | 253 |
| | | CAACTG | |
| | reverse primer | AATAGGATCCACAATTTGCCTGTGGCAGTTGTGGTTCTGGTGGT | 254 |
| | | TCCGGAG | |
| v45 | forward primer | AATAAAGCTTATGTGTCCCGCAGATCTGCAACAACCAGAAACA | 255 |
| | | TCACTG | |
| | reverse primer | AATAGGATCCACAATTTTCCTCTGGCAGTGATGTTTCTGGTTGT | 256 |
| | | TGCAGAT | |
| v46 | forward primer | AATAAAGCTTATGTGTTCCGTAGATCTGCAACCACCAGCACGA | 257 |
| | | CTACG | |
| | reverse primer | ACAATTTACCATTGGCCGTAGTCGTGCTGGTGGTTGCAGAT | 326 |
| v47 | forward primer | AATAAAGCTTATGTGCACCGAGCTTTGGAACCACCAGAACCAC | 327 |
| | | CACTG | |
| | reverse primer | AATAGGATCCACAATTTTCCTTTGTCAGTGGTGGTTCTGGTGGT | 260 |
| | | TCCAAAG | |
| v48 | forward primer | AATAAAGCTTATGTGTACCGAAGCTATGGAACCACCAGAACCA | 261 |
| | | CCACTG | |
| | reverse primer | AATAGGATCCACAACTTTCCCTTGCCAGTGGTGGTTCTGGTGGT | 262 |
| | | TCCATAG | |
| v49 | forward primer | AATAAAGCTTATGTGTACCGCAGATCTGCAACCACCAGAAGCA | 263 |
| | | TCACTG | |
| | reverse primer | AATAGGATCCACAATTTTGCTGTGGCAGTGATGCTTCTGGTGGT | 264 |
| | | TGCAGA | |
| v50 | forward primer | AATAAAGCTTATGTGTACCGCAGCTCCGGAACCACCAGAACCA | 265 |
| | | CGACTGCCA | |
| | reverse primer | AATAGGATCCACAATTTCCCTCTGGCAGTCGTGGTTCTGGTGGT | 266 |
| | | TCC | |
| v51 | forward primer | AATAAAGCTTATGTGTACCAAAGATTTGGCACCACAAGCACCA | 267 |
| | | CCATTGCT | |
| | reverse primer | AATAGGATCCACAATTTTCCTTTAGCAATGGTGGTGCTTGTGGT | 268 |
| | | GCCAAA | |

### Example 5—Inhibition of the CTDSP1 Phosphatase Activity by RPP and RPP Variants

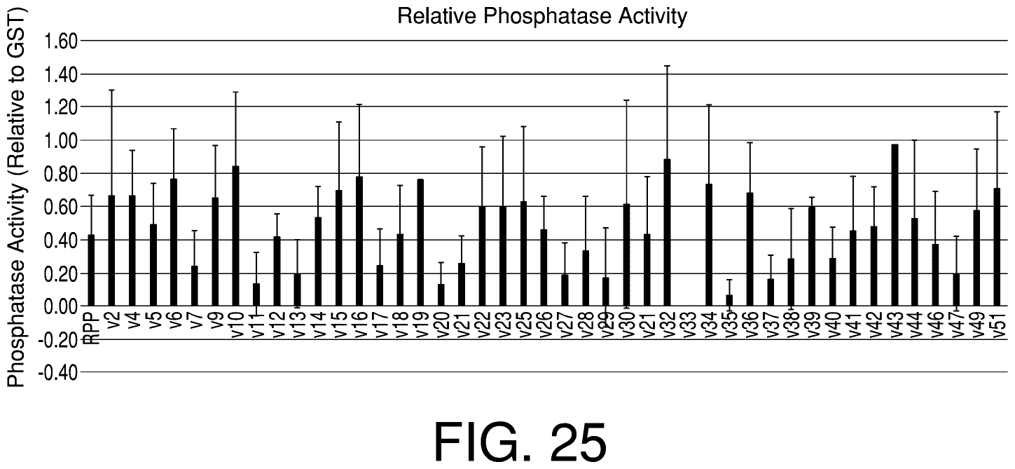

Results: FIG. 25—The top 51 most abundant RPP variants identified in the RNA display/protein evolution screen are listed in-Table 3 in the order of most (V1) to least (V51) abundant peptides. The primers used to make these peptides are listed in-Table 5. A phosphatase activity screen (Table 6, FIG. 25) was used to assess the ability of the top 51 RPPv to inhibit CTDSP1 phosphatase activity at amino acids 861 and 864 on the endogenous phosphorylated REST peptide (TEDpSPPpSPPLPKEN (SEQ ID NO: 329)).

The phosphorylated REST peptide (TEDpSPPpSPPLPKEN (SEQ ID NO: 329)) was synthesized by GeneScript. Phosphatase reactions were performed with 10 mM Tris pH 8, 10 mM MgCl₂, 100 nM CTDSP1, 0.5 UM phosphate sensor (Thermo Fischer), 50 μM of REST peptide and various concentrations of peptide-GST fusions at room temperature for 10 min. All assays were performed in 96-well pates (Corning P/N 3686) rinsed with water 10 times. Fluorescence was measured by BioTek Synergy HTX using kinetic read with excitation at 420/27 nm and emission at 485/20 nm.

The screen showed RPP (V1) inhibited phosphatase activity by approximately 60%, relative to control (GST), at 1 μM (Table 6, FIG. 25). Several variants inhibited phosphatase activity better than RPP (Table 6, FIG. 25). The most potent inhibitors were v33, with 100% inhibition, and v35, with approximately 90% inhibition of phosphatase activity (Table 6, FIG. 25).

TABLE 6
Inhibition of CTDSP1 phosphatase
acclivity by RPP variants

| | Relative | Standard | Number |
|---|---|---|---|
| RPP | Phosphatase | deviation, | of |
| variant | activity | P < 0.05 | replicas |
| RPP | 0.42 | 0.25 | 5 |
| v2 | 0.66 | 0.64 | 6 |
| v4 | 0.67 | 0.28 | 3 |
| v5 | 0.49 | 0.25 | 6 |
| v6 | 0.77 | 0.30 | 4 |
| v7 | 0.24 | 0.21 | 5 |
| v9 | 0.65 | 0.32 | 4 |
| v10 | 0.84 | 0.45 | 4 |
| v11 | 0.13 | 0.19 | 3 |
| v12 | 0.41 | 0.14 | 4 |
| v13 | 0.20 | 0.20 | 4 |
| v14 | 0.54 | 0.19 | 2 |
| v15 | 0.70 | 0.41 | 4 |
| v16 | 0.79 | 0.43 | 4 |
| v17 | 0.24 | 0.22 | 6 |
| v18 | 0.43 | 0.30 | 4 |
| v19 | 0.76 | 0.00 | 1 |
| v20 | 0.13 | 0.13 | 2 |
| v21 | 0.26 | 0.17 | 4 |
| v22 | 0.59 | 0.37 | 2 |
| v23 | 0.60 | 0.42 | 4 |
| v25 | 0.63 | 0.45 | 4 |
| v26 | 0.46 | 0.20 | 2 |
| v27 | 0.19 | 0.19 | 2 |
| v28 | 0.33 | 0.33 | 2 |
| v29 | 0.17 | 0.30 | 4 |
| v30 | 0.62 | 0.63 | 4 |
| v31 | 0.43 | 0.35 | 3 |
| v32 | 0.89 | 0.56 | 4 |
| v33 | 0.00 | 0.00 | 3 |
| v34 | 0.74 | 0.48 | 4 |
| v35 | 0.07 | 0.09 | 4 |
| v36 | 0.68 | 0.31 | 3 |
| v37 | 0.16 | 0.15 | 5 |
| v38 | 0.28 | 0.30 | 3 |
| v39 | 0.59 | 0.07 | 3 |
| v40 | 0.29 | 0.19 | 3 |
| v41 | 0.46 | 0.33 | 6 |
| v42 | 0.48 | 0.24 | 7 |
| v43 | 0.97 | 0.00 | 1 |
| v44 | 0.53 | 0.47 | 3 |
| v46 | 0.37 | 0.32 | 7 |
| v47 | 0.20 | 0.22 | 3 |
| v49 | 0.58 | 0.37 | 4 |
| v51 | 0.71 | 0.46 | 3 |

FIG. 26, Table 13-RPP, SEQ ID NO: 12, inhibited CTDSP1 activity with an EC₅₀ of approximately 20 nM, but did not affect the activity of several other phosphatases.

Assay: The ability of RPP SEQ ID NO: 12 to inhibit CTDSP1 activity at amino acids 861 and 864 on the endogenous phosphorylated REST peptide (TEDpSPPpSPPLPKEN (SEQ ID NO: 329)) was assessed as described for FIG. 26 above.

TABLE 13
CTDSP1 and off target phosphatase acclivity
after RPP (SEQ ID NO: 12) dosing

| | RPP SEQ | Average | |
|---|---|---|---|
| | ID NO: 12 | N = 5 | Standard |
| Phosphatase | nM | % Activity | Deviation |
| CTDSP1 | 0 | 100 | 0 |
| CTDSP1 | 10 | 89.97 | 15.64 |
| CTDSP1 | 20 | 48.7 | 14.65 |
| CTDSP1 | 40 | 34.46 | 11.58 |
| CTDSP1 | 60 | 31.17 | 21.23 |
| CTDSP1 | 80 | 22.67 | 10.2 |
| CTDSP1 | 100 | 13.72 | 13.35 |
| PPA1 | 10000 | 101.125 | 1.611 |
| PPM1H | 10000 | 98.279 | 2.406 |
| PPM1A | 10000 | 102.786 | 2.559 |
| PP3CA | 10000 | 100.222 | 2.897 |
| PPP1CA | 10000 | 101.136 | 3.711 |
| PP5CA | 10000 | 97.902 | 5.245 |

### Example 6—Binding Affinity of RPP Assessed Using Monolith (NanoTemper) and Biacore (GE Healthcare Life Sciences)

Expression constructs for His-CTDSP1 (Table 2; SEQ ID NO: 162) and GST-RPP (Table 4; SEQ ID NO: 164) were constructed and purified as described in Example 2. The His tag on CTDSP1 (40 nM) was labelled with RED-tris NTA dye (20 nM), with a 3:1 dye to CTDSP1 ratio, and unbound dye was removed by gravity-flow size exclusion. Binding affinity of the linear GST-RPP (Table 4; SEQ ID NO: 165) to His-CTDSP1 (Table 2; SEQ ID NO: 163) was determined using Monolith (NanoTemper), following manufacturer recommendations. Results of the binding assay are presented in FIG. 2: The RPP (Table 4; SEQ ID NO: 165) was assessed for binding to His-CTDSP1 at several concentrations ranging from low pM up to 0.5 μM. K_{D} corresponding to the binding of linear RPP to CTDSP1 was calculated at 130 pM.

Binding affinity of the linear GST-RPP (Table 4; SEQ ID NO: 165) to His-CTDSP1 (Table 2; SEQ ID NO: 163) was measured using Biacore by GE Healthcare Life Sciences. 20 μL (5 μg/mL) of His-tagged CTDSP1 in 10 mM sodium acetate, pH 5.0 was immobilized on a CM5 chip (GE Healthcare Life Science) using an amine coupling following a protocol recommended by Biacore (cat #BR-1000-50, GE Healthcare Life Science). This purification yielded approximately 1300 RU of protein bound to the CM5 chip. The CM5 chip was then washed with HBS-EP buffer (pH 7.4), 120 μL of the analyte: GST-RPP or GST alone (negative control) was injected at a concentration of 500 nM in HBS-EP buffer (pH 7.4) at flow rate of 30 μL/min. The association time was 2 minutes and dissociation time is 3 minutes. Finally, bound protein was washed with 10 mM glycine-HCl, pH1.5 (BR-1003-54, GE Health science) for 25 second at 50 μL/min flow rate. The binding of linear RPP to CTDSP1 resulted in: K_{D}=1.7 pM (FIG. 3B, the negative control is shown in FIG. 3A).

### Example 7—RPP and RPPv Fused with the Cell-Penetrating Peptides (CPPs) and/or Peptides Facilitating Endosome Escape

Several RPP and RPPv were synthesized with attachments of the cell-penetrating peptides (CPPs) and/or peptides facilitating endosome escape. The list of CPPs and linkers we used is shown in Table 7.

TABLE 7
Additional peptide allowing cell penetration and escape from endosomes

| | | SEQ | | SEQ |
|---|---|---|---|---|
| Name | Protein Sequence (a) | ID NO: | RI sequence (b) | ID NO: |
| CPP | GRKKRRQRRR | 118 | rrrqrrkkrg | 140 |
| CPP | GDIMGEWGNEIFGAIAGFLGYGR | 119 | rrrqrrkkrgyglfgaiagfiengwegmgmidg | 141 |
| | KKRRQRRR | | | |
| CPP | RRRRRRRR | 120 | rrrrrrrr | 142 |
| CPP | KKKKKKKK | 121 | kkkkkkkk | 143 |
| CPP | DIMGEWGNEIFGAIAGFLG | 122 | glfgaiagfiengwegmid | 144 |
| CPP | CHHHHHRKKRRQRRRRHHHHHC | 123 | chhhhhrrrrqrrkkrhhhhhc | 145 |
| CPP | CHHHHHRRRRRRRRRHHHHHC | 124 | chhhhhrrrrrrrrrhhhhhc | 146 |
| CPP | FFLIPKGRRRRRRRRGC | 125 | cgrrrrrrrrgkpilffc | 147 |
| CPP | FΦRRRR | 126 | rrrrΦf | 148 |
| CPP | RRWWRRWRRRRWWRr | 127 | rrwwrrrrwrrwwrr | 149 |
| CPP | RWWRRRRWRRWWRr | 128 | rrwwrrwrrrrwwr | 150 |
| CPP | RRWWRRWRRRRWWr | 129 | rwwrrrrwrrwwrr | 151 |
| CPP | RRWWRRWRRRr | 130 | rrrrwrrwwrr | 152 |
| CPP | RRWWRRWRr | 131 | rrwrrwwrr | 153 |
| CPP | RRWWRRWRRR | 132 | rrrwrrwwrr | 154 |
| CPP | RRRRRRCᵢ₄RRWWRRr | 133 | rrrwwrrC₁₄rrrrrr | 155 |
| CPP | YALTSAISRIITHHHHHH | 134 | hhhhhhtiirsiastlay | 156 |
| CPP | RRRRRC₁₄RRWWRR- | 135 | rrwwrrC₁₄rrrrr | 157 |
| CPP | RRC₁₄RRC₁₄RRRRR | 136 | rrC₁₄rrC₁₄rrrrr | 158 |
| CPP | RRC₁₄RRR | 137 | rrC₁₄rrr | 159 |
| Linker | GS | 138 | sg | 160 |
| 1 | | | | |
| Linker | GSGS | 139 | sgsg | 161 |
| 2 | | | | |

Φ is L-2-naphthylalanine
Lower case = D-amino acids
C₁₄ = 2-amino-tetradecanoic acid

### Example 8—RPP is Internalizalized by Mesenchymal Progenitor Cells and Sciatic Nerve

### Mesenchymal Progenitor Cells (MPCs)

Result. After a 4 hour incubation with RPP (SEQ ID NO.: 4), followed by 6 days in culture, RPP remains internalized in mesenchymal progenitor cells (MPCs) (FIG. 4, C and D) and localizes in the nucleus over the 6 day culture period (FIG. 4, E and F).

### Materials and Methods

Dosing and Cell Culture. MPCs were cultured as previously described¹¹⁶, plated at 1.0×10⁴ cells/cm² onto glass coverslips in a 24-well plate, incubated overnight at 37° C., dosed with 100 nM RPP (SEQ ID NO: 4) for 4 hours, followed by a change in media. Cell were cultured for 6 days with a media change every two days. Interim MPC harvesting occurred at 24 and 72 hours to assess intracellular RPP localization.

Immunohistochemistry. Phalloidin (Invitrogen) was used for cytoskeletal staining, Hoechst 333429 (Calbiochem) dye was used to visualize the nucleus, and FLAG antibody (Sigma-Aldrich) was used to visualize the RPP. Images were taken with a confocal laser scanning microscope.

### Sciatic Nerve

Result. Linear RPP accumulates in the nucleus of sciatic nerve tissue (FIG. 5, A and B).

### Materials and Methods

Dosing and Procedure. FIG. 5: The sciatic nerve of a Sprague Dawley rat (L3 and L4 region) was exposed (Schmitz and Beer, 2001)¹¹⁷ and a 0.7 cm section remove to create an approximately 1 cm segmental defect following retraction of the nerve stumps (Hems and Glasby, 1993)¹¹⁸. 1 mg of RPP (SEQ ID NO: 4) in PBS was injected into the site of injury and the surgical incision was closed. After 48 hours, the animal was sacrificed and the spinal cord from the L4 to L6 region was sectioned coronally.

Rational for dose levels. Maximum feasible dose.

Test Article Identification. RPP (SEQ ID NO: 4)

Purity. ≥95%

Immunohistochemistry. Hoechst 333429 (Calbiochem) dye was used to visualize the nucleus, and FLAG antibody (Sigma-Aldrich) was used to visualize the RPP. Images were taken with a light microscope.

### Example 9—RPP Induced Degradation of REST Protein

Result. RPP (SEQ ID NO: 4) decreased REST protein levels by 58% compared to vehicle control (FIG. 6).

### Materials and Methods

Test Article Identification. RPP (SEQ ID NO: 4)

Purity. ≥95%

Dosing and Cell Culture. HEK 293 cells were grown in Dulbecco's

Modified Eagle's Medium (DMEM) supplemented with 10% bovine calf serum. Transfection of HEK 293 cells, with 100 nM of full-length human REST protein², was performed in 80 to 90% confluent cultures in 35 mm dishes using lipofectamine2000 (Life Technologies), following manufacturer recommendations. 24 hours post transfection, cells were dosed with 100 nM RPP (SEQ ID NO: 4) or vehicle control (PBS) overnight.

Western Blot Analysis. Whole-cell lysates were prepared following the procedures in Ballas et. al., 2001¹¹⁹. Western blots were performed by standard procedures using anti-REST-C⁶⁴, anti-GAPDH (abcam [6C5]), and anti-IgG conjugated to infrared dyes (Thermo Fisher), and analyzed on an Odyssey infrared fluorescence imager (LiCor). The bar graph is a quantification of the Western Blot using ImageJ (https://imagej.nih.gov/ij).

### Example 10—RPP can be Used to Induce Expression of BDNF, NGF, Kv4.3, Kv7.2, Nav1.8, and OPRM1 mRNR and is not Neurotoxic

Result. RPP increases expression of BDNF, NGF, Kv4.3, Kv7.2, Nav1.8, and OPRM1 (FIGS. 7, 8, 9, 10, 14, 15, and 16; Table 15). RPP (SEQ ID NO: 12) does not cause necrosis in DRG neurons (FIG. 17), as demonstrated by an LDH cytoxicity assay in DRG neurons incubated with various concentrations of RPP for 48 h. ***=p<0.001, n=4, one-way ANOVA and Dunnett's test, error bars=SD.

TABLE 15
RPP induced expression of BDNF, NGF, Kv4.3, Kv7.2, Navi.8, and OPRM1 mRNA

| Target | Figure | Increase | Cell Type | RPP SEQ ID NO | Dose | Duration |
|---|---|---|---|---|---|---|
| BDNF | 7 | 2.4x | HEK 293 | 4 | 100 nM | 24 h |
| BDNF | 9 | 5x | NBFL | 2 | 1 μM | 16h |
| BDNF | 9 | 2.3x | NBFL | 2 | 1 μM | 48h |
| BDNF | 9 | 2.3x | NBFL | 4 | 1 μM | 48 h |
| BDNF | 10 | 2x | MPC | 9 | 3 μM | 48 h |
| BDNF | 16 | 1.4x | DRG | 12 | 0.3 μM | 48 h |
| BDNF | 16 | 1.3x | DRG | 12 | 1 μM | 48 h |
| BDNF | 16 | 1.3x | DRG | 12 | 3 μM | 48 h |
| BDNF | 16 | 1.7 | DRG | 12 | 10 μM | 48 h |
| NGF | 9 | 3.2x | NBFL | 2 | 1 μM | 16 h |
| NGF | 9 | 1.7x | NBFL | 2 | 1 μM | 48 h |
| NGF | 9 | 1.9x | NBFL | 4 | 1 μM | 48 h |
| NGF | 10 | 2.9x | MPC | 9 | 3 μM | 48 h |
| NGF | 16 | — | DRG | 12 | 0.3 μM | 48 h |
| NGF | 16 | 0.9x | DRG | 12 | 1 μM | 48 h |
| NGF | 16 | 1.25 | DRG | 12 | 3 μM | 48 h |
| NGF | 16 | 1.9 | DRG | 12 | 10 μM | 48 h |
| K_{V}4.3 | 8 | 3x | NBFL | 1 | 2 μg (transfected) | 48 h |
| K_{V}4.3 | 9 | 5x | NBFL | 2 | 1 μM | 16 h |
| K_{V}4.3 | 9 | 5x | NBFL | 2 | 1 μM | 48 h |
| K_{V}4.3 | 9 | 19.5x | NBFL | 4 | 1 μM | 48 h |
| K_{V}4.3 | 14 | 0x | DRG | 12 | 1 μM | 48 h |
| K_{V}4.3 | 14 | 0x | DRG | 12 | 3 μM | 48 h |
| K_{V}4.3 | 14 | 1.9x | DRG | 12 | 10 μM | 48 h |
| K_{V}7.2 | 14 | 0 | DRG | 12 | 1 μM | 48 h |
| K_{V}7.2 | 14 | 6.7x | DRG | 12 | 3 μM | 48 h |
| K_{V}7.2 | 14 | 7.5x | DRG | 12 | 10 μM | 48 h |
| Navi.8 | 13 | 0.8x | DRG | 12 | 1 μM | 48 h |
| Navi.8 | 14 | 2.6x | DRG | 12 | 3 μM | 48 h |
| Navi.8 | 14 | 4x | DRG | 12 | 10 μM | 48 h |
| Navi.8 | 15 | 6x | DRG | 13 | 1 μM | 48 h |
| Navi.8 | 15 | 13x | DRG | 14 | 1 μM | 48 h |
| OPRM1 | 14 | 1.2x | DRG | 12 | 1 μM | 48 h |
| OPRM1 | 14 | 3x | DRG | 12 | 3 μM | 48 h |
| OPRM1 | 14 | 3.9x | DRG | 12 | 10 μM | 48 h |

### FIG. 7 Materials and Methods

Test Article Identification. RPP (SEQ ID NO: 4), purity. ≥95%

Dosing and Cell Culture. 100 nM RPP; HEK 293 cells,

mRNA extraction and quantification protocol. Cells were lysed in QIAzol (Qiagen) and total RNA was extracted using RNeasy Midi Kit (Qiagen) according to manufacturer's instructions. Purified RNA was quantified with the NanoDrop 2000 (ThermoFIsher), and reverse transcription was run using High Capacity cDNA Reverse Transcription Kit (Applied Biosystems) and 2 ng/μL RNA per reaction. For qRT-PCR, 5 μL of cDNA (corresponding to 10 ng of RNA) were mixed with to 10 μL SsoAdvanced™ Universal SYBR® Green Supermix (BioRad) and 1 μL of each primer (final primer concentration 500 nM each). Reactions were run in triplicates in a QuantStudio™ 7 Flex Real-Time PCR system (Applied Biosystems). Amplification data were analyzed using the comparative cycle threshold (ΔDCₜ) method and β-actin as calibrator. The primers used were as follows: β-actin forward: 5′-AGAGCACGAGCTGCCTGAC-3′ (SEQ ID NO: 332), β-actin reverse: 5′-GGATGCCACAGGACTCCA-3′ (SEQ ID NO: 333), BDNF forward: 5′TATTAGTGAGTGGGTAACGGCG3′ (SEQ ID NO: 334), and BDNF reverse: 5′GAAGTATTGCTTCAGTTGGCCTT3′ (SEQ ID NO: 335).

### FIG. 8 Material and Methods

Transient Transfections and Cell Culture. NBFL cells were grown as previously described¹²⁰. 0.5 M NFBL cells were seeded on a 35 mm dish, incubated at 37° C. overnight, and transfected with 2 μg of REST (SEQ. ID NO: 1)-IRES-GFP cDNA² using lipofectamine2000 (Life Technologies), following manufacturer recommendations. After a 48 hour incubation period, cell were sorted by fluorescence activated cell sorting (FACS),

mRNA extraction and quantification. mRNA was extracted, as described in Example 10, from GFP + and − cells; mRNA levels of Kv4.3 were determined using real-time RT-PCR¹¹⁶. Gene expression was normalized using β-actin (ACTB) as an internal housekeeping control. The β-actin primers used are the same as Example 10. The Kv4.3 primers were: forward: CTCACTACCACCTGCTGCTC (SEQ ID NO: 336) and reverse: TCAGTCCGTCGTCTGCTTTC (SEQ ID NO: 337).

### FIG. 9: Material and Methods

Plasmids. RPP (SEQ ID NOS: 2 and 4): One segment of this construct containing T7 promoter, CPP, and His tag was synthesized as a gBlock (Table 8) by IDT and amplified with primers described in Table 8. The PCR reaction (20 μL) was initially heated at 95° C. for 2.5 min followed by 30 cycles of denaturation at 94° C. for 15 sec., annealing at 55° C. for 15 sec., and extension at 72° C. for 30 seconds. Following amplification, PCR fragment was gel-purified by the QIAGEN gel-band purification kit according to manufacturer's protocol.

pET29 plasmid was amplified with primers: forward: P12 and P14 (Table 1) introducing SbfI and XhoI restriction sites. The PCR reaction (20 μL) was initially heated at 95° C. for 2.5 min followed by 30 cycles of denaturation at 94° C. for 15 sec., annealing at 55° C. for 15 sec. and extension at 72° C. for 6 min. Following amplification, PCR fragment was gel-purified by the QIAGEN gel-band purification kit and digested with SbfI and XhoI restriction enzymes. All digested fragments were purified with a QIAGEN kit and ligated in appropriate combination using T4 DNA ligase (NEB) according to the manufacturer's recommendations. Ligated fragments were transformed in 10G chemically competent cells and sequenced.

The nucleotide sequence of RPP (Table 8) was assembled and digested by HindIII and BamHI restriction enzymes. This fragment was cloned in pET vector described above in-frame with the CPP and His tag.

Control peptide: The control peptide (CP) was incorporated into the gBlock sequence shown in Table 9 and then cloned into the pET vector. The CP sequences are shown in Table 9.

In vitro Expression. RPP (Table 8) or CP (Table 9) were expressed in vitro using the PURE Express In Vitro Protein Synthesis Kit (New England Biolabs). The RPP (SEQ ID NOS: 2 and 4) or CP sequence was amplified from the RPP (SEQ ID NOS: 2 and 4) or CP expression constructs (Table 8 and 9) with primers listed in Table 8, and gel-purified by the QIAGEN gel-band purification kit. The PCR reaction (20 μL) was initially heated at 95° C. for 2.5 min followed by 30 cycles of denaturation at 94° C. for 15 sec., annealing at 55° C. for 15 sec., and extension at 72° C. for 30 sec. Then, 15 μL of the purified PCR fragment was mixed with 10 μL of Solution A and 7.5 μL of Solution B of the PURE Express In Vitro Protein Synthesis Kit (New England Biolabs) and incubated 2 hours at 37° C. Next, 2 μL of DNAse I was added and incubated for 20 min at 37° C.

TABLE 8
RPP-CPP-His_pET cassette

Nucleotide Sequence (SEQ ID NO: 316)
cctgcaggTAATACGACTCACTATAGGGTTAACTTTAGTAAGGAGGACAG
CTAAaagcttATGTGTACCGAAGATCTGGAACCACCAGAACCACCACTGC
CAAAGGAAAATTGTggatccGGCTCTGGTCGTAAAAAGCGCCGTCAGCGT
CGGCGTGGTGGCTCCGGTAGCTTAGGTCACCATCACCATCACCACGGCAC
CGGCTATTAActcgag
Coding Sequence (SEQ ID NO: 317)
ATGTGTACCGAAGATCTGGAACCACCAGAACCACCACTGCCAAAGGAAAA
TTGTggatccGGCTCTGGTCGTAAAAAGCGCCGTCAGCGTCGGCGTGGTG
GCTCCGGTAGCTTAGGTCACCATCACCATCACCACGGCACCGGCTATTAA
Protein Sequence (SEQ ID NO: 2)
MCTEDLEPPEPPLPKENCGSGSGRKKRRQRRRGGSGSLGHHHHHHGTGY
Amplification Primers
Forward: aaatcctgcaggTAATACGACTCACTATAGGGTTAAC
(SEQ ID NO: 318)
Reverse: ATTATTctcgagTTAATAGCCGGTGCCGTGGTGATGGTGAT
GGTGACCTA (SEQ ID NO: 319)
gBlock (SEQ ID NO: 320)
tataccctgcaggTAATACGACTCACTATAGGGTTAACTTTAGTAAGGAG
GACAGCTAAaagcttATGTGTGAAGACGCCAAAAACATAAAGAAAGGCCC
GGCGCCATTCTATCCGTGTggatccGGCTCTGGTCGTAAAAAGCGCCGTC
AGCGTCGGCGTGGTGGCTCCGGTAGCTTAGGTCACCATCACCATCACCAC
GGCACCGGCTATTAActcgagagatc
The restriction sites: small font
RPP, CPP and His tag: underlined

TABLE 9
Control-CPP-His pET cassette

Nucleotide Sequence (SEQ ID NO: 321)
cctgcaggTAATACGACTCACTATAGGGTTAACTTTAGTAAGGAGGACAG
CTAAaagcttATGTGTGAAGACGCCAAAAACATAAAGAAAGGCCCGGCGC
CATTCTATCCGTGTggatccGGCTCTGGTCGTAAAAAGCGCCGTCAGCGT
CGGCGTGGTGGCTCCGGTAGCTTAGGTCACCATCACCATCACCACGGCAC
CGGCTATTAActcgag
Coding Sequence (SEQ ID NO: 322)
ATGTGTGAAGACGCCAAAAACATAAAGAAAGGCCCGGCGCCATTCTATCC
GTGTggatccGGCTCTGGTCGTAAAAAGCGCCGTCAGCGTCGGCGTGGTG
GCTCCGGTAGCTTAGGTCACCATCACCATCACCACGGCACCGGCTATTAA
Protein Sequence (SEQ ID NO: 3)
MCEDAKNIKKGPAPFYPCGSGSGRKKRRQRRRGGSGSLGHHHHHHGTGY
Amplification Primers
Forward: aaatcctgcaggTAATACGACTCACTATAGGGTTAAC
(SEQ ID NO: 323)
Reverse: ATTATTctcgagTTAATAGCCGGTGCCGTGGTGATGGTGAT
GGTGACCTA (SEQ ID NO: 324)
The restriction sites: small font
Control peptide, CPP and His tag: underlined

Purification. RPP (SEQ ID NOS: 2 and 4) and CP: The final solution from the in vitro expression step was diluted to 100 μL and filtered through the Amicon Ultracel 0.5 mL-100K columns (Sigma) to remove ribosomes. The flow through was added to 100 μL of washed Ni-NTA Magnetic Agarose Beads (Qiagen) and was washed with wash buffer as described by the manufacturer.

Cyclization. The RPP (SEQ ID NO: 2) or was cyclized by mixing the beads with 500 μL of the cyclization solution which was made by mixing 2.65 mL of the 1.33×PBS (66.5 mM phosphate buffer, 400 mM NaCl) with 1.32 mL of dibromo-m-xylene solution in acetonitrile (2.5 mg/mL). Following incubation with the cyclization solution beads were washed once with wash buffer I (PBS with 25 mM imidazole, 0.5 μL/mL of mercaptoethanol), once with wash buffer II (PBS with 25 mM imidazole, 0.5 mM TCEP), twice with wash buffer III (PBS with 25 mM imidazole) and eluted with 50 μL of the elution buffer (PBS with 500 mM imidazole). Imidazole was removed using Bio-Rad Micro Bio-Spin Chromatography columns.

Dosing and Cell Culture. NBFL cells (FIG. 9) grown in 6 well were dosed with 1 μM of linear (SEQ ID NO: 2) or cyclic (SEQ ID NO: 2) RPP (Table 8) or control peptide (Table 9) for 16 or 48 hours.

Mesenchymal progenitor cells (MPCs) (FIG. 10) from two patients were plated on 12 well plates and cultured in general medium (GM) (DMEM, 10% FBS, PSF). Lyophilized RPP (SEQ ID NO: 9) was dissolved in water at a concentration of 5 mM (1 mg/34.89, diluted in medium to 3 μM, and then incubated with the cell for 48 hours. The control was water.

mRNA Extraction and Quantification.

After dosing, cells were lysed and assessed for BDNF, NGF, and Kv4.3 mRNA levels (FIG. 9). All of the primers used have been described except for NGF: forward: 5′TATCCTGGCCACACTGAGGT3′ (SEQ ID NO: 338) and reverse 5′TCCTGCAGGGACATTGCTC3′ (SEQ ID NO: 339).

### FIG. 10 Materials and Methods

MPCs were cultured as described in Gervasi et al., 2020⁷⁶.

### FIGS. 14, 15, and 16 Material and Methods

L5 DRGs were dissected from adult male rats and plated in 12-well plates pre-coated with poly-D-Lysine (PDL) and laminin in a minimal volume of medium (250 ul Neurobasal™-A medium (GIBCO) supplemented with B27 (GIBCO), 50 ng/ml NGF (Sigma-Aldrich) and penicillin streptomycin) to allow attachment to the culture plate. One day after plating, DRGs were incubated with RPP (SEQ. ID NO: 12: 1 μM, 3 μM or 10 μM in culture medium, FIGS. 14 and 16; SEQ ID NOS: 13 and 14:3 μM, FIG. 15) for 48 h. For RNA expression analysis, DRGs were lysed in 300 μl of Qiazol in a 1.5 ml tube containing Bullet Blender Pink Beads using a Bullet Blender Tissue Homogenizer (NextAdvance). Total RNA was extracted using RNeasy Midi Kit (Qiagen) according to manufacturer's instructions. Purified RNA was quantified with NanoDrop 2000 (ThermoFIsher), and reverse transcription was run using High Capacity cDNA Reverse Transcription Kit (Applied Biosystems) and 2 ng/ul RNA per reaction. For qRT-PCR, 5 ul of cDNA (corresponding to 10 ng of RNA) were mixed with to 10 ul SsoAdvanced™ Universal SYBR® Green Supermix (BioRad) and 1 ul of each primer (final primer concentration 500 nM each). Reactions were run in triplicates in a QuantStudio™ 7 Flex Real-Time PCR system (Applied Biosystems). Amplification data were analyzed using the comparative cycle threshold (ΔΔCt) method and β-actin as calibrator. Primers were as follows: β-actin: F-AGAGCTATGAGCTGCCTGAC (SEQ ID NO: 340), R-GGATGCCACAGGACTCCA (SEQ ID NO: 333); Kv4.3: F-AGCTGTGCCTCAGAACTAGGCTTT (SEQ ID NO: 341), R-TACCAGAAAGACGCAGGGATGCTT (SEQ ID NO: 342); Kv7.2 F-CCGGCAGAACTCAGAAGA AG (SEQ ID NO: 343), R-TTTGAGGCCAGGGGTAAGAT (SEQ ID NO: 344); OPRM1: F-TTCCTGGTCATGTATGTGATTGTA (SEQ ID NO: 345), R-GGGCAGTGTACTG GTCGCTAA (SEQ ID NO: 346).

### FIG. 17 Materials and Methods

Cytotoxicity Assay: Cytotoxicity of RPP (SEQ. ID NO: 12) was assessed with LDH-Glo™ Cytotoxicity Assay (Promega) according to the manufacturer's instructions. Medium was collected 48 h after treatment with RPP or 15 min incubation with 2% triton X-100 (which induces necrosis). Medium was diluted 1:50 in LDH storage buffer (200 mM Tris-HCl (pH 7.3), 10% Glycerol, 1% BSA). To verify the linear range of the assay, LDH titration curve was also run together with the experimental samples. 50 μl of diluted medium or LDH serial dilutions were incubated with 50 μl of LDH Detection Reagent (50 μl LDH Detection Enzyme Mix, 0.25 μl Reductase Substrate) for 40 min. Luminescence was recorded with an Infinite M200 Pro (Tecan) instrument.

Example 11—RPP (SEQ ID NOS: 1 and 15 Through 17) or RPPv (SEQ ID NOS: 18 Through 117) Peptide Fused to a CPP (SEQ ID NOS: 118 Through 137 and 140 Through 159) can be Used to Induce Neurodifferentiation

Approach 1. To determine if RPP (SEQ ID NOS: 1 and 15 through 17) or RPPv (SEQ ID NOS: 18 through 117) peptide fused to a CPP (SEQ ID NOS: 118 through 137 and 140 through 159) are the most effective in promoting human neurogenesis and differentiation, an in vitro screening assay using neural stem progenitor cells (NSPCs) from the NCRM-1/XCL-1 iPS cell line will be used. NCRM-1/XCL-1 iPS cells were generated from CD34+ human cord blood cells by episomal plasmid reprogramming¹²¹ and differentiated to neural stem progenitor cells (NSPCs) through an embryoid body (EB) method¹²². NCRM-1/XCL-1 NSPCs are positionally naïve NSPCs that can be rapidly differentiated to neurons¹²³ making them ideal for broad-based toxicology and phenotypic screening platforms¹²⁴⁻¹²⁶.

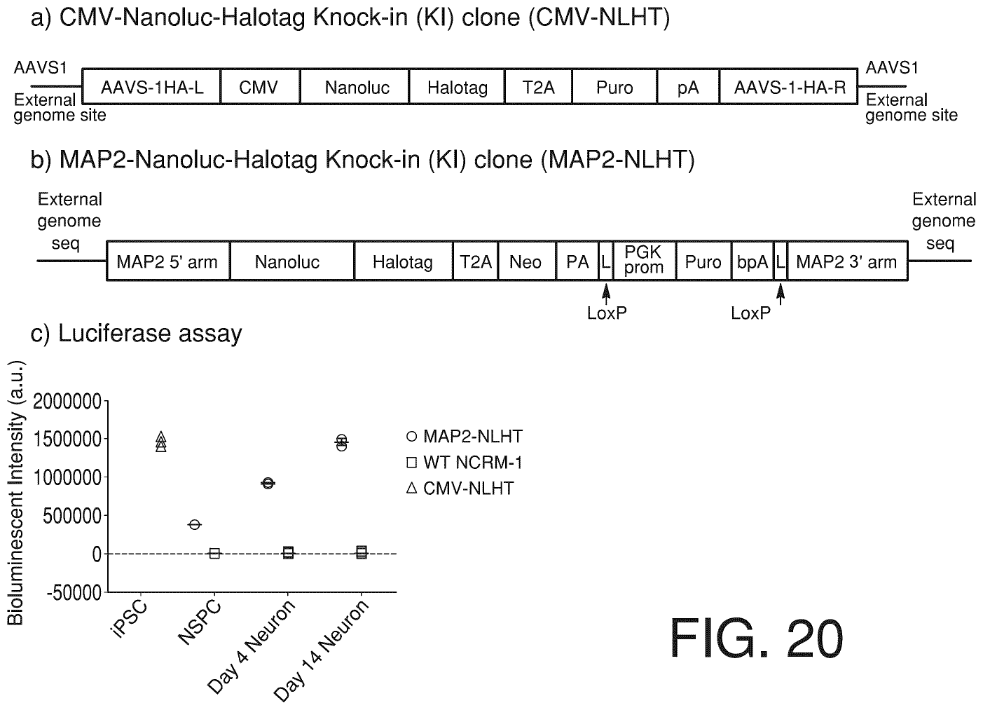

We will use two engineered NCRM-1/XCL-1 lines for high-throughput screening in 96-well plate assays. To determine if the RPP (SEQ ID NOS: 1 and 15 through 17) or RPPv (SEQ ID NOS: 18 through 117) peptide fused to a CPP (SEQ ID NOS: 118 through 137 and 140 through 159) is cytotoxic, we will use an NCRM-1/XCL-1 line that constitutively expresses NanoLuc® luciferase under the control the CMV promoter. In this line, a CMV-Nanoluciferase-Halotag (CMV-NLHT) construct was inserted into the safe harbor AAVS1 locus of Chr. 19q by transcription activator-like effector nucleases (TALENS) (FIG. 20a). We will use luciferase activity as a rapid surrogate readout of CMV-NLHT cell number. Using this approach, we will collect time-course data to measure toxicity over prolonged exposures to the drug. If the RPP (SEQ ID NOS: 1 and 15 through 17) or RPPv (SEQ ID NOS: 18 through 117) peptide fused to a CPP (SEQ ID NOS: 118 through 137 and 140 through 159) shows toxicity levels resulting in ≥10% loss of luciferase signal, this will be confirmed using CellTox™ Green Cytotoxicity Assay.

To assess the pro-neuronal differentiation effect of RPP (SEQ ID NOS: 1 and 15 through 17) or RPPv (SEQ ID NOS: 18 through 117) peptide fused to a CPP (SEQ ID NOS: 118 through 137 and 140 through 159), we will use a second engineered NCRM-1/XCL-1 line. In this line, Nanoluciferase-Halotag (NLHT) is knocked-into the MAP2 transcriptional start site (TSS) (Chr. 2) using zinc finger nucleases (ZFN) (FIG. 20b). MAP2 expression increases with neuronal differentiation in NCRM-1/XCL-1 cultures¹²⁷ and this is replicated by increasing luciferase activity in MAP2-NHLT culture media (FIG. 20c). We will use luciferase activity in media as a surrogate readout of MAP2-NLHT neurodifferentiation and collect time-course data to track RPP (SEQ ID NOS: 1 and 15 through 17) or RPPv (SEQ ID NOS: 18 through 117) peptide fused to a CPP (SEQ ID NOS: 118 through 137 and 140 through 159) differentiation effects over real-time. This approach will allow us to rapidly compare the neurodifferentiation rates in cultures treated with RPP (SEQ ID NOS: 1 and 15 through 17) or RPPv (SEQ ID NOS: 18 through 117) peptide fused to a CPP (SEQ ID NOS: 118 through 137 and 140 through 159) or control peptide. Positive neurodifferentiation effects will be independently validated by qRT-PCR expression screening of two cohorts of cell fate marker genes¹²¹; a cohort of NSPC and multipotency marker genes of LIN28A, RPS27L, IFITM2, IGFBP3, ANXA1; and neuronal marker genes C1ORF61, IGLON5, IGSF11, CHL1, SOX9. Luciferase activity and qRT-PCR values will be reported as mean #standard error of the mean (SEM). Statistically significant drug effects on the rate of neuronal differentiation will be determined by one-way analysis of variance (ANOVA) with Tukey's multiple comparison test.

PREDICTED RESULT & ALTERNATIVE STRATEGY. We predict that dosing of human NSPCs with RPP (SEQ ID NOS: 1 and 15 through 17) or RPPv (SEQ ID NOS: 18 through 117) peptide fused to a CPP (SEQ ID NOS: 118 through 137 and 140 through 159) will accelerate neuronal differentiation as measured by increased MAP2-luciferase activity, decreased expression of NSPC/multipotency markers and increased expression of neuronal markers when compared to vehicle controls.

If the RPP (SEQ ID NOS: 1 and 15 through 17) or RPPv (SEQ ID NOS: 18 through 117) peptide fused to a CPP (SEQ ID NOS: 118 through 137 and 140 through 159) effects shift the temporal profile NCRM-1/XCLs marker expression, this may necessitate data collection at alternate time-points. If the luciferase screen lacks the sensitivity to assess the pro-differentiation effects of the RPP (SEQ ID NOS: 1 and 15 through 17) or RPPv (SEQ ID NOS: 18 through 117) peptide fused to a CPP (SEQ ID NOS: 118 through 137 and 140 through 159), qRT-PCR data can be used to assess neural differentiation.

### Material and Methods

Plasmids. CMV-Nanoluciferase-Halotag (CMV-NLHT) is commercially available from Promaga.

Cell lines and cell culture. NCRM-1/XCL-1 iPS cells were generated from CD34+ human cord blood cells by episomal plasmid reprogramming¹²¹ and differentiated to neural stem progenitor cells (NSPCs) through an embryoid body (EB) method¹²² and neurons¹²³ as previously described. The method for inserting a CMV-Nanoluciferase-Halotag (CMV-NLHT) construct into the safe harbor AAVS1 locus of Chr. 19q (NCRM-1/XCL-1 cell line) by transcription activator-like effector nucleases (TALENS) was previously described by Papapetrou et all., 2016128. The method for knocking in Nanoluciferase-Halotag (NLHT) into the MAP2 transcriptional start site (TSS) (Chr.2, of the NCRM-1/XCL-1 cell line) using zinc finger nuclease (ZFN) has been previously described¹²⁷.

Luciferase assay. The methods for determining CMV-NLHT cell number and neurodifferentiation (MAP2-NLHT) have been previously described by Fritz et al., 2017¹²⁷ and He et al., 2011¹²⁹.

qRT-PCR screen. The qRT-PCR methods for screening cell fate marker genes has been previously described by Chou et al., 2011¹²¹.

Approach 2: Induced pluripotent stem cells (neural stem cells (NSC)-NL5) differentiate after 7 Days of RPP dosing (FIG. 11 (SEQ ID NOS: 5-11, 3 μM); FIG. 12 (SEQ ID NO: 9, 1 μM) FIG. 13 (SEQ ID NO: 13 (FIG. 27), 1 μM)) using the following “Neuron to Blank” protocol:
1. 24 well culture plates (Black Visiplate) were coated with Matrigel (12 mL/well for 30 minutes at 37° C.).
2. NSCs-NLS (see media below) were plated at 0.5×10⁵ per well.

| | Final | | |
|---|---|---|---|
| Component | Concentration | Amount | |
| DMEM/12 Medium | 1× | 97 | mL |
| N2 (100×) | 1× | 1 | mL |
| B27 Supplement | 2% | 2 | mL |
| bFGF (prepared as | 10 ng/mL | 10 | μL |
| 100 ug/mL stock) | | | |

3. After 24 hours (cells should be >70% confluent), the media was changed to neuronal differentiation medium (below) with various concentrations of test peptides or water control added (FIGS. 11, 12, and 13, Table 14). The neuronal differentiation medium, with new test peptides and control, was changed every 2 days for 5 days.

| | Final | | | |
|---|---|---|---|---|
| Component | Concentration | | Amount | |
| D-MEM/F-12 | 1× | | 88.6 | mL |
| GlutaMAX ™ -I Supplement | 2 | mM | 1 | mL |
| Bovine Serum Albumin (at 25%) | 1.8% | | 7.2 | mL |
| hESC Supplement | 2% | | 2 | mL |
| BDNF (prepared as 25 ug/mL stock) | 10 | ng/mL | 40 | μL |
| GDNF (prepared as 100 ug/mL stock) | 10 | ng/mL | 10 | μL |

4. On Day 7 cells were fixed 4% formaldehyde in 1×PBS for 30 minutes.
5. Cells were immunolabeled with mouse anti-TUJ1 (1:1000) and rabbit anti-Map2 (1:500) overnight. Then label with 2^{nd} antibody (anti-mouse488, anti-rabbit568 and DAPi) for 1 hour.
6. Data was collected on a BioTek plate reader
   a. Dapi: Excitation 360/40; Emission 460/20, Gain 35;
   b. Alexa 488: Excitation 485/20; Emission 528/20, Gain 50;
   c. Alexa 568: Excitation 560/20; Emission 620/10, Gain 75).

Results: In FIG. 11, RPPs SEQ ID NOS: 5 through 11 at 3 μM increased (1.7- to 2.7-times) NSC-NL5 differentiation after 7 days, as measured by MAP2 (mature neuron marker) expression normalized to DAPI (nucleus) and relative to control. In FIG. 12, RPP SEQ ID NO: 9 at 1 μM increased NSC-NL5 differentiation approximately 35% after 7 days, as measured by TUJ1 and MAP2 neuronal markers (normalized to DAPI (nucleus)) and relative to control. In FIG. 13, RPP SEQ ID NO: 13 at 0.1 and 1 μM increased NSC-NL5 differentiation after 7 days, as measured by TUJ1 (increased 60% and 3-times, respectively) and MAP2 (increased 27% and 2-times, respectively) neuronal markers (normalized to DAPI (nucleus) and relative to control). The basis for using this screen is that eliminating REST in neural progenitors cells has been shown to induce neuronal differentiation²², which is a readout for ensemble derepression of neuronal genes.

Example 12—RPP (SEQ ID NOS: 1 and 15 Through 17) or RPPv (SEQ ID NOS: 18 Through 117) Peptide Fused to a CPP (SEQ ID NOS: 118 Through 137 and 140 Through 159) can be Used to Improve Motor and Cognitive Function after Traumatic Brain Injury

Approach. We will determine whether treatment with the RPP (SEQ ID NOS: 1 and 15 through 17) or RPPv (SEQ ID NOS: 18 through 117) peptide fused to a CPP (SEQ ID NOS: 118 through 137 and 140 through 159), in a rat model of TBI alters neuronal survival and the amount of neurogenesis. We will correlate these measures with histopathological and functional recovery outcomes to determine the effectiveness of the REST peptide after TBI. We have shown that REST mRNA increases around the lesion after injury (FIG. 19). We will use a well-characterized rat model of controlled cortical impact injury (CCI)^{6,130-134}.

Male and female Sprague-Dawley rats (approximately 250-300 g males, 150-200 g females) will be injured by CCI unilaterally over the right parietal cortex (5 mm diameter, 4 m/s, 2 mm depth). Sham animals will undergo identical procedures without impact. Six hours after CCI, rats will have a cannula implanted in their right ventricle that will attach to an osmotic minipump for intraventricular catheter (ICV) delivery of the control or RPP (SEQ ID NOS: 1 and 15 through 17) or RPPv (SEQ ID NOS: 18 through 117) peptide fused to a CPP (SEQ ID NOS: 118 through 137 and 140 through 159) (0.17 μg/h). Six hours is a realistic initial therapeutic window with translational relevance to determine the efficacy of the RPP (SEQ ID NOS: 1 and 15 through 17) or RPPv (SEQ ID NOS: 18 through 117) peptide fused to a CPP (SEQ ID NOS: 118 through 137 and 140 through 159) will be infused until sacrifice. We will sacrifice rats at two different time points—the first cohort at 3 dpi to examine acute effects of the RPP (SEQ ID NOS: 1 and 15 through 17) or RPPv (SEQ ID NOS: 18 through 117) peptide fused to a CPP (SEQ ID NOS: 118 through 137 and 140 through 159) on neuronal death and proliferation, and the second cohort at 30 dpi to determine effects of the RPP (SEQ ID NOS: 1 and 15 through 17) or RPPv (SEQ ID NOS: 18 through 117) peptide fused to a CPP (SEQ ID NOS: 118 through 137 and 140 through 159) on functional and morphological recovery and neurogenesis. For each cohort, there will be 12 groups in a 3 (control peptide, two doses of RPP (SEQ ID NOS: 1 and 15 through 17) or RPPv (SEQ ID NOS: 18 through 117) peptide fused to a CPP (SEQ ID NOS: 118 through 137 and 159 through 143)×2 (sham, CCI)×2 (male/female) design. Male and female rats will be run on different days to enable identification of any sex-specific differences in injury, behavior or response to the RPP (SEQ ID NOS: 1 and 15 through 17) or RPPv (SEQ ID NOS: 18 through 117) peptide fused to a CPP (SEQ ID NOS: 118 through 137 and 140 through 159). Rats will be injected intraperitoneal (i.p.) with BrdU (50 mg/kg) daily during the first week to determine proliferation and maturational cell fate (1^{st} cohort-daily injections 1-3 dpi, sacrifice 2 h after final injection; 2nd cohort-daily 1-7 dpi). Our prior experience suggests that we will need 16 rats/group for significant behavioral data (2nd cohort), 8 rats/group for immunohistochemistry (1st cohort).

In the second cohort, to determine the functional consequence of treatment with the RPP (SEQ ID NOS: 1 and 15 through 17) or RPPv (SEQ ID NOS: 18 through 117) peptide fused to a CPP (SEQ ID NOS: 118 through 137 and 140 through 159) compared to controls, we will examine motor and cognitive behavior at different time points post-CCI or sham injury. We will train rats before injury to obtain baseline readings for motor function on the beamwalk, rotorod, and open field tests. Rats will be tested for recovery of motor function on both beamwalk and rotorod on days 1, 3, 7, and 10 days post-injury (dpi). Open field testing will be conducted on days 4, 12, and 22 dpi. Recognition memory will be assessed with a novel object recognition test performed at 20 dpi. Data will be analyzed by repeated measure two-way ANOVA with Dunnets post hoc correction. Spatial memory and learning will be determined by the Morris Water Maze (MWM) assay starting at 25 dpi. Swim speed and latency to find a hidden platform will be recorded for all trials. The probe trial, conducted on the fifth day of training, will determine the time the rat spends in the quadrant that previously contained the hidden platform. A visible platform test will also be performed on each rat to ensure that there is little difference in visual acuity between the animals. A two-way ANOVA with Tukey's multiple comparison correction will determine if the RPP (SEQ ID NOS: 1 and 15 through 17) or RPPv (SEQ ID NOS: 18 through 117) peptide fused to a CPP (SEQ ID NOS: 118 through 137 and 140 through 159) significantly alters cognitive function in the MWM in either the sham or injured rats in comparison with control peptide¹³⁵.

After the functional assessments, 8 rats/group will be sacrificed by transcardial perfusion, and the brains removed and processed. We will determine lesion size at both 3 and 30 dpi by staining sections with cresyl violet and measuring 12 sections spaced at 500 μm intervals through the lesion. Lesion volume will be expressed as a percentage of the volume of the ipsilateral hemisphere. Serial coronal brain sections through the frontoparietal cortex and dorsal hippocampus containing the subventricular zone (SVZ) and the dentate gyrus (DG) will be examined. The total number of BrdU positive cells (detected with anti-BrdU) in different hippocampal regions and the SVZ will be counted using stereology to determine whether the RPP (SEQ ID NOS: 1 and 15 through 17) or RPPv (SEQ ID NOS: 18 through 117) peptide fused to a CPP (SEQ ID NOS: 118 through 137 and 140 through 159) alters the survival of newly proliferated cells after injury. Sections will be co-stained with different cell-specific markers to determine the identity of the proliferated cells at 3 dpi and their maturational fate at 30 dpi. Cell specific markers include NeuN (mature neurons), SOX2 (neural stem cells), doublecortin (DCX) (neuronal progenitors), GFAP or ALDHIL1 (astrocytes), Iba1 (microglia), NG2 (oligodendrocyte precursor cells, OPCs), and APC or GSTpi (mature oligodendrocytes). The number of BrdU/NeuN double positive cells in the DG and olfactory bulb at 30 dpi will be compared between the different treatment groups to determine whether the RPP (SEQ ID NOS: 1 and 15 through 17) or RPPv (SEQ ID NOS: 18 through 117) peptide fused to a CPP (SEQ ID NOS: 118 through 137 and 140 through 159) increases post injury neurogenesis. We will also determine whether there is migration of BrdU+ neuronal progenitors or mature neurons to different regions of the hippocampus-away from the DG, or from the SVZ towards the lesion area or in the rostral migratory stream (RMS). Examination of the total number of each BrdU+ cell type within the perilesional area in addition to the neurogenic niches will determine whether the RPP (SEQ ID NOS: 1 and 15 through 17) or RPPv (SEQ ID NOS: 18 through 117) peptide fused to a CPP (SEQ ID NOS: 118 through 137 and 140 through 159) alters pathology after injury. As we expect that the RPP (SEQ ID NOS: 1 and 15 through 17) or RPPv (SEQ ID NOS: 18 through 117) peptide fused to a CPP (SEQ ID NOS: 118 through 137 and 140 through 159) will increase neuronal survival, we will quantify the number of degenerating neurons (Fluorojade C), and the number of surviving neurons (NeuN) in the peri-lesional area at both time points. We will examine the corpus callosum with Luxol fast blue to indicate any differences in the amount of myelin. The unbiased optical fractionator method will be used to count cells in the neurogenic areas, peri-lesional area, and the rostromigratory stream and olfactory bulb to obtain accurate cell specific and proliferating cell counts. All data will be analyzed by two-way ANOVA with Dunnetts post hoc correction. The distribution of RPP (SEQ ID NOS: 1 and 15 through 17) or RPPv (SEQ ID NOS: 18 through 117) peptide fused to a CPP (SEQ ID NOS: 118 through 137 and 140 through 159) will be examined in sections by staining with the FLAG antiserum. The remaining 8 rats per group (2nd cohort) will be sacrificed and their brains quickly removed. Brain regions that are both ipsilateral and contralateral to the lesion will be punched out and snap frozen for RNA and protein isolation. We will determine REST levels by western blot and BDNF expression by qRT-PCR around the lesion or in neurogenic niches in comparison to control treated rat brains.

PREDICTED RESULT & ALTERNATIVE STRATEGY. We expect the RPP (SEQ ID NOS: 1 and 15 through 17) or RPPv (SEQ ID NOS: 18 through 117) peptide fused to a CPP (SEQ ID NOS: 118 through 137 and 140 through 159) will reduce the lesion volume after CCI at both 3 dpi and 30 dpi by enhancing neuronal survival. Degenerating and surviving neurons will be examined at both time points to directly assess the effect of RPP (SEQ ID NOS: 1 and 15 through 17) or RPPv (SEQ ID NOS: 18 through 117) peptide fused to a CPP (SEQ ID NOS: 118 through 137 and 140 through 159) treatment on neuronal survival. A significant early neuroprotective effect of the RPP (SEQ ID NOS: 1 and 15 through 17) or RPPv (SEQ ID NOS: 18 through 117) peptide fused to a CPP (SEQ ID NOS: 118 through 137 and 140 through 159) in the perilesional area may result in improved motor function in the first week. Although we have previously found that the greatest neuronal death was at 3 dpi after CCI in the mouse¹³⁶, it is possible that we may need to examine a different time point in the rat. We also expect that animals treated with the RPP (SEQ ID NOS: 1 and 15 through 17) or RPPv (SEQ ID NOS: 18 through 117) peptide fused to a CPP (SEQ ID NOS: 118 through 137 and 140 through 159) will show increased neurogenesis in comparison to those treated with control peptide. Our data will show whether treatment with RPP (SEQ ID NOS: 1 and 15 through 17) or RPPv (SEQ ID NOS: 18 through 117) peptide fused to a CPP (SEQ ID NOS: 118 through 137 and 140 through 159) results in an increase in DCX+ precursors in different brain regions at 3 dpi or an increase in BrdU+/NeuN+ mature neurons in the hippocampus, olfactory bulb or around the lesion at 30 dpi. We will be able to correlate these data will behavioral outcomes, to see whether treatment with the RPP (SEQ ID NOS: 1 and 15 through 17) or RPPv (SEQ ID NOS: 18 through 117) peptide fused to a CPP (SEQ ID NOS: 118 through 137 and 140 through 159) will result in improved performance in the MWM assay or novel object recognition assay¹³⁵. In this way, we will be able to determine whether the RPP (SEQ ID NOS: 1 and 15 through 17) or RPPv (SEQ ID NOS: 18 through 117) peptide fused to a CPP (SEQ ID NOS: 118 through 137 and 140 through 159) has any negative effect on inflammation or cell proliferation.

### Materials and Methods

### Test System.

Species. Male and female Sprague-Dawley rat.

Age at Study start. Approximately 22 to 29 weeks

Weight at study start. Approximately 250 to 300 g for males and 150 to 200 g for females.

Study Design. For each cohort (1 and 2), there will be 12 groups in a 3 (control peptide, two doses of RPP (SEQ ID NOS: 1 and 15 through 17) or RPPv (SEQ ID NOS: 18 through 117) peptide fused to a CPP (SEQ ID NOS: 118 through 137 and 140 through 159)×2 (sham, CCI)×2 (male/female) design. In cohort 1 (immunohistochemistry), there will be 8 rats/group and in cohort 2 (behavior), there will be 16 rats/group.

Rational for dose levels. The high dose will be the maximum feasible dose (approximately 0.17 μg/h) and the lower dose will be the estimated efficacious dose based on cerebral spinal fluid (CSF) drug levels that approximate the efficacious dose determined in Example 13.

Route of Administration. A cannula implanted in the right ventricle attach to an osmotic minipump for intraventricular catheter (ICV) delivery.

Frequency of Administration. Continuous infusion until sacrifice.

Period of Dosing. Cohort 1:3 dpi; Cohort 2:30 dpi.

Environmental Conditions. 2 Rats/cage, with food and water provided ad libitum.

Test Article Identification. RPP (SEQ ID NOS: 1 and 15 through 17) or RPPv (SEQ ID NOS: 18 through 117) peptide fused to a CPP (SEQ ID NOS: 118 through 137 and 140 through 159)

Purity. ≥95%

Preparation of Dose Formulation. In bulk every 4 days.

Dose Formulation Assay and Stability. Dose formulations will be assessed for stability and concentration on Day 1 and 7 in the first week of the study. The acceptable concentration range is ±10% of nominal.

Controlled Cortical Impact (CCI) Model. The CCI model has been previously described^{6,130-134}. RPP (SEQ ID NOS: 1 and 15 through 17) or RPPv (SEQ ID NOS: 18 through 117) peptide fused to a CPP (SEQ ID NOS: 118 through 137 and 140 through 159) or control (vehicle only) administration will initiate 6 h after CCI.

Immunohistochemistry. Methods have been previously described by Xiong, Y et al., 2007 and 2008^{135,137}.

Morris Water Maze. Methods have been previously described by Choi et al., 2006¹³⁸.

Example 13—RPP (SEQ ID NOS: 1 and 15 Through 17) or RPPv (SEQ ID NOS: 18 Through 117) Peptide Fused to a CPP (SEQ ID NOS: 118 Through 137 and 140 Through 159) can be Used to Improve Regeneration after Peripheral Nerve Injury

Approach. Neurotrophic factors (NTFs) are needed for functional recovery after a peripheral nerve injury (PNI) and their presence, or lack thereof, are biomarkers of the strength of the regenerative response^{139,140}. Several NTF genes, including brain-derived neurotrophic factor (BDNF), nerve growth factor (NGF), pleiotrophin (PTN), and neurotrophin-3 (NTF-3), are known to be repressed by injury-induced expression of REST and we have demonstrated that RPP (SEQ ID NO: 2) can reverse this transcriptional repression, at least in the case of BDNF and NGF (FIGS. 7, 9, 10, and 16)^{141,142}.

1) Real-time quantitative reverse transcription PCR (qRT-PCR) analysis showed that NBFL cells dosed with 1 μM linear (SEQ ID NO: 4) or cyclic (SEQ ID NO: 2) RPP for 16 or 48 hours, evoke an increase in BDNF and NGF mRNA expression (FIG. 9, A and B and table below).

NTF response to 1 μM RPP in FIG. 9

| | | Increase | |
|---|---|---|---|
| NTF | RPP | 16 h | 48 h |
| BDNF | Linear | 5× | 2.25× |
| BDNF | Cyclic | NA | 1.75× |
| NGF | Linear | 3.2× | 1.66× |
| NGF | Cyclic | NA | 1.87 |

NA = Not assessed

2) RPP (SEQ ID NO: 9, 3 μM) increased NTFs BDNF and NGF approximately 2- and 3-times, respectively, relative to water control, after 48 hours. This finding confirmed that RPP is active in MPCs (FIG. 10). MPCs were cultured in general medium as previously described by Gerevasi et al., 2020⁷⁶.

3) We will quantify the effect of RPP (SEQ ID NOS: 1 and 15 through 17) or RPPv (SEQ ID NOS: 18 through 117) peptide fused to a CPP (SEQ ID NOS: 118 through 137 and 140 through 159) on the expression of NGF, BDNF, PTN, and NTF-3 in mesenchymal progenitor cells (MPCs) isolated from male and female patients with musculoskeletal trauma. MPCs are present at the site of peripheral nerve injury and are extensively characterized¹⁴³. MPCs will be harvested during the course of normal and pre-planned surgical treatments using a standard isolation protocol developed by Dr. Leon Nesti^{116,143} MPCs from 3 different subjects will be expanded in culture. First, we will passage cells four times without neuronal induction to establish a baseline for the expression of REST and NTFs transcript and protein levels by qRT-PCR and Western Blot (WB). Next, we will repeat expansion with neuronal induction as described by Bulken-Hoover, et al.¹⁴⁴. Briefly, MPCs are plated in pre-induction media, for 2 days, then augmented with all-trans-retinoic acid (RA) for 1 day, followed by 7 days in the neuroinductive media. The neuroinductive media will be changed every third day. On day 7-post induction, REST and NTFs levels will be assessed. We expect that neuronal induction should result in decreased REST expression with a corresponding increase in expression of NTFs.

To assess the potency of RPP (SEQ ID NOS: 1 and 15 through 17) or RPPv (SEQ ID NOS: 18 through 117) peptide fused to a CPP (SEQ ID NOS: 118 through 137 and 159 through 143) induction of NTFs expression in MPCs, we will first need to establish dosing by assessing RPP (SEQ ID NOS: 1 and 15 through 17) or RPPv (SEQ ID NOS: 18 through 117) peptide fused to a CPP (SEQ ID NOS: 118 through 137 and 140 through 159) cytotoxicity. Briefly, MPCs will be plated in a modified pre-induction media substituting RA with RPP (SEQ ID NOS: 1 and 15 through 17) or RPPv (SEQ ID NOS: 18 through 117) peptide fused to a CPP (SEQ ID NOS: 118 through 137 and 140 through 159) at 4 dose levels up to 10 μM or pre-induction media (with RA) for 1-day. There will be three technical and three biological replicates for each dosing group and cytotoxicity will be determined using the MTT assay¹⁴⁵. To determine the optimal concentration of RPP (SEQ ID NOS: 1 and 15 through 17) or RPPv (SEQ ID NOS: 18 through 117) peptide fused to a CPP (SEQ ID NOS: 118 through 137 and 140 through 159) for decreasing levels of REST and increasing levels of NTFs, we will perform a dose-response (3 dose levels) in neuroinductive media with RPP (SEQ ID NOS: 1 and 15 through 17) or RPPv (SEQ ID NOS: 18 through 117) peptide fused to a CPP (SEQ ID NOS: 118 through 137 and 140 through 159) replacing RA at 1, 3, and 6-days post-treatment (dpt). RPP (SEQ ID NOS: 1 and 15 through 17) or RPPv (SEQ ID NOS: 18 through 117) peptide fused to a CPP (SEQ ID NOS: 118 through 137 and 140 through 159) doses will be based on the findings of the MTT assay¹⁴⁵. Our negative and positive controls will be neuroinductive media − or +RA, respectively. We will quantify the gene expression and protein levels of REST and NTFs with qRT-PCR and WB^{139,146} Cells will be harvested at 1, 4, and 7-dpt and RNA and protein will be isolated from the cell lysates. A two-way ANOVA analysis, followed by a Tukey's post hoc test, will be performed to make comparisons between − and + controls and RPP (SEQ ID NOS: 1 and 15 through 17) or RPPv (SEQ ID NOS: 18 through 117) peptide fused to a CPP (SEQ ID NOS: 118 through 137 and 140 through 159) dosing groups for indicated time points. Means of populations will be reported as mean±SD, with a p-value of 0.05 or less considered statistically significant. Statistical analysis will be performed using the SAS statistical package (SAS Institute, Cary NC).

4) RPP (SEQ ID NO: 12) was assessed at 0, 0.3, 1, 3, 10 μM in an ex vivo culture of whole DRG neurons (L5, from male SD rats) for its potential to induce expression of BDNF and NGF (FIG. 16). RPP increased BDNF expression approximately 25% at 0.3, 1, and 3 μM and 75% at 10 μM. RPP increased NGF approximately 50% and 2-times at 3 and 10 μM, respectively (FIG. 15). No affect was observed at 1 μM, and 0.3 μM was inconclusive due to an n=1 (FIG. 16).

5) RPP (SEQ ID NO: 12) was assessed in an LDH cytotoxicity assay conducted on ex vivo cultured whole DRG neurons (L5, from male SD rats). RPP showed no toxicity at 0, 1, 3, or 10 μM (FIG. 17). As expected, the positive control (2% triton) was neurotoxic, as demonstrated by an increase of 90,000 RLU.

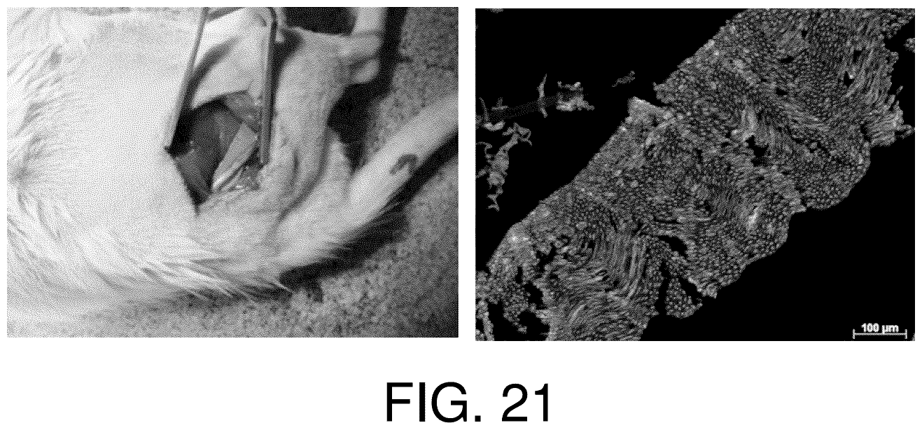

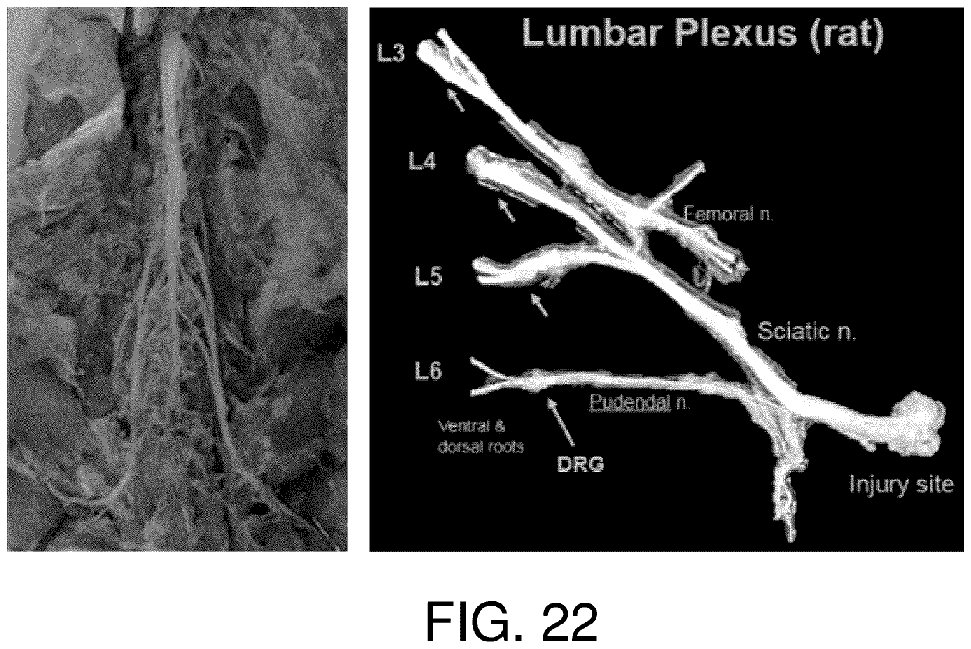

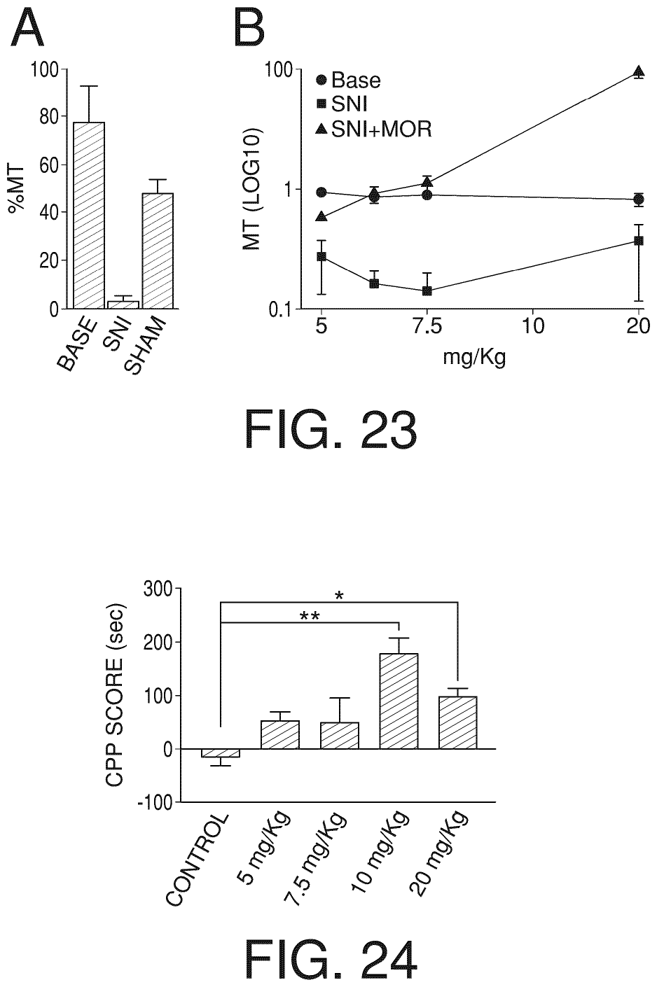

6) We will assess the ability of RPP (SEQ ID NOS: 1 and 15 through 17) or RPPv (SEQ ID NOS: 18 through 117) peptide fused to a CPP (SEQ ID NOS: 118 through 137 and 140 through 159) to induce NTF expression and stimulate regeneration in both motor and sensory ex vivo nerve models through the respective use of spinal cord and isolated dorsal root ganglion (DRG) explants¹⁴⁷. Both models are used extensively to study neuroprotective and trophic properties of growth factors¹⁴⁸. A 10 mm segment of intact DRG will be extracted (from 6 rats) from both sides of the spinal column (L4-L6) and cultured (FIGS. 21 & 22)^{149,150}. The remaining spinal column material will be preserved for motor-neuron linked spinal cord slice cultures. These spinal columns will be sectioned transversely at 300 μm intervals with a microtome and the slices will be transferred to culture inserts with semipermeable membranes and allowed to acclimatize in culture conditions for a week^{149,150}. The culture inserts with week-old organotypic spinal cord slices, having a stable population of surviving motor neurons, will then be transferred to 6-well plate for an additional 7 days in culture.

Both ex vivo explant cultures will be maintained for 2 weeks in neurobasal/B27 medium to allow extensive elongation of neuronal processes¹⁴⁷. To induce a physical injury, a glass Pasteur pipette will be used to create a scratch through the elaborated neuronal processes 6 mm from the perimeter of the DRG ganglia and from the gray-white matter junction of the ventral spinal cord slice. The ex vivo cultures will be treated with RPP (SEQ ID NOS: 1 and 15 through 17) or RPPv (SEQ ID NOS: 18 through 117) peptide fused to a CPP (SEQ ID NOS: 118 through 137 and 140 through 159) (0, 1, 3, or 10 μM) starting 24 hours post-injury.

The regenerative capacity of injured neurites (motor and sensory) will be determined by monitoring cell viability and measuring axonal outgrowth. Cell viability will be assessed 7-dpt by labeling live and dead cells respectively with calcein-AM and ethidium homodimer-1. Neurite extension, an indicator of neuron regeneration, will be measured at 1, 4, and 7-dpt. Gray-scaled micrographs of the scratch region will be acquired with a Zeiss AxioObserver microscope coupled to a monochrome digital camera¹⁵¹. The number, length, and total area of regenerated sprouts within the scratch region will be analyzed with ImageJ¹⁵².

NGF exerts much of its functional activity through its receptor, the TrkA receptor in the DRG¹⁵³, while PTN has been found to cause increased axonal outgrowth primarily in motor neurons^{150,154} BDNF and NTF-3 are present in both motor and sensory neurons. We will quantify the gene expression and protein levels of NTFs with qRT-PCR and WB^{139,146} Tissue from each ex vivo system harvested at 1, 4, and 7-dpi and RNA and protein lysates isolated. A two-way ANOVA analysis, followed by a Tukey's post hoc test, will be performed to make comparisons between control and RPP (SEQ ID NOS: 1 and 15 through 17) or RPPv (SEQ ID NOS: 18 through 117) peptide fused to a CPP (SEQ ID NOS: 118 through 137 and 140 through 159) dosing groups for indicated time points. Means of populations will be reported as mean±SD, with a p-value of 0.05 or less considered statistically significant. Statistical analysis will be performed using SAS software (SAS Institute, Cary NC).

7) RPP (SEQ ID NOS: 1 and 15 through 17) or RPPv (SEQ ID NOS: 18 through 117) peptide fused to a CPP (SEQ ID NOS: 118 through 137 and 140 through 159) will be assessed for the ability to improve regeneration and functional recovery after sciatic nerve defect in vivo in 100 rats. We have demonstrated that RPP accumulates in the nuclei of the sciatic nerve neurons in the spinal cord 48 hours after RPP was injected at the sciatic nerve injury site (FIG. 4). RPP (SEQ ID NOS: 1 and 15 through 17) or RPPv (SEQ ID NOS: 18 through 117) peptide fused to a CPP (SEQ ID NOS: 118 through 137 and 140 through 159) will be administered intravenously (IV), which will allow us to test a higher maximum feasible dose (MFD) as compared to intramuscular (IM) or subcutaneous (SC) administration. Administration by IV also avoids first-pass metabolism associated with oral and intraperitoneal (IP) administration. Dosing concentrations for the regeneration and functional recovery study will be based on the findings of a one-month single IV dose range-finding toxicity and toxicokinetic study in Sprague-Dawley rats. The dose range-finding toxicity study will be initiated upon completion after completion of 1). Briefly, the study will use four dosing levels up to 1000 mg/kg or max tolerated dose or MFD, as recommended by FDA guidance (M3 (R2), 2009). The number of animals allocated per group will be as follows: Main Study: 10/sex/group; TK: 3/sex/control group and 9/sex/dosing group. Animals will be assessed for body weight, food consumption, clinical chemistry, hematology, urinalysis, organ weights, histopathology (8 core tissues), and toxicokinetics.

In the regeneration and functional recovery study, Sprague-Dawley rats will be administered RPP (SEQ ID NOS: 1 and 15 through 17) or RPPv (SEQ ID NOS: 18 through 117) peptide fused to a CPP (SEQ ID NOS: 118 through 137 and 140 through 159) weekly, by IV using a long-term venous catheter¹⁵⁵, for 6-weeks in 3 dosing groups (TBD)+control (10/sex/group). All animals will be anesthetized, prior to exposing the sciatic nerve¹⁵⁶ and removing a 0.7 cm section to create an approximately 1 cm segmental defect following retraction of the nerve stumps¹⁵⁷. The nerve will be repaired using the decellularized nerve graft. The surgical incision will then be closed, and the nerve will be allowed to regenerate for 6 weeks. Only the dosing groups will be given RPP (SEQ ID NOS: 1 and 15 through 17) or RPPv (SEQ ID NOS: 18 through 117) peptide fused to a CPP (SEQ ID NOS: 118 through 137 and 140 through 159). During the 6-week period, we will evaluate functional recovery and tolerance to RPP (SEQ ID NOS: 1 and 15 through 17) or RPPv (SEQ ID NOS: 18 through 117) peptide fused to a CPP (SEQ ID NOS: 118 through 137 and 140 through 159) dosing. Each week the rats will be weighed, temperature recorded, and toe-spreading reflex assay (to determine the maximum footprint width of the injured leg testing motor nerve function) will be performed¹⁵⁶. Walking track analysis (Digigait) will be performed every 2 weeks, and electrophysiological studies¹⁵⁸ will be conducted. Briefly, electrophysiology testing will be performed on the rat sciatic nerve. Electrical stimuli are applied using single-pulse shocks (1 mA, 0.1 ms) to the native sciatic nerve trunk at the point 5 mm proximal to the graft suturing point. Compound muscle action potentials (CMAPs) are recorded on the gastrocnemius belly from 1V to 12V or until a supramaximal CMAP is reached. Normal CMAPs from the un-operated contralateral side of sciatic nerve are also recorded for comparison. The recovery rate will be determined by the ratio of injured hindlimb's CMAP to contralateral normal hindlimb's CMAP^{159,160}.

At the end of week 6, the sciatic nerve as well as the ventral horn (VH) of the spinal cord and DRG associated with the L4 to L6 nerve roots will be collected^{161,162}. Nerve regeneration will be evaluated histologically. The proximal and distal ends and the center of the grafts will be prepared for transmission electron microscopy (TEM) through the cross section to evaluate myelination along the graft^{163,164}. Briefly, ultrathin sections of ˜70 nm will be cut from the nerve using an ultramicrotome, then stained with uranyl acetate and lead citrate. 10-15 fields from random sections will be selected for analysis and the number, and the size of myelinated fibers will be quantified. Mean fiber density will be calculated as previously described⁷³. In addition to TEM, more sections from the same nerve will be prepared for IHC to visualize β-tubulin III in order to measure the direct growth of the axons, S100 to measure the penetration and growth of Schwann cells into the graft, von Willebrand factor to assess for angiogenesis and capillary infiltration of the healing nerve, and Luxol blue staining^{165,166} to measure myelin formation within the graft. The ventral horns and DRG will be assess for RPP (SEQ ID NOS: 1 and 15 through 17) or RPPv (SEQ ID NOS: 18 through 117) peptide fused to a CPP (SEQ ID NOS: 118 through 137 and 140 through 159) associated changes within the cell bodies of the motor and sensory nerves that were injured. Images of the stained tissues will be taken with a light microscope and quantification of intensity and area fraction of the positive reactions in anatomically matched tissues will be performed using the ImageJ software (https://imagej.nih.gov/ij), to compare nerve regeneration rates between the groups¹⁶⁵. DRG neurons of the injured nerve will be counted and compared to the contralateral uninjured DRG as previously described¹⁶⁷. NTFs (BDNF, NGF, PTN, and NTF-3), CTDSP1 and REST will be examined via qPCR and WB.

PREDICTED RESULT & ALTERNATIVE STRATEGY. We expect that mesenchymal progenitor cells (MPCs) treated with RPP (SEQ ID NOS: 1 and 15 through 17) or RPPv (SEQ ID NOS: 18 through 117) peptide fused to a CPP (SEQ ID NOS: 118 through 137 and 140 through 159)+neuroinductive media (−retinoic acid, RA) will increase the expression of neurotrophic factors (NTFs) and decrease the levels of REST, compared to neuroinductive media −RA, but similar to neuroinductive media +RA. We expect that the ex vivo models treated with RPP (SEQ ID NOS: 1 and 15 through 17) or RPPv (SEQ ID NOS: 18 through 117) peptide fused to a CPP (SEQ ID NOS: 118 through 137 and 140 through 159) will show an increase in neurite outgrowth in comparison to the no treatment group due to decreased REST levels. It is possible that the regenerative effects of RPP (SEQ ID NOS: 1 and 15 through 17) or RPPv (SEQ ID NOS: 18 through 117) peptide fused to a CPP (SEQ ID NOS: 118 through 137 and 140 through 159) may not be pronounced in the absence of support cells, which can secrete neurotrophic factors in vivo following neuronal injury. An alternative experiment will include the co-culture of neuro-supportive cells (e.g., Schwann cells and mesenchymal stem cells) with the dorsal root ganglion (DRG) and spinal cord explants, enriched with motor and sensory neurons, prior to injury and the subsequent RPP (SEQ ID NOS: 1 and 15 through 17) or RPPv (SEQ ID NOS: 18 through 117) peptide fused to a CPP (SEQ ID NOS: 118 through 137 and 140 through 159) treatment. Successively we will analyze the ability of the RPP (SEQ ID NOS: 1 and 15 through 17) or RPPv (SEQ ID NOS: 18 through 117) peptide fused to a CPP (SEQ ID NOS: 118 through 137 and 140 through 159) to help the regeneration of the severed nerve in a rat model. We expect that the rats treated with RPP (SEQ ID NOS: 1 and 15 through 17) or RPPv (SEQ ID NOS: 18 through 117) peptide fused to a CPP (SEQ ID NOS: 118 through 137 and 140 through 159) will have improved recovery as compared to control rats as determined by histological, electrophysiological, and functional assessments. We also expect RPP (SEQ ID NOS: 1 and 15 through 17) or RPPv (SEQ ID NOS: 18 through 117) peptide fused to a CPP (SEQ ID NOS: 118 through 137 and 140 through 159) to increase NTFs expression and decrease REST protein levels. If we do not observe signs of recovery and removal of the REST blockade, we will increase the local concentration of RPP (SEQ ID NOS: 1 and 15 through 17) or RPPv (SEQ ID NOS: 18 through 117) peptide fused to a CPP (SEQ ID NOS: 118 through 137 and 140 through 159) by direct injection to the target site (i.e., graft site or intrathecal).

### Materials and Methods

Cell Culture. MPC harvesting, culturing, and neurotrophic induction have been previously described¹⁶⁸. Briefly, MPCs are plated in pre-induction media, for 2 days, then augmented with all-trans-retinoic acid (RA) for 1 day, followed by 7 days in the neuroinductive media. The neuroinductive media will be changed every third day. On day 7-post induction, REST and NTFs levels will be assessed.

qRT-PCR. Methods described in Example 10.

Western Blot Analysis. Whole-cell lysates were prepared following the procedures in Ballas et. al., 2001¹¹⁹. Western blots will be performed by standard procedures using anti-REST-C⁶⁴, anti-NTFs (using commercially available antibodies), anti-GAPDH (abcam [6C5]), and anti-IgG conjugated to infrared dyes (Thermo Fisher), and analyzed on an Odyssey infrared fluorescence imager (LiCor).

Test Article Identification. RPP (SEQ ID NOS: 1 and 15 through 17) or RPPv (SEQ ID NOS: 18 through 117) peptide fused to a CPP (SEQ ID NOS: 118 through 137 and 140 through 159), Purity. ≥95%

Preparation of Dose Formulation. In bulk every 4 days.

Dose Formulation Assay and Stability. Dose formulations will be assessed for stability and concentration on Day 1 and 7 in the first week of the study. The acceptable concentration range is ±10% of nominal.

Vertebrate Animals. Male and female adult Sprague-Dawley rats, (approximately 250-300 g males, 150-200 g females) will be used to determine the efficacy of the RPP (SEQ ID NOS: 1 and 15 through 17) or RPPv (SEQ ID NOS: 18 through 117) peptide fused to a CPP (SEQ ID NOS: 118 through 137 and 140 through 159) in improving recovery from PNI. The studies will require 100 rats, 8-11 weeks old. Rats will be obtained from Charles River Laboratories.

### Description of Procedures:

Surgery: Rats will be anesthetized with isofluorane (4% induction, 3% maintenance) prior to all surgical procedures. In 2) organotypic spinal cord cultures will be prepared from the lumbar spinal cord of postnatal-day-8 rats (Sprague Dawley) using techniques as described previously (Rothstein et al., 1993; Corse et al., 1999). Briefly, rats will be quickly sacrificed, and the lumbosacral spinal cord will be removed and placed in Gey's balanced salt solution (Gibco) containing glucose (6.4 mg/L). In a laminar flow hood using sterile technique, the meninges will be carefully removed under magnification, and the lumbar spinal nerve roots will be transected. The cord will be placed on Aclar film and sectioned at 300 μm intervals from L2 to L5 with a Mcllwain tissue chopper. Individual cord sections will be carefully transferred, using the Gey's balanced salt/glucose solution, to Millicell-CM (Millipore) permeable membranes in 6-well culture plates. Five cord sections will be placed on each membrane. Each well contained 1 mL of medium consisting of 50% minimal essential medium plus 25 mM Hepes; 25% Hanks balanced salt solution with D-glucose (25.6 mg/L); 25% heat-inactivated horse serum; and 2 mM L-glutamine. Cultures will be maintained at 37° C. under 5% CO2 in a humidified incubator for a week with medium being changed every 3 days. After a week, the slices will be transferred to another 6-well culture plates and allowed to stabilize and extend neurites for another 7 days in culture prior to any treatments.

In 3) we will expose the sciatic nerve¹¹⁷ (FIGS. 21 & 22) and remove a 0.7 cm section to create an approximately 1 cm segmental defect following retraction of the nerve stumps (Hems and Glasby, 1993). The nerve will be repaired using the decellularized nerve graft. The surgical incision will then be closed, and the nerve will be allowed to regenerate for 6 weeks. Group one (control) will undergo approximately 1 cm sciatic nerve resection and will be grafted with a 1 cm decellularized allograft nerve. Group two through five will undergo a 1 cm sciatic nerve resection, grafted with a 1 cm decellularized allograft and given weekly IV injections of RPP (SEQ ID NOS: 1 and 15 through 17) or RPPv (SEQ ID NOS: 18 through 117) peptide fused to a CPP (SEQ ID NOS: 118 through 137 and 140 through 159) at drug concentrations to be determined from the dose range-finding study. All animals will be sacrificed at week 6.

Behavioral Assessments: We will monitor the animals for up to 6 weeks to evaluate functional recovery and tolerance to peptide treatment. Each week the rats will be weighed, temperature recorded and toe-spreading reflex assay (to determine the maximum footprint width of the injured leg testing motor nerve function) will be performed¹¹⁷. Walking track analysis will be performed every 2 weeks. and Electrophysiological studies will be conducted prior to sacrifice at 6 weeks.

Electrophysiology Assessments: Electrophysiology testing will be performed following previous methods¹⁵⁸. In brief, the rat sciatic nerve is re-exposed and electrical stimuli (single-pulse shocks, 1 mA, 0.1 ms) are applied to the native sciatic nerve trunk at the point 5 mm proximal to the graft suturing point. CMAPs are recorded on the gastrocnemius belly from 1V to 12V or until a supramaximal CMAP is reached. Normal CMAPs from the un-operated contralateral side of sciatic nerve are also recorded for comparison. Grass Tech S88X Stimulator (Astro-Med Inc.) is used for the test and PolyVIWE16 data acquisition software (Astro-Med, Inc.) is used for recording. Recovery rate is the ratio of injured hindlimb's CMAP to contralateral normal hindlimb's CMAP of a rat¹⁵⁸.

Sacrifice: Rats will be sacrificed at 6 weeks post injury. After sacrifice, we will use histology to evaluate axon growth through the graft, remyelination, MPC cell activity at the site of injury, anterior horn cell and DRG activity and vascularization of the regenerating nerve. We will harvest the gastrocnemius and tibialis anterior muscle of each rat after sacrifice to assess gross weight to quantify atrophy.

Justification: After PNI, there is a complex interplay between cells and molecules in the vasculature, the immune system and all of the cells endogenous to the peripheral nervous system. We do not completely understand the sequence nor the interactions between the different cells, signaling molecules, cell-cell interactions and circuitry that occurs after traumatic injury, so it is impossible to model it in vitro at the current time. Additionally, the RPP (SEQ ID NOS: 1 and 15 through 17) or RPPv (SEQ ID NOS: 18 through 117) peptide fused to a CPP (SEQ ID NOS: 118 through 137 and 140 through 159) that is the subject of investigation in this proposal may impact neural precursor cells, adult neurons as well as other cell types, and so it is critical to evaluate it in vivo in the context of PNI. The FDA also requires in vivo proof of efficacy before allowing IND status for a biologic, so this provides further justification for the in vivo nature of these experiments. Rats are used because of a significant body of behavioral research after PNI has indicated their reliability in these types of experiments. Thus, there is an extensive literature that provides a detailed framework for design and execution of experiments. Rats are more intelligent than mice, and are larger, enabling a greater ability to discern changes in functional recovery after injury. Rat physiology has more similarities to humans than the mouse, allowing for better translational relevance of experimental data. Further, the REST pathway we are targeting is conserved in Rats.

Minimization of Pain and Distress: Rats will be anesthetized with isoflurane (3-4%) for the segmental peripheral nerve defect. Following surgery animals will be allowed to recover on a heat pad until mobile. Rats will be administered acetaminophen (6 mg/ml in drinking water) for 2 days after the surgery, and additionally if signs of pain or distress are observed. Rats will be observed daily by both laboratory and veterinary staff, and animals deemed to be in distress will be treated or euthanized according to veterinary recommendations.

Euthanasia: The euthanasia used is consistent with the recommendations of the American Veterinary Medical Association Guidelines for the euthanasia of animals.

Example 14-RPP (SEQ ID NOS: 1 and 15 Through 17) or RPPv (SEQ ID NOS: 18 Through 117) Peptide Fused to a CPP (SEQ ID NOS: 118 Through 137 and 140 Through 159) can be Used for the Prevention and Treatment of Chronic Pain

### Completed Assessments:

The activation of REST after nerve injury results in decreased expression of several genes required for normal excitability of sensory neurons, including the potassium channels Kv4.3 (Kcnd3) and Kv7.2 (Kcnq2), the sodium channel Nav1.8 (Scn10a), and the mu opioid receptor Oprm1^{60,72-74} The basis for blocking REST to alleviate chronic pain are published studies using mouse and rat peripheral nerve injury (PNI) models^{60,72-74}.

1) Real-time quantitative reverse transcription PCR (qRT-PCR) analysis showed that NBFL cells dosed with 1 μM linear (SEQ ID NO: 4) or cyclic (SEQ ID NO: 2) RPP for 16 or 48 hours, evoke an increase in Kv4.3 mRNA expression (FIG. 9, C and Table 16 below).

TABLE 16
Kv4.3 response to 1 μM RPP in FIG. 9

| | Increase | |
|---|---|---|
| RPP | 16 h | 48 h |
| Linear | 5× | 5× |
| Cyclic | NA | 25× |

NA = Not assessed

4) RPP (SEQ ID NO: 12; 0 (water), 1, 3, or 10 μM) was incubated for 48 hours in an ex vivo culture of whole DRGs (L5, from male SD rats) and assessed for its potential to induce expression of chronic pain associated genes Kv4.3, Kv7.2, Nav1.8, and OPRM1 (FIG. 14). RPP increased Kv4.3 expression 2-times at 10 μM, compared to control; increased Kv7.2 express 7.5- and 9-times at 3 and 10 μM, respectively; increased Nav1.8 expression 2.9- and 4.8-times at 3 and 10 μM, respectively; increased OPRM1 expression at 3 and 10 μM respectively (FIG. 14). RPP sequence ID numbers 13 and 14 were assessed at 3 μM after a 48 hour dosing period in an ex vivo culture of whole DRGs (L5, from male SD rats) and assessed for Nav1.8. Sequence 13 and 14 increased Nav1.8 expression 6- and 13-times, respectively, compared to control (FIG. 15).

5) RPP (SEQ ID NO:12) was not neurotoxic in a LDH assay conducted on ex vivo cultured whole DRGs neurons (L5, from male SD rats). RPP did not increase RLU levels compared to control. As expected the positive control (2% triton) increased RLU 90,000-times (FIG. 17).

### Planned Assessments:

### AIM 1: Determine the Pharmacokinetics, Distribution, and Dosing of RPP (SEQ ID NOS: 1 and 15 Through 17) or RPPv (SEQ ID NOS: 18 Through 117) Peptide Fused to a CPP (SEQ ID NOS: 118 Through 137 and 140 Through 159)

SUBAIM 1.1 Development and validation of liquid chromatography-tandem mass spectrometry (LC-MS/MS) methods for detecting RPP (SEQ ID NOS: 1 and 15 through 17) or RPPv (SEQ ID NOS: 18 through 117) peptide fused to a CPP (SEQ ID NOS: 118 through 137 and 140 through 159), (in a range between 10 and 10,000 ng/mL) in rat and monkey blood plasma, CSF, and tissue.

SUBAIM 1.2. To determine the route of administration in the in vivo animal studies and the clinic, an exploratory pharmacokinetics (PK) study in SD rats will be conducted. Blood plasma samples will be collected after IV, SC, or PO administration of 1000 mg/kg RPP (SEQ ID NOS: 1 and 15 through 17) or RPPv (SEQ ID NOS: 18 through 117) peptide fused to a CPP (SEQ ID NOS: 118 through 137 and 140 through 159) at 5 min, 30 min, 4 h, 8 h, 24 h, and 48 h (2/sex/time point) and assessed for peak drug concentration (Cₘₐₓ), area under the curve (AUC), and half-life (t_{1/2}). To assess RPP (SEQ ID NOS: 1 and 15 through 17) or RPPv (SEQ ID NOS: 18 through 117) peptide fused to a CPP (SEQ ID NOS: 118 through 137 and 140 through 159), penetration to the central and peripheral nervous system, target tissues (CSF, brain, lumbar spinal cord and dorsal root ganglia (DRG)) will also be collected at euthanasia and measured for drug concentrations. RPP (SEQ ID NOS: 1 and 15 through 17) or RPPv (SEQ ID NOS: 18 through 117) peptide fused to a CPP (SEQ ID NOS: 118 through 137 and 140 through 159), will be detected using validated LS-MS/MS methods (SUBAIM 1.1)

SUBAIM 1.3. To determine dosing in the rat efficacy study (Aim 2), a single dose range-finding study in SD rat using the optimal route of administration identified in SUBAIM 1.1 will be conducted. Four dose levels+vehicle control (3/sex/group) will be selected, based on the PK data from SUBAIM 1.1. The high dose (HD) should identify the limit dose (maximum feasible dose (MFD)), maximum tolerated dose (MTD), and/or saturation of exposure). The lower doses will be spaced in ⅓ increments. Animals will be assessed daily for survival and clinical signs (abnormalities and signs of pain or distress). Blood will be collected post dose at 6 time points selected based on the PK data from SUBAIM 1.1 and assessed for Cₘₐₓ, AUC, and t_{1/2}. Tissues that will be assessed for gene changes in the efficacy study (DRGs, lumbar spinal cord, brain) will be collected at euthanasia and assessed for RPP (SEQ ID NOS: 1 and 15 through 17) or RPPv (SEQ ID NOS: 18 through 117) peptide fused to a CPP (SEQ ID NOS: 118 through 137 and 140 through 159), concentration.

### AIM 2: Assessing the Effect OF RPP (SEQ ID NOS: 1 and 15 Through 17) or RPPv (SEQ ID NOS: 18 Through 117) Peptide Fused to a CPP (SEQ ID NOS: 118 Through 137 and 140 Through 159) on Chronic Pain.

We have demonstrated that RPP accumulates in the nuclei of rat motoneurons in the lumbar (L4-L6) spinal cord 48 hours after administration at the site of sciatic nerve transection (FIG. 4). To assess the effects of our drug on pain, we will use the preclinical SNI model using the sciatic nerve. SNI is a well-established animal model for neuropathic pain that produces robust and prolonged changes in thermal sensitivities, peripheral and central morphine analgesia, and c-fiber hypoesthesia⁷⁴. Furthermore, SNI has previously been shown by us and others to induce expression of REST and its target genes implicated in chronic pain (FIG. 18 and Table 12, respectively)^{60,72-74}. Additionally, we observed increased CTDSP1 levels, as we predicted (FIG. 18).

Efficacy of RPP (SEQ ID NOS: 1 and 15 through 17) or RPPv (SEQ ID NOS: 18 through 117) peptide fused to a CPP (SEQ ID NOS: 118 through 137 and 140 through 159) to decrease chronic pain will be done in Phase 1 and durability of the pain reduction will be determined in Phase 2. The SNI sciatic nerve rat model will be used to test if RPP (SEQ ID NOS: 1 and 15 through 17) or RPPv (SEQ ID NOS: 18 through 117) peptide fused to a CPP (SEQ ID NOS: 118 through 137 and 140 through 159), decreases chronic neuropathic pain. Administration of RPP (SEQ ID NOS: 1 and 15 through 17) or RPPv (SEQ ID NOS: 18 through 117) peptide fused to a CPP (SEQ ID NOS: 118 through 137 and 140 through 159), or vehicle or a standard of care drug used to treat neuropathic pain, oxycodone will be done. The experimental procedure for phase 1 is summarized in Table 17 below:

TABLE 17

| | | | Euthanasia | |
|---|---|---|---|---|
| SNI and | | | Day Post- | |
| Sham | | | Start | |
| Surgery | | | of rpp | |
| Groups | | Rat # | dosing | Assessment |
| 1 | rpp LD | 12 SNI | 14 | All rats behavior testing |
| | | 12 Sham | | 8 rats morphological analysis |
| | | | | (4 SNI/4 sham) |
| | | | | 16 rats qPCR analysis |
| | | | | (4 SNI/4 sham) |
| | | 12 SNI | 28 | All rats behavior testing |
| | | 12 Sham | | 8 rats morphological analysis |
| | | | | (4 SNI/4 sham) |
| | | | | 16 rats qPCR analysis |
| | | | | (4 SNI/4 sham) |
| 2 | rpp MD | 12 SNI | 14 | All rats behavior testing |
| | | 12 Sham | | 8 rats morphological analysis |
| | | | | (4 SNI/4 sham) |
| | | | | 16 rats qPCR analysis |
| | | | | (4 SNI/4 sham) |
| | | 12 SNI | 28 | All rats behavior testing |
| | | 12 Sham | | 8 rats morphological analysis |
| | | | | (4 SNI/4 sham) |
| | | | | 16 rats qPCR analysis |
| | | | | (4 SNI/4 sham) |
| 3 | rpp HD | 12 SNI | 14 | All rats behavior testing |
| | | 12 Sham | | 8 rats morphological analysis |
| | | | | (4 SNI/4 sham) |
| | | | | 16 rats qPCR analysis |
| | | | | (4 SNI/4 sham) |
| | | 12 SNI | 28 | All rats behavior testing |
| | | 12 Sham | | 8 rats morphological analysis |
| | | | | (4 SNI/4 sham) |
| | | | | 16 rats qPCR analysis |
| | | | | (4 SNI/4 sham) |
| 4 | Oxycodone | 12 SNI | 14 | All rats behavior testing |
| | | 12 Sham | | 8 rats morphological analysis |
| | | | | (4 SNI/4 sham) |
| | | | | 16 rats qPCR analysis |
| | | | | (4 SNI/4 sham) |
| | | 12 SNI | 28 | All rats behavior testing |
| | | 12 Sham | | 8 rats morphological analysis |
| | | | | (4 SNI/4 sham) |
| | | | | 16 rats qPCR analysis |
| | | | | (4 SNI/4 sham) |
| 5 | Vehicle | 12 SNI | 14 | All rats behavior testing |
| | | 12 Sham | | 8 rats morphological analysis |
| | | | | (4 SNI/4 sham) |
| | | | | 16 rats qPCR analysis |
| | | | | (4 SNI/4 sham) |
| | | 12 SNI | 28 | All rats behavior testing |
| | | 12 Sham | | 8 rats morphological analysis |
| | | | | (4 SNI/4 sham) |
| | | | | 16 rats qPCR analysis |
| | | | | (4 SNI/4 sham) |

A total of 312 2-month old SD rats (equal numbers of males and females) will be needed for this aim. A sample size of 12 rats per group will have 80% power to detect a difference of 1.20 standard deviations in pain assessments. Power analysis is based on a t-test for independent samples with a 5%, 2-sided significance level. For the efficacy study, the rats will be randomly assigned to the experimental groups (n=12/group) designated into the table on the right. Rats from each of the ten randomized groups will be examined with stimulus evoked and non-stimulus evoked behavior tests, morphological, immuno-histochemical analysis, quantitative RT-PCR (qRT-PCR) analysis, and WB analysis. At euthanasia, blood will be drawn at 2 time points (14 and 28 days) post-start of drug delivery and assessed for standard PK parameters. The DRG and brain will be extracted to determine Oprm1, Nav1.8, Kv4.3, and Kv7.2 levels. In addition, tissue will be collected for LC-MS/MS analysis to determine rpp concentration.

Surgery: SNI or sham surgery (surgery which exposes the nerve branches without nerve injury) will be performed as described previously¹⁶⁹. Briefly, the rats will be anesthetized and the common peroneal and tibial nerves ligated. A 2-4 mm segment of each nerve distal to the ligation will be cut and removed, leaving the third branch of the sciatic nerve, the sural nerve intact. The sural nerve is spared to produce an increased response to noxious and non-noxious stimuli in the ipsilateral innervated region beginning at 4-days post-injury, stabilizing at 7 days and maintained up to 6 months. 7-days post injury is the accepted time point for development of chronic neuropathic pain and is translationally relevant.

Drug/Vehicle: Administration will begin on day 5 post-surgery. For RPP (SEQ ID NOS: 1 and 15 through 17) or RPPv (SEQ ID NOS: 18 through 117) peptide fused to a CPP (SEQ ID NOS: 118 through 137 and 140 through 159), the route, frequency, and drug concentrations to be tested will be based on the PK studies in Specific Aim 1. For oxycodone, the route of administration and efficacious drug exposure in the rat that approximates efficacious exposure in the humans is known: (oxycodone at a dose of 0.56 mg/kg and 0.2 mL vehicle)¹⁷⁰.

Behavior Tests: The effects on pain transmission and motor function will be assessed one day prior to surgery and every other day beginning on day 4 post-surgery. Both stimulus evoked and non-stimulus evoked behavior tests will be used to determine the functional effectiveness of the RPP (SEQ ID NOS: 1 and 15 through 17) or RPPv (SEQ ID NOS: 18 through 117) peptide fused to a CPP (SEQ ID NOS: 118 through 137 and 140 through 159) compared to oxycodone and vehicle. In recent years concerns over the translatability of some stimulus evoked behavior tests has led to the increasing use of stimulus evoked, non-stimulus evoked, and operant or voluntary behavior tests. For this proposal we will use the most widely used and translatable stimulus evoked behavior tests: the electronic Von Frey and heat hyperalgesia tests, a non-stimulus evoked gait analysis, and a place escape/avoidance paradigm to measure the aversive state of pain¹⁷¹. Recent studies have indicated the value in including gait analysis to detect subtle improvements and deteriorations in animal models¹⁷². The rat handlers and assessors performing the behaviors tests will be blinded to which experimental/control group the rats were randomly assigned. Each animal will have a unique animal number. A secret color code will be used to designate control and experimental groups. Only after analysis will this code be revealed.

Stimulus Evoked Behavior Tests: Hypersensitivity to mechanical stimulation: An electronic von Frey (Bioseb, Chaville, France) consisting of a hand-held force transducer fitted with a plastic tip will be used to test the mechanical withdrawal threshold of the left and right hind paws. The plastic tip of the force transducer will be applied to the medial plantar surface of the paw for testing the saphenous nerve and to the lateral plantar surface of the paw to test the sciatic (peroneal nerve).

Heat hyperalgesia will be measured based on a published method that we modified to use a laser as the heat source¹⁷³. An 808 nm wavelength laser will be used as the heat source (2 W output power, the beam diameter on the skin=3 mm) and will be applied to the relevant dermatome are for the nerve being tested. The small beam size allows the involved dermatomes to be specifically targeted. For the sciatic nerve and saphenous nerves, limb withdrawal latency induced by thermal stimulation will be recorded with a cutoff point of 10 seconds.

Non-Stimulus Evoked Behavior Test: Gait Analysis: Sciatic nerve motor function will be assessed using the Digigait and Sciatic Nerve Index¹⁷². Briefly, utilizing the Digigait equipment available in the Rodent Behavioral Core, which has been characterized and optimized by our laboratory, sciatic nerve function will be determined pre-injury and on post-surgical days 7, 14 and 28. Rats will be placed on the motorized treadmill within the Digigait holder and a speed of 20 cm/sec set for recording of all animals. The Sciatic Nerve Index program will be used to analyze the function of the affected hind limb.

Place Escape/Avoidance: This procedure is designed to test the hypothesis that rats will avoid the environment that is associated with mechanical stimulation to the hyperalgesic area. It has been reported that a sensitivity to change in escape/avoidance behavior occurs in injured rats after treatment with analgesics (Baastrup et al 2010) 174. The cerebral-dependent place escape/avoidance paradigm test has been described by LaBuda and Fuchs (2000) 171 and depends on escape/avoidance learning to a novel aversive environment. The animals will be tested only once 3 to 5 weeks post-injury by the same trained investigator. Testing will be done in the USU Rat Behavior Core. The animals will be allowed to habituate to ambient light and noise conditions in the test room for a minimum of 1 hour. For this test, the rats will have free access between the ‘non-aversive’ dark and ‘aversive’ light side of an enclosed chamber with a mesh floor which is readily accessible by a von Frey filament from below. Either the injured or non-injured hind paw will be routinely stimulated if the rat is in the dark or light area, respectively. A mechanical stimulus (von Frey filament) that is adequate to elicit a withdrawal response on the injured paw will be applied every 15 sec to the lateral plantar surface of one of the hind paws of the animal, depending on the location of the animal during that time, for a period of 30 min. Escape/avoidance behavior will be defined as a shift from the dark to the light area. The percentage of time spent in the white side of the box and the number of crossings between the black and white sides will be recorded. The cumulated time in the white side and the total number of crossings will be used as an indication of escape/avoidance learning.

Euthanasia: For morphological and immunohistochemical analysis, the rats will be anesthetized with ketamine/xylazine (80 to 100 mg/kg+10 mg/kg, i.p., 21 gauge needle) and perfused transcardially with 300 ml phosphate buffered saline (pH 7.4) followed by 300 ml of 4% paraformaldehyde in 0.1 M phosphate buffer. After perfusion, nerves, DRG and spinal cord will be dissected, post-fixed for 24 hours in 4% paraformaldehyde and cryoprotected for 24 hours in 30% sucrose. The tissue samples will be cut into 10 μm sections using a cryostat (Leica CM3050 S, Leica Biosystems, Wetzlar, Germany).

Histology: After euthanasia and nerve sampling, sections will be stained with Hematoxylin and Eosin. The expression levels and distribution of the REST-target genes Oprm1, Nav1.8, Kv4.3, and Kv7.2 will be assessed by immunofluorescence.

Gene Expression Analysis: For gene expression analysis of the sciatic nerve and its associated DRG and spinal cord segments, REST-target genes Oprm1, Nav1.8, Kv4.3, and Kv7.2 and control genes (Hprt, Gapd, Rn18s) will be assessed by qRT-PCR.

Protein Expression Analysis: Protein expression analysis of the sciatic nerve associated DRGs, will be assessed by SDS-PAGE immunoblotting of Oprm1, Nav1.8, Kv4.3, and Kv7.2. GAPDH will be used as loading control.

Statistical Approach: For behavior data, results will be presented as mean±standard error of the mean (SEM). A 2 factor ANOVA will be used to assess groups of animals based on time post-treatment. The Bonferroni-Holm method will be used to adjust for multiple comparisons. Two-tailed statistical significance will be established as P<0.05. For immunohistochemical analysis, Two-way ANOVA will be used to compare means for interaction effects as well as effect of group and time. For qRT-PCR, values will be reported as mean±standard error of the mean (SEM). Statistically significant drug effects on the gene expression will be determined by one-way analysis of variance (ANOVA) with Tukey's multiple comparison test.

The experimental procedure for Phase 2 is outlined in Table 18 below:

TABLE 18

| | | | Euthanasia | |
|---|---|---|---|---|
| SNI and | | | Day | |
| Sham | | | Post- | |
| Surgery | | | Start of | |
| Groups | | Rat # | rpp dosing | Assessment |
| 1 | rpp (TBD) | 12 SNI | 14 | All rats behavior testing |
| | | 12 Sham | | 8 rats morphological analysis |
| | | | | (4 SNI/4 sham) |
| | | | | 16 rats qPCR analysis |
| | | | | (4 SNI/4 sham) |
| | | 12 SNI | 28 | All rats behavior testing |
| | | 12 Sham | | 8 rats morphological analysis |
| | | | | (4 SNI/4 sham) |
| | | | | 16 rats qPCR analysis |
| | | | | (4 SNI/4 sham) |
| 4 | Oxycodone | 12 SNI | 14 | All rats behavior testing |
| | | 12 Sham | | 8 rats morphological analysis |
| | | | | (4 SNI/4 sham) |
| | | | | 16 rats qPCR analysis |
| | | | | (4 SNI/4 sham) |
| | | 12 SNI | 28 | All rats behavior testing |
| | | 12 Sham | | 8 rats morphological analysis |
| | | | | (4 SNI/4 sham) |
| | | | | 16 rats qPCR analysis |
| | | | | (4 SNI/4 sham) |
| 5 | Vehicle | 12 SNI | 14 | All rats behavior testing |
| | | 12 Sham | | 8 rats morphological analysis |
| | | | | (4 SNI/4 sham) |
| | | | | 16 rats qPCR analysis |
| | | | | (4 SNI/4 sham) |
| | | 12 SNI | 28 | All rats behavior testing |
| | | 12 Sham | | 8 rats morphological analysis |
| | | | | (4 SNI/4 sham) |
| | | | | 16 rats qPCR analysis |
| | | | | (4 SNI/4 sham) |

In Phase 2, the durability of the pain reduction of RPP (SEQ ID NOS: 1 and 15 through 17) or RPPv (SEQ ID NOS: 18 through 117) peptide fused to a CPP (SEQ ID NOS: 118 through 137 and 140 through 159) will be examined. For this study, the RPP (SEQ ID NOS: 1 and 15 through 17) or RPPv (SEQ ID NOS: 18 through 117) peptide fused to a CPP (SEQ ID NOS: 118 through 137 and 140 through 159) dose (low, medium, or high) and the period of drug delivery (14 or 28 days) that was determined in Phase 1 to be most efficacious will be used. The same methodology described above for the Phase 1 efficacy study will be followed. The difference being that after the drug administration period (14 or 28 days) the rats will be monitored and behaviorally assessed for pain response and motor function every other day for 30 days. The rats will be euthanized on day 30. A total of 72 2-month old, SD rats (equal numbers of males and females) will be needed for this study. A sample size of 12 rats per group will have 80% power to detect a difference of 1.20 standard deviations in pain assessments. Power analysis is based on a t-test for independent samples with a 5%, 2-sided significance level. The rats will be randomly assigned to the experimental groups designated in the table on the right. Rats from each of the ten randomized groups will be examined with stimulus evoked and non-stimulus evoked behavior tests, morphological, immuno-histochemical analysis, quantitative RT-PCR (qRT-PCR) analysis, and WB analysis.

### Aim 3. Toxicology and Safety Pharmacology Assessments of RPP (SEQ ID NOS: 1 and 15 Through 17) or RPPv (SEQ ID NOS: 18 Through 117) Peptide Fused to a CPP (SEQ ID NOS: 118 Through 137 and 140 Through 159).

To support first-in-human dosing, we will assess in vitro cardiac and genetic toxicity and in vivo toxicology, safety pharmacology, and toxicokinetics of RPP (SEQ ID NOS: 1 and 15 through 17) or RPPv (SEQ ID NOS: 18 through 117) peptide fused to a CPP (SEQ ID NOS: 118 through 137 and 140 through 159) in two species, as recommended by FDA guidances M3 (R2), S2B, S6 (R1), S7A, and S7B175-179. In both species, the route of administration will be selected based on the results of the PK assessment in AIM 1.

SUBAIM 3.1. Single dose range-finding study in monkey: Four dose levels+vehicle control will be assessed in 3 monkeys/sex/group. The HD should identify the MTD, MFD, and/or saturation of exposure. The lower doses will be spaced in ⅓ increments. Blood will be collected at 6 time points (TBD base on PK data collected in Aim 1) and assessed for Cₘₐₓ, AUC, and t_{1/2}. There will be daily assessments for survival and clinical signs.

SUBAIMs 3.2 & 3.3. Toxicology studies in rat (3.2) and monkey (3.3). The dosing period in both species will be 4 weeks, to support dosing in humans of the same duration+a 6-week recovery period, selected because of the high stability of rpp observed in the cellular assays⁷⁷. There will be three dose levels+vehicle control. The high doses will be the maximum tolerated doses identified in the dose range-finding studies (rat AIM 1.2 and monkey Aim 3.1). The MD will be ⅓ lower than the HD, based on AUC in the range-finding studies. The LD will approximate the efficacious dose in the in vivo efficacy study (Aim 2). The dosing interval will be the t_{1/2} of RPP (SEQ ID NOS: 1 and 15 through 17) or RPPv (SEQ ID NOS: 18 through 117) peptide fused to a CPP (SEQ ID NOS: 118 through 137 and 140 through 159) in each species. In the rat study, there will be 15/sex/group. 10/sex/group will be sacrificed at the end of the Dosing Period and 5/sex/vehicle will be sacrificed at the end of the Recovery Period. Additionally, 9/sex/rpp groups and 3/sex/vehicle group will be assigned to the toxicokinetic portion of the study. In the monkey study, all animals will be assessed for toxicology and TK (3/sex/group in the Main Study and 2/sex/group in the Recovery Period). The following toxicological parameters will be assessed in both species: clinical observations, body weight, food consumption, ophthalmoscopy (fundoscopy and slit lamp examinations), and a standard battery of clinical chemistry, organ weights, and histological parameters will be assessed. Monkeys will undergo assessments of respiratory (e.g. tidal volume and hemoglobin oxygen saturation) and cardiac electrocardiogram recordings measured using non-surgical telemetry functions¹⁷⁹, as recommended by FDA guidance S7A¹⁷⁹. The cardiovascular assessment will occur, twice during the pre-treatment, after the first dose, and at the end of the Recovery Period (if needed). The recordings will be evaluated qualitatively by a consultant board certified cardiologist. All waveforms will be qualitatively evaluated to detect rhythm or conduction disturbances, including evaluations of PR and QRS intervals. Rats will be assessed for CNS functions using the modified Irwin's functional observational battery^{179,180}. TK assessments (Cₘₐₓ, AUC, and t_{1/2}) from blood plasma will be made at 6 time points (determined by the rang-finding studies).

SUBAIM 3.4. In vitro hERG assay¹⁷⁷. Inhibition of hERG channels is a common cause of long QT syndrome and is also correlated with arrhythmias that can lead to ventricular fibrillation and sudden cardiac death. To test whether RPP (SEQ ID NOS: 1 and 15 through 17) or RPPv (SEQ ID NOS: 18 through 117) peptide fused to a CPP (SEQ ID NOS: 118 through 137 and 140 through 159) inhibits hERG channel current, we will contract an electrophysiological evaluation on hERG channel current in CHO cells treated with a high concentration of RPP (SEQ ID NOS: 1 and 15 through 17) or RPPv (SEQ ID NOS: 18 through 117) peptide fused to a CPP (SEQ ID NOS: 118 through 137 and 140 through 159) (10 μM) or positive control (cisapride, 0.03 μM). Each cell will serve as its own positive control. Whole cell recordings will be conducted using conventional voltage clamp techniques.

SUBAIM 3.5. Genotoxicity. RPP (SEQ ID NOS: 1 and 15 through 17) or RPPv (SEQ ID NOS: 18 through 117) peptide fused to a CPP (SEQ ID NOS: 118 through 137 and 140 through 159) will be assessed in in vitro OECD compliant Ames and in vitro chromosomal aberration assays (S2B, November 1997) 176 to determine its genotoxic potential.

### AIM 4. RPP (SEQ ID NOS: 1 and 15 Through 17) or RPPv (SEQ ID NOS: 18 Through 117) Peptide Fused to a CPP (SEQ ID NOS: 118 Through 137 and 140 Through 159) Abuse Potential Assessment.

Because opioid receptor gene expression is inhibited by REST, it is possible that RPP (SEQ ID NOS: 1 and 15 through 17) or RPPv (SEQ ID NOS: 18 through 117) peptide fused to a CPP (SEQ ID NOS: 118 through 137 and 140 through 159) could augment the effects of endogenous opioid receptor ligands such as endorphins^{181,182}. Additionally, recent studies do show an increase in expression of REST in the DRG and the periaqueductal gray area (PAG) during conditions involving pain^{183,184} To determine if RPP (SEQ ID NOS: 1 and 15 through 17) or RPPv (SEQ ID NOS: 18 through 117) peptide fused to a CPP (SEQ ID NOS: 118 through 137 and 140 through 159) produces reward-seeking behavior we will evaluate its effects on rats using conditioned place preference (CPP), a widely used behavioral assay to test the rewarding properties of drugs¹⁸⁵⁻¹⁸⁹. CPP is a well-established method that has demonstrated morphine reward-seeking behavior in an SNI animal model.

First, RPP (SEQ ID NOS: 1 and 15 through 17) or RPPv (SEQ ID NOS: 18 through 117) peptide fused to a CPP (SEQ ID NOS: 118 through 137 and 140 through 159) will be administered (route TBD) to uninjured rats and then CPP will be performed. Oxycodone will be used in place of morphine as a positive control for consistency with Aim 2 and saline will be injected into separate rats as a negative control.

The apparatus consists of a three-compartment chamber: the two outer compartments are designed to have different characteristics (e.g. white vs. black walls), and the central compartment has no special characteristics. Each experiment consists of three phases. Phase I Pre-test (Day 1); After administration of saline, rats will be placed in the center compartment with gates open to provide access to both the other compartments. Rats will be monitored for 15 min to determine baseline preference by recording the time spent in each chamber. The mean spontaneous preference times for the compartments will be determined. Rats that spend more than 60% of the test time in any one chamber will be considered biased and will be removed from the experiment. Phase II Conditioning (Days 2-7); Rats will be administered vehicle or RPP (SEQ ID NOS: 1 and 15 through 17) or RPPv (SEQ ID NOS: 18 through 117) peptide fused to a CPP (SEQ ID NOS: 118 through 137 and 140 through 159), and then they will be randomly assigned/conditioned to receive RPP (SEQ ID NOS: 1 and 15 through 17) or RPPv (SEQ ID NOS: 18 through 117) peptide fused to a CPP (SEQ ID NOS: 118 through 137 and 140 through 159) in one compartment and vehicle in the other. The dose of RPP (SEQ ID NOS: 1 and 15 through 17) or RPPv (SEQ ID NOS: 18 through 117) peptide fused to a CPP (SEQ ID NOS: 118 through 137 and 140 through 159) used will be based on the concentration that produces measurable effects on mechanical and thermal hyperalgesia determined in AIM 2. Treatment compartment and the order of drug and vehicle administration will be counterbalanced for all groups to avoid biases. This phase consists of six consecutive conditioning sessions 30 min in length. Conditioning will also be done with oxycodone for a separate group. Phase III Test (Day 8); rats will be injected with saline and placed in the center of the CPP box and monitored for 15 min. The difference between the time spent in the drug-paired compartment during post-conditioning and that spent during pre-conditioning will be used to assess the degree of place conditioning elicited by RPP (SEQ ID NOS: 1 and 15 through 17) or RPPv (SEQ ID NOS: 18 through 117) peptide fused to a CPP (SEQ ID NOS: 118 through 137 and 140 through 159) 190.

RPP (SEQ ID NOS: 1 and 15 through 17) or RPPv (SEQ ID NOS: 18 through 117) peptide fused to a CPP (SEQ ID NOS: 118 through 137 and 140 through 159) will be assessed using 8-groups of 12 injured rats (SNI vs. SHAM, conditioning with oxycodone, RPP (SEQ ID NOS: 1 and 15 through 17) or RPPv (SEQ ID NOS: 18 through 117) peptide fused to a CPP (SEQ ID NOS: 118 through 137 and 140 through 159), and saline). A sample size of 12 rats per group will have 80% power to detect a difference of 1.20 standard deviations in pain assessments. Power analysis is based on a t-test for independent samples with a 5%, 2-sided significance level. The rats will be randomly assigned to the experimental groups. Rats will be assigned to undergo SNI or SHAM surgery randomly. The von Frey test will be performed one day before and 4 and 8-days post-injury to monitor the pain state. At 8-days post-injury the CPP test will be conducted as previously described. The research associate performing the behavior tests will be blinded to which experimental group/control group the rat was randomly assigned. Each animal will have a unique animal number. A secret color code will be used to designate control and experimental groups. Only after analysis will this code be revealed. Animals will be euthanized, and samples analyzed as described in AIM 2.

PREDICTED RESULTS & ALTERNATIVE STRATEGIES. We predict that the RPP (SEQ ID NOS: 1 and 15 through 17) or RPPv (SEQ ID NOS: 18 through 117) peptide fused to a CPP (SEQ ID NOS: 118 through 137 and 140 through 159) will reduce REST levels in injured neurons and restore proper expression of ion channels required for excitability. At the behavioral level, RPP (SEQ ID NOS: 1 and 15 through 17) or RPPv (SEQ ID NOS: 18 through 117) peptide fused to a CPP (SEQ ID NOS: 118 through 137 and 140 through 159) should return the stimulus or non-stimulus evoked behavior tests to baseline in SNI rats. Furthermore, given its mode of action, we do not expect RPP (SEQ ID NOS: 1 and 15 through 17) or RPPv (SEQ ID NOS: 18 through 117) peptide fused to a CPP (SEQ ID NOS: 118 through 137 and 140 through 159) to have addictive properties. However, if drug administration shows abuse potential, we will confirm this positive result with a progressive ratio (PR) schedule of reinforcement during RPP (SEQ ID NOS: 1 and 15 through 17) or RPPv (SEQ ID NOS: 18 through 117) peptide fused to a CPP (SEQ ID NOS: 118 through 137 and 140 through 159) administration.

Example 15-RPP (SEQ ID NOS: 1 and 15 Through 17) or RPPv (SEQ ID NOS: 18 Through 117) Peptide Fused to a CPP (SEQ ID NOS: 118 Through 137 and 140 Through 159) can be Used to Prevent Relapse of Glioblastoma Multiforme (GBM)

Assess the neurogenic and anti-oncogenic potency of RPP (SEQ ID NOS: 1 and 15 through 17) or RPPv (SEQ ID NOS: 18 through 117) peptide fused to a CPP (SEQ ID NOS: 118 through 137 and 140 through 159) on brain tumor initiating cells (BTICs) and in vivo. Test article (RPP (SEQ ID NOS: 1 and 15 through 17) or RPPv (SEQ ID NOS: 18 through 117) peptide fused to a CPP (SEQ ID NOS: 118 through 137 and 140 through 159) activity will be assessed in primary cultured BTICs derived from 10 grade IV GBM patients obtained at the Mayo Clinic using methods we have established¹⁹¹⁻¹⁹³. Cells (0.25×10⁴) will be plated (3.8 cm2 TC dish) in media (250 μL) dosed with 5 μL vehicle or with 3 dose levels of test article at the optimal dose range (determined in the neuronal differentiation study described in Example 13) for 0, 2, 4, or 8 days (Main Study) and then allowed to recover after wash out of test article for 8 days (Recovery Period). Markers for pluripotency (Sox2 and Nestin), proliferation (Ki67), cell death (caspase 3), and differentiation (MAP2, neuron and GFAP, glia) will be monitored on Days 0, 2, 4, and 8 during the dosing period (Main Study) and on Days 10, 13, and 16 during the Recovery Period (3/TC wells/group for each day) by WB and RT-PCR.

Primary cultured BTICs derived from 10 grade IV GBM patients will be used in an intracranial xenograph tumorigenicity nude mouse model as described previously by us and others^{191,192,194}. Briefly, cells (5×10⁴) will be suspended in 5 μL PBS (control) or PBS containing one of 3 TBD (based on BTIC neurogenic potency assay above) test article dose levels and delivered using an automated syringe pump with a guide-screw system^{112,195}. The injection coordinates will be X: 1.5; Y: 1.34; Z: −3.5 targeting the mouse striatum; a highly reliable site for tumor engraftment¹⁹⁶. Mice will be assayed for survival during weeks 4, 8, 16, and 20 using the Kaplan-Meier method¹⁹⁷ followed by postmortem histopathology to evaluate tumor size and invasiveness (H&E stain), cell death (TUNEL), and proliferation (anti-human nuclei) (3/sex/group for all procedures).

Statistical approach: Values will be reported as mean±standard error of the mean (SEM) or standard deviation (SD) as indicated. Statistical significance will be determined by applying appropriate parametric or non-parametric variations of the student's t test or one-way analysis of variance (ANOVA). Briefly, the ratio of total REST to phosphorylated REST at different control and drug candidate treatments will be statistically tested using the Kruskal-Wallis test. Differences in marker expression between BTICs control and drug candidate cultures and orthotopic xenographs will be statistically tested using the Tukey multiple comparison test. Differences in tumor size and invasiveness between mice injected with BTICs treated with control or test article will be statistically tested using the Mann-Whitney U test, and differences in survival will be statistically tested using the log-rank test and Cox proportion hazard test¹⁹⁸.

Predicted results: At every time point, we expect mice with xenografts of test article dosed BTICs to present tumor free or with smaller less invasive tumors, with more cell death (TUNEL), and less GBM proliferation (anti-human nuclei) as compared to control. Additionally, we expect increased survival time in mice injected with test article and BTICs compared to BTICs alone.

### Materials and Methods

### Test Systems

Cells. Primary cultured brain tumor initiating cells (BTICs) were derived from 10 grade IV GBM patients. The methods for extracting and propagating the BTICs have been previously described by Dr. Alfredo Quinones-Hinojosa and his research team¹⁹¹⁻¹⁹³.

Species/Strain. Nu/nu Harlan Sprague Dawley mice

Age: At study start: 6 weeks

Weight: At study start: Males: 20-30 g; Females: 18-35 g

### Test Article Identification.

RPP (SEQ ID NOS: 1 and 15 through 17) or RPPv (SEQ ID NOS: 18 through 117) peptide fused to a CPP (SEQ ID NOS: 118 through 137 and 140 through 159)

Purity: ≥95% HPLC Dose Formulation preparation and stability: RPP (SEQ ID NOS: 1 and 15 through 17) or RPPv (SEQ ID NOS: 18 through 117) peptide fused to a CPP (SEQ ID NOS: 118 through 137 and 140 through 159) and vehicle control will be prepared in bulk every week. Dose formulation concentrations will be assessed on at the beginning of every week. The acceptable concentration range is +10% of nominal.

### Vehicle: Water

### Study Design

### Neurogenic Potency of Test Article Study:

Dose: 3 test article dose levels+vehicle control. The test article doses will be determined based on the results of the neuronal differentiation study described in Example 11.

Administration: Doses will be administered every 2 days with media change.

Duration: Main study: 0, 2, 4, or 8 days; recovery period: 2, 5, or 8 days.

Number of replicates. 3 tissue culture (TC) wells per group.

Dosing volume. 5 μL

Parameters analyzed. Markers for pluripotency (Sox2 and Nestin), proliferation (Ki67), cell death (caspase 3), and differentiation (MAP2, neuron and GFAP, glia) will be monitored on Days 0, 2, 4, and 8 during the dosing period (Main study) and on Days 10, 13, and 16 during the recovery period (3/TC wells/group for each day) by WB and RT-PCR (Table 10).

TABLE 10
Assessment of neurogenic potency of test article on BTICs

| Test | | No. of replicates (TC wells (3.8 cm²)) | | | | | | |
|---|---|---|---|---|---|---|---|---|
| Article* | | Main Study | | | | Recovery Period | | |
| Group | Dose | Day | Day | Day | Day | Day | Day | Day |
| No. | | 0 | 2 | 4 | 8 | 10 | 13 | 16 |
| 1 | 0 | 3 | 3 | 3 | 3 | 3 | 3 | 3 |
| 2 | TBD | 3 | 3 | 3 | 3 | 3 | 3 | 3 |
| 3 | TBD | 3 | 3 | 3 | 3 | 3 | 3 | 3 |
| 4 | TBD | 3 | 3 | 3 | 3 | 3 | 3 | 3 |

*The test article will be RPP (SEQ ID NOS: 1 and 15 through 17) or RPPv (SEQ ID NOS: 18 through 117) peptide fused to a CPP (SEQ ID NOS: 118 through 137 and 140 through 159)

### Anti-Oncogenic Potency of Test Article on BTICs in Mice:

Dose: 3 doses of test article+vehicle control. Test article doses will be based on the results of the neurogenic potency study.

Frequency of administration: Single dose

Route: Automated syringe pump with a guide-screw system^{112,195} The injection coordinates will be X: 1.5; Y: 1.34; Z: −3.5 targeting the mouse striatum; a highly reliable site for tumor engraftment¹⁹⁶.

Vehicle: Phosphate-buffered saline (PBS)

Number of animals. 3/sex/group

Dosing volume. 5 μL

Parameters analyzed. Mice will be assayed for survival during weeks 4, 8, 16, and 20 using the Kaplan-Meier method¹⁹⁷ followed by postmortem histopathology to evaluate tumor size and invasiveness (H&E stain), cell death (TUNEL), and proliferation (anti-human nuclei) (Table 11).

TABLE 11
Assessment of anti-oncogenic potency of test
article on BTICs in mice

| | | | | Mouse No. (M/F) | | | |
|---|---|---|---|---|---|---|---|
| Test Article* | | Injection Solution | | Weeks | | | |
| Group No. | Dose | BTIC No. | PBS μL | 4 | 8 | 16 | 20 |
| 1 | 0 | 5 × 10⁴ | 5 | 3/3 | 3/3 | 3/3 | 3/3 |
| 2 | TBD | 5 × 10⁴ | 5 | 3/3 | 3/3 | 3/3 | 3/3 |
| 3 | TBD | 5 × 10⁴ | 5 | 3/3 | 3/3 | 3/3 | 3/3 |
| 4 | TBD | 5 × 10⁴ | 5 | 3/3 | 3/3 | 3/3 | 3/3 |

*RPP (SEQ ID NOS: 1 and 15 through 17) or RPPv (SEQ ID NOS: 18 through 117) peptide fused to a CPP (SEQ ID NOS: 118 through 137 and 140 through 159)

### Environmental Conditions

Housing. 2 animals/cage, 12 hour light/dark cycle.

Diet. Ad libitum food and water.

Table 19 below shows the sequences for SEQ ID NOS: 1 through 324:

TABLE 19

| Sequence | | |
|---|---|---|
| ID | Sequence | Name |
| 1 | TEDLEPPEPPLPKEN | — |
| 15 | EDLEPPEPPLPK | — |
| 16 | nekplppeppeldet | — |
| 17 | kplppeppelde | — |
| 18 | MCTEDLEPPEPPLPKENC | v1 |
| 19 | MCTEAPAPPEPALPKKKKKNC | v2 |
| 20 | MCTEDLQPPTAVPQENC | v3 |
| 21 | MCTEAPAPPEPALPKKKKNC | v4 |
| 22 | MCTADLEPPEPRMEKKKVDC | v5 |
| 23 | MCTGDLQPPKTTVSKKDC | v6 |
| 24 | MCTEDLQSPKTTMTKENC | v7 |
| 25 | MCTEDLEPPEPPLPKEDC | v8 |
| 26 | MCTEDQEQQEEQLPEENC | v9 |
| 27 | MCTADLKPPKTTMTKQNC | v10 |
| 28 | MCPGDLKQPEPPMPKEYC | v11 |
| 29 | MCTEDLEPPKATMTKKDC | V12 |
| 30 | MCTEDQERPPVTKEDC | v13 |
| 31 | MCIADPEPPEAQLPEGNC | v14 |
| 32 | MCTGVQEPPEATLPKKNC | v15 |
| 33 | MCSEAQEPPESRLPQVNC | v16 |
| 34 | MCTKHLEPPGPPLPQENC | v17 |
| 35 | MCTAAPEPPEPPVSKEYC | v18 |
| 36 | MCTEDLQLPKTTMTKEYC | v19 |
| 37 | MCSVDLQPPARLRPMVNC | v20 |
| 38 | MCTGDLQPPESRQPQVNC | v21 |
| 39 | MCTGDLQPPEAQVIEVNC | v22 |
| 40 | MCTEDLQPPEPQLPEVNC | v23 |
| 41 | MCTEDMEPRKTTMTKKYC | v25 |
| 42 | MCTEAPAPPEPALPKKKKKKNC | v26 |
| 43 | MCTEAPAPPEPALPKKKKKNC | v27 |
| 44 | MCTEDLQSPKTTMTKENC | v28 |
| 45 | MCTGDLKLPEPPMSKKKKKNC | v29 |
| 46 | MCTEDLQPPKTTMAEKYC | v30 |
| 47 | MCSEDPEPPKTTMTKKNC | v31 |
| 48 | MCTEDLKPPEASLPEENC | v32 |
| 49 | MCAGDLEQPEPPVAKKKKKNC | v33 |
| 50 | MCNGDLERPEPPVAKEYC | v34 |
| 51 | MCTEDLKPPEPPLPKENC | v35 |
| 52 | MCNEALEPPPLRKEHC | v36 |
| 53 | MCPEDLERPPLTKEHC | v37 |
| 54 | MCTEDLEPPERPLPREIC | v38 |
| 55 | MCAGDLKPPETTMSKKNC | v39 |
| 56 | MCTEDLQQPERSQPMESC | v40 |
| 57 | MCPEDLQPPEPALPEKKKKKIVVLALVVKSAVSVGVV | v41 |
| 58 | MCTEVLVPRTTSGKGRLWFWLWSQKGHPSASACGS | v42 |
| 59 | MCAEDLQPPPLLEAHCGSDSGRKKRRQC | v43 |
| 60 | MCTAAPEPPEPQLPQANC | v44 |
| 61 | MCPADLQQPETSLPEENC | v45 |
| 62 | MCSVDLQPPARLRPMVNC | v46 |
| 63 | MCTEALEPPEPPLTKENC | v47 |
| 64 | MCTEAMEPPEPPLARESC | v48 |
| 65 | MCTADLQPPEASLPQQNC | v49 |
| 66 | MCTAAPEPPEPRLPEGNC | v50 |
| 67 | MCTKDLAPQAPPLLKENC | v51 |
| 68 | cnekplppeppeldetcm | v1^{RI} |
| 69 | cnkkkkkplapeppapaetcm | v2^{RI} |
| 70 | cneqpvatppqldetcm | v3^{RI} |
| 71 | cnkkkkplapeppapaetcm | v4^{RI} |
| 72 | cdkksvttkppqldgtcm | v5^{RI} |
| 73 | cdkksvttkppqldgtcm | v6^{RI} |
| 74 | cnektmttkpsqldetcm | v7^{RI} |
| 75 | cdekplppeppeldetcm | v8^{RI} |
| 76 | cneeplqeelleldetcm | v9^{RI} |
| 77 | cnqktmttkppkldgpcm | v10^{RI} |
| 78 | cyekpmppepqldgpcm | v11^{RI} |
| 79 | cdekktmtakppeldetcm | v12^{RI} |
| 80 | cdektvppreqdetcm | v13^{RI} |
| 81 | cngeplqaeppepdaicm | v14^{RI} |
| 82 | cnkkpltaeppeqvgtcm | v15^{RI} |
| 83 | cnvqplppgppelhktcm | v16^{RI} |
| 84 | cneqplppgppepaatcm | v17^{RI} |
| 85 | cyeksvppeppepaatcm | v18^{RI} |
| 86 | cyektmttkplqldetcm | v19^{RI} |
| 87 | cnvmprlrappqldvscm | v20^{RI} |
| 89 | cnvqpqrseppqldgtcm | v21^{RI} |
| 89 | cnveivqaeppqldgtcm | v22^{RI} |
| 90 | cnveplqpeppqldetcm | v23^{RI} |
| 91 | cykktmttkppemdetcm | v25^{RI} |
| 92 | cnkkkkkkpklapeppapaetcm | v26^{RI} |
| 93 | cnkkkkkplapeppapaetcm | v27^{RI} |
| 94 | cnektmttkpsqldetcm | v28^{RI} |
| 95 | cnkkkkksmppepplkldgtcm | v29^{RI} |
| 96 | cykeamttkppqldetcm | v30^{RI} |
| 97 | cnkktmttkppepdescm | v31^{RI} |
| 98 | cneeplsaeppkldetcm | v32^{RI} |
| 99 | cnkkkkkavppepqeldgacm | v33^{RI} |
| 100 | cyekavppepreldgncm | v34^{RI} |
| 101 | cnekplppeppkldetcm | v35^{RI} |
| 102 | chekrlpppelaencm | v36^{RI} |
| 103 | chektlppreldepcm | v37^{RI} |
| 104 | cierplpreppeldetcm | v38^{RI} |
| 105 | cnkksmtteppkldgacm | v39^{RI} |
| 106 | csmpqsrepqqledetcm | v40^{RI} |
| 107 | cvvgvsvaskvvlalvvikkkkkeplapeppqldepcm | v41^{RI} |
| 108 | csgcasasphgkqswlwfwlrgkgsttrpvlvetcm | v42^{RI} |
| 109 | cqrrkkrgsdsgchaellpppqldeacm | v43^{RI} |
| 110 | cnaqplqpeppepaatcm | v44^{RI} |
| 111 | cneeplstepqqldapcm | v45^{RI} |
| 112 | cnvmprlrappqldvscm | v46^{RI} |
| 113 | cnektlppeppelaetcm | v47^{RI} |
| 114 | cseralppeppemaetcm | v48^{RI} |
| 115 | cnqqplsaeppqldatcm | v49^{RI} |
| 116 | cngeplrpeppepaatcm | v50^{RI} |
| 117 | cnekllppaqpaldktcm | v51^{RI} |
| 118 | GRKKRRQRRR | — |
| 119 | GDIMGEWGNEIFGAIAGFLGYGRKKRRQRRR | — |
| 120 | RRRRRRRR | — |
| 121 | KKKKKKKK | — |
| 122 | DIMGEWGNEIFGAIAGFLG | — |
| 123 | CHHHHHRKKRRQRRRRHHHHHC | — |
| 124 | CHHHHHRRRRRRRRRHHHHHC | — |
| 125 | FFLIPKGRRRRRRRRGC | — |
| 126 | FΦRRRR | — |
| 127 | RRWWRRWRRRRWWRr | — |
| 128 | RWWRRRRWRRWWRr | — |
| 129 | RRWWRRWRRRRWWr | — |
| 130 | RRWWRRWRRRr | — |
| 131 | RRWWRRWRr | — |
| 132 | RRWWRRWRRR | — |
| 133 | RRRRRRC₁₄RRWWRRr | — |
| 134 | YALTSAISRIITHHHHHH | — |
| 135 | RRRRRC₁₄RRWWRR | — |
| 136 | RRC₁₄RRC₁₄RRRRR | — |
| 137 | RRC₁₄RRR | — |
| 138 | GS | — |
| 139 | GSGS | — |
| 140 | rrrqrrkkrg | — |
| 141 | rrrqrrkkrgyglfgaiagfiengwegmgmidg | — |
| 142 | rrrrrrrr | — |
| 143 | kkkkkkkk | — |
| 144 | glfgaiagfiengwegmid | — |
| 145 | chhhhhrrrrqrrkkrhhhhhc | — |
| 146 | chhhhhrrrrrrrrrhhhhhc | — |
| 147 | cgrrrrrrrrgkpilffc | — |
| 148 | rrrrΦf | — |
| 149 | rrwwrrrrwrrwwrr | — |
| 150 | rrwwrrwrrrrwwr | — |
| 151 | rwwrrrrwrrwwrr | — |
| 152 | rrrrwrrwwrr | — |
| 153 | rrwrrwwrr | — |
| 154 | rrrwrrwwrr | — |
| 155 | rrrwwrrC14rrrrrr | — |
| 156 | hhhhhhtiirsiastlay | — |
| 157 | rrwwrrC₁₄rrrrr | — |
| 158 | rrC₁₄rrC₁₄rrrrr | — |
| 159 | rrC₁₄rrr | — |
| 160 | sg | — |
| 161 | sgsg | — |
| 162 | ATGGATAGTAGTGCGGTGATCACACAAATCTCCAAGGAGGAA | — |
| | GCCCGTGGGCCGCTGCGGGGGAAGGGTGATCAAAAATCGGCA | |
| | GCTAGTCAAAAACCTCGCTCTCGTGGGATACTTCATTCGCTGT | |
| | TTTGCTGCGTCTGCCGCGATGACGGAGAAGCATTGCCTGCGCA | |
| | TTCAGGGGCGCCTTTACTTGTTGAGGAAAATGGTGCAATTCCT | |
| | AAACAAACTCCAGTACAATACTTACTGCCGGAGGCAAAGGCA | |
| | CAAGACAGTGATAAGATATGTGTAGTAATAGACTTAGATGAA | |
| | ACACTGGTACATTCGTCATTCAAACCTGTTAATAATGCGGATT | |
| | TCATCATACCTGTAGAAATCGACGGGGTTGTCCATCAGGTTTA | |
| | CGTCCTGAAGCGGCCTCATGTAGATGAATTTTTACAGCGGATG | |
| | GGCGAGTTATTTGAATGTGTGCTGTTTACAGCTAGTCTTGCCA | |
| | AGTACGCGGATCCTGTCGCGGATTTGCTTGATAAGTGGGGTGC | |
| | GTTTCGGGCGAGATTATTTCGCGAATCTTGCGTTTTTCACAGA | |
| | GGTAACTACGTGAAGGACCTTAGTCGTCTGGGTAGAGATCTTA | |
| | GAAGAGTGCTGATCCTTGACAACAGCCCAGCCAGCTATGTCTT | |
| | TCATCCGGATAACGCAGTACCCGTGGCGTCTTGGTTCGACAAT | |
| | ATGTCGGACACGGAGCTGCATGACCTGTTGCCGTTCTTTGAGC | |
| | AGTTGAGTCGCGTTGATGACGTTTACTCGGTTTTGCGTCAACC | |
| | CCGTCCGGGATCTGGTTCTGGCTCTCACCATCACCATCACCAC | |
| | TAG | |
| 163 | MDSSAVITQISKEEARGPLRGKGDQKSAASQKPRSRGILHSLFCC | — |
| | VCRDDGEALPAHSGAPLLVEENGAIPKQTPVQYLLPEAKAQDSD | |
| | KICVVIDLDETLVHSSFKPVNNADFIIPVEIDGVVHQVYVLKRPHV | |
| | DEFLQRMGELFECVLFTASLAKYADPVADLLDKWGAFRARLFRE | |
| | SCVFHRGNYVKDLSRLGRDLRRVLILDNSPASYVFHPDNAVPVA | |
| | SWFDNMSDTELHDLLPFFEQLSRVDDVYSVLRQPRPGSGSGSHH | |
| | HHHH | |
| 164 | ATGTGTACCGAAGATCTGGAACCACCAGAACCACCACTGCCA | — |
| | AAGGAAAATTGTGGATCCGGTTCTGGCTCAGGTTCTTCCCCTA | |
| | TACTAGGTTATTGGAAAATTAAGGGCCTTGTGCAACCCACTCG | |
| | ACTTCTTTTGGAATATCTTGAAGAAAAATATGAAGAGCATTTG | |
| | TATGAGCGCGATGAAGGTGATAAATGGCGAAACAAAAAGTTT | |
| | GAATTGGGTTTGGAGTTTCCCAATCTTCCTTATTATATTGATGG | |
| | TGATGTTAAATTAACACAGTCTATGGCCATCATACGTTATATA | |
| | GCTGACAAGCACAACATGTTGGGTGGTTGTCCAAAAGAGCGT | |
| | GCAGAGATTTCAATGCTTGAAGGAGCGGTTTTGGATATTAGAT | |
| | ACGGTGTTTCGAGAATTGCATATAGTAAAGACTTTGAAACTCT | |
| | CAAAGTTGATTTTCTTAGCAAGCTACCTGAAATGCTGAAAATG | |
| | TTCGAAGATCGTTTATGTCATAAAACATATTTAAATGGTGATC | |
| | ATGTAACCCATCCTGACTTCATGTTGTATGACGCTCTTGATGTT | |
| | GTTTTATACATGGACCCAATGTGCCTGGATGCGTTCCCAAAAT | |
| | TAGTTTGTTTTAAAAAACGTATTGAAGCTATCCCACAAATTGA | |
| | TAAGTACTTGAAATCCAGCAAGTATATAGCATGGCCTTTGCAG | |
| | GGCTGGCAAGCCACGTTTGGTGGTGGCGACCATCCTCCAAAAT | |
| | AA | |
| 165 | MCTEDLEPPEPPLPKENCGSGSGSGSSPILGYWKIKGLVQPTRLLL | — |
| | EYLEEKYEEHLYERDEGDKWRNKKFELGLEFPNLPYYIDGDVKL | |
| | TQSMAIIRYIADKHNMLGGCPKERAEISMLEGAVLDIRYGVSRIA | |
| | YSKDFETLKVDFLSKLPEMLKMFEDRLCHKTYLNGDHVTHPDF | |
| | MLYDALDVVLYMDPMCLDAFPKLVCFKKRIEAIPQIDKYLKSSK | |
| | YIAWPLQGWQATFGGGDHPPK | |
| 166 | AATAAAGCTTATGTGTACCGAAGATCTGGAACCACCAGAACC | v1f |
| | ACCACTGCCAA | |
| 167 | AATAGGATCCACAATTTTCCTTTGGCAGTGGTGGTTCTGGTGG | v1r |
| | TTCCA | |
| 168 | AATAAAGCTTATGTGTACCGAAGCTCCGGCACCACCAGAACC | v2f |
| | AGCACTGCCAAAG | |
| 169 | AATAGGATCCACAATTTTTTTTTTTTTTCTTTGGCAGTGCTGGT | v2r |
| | TCTGGTGGTGC | |
| 170 | AATAAAGCTTATGTGTACCGAAGATCTGCAACCACCAACAGC | v3f |
| | AGTGCCAC | |
| 171 | AATAGGATCCACAATTTTCCTGTGGCACTGCTGTTGGTGGTTG | v3r |
| | CAGATCTTC | |
| 172 | AATAAAGCTTATGTGTACCGAAGCTCCGGCACCACCAGAACC | v4f |
| | AGCACTGCCAAAG | |
| 173 | AATAGGATCCACAATTTTTTTTTTTCTTTGGCAGTGCTGGTTCT | v4r |
| | GGTGGTGC | |
| 174 | ATGTGTACCGCAGATCTGGAACCACCAGAACCACGAATGGAA | v5f |
| 175 | AATAGGATCCACAATCTACCTTTTTTTTTTCCATTCGTGGTTCT | v5r |
| | GGTGGTTCCA | |
| 176 | AATAAAGCTTATGTGTACCGGAGATCTGCAACCACCAAAAAC | v6f |
| | AACAGTGTCAA | |
| 177 | AATAGGATCCACAATCTTTCTTTGACACTGTTGTTTTTGGTGGT | v6r |
| | TGCAGA | |
| 178 | AATAAAGCTTATGTGTACCGAAGATCTGCAATCACCAAAAAC | v7f |
| | AACAATGACAA | |
| 179 | AATAGGATCCACAATTTTCCTTTGTCATTGTTGTTTTTGGTGAT | v7r |
| | TGCAGA | |
| 180 | AATAAAGCTTATGTGTACCGAAGATCTGGAACCACCAGAACC | v8f |
| | ACCACTGCCAA | |
| 181 | AATAGGATCCCACAATCTTCCTTTGGCAGTGGTGGTTCTGGTG | v8r |
| | GTTCCAGA | |
| 182 | AATAAAGCTTATGTGTACCGAAGATCAGGAACAACAAGAAGA | v9f |
| | ACAACTG | |
| 183 | AATAGGATCCACAATTTTCCTCTGGCAGTTGTTCTTCTTGTTGT | v9r |
| | TCCTGATC | |
| 184 | AATAAAGCTTATGTGTACCGCAGATCTGAAACCACCAAAAAC | v10f |
| | AACAATGACAA | |
| 185 | AATAGGATCCACAATTTTGCTTTGTCATTGTTGTTTTTGGTGGT | v10r |
| | TTCAGA | |
| 186 | AATAAAGCTTATGTGTCCCGGAGATCTGAAACAACCAGAACC | v11f |
| | ACCAATGCCAA | |
| 187 | AATAGGATCCACAATATTCCTTTGGCATTGGTGGTTCTGGTTG | v11r |
| | TTTCAGA | |
| 188 | AATAAAGCTTATGTGTACCGAAGATCTGGAACCACCAAAAGC | v12f |
| | AACAATGACAA | |
| 189 | AATAGGATCCACAATCTTTCTTTGTCATTGTTGCTTTTGGTGGT | v12r |
| | TCCAGA | |
| 190 | AATAAAGCTTATGTGTACCGAAGATCAGGAACGACCACCAGT | v13f |
| | GACAAAG | |
| 191 | AATAGGATCCACAATCTTCCTTTGTCACTGGTGGTCGTTCCTG | v13r |
| | ATCTTC | |
| 192 | AATAAAGCTTATGTGTATCGCAGATCCGGAACCACCAGAAGC | v14f |
| | ACAACTG | |
| 193 | AATAGGATCCACAATTTCCCTCTGGCAGTTGTGCTTCTGGTGG | v14r |
| | TTCCGGATCTG | |
| 194 | AATAAAGCTTATGTGTACCGGAGTTCAGGAACCACCAGAAGC | v15f |
| | AACACTG | |
| 195 | AATAGGATCCACAATTTTTCTTTGGCAGTGTTGCTTCTGGTGGT | v15r |
| | TCCTGAAC | |
| 196 | AATAAAGCTTATGTGTAGCGAAGCTCAGGAACCACCAGAATC | v16f |
| | ACGACTG | |
| 197 | AATAGGATCCACAATTTACCTGTGGCAGTCGTGATTCTGGTGG | v16r |
| | TTCCTGAG | |
| 198 | AATAAAGCTTATGTGTACCAAACATCTGGAACCACCAGGACC | v17f |
| | ACCATTGC | |
| 199 | AATAGGATCCACAATTTTCCTGTGGCAATGGTGGTCCTGGTGG | v17r |
| | TTCCAGATG | |
| 200 | AATAAAGCTTATGTGTACCGCAGCTCCGGAACCACCAGAACC | v18f |
| | ACCAGTGT | |
| 201 | AATAGGATCCACAATATTCCTTTGACACTGGTGGTTCTGGTGG | v18r |
| | TTCCGGAG | |
| 202 | AATAAAGCTTATGTGTACCGAAGATCTGCAACTACCAAAAAC | v19f |
| | AACAATGACA | |
| 203 | AATAGGATCCACAATATTCCTTTGTCATTGTTGTTTTTGGTAGT | v19r |
| | TGCAGA | |
| 204 | AATAAAGCTTATGTGTTCCGTAGATCTGCAACCACCAGCACGA | v20f |
| | CTACGGCCAA | |
| 205 | AATAGGATCCACAATTTACCATTGGCCGTAGTCGTGCTGGTGG | v20r |
| | TTGCAGAT | |
| 206 | AATAAAGCTTATGTGTACCGGAGATCTGCAACCACCAGAATC | v21f |
| | ACGACAGCCA | |
| 207 | AATAGGATCCACAATTTACCTGTGGCTGTCGTGATTCTGGTGG | v21r |
| | TTGCAGAT | |
| 208 | AATAAAGCTTATGTGTACCGGAGATCTGCAACCACCAGAAGC | v22f |
| | ACAAGTGA | |
| 209 | AATAGGATCCACAATTTACCTGTGGCTGTCGTGATTCTGGTGG | v22r |
| | TTGCAGAT | |
| 210 | AATAAAGCTTATGTGTACCGAAGATCTGCAACCACCAGAACC | v23f |
| | ACAACTGCCA | |
| 211 | AATAGGATCCACAATTTACCTCTGGCAGTTGTGGTTCTGGTGG | v23r |
| | TTGCAGAT | |
| 212 | AATAAAGCTTATGTGTACCGAAGATATGGAACCACGAAAAAC | v25f |
| | AACAATGA | |
| 213 | AATAGGATCCACAATATTTCTTTGTCATTGTTGTTTTTCGTGGT | v25r |
| | TCCATAT | |
| 214 | AATAAAGCTTATGTGTACCGAAGCTCCGGCACCACCAGAACC | v26f |
| | AGCACTGCCAAAG | |
| 215 | AATAGGATCCACAATTTTTTTTTTTTTTTTTCTTTGGCAGTGCT | v26r |
| | GGTTCTGGTGGT | |
| 216 | AATAAAGCTTATGTGTACCGAAGCTCCGGCACCACCAGAACC | v27f |
| | AGCACTGCCAAAG | |
| 217 | AATAGGATCCACAATTTTTTTTTTTTTTCTTTGGCAGTGCTGGT | v27r |
| | TCTGGTGGTG | |
| 218 | AATAAAGCTTATGTGTACCGAAGATCTGCAATCACCAAAAAC | v28f |
| | AACAATG | |
| 219 | AATAGGATCCACAATTTTCCTTTGTCATTGTTGTTTTTGGTGAT | v28r |
| | TGCAGA | |
| 220 | AATAAAGCTTATGTGTACCGGAGATCTGAAACTACCAGAACC | v29f |
| | ACCAATGTCAAAG | |
| 221 | AATAGGATCCACAATTTTTTTTTTTTTTCTTTGACATTGGTGGT | v29r |
| | TCTGGTAG | |
| 222 | AATAAAGCTTATGTGTACCGAAGATCTGCAACCACCAAAAAC | v30f |
| | AACAATG | |
| 223 | AATAGGATCCACAATATTTCTCTGCCATTGTTGTTTTTGGTGGT | v30r |
| | TGCAGAT | |
| 224 | AATAAAGCTTATGTGTAGCGAAGATCCGGAACCACCAAAAAC | v31f |
| | AACAATGAC | |
| 225 | AATAGGATCCACAATTTTTCTTTGTCATTGTTGTTTTTGGTGGT | v31r |
| | TCCGGAT | |
| 226 | AATAAAGCTTATGTGTACCGAAGATCTGAAACCACCAGAAGC | v32f |
| | ATCACTGC | |
| 227 | AATAGGATCCACAATTTTCCTCTGGCAGTGATGCTTCTGGTGG | v32r |
| | TTTCAGAT | |
| 228 | AATAAAGCTTATGTGTGCCGGAGATCTGGAACAACCAGAACC | v33f |
| | ACCAGTGGCAAA | |
| 229 | AATAGGATCCACAATTTTTTTTTTTTTTCTTTGCCACTGGTGGT | v33r |
| | TCTGGTTGTTC | |
| 230 | AATAAAGCTTATGTGTAACGGAGATCTGGAACGACCAGAACC | v34f |
| | ACCAGTG | |
| 231 | AATAGGATCCACAATATTCCTTTGCCACTGGTGGTTCTGGTCG | v34r |
| | TTCCAGAT | |
| 232 | AATAAAGCTTATGTGTACCGAAGATCTGAAACCACCAGAACC | v35f |
| | ACCACTGC | |
| 233 | AATAGGATCCACAATTTTCCTTTGGCAGTGGTGGTTCTGGTGG | v35r |
| | TTTCAGAT | |
| 234 | AATAAAGCTTATGTGTAACGAAGCTCTGGAACCACCACCACTG | v36f |
| | CGAAAG | |
| 235 | AATAGGATCCACAATGTTCCTTTCGCAGTGGTGGTGGTTCCAG | v36r |
| | AGCTTC | |
| 236 | AATAAAGCTTATGTGTCCCGAAGATCTGGAACGACCACCATTG | v37f |
| | ACAAAG | |
| 237 | AATAGGATCCACAATGTTCCTTTGTCAATGGTGGTCGTTCCAG | v37r |
| | AT | |
| 238 | AATAAAGCTTATGTGTACCGAAGATCTGGAACCACCAGAACG | v38f |
| | ACCACTGC | |
| 239 | AATAGGATCCACAAATTTCCCTTGGCAGTGGTCGTTCTGGTGG | v38r |
| | TTCCAGA | |
| 240 | AATAAAGCTTATGTGTGCCGGAGATCTGAAACCACCAGAAAC | v39f |
| | AACAATGTC | |
| 241 | AATAGGATCCACAATTTTTCTTTGACATTGTTGTTTCTGGTGGT | v39r |
| | TTCAGA | |
| 242 | AATAAAGCTTATGTGCACCGAAGATCTGCAACAACCAGAACG | v40f |
| | ATCACAGC | |
| 243 | AATAGGATCCACAACTTTCCATTGGCTGTGATCGTTCTGGTTG | v40r |
| | TTGCAGA | |
| 244 | AATAAAGCTTATGTGTCCCGAAGATCTGCAACCACCAGAACC | v41f |
| | AGCACTGCCAGAGAAAAA | |
| 245 | AATAGGATCCGACCACGCCGACGCTGACGGCGCTTTTTACGAC | v41r |
| | CAGAGCCAGAACCACAA | |
| 246 | ACCAGAACCAGCACTGCCAGAGAAAAAAAAAAAAAAAATTGT | v41 |
| | GGTTCTGGCTCTGGTCGT | |
| 247 | AATAAAGCTTATGTGTACCGAAGTTCTGGTACCACGAACCACC | v42f |
| | AGTGGCAAAGGAAG | |
| 248 | AATAGGATCCGGAGCCACACGCCGATGCTGACGGATGGCCTT | v42r |
| | TTTGCGACCAGAGCCAGAA | |
| 249 | ACCACGAACCACCAGTGGCAAAGGAAGATTGTGGTTCTGGCT | v42 |
| | CTGGTCGCAAAAAGGCCA | |
| 250 | AATAAAGCTTATGTGTGCCGAAGATCTGCAACCACCACCACTG | v43f |
| | CTAGAGGCA | |
| 251 | AATAGGATCCACACTGACGGCGCTTTTTACGACCAGAGTCAGA | v43r |
| | ACCACAATGTGCCTCT | |
| 252 | ATCTGCAACCACCACCACTGCTAGAGGCACATTGTGGTTCTGA | v43 |
| | CTCTGGTCGTAAAAAG | |
| 253 | AATAAAGCTTATGTGTACCGCAGCTCCGGAACCACCAGAACC | v44f |
| | ACAACTG | |
| 254 | AATAGGATCCACAATTTGCCTGTGGCAGTTGTGGTTCTGGTGG | v44r |
| | TTCCGGAG | |
| 255 | AATAAAGCTTATGTGTCCCGCAGATCTGCAACAACCAGAAAC | v45f |
| | ATCACTG | |
| 256 | AATAGGATCCACAATTTTCCTCTGGCAGTGATGTTTCTGGTTGT | v45r |
| | TGCAGAT | |
| 257 | AATAAAGCTTATGTGTTCCGTAGATCTGCAACCACCAGCACGA | v46f |
| | CTACG | |
| 258 | AATAGGATCCACAATTTACCATTGGCCGTAGTCGTGCTGGTGG | v46r |
| | TTGCAGAT | |
| 259 | AATAAAGCTTATGTGCACCGAgGCTTTGGAACCACCAGAACCA | v47f |
| | CCACTG | |
| 260 | AATAGGATCCACAATTTTCCTTTGTCAGTGGTGGTTCTGGTGG | v47r |
| | TTCCAAAG | |
| 261 | AATAAAGCTTATGTGTACCGAAGCTATGGAACCACCAGAACC | v48f |
| | ACCACTG | |
| 262 | AATAGGATCCACAACTTTCCCTTGCCAGTGGTGGTTCTGGTGG | v48r |
| | TTCCATAG | |
| 263 | AATAAAGCTTATGTGTACCGCAGATCTGCAACCACCAGAAGC | v49f |
| | ATCACTG | |
| 264 | AATAGGATCCACAATTTTGCTGTGGCAGTGATGCTTCTGGTGG | v49r |
| | TTGCAGAT | |
| 265 | AATAAAGCTTATGTGTACCGCAGCTCCGGAACCACCAGAACC | v50f |
| | ACGACTGCCA | |
| 266 | AATAGGATCCACAATTTCCCTCTGGCAGTCGTGGTTCTGGTGG | v50r |
| | TTCC | |
| 267 | AATAAAGCTTATGTGTACCAAAGATTTGGCACCACAAGCACC | v51f |
| | ACCATTGCT | |
| 268 | AATAGGATCCACAATTTTCCTTTAGCAATGGTGGTGCTTGTGG | v51r |
| | TGCCAAA | |
| 269 | AAATAAGCTTGTATATCTCCTTCTTAAAGTTAAACA | P12 |
| 270 | AATAACTCGAGAGATCCGGCTGCTAACAAAGC | P14 |
| 271 | ATAAGCTTTAATACGACTCACTATAGGGTTAACTTTAGTAAGG | P17 |
| | AGGACAGCTAA | |
| 272 | GTGGTGATGGTGATGGTGACCTA | P19 |
| 273 | ATTATTCTCGAGTTAATAGCCGGTGCCGTGGTGATGGTGATGG | P20 |
| | TGACCTA | |
| 274 | AAATACTCGAGTCGTTTTATCTGTTGTTTGTCGGT | P21 |
| 275 | AAATAAAGCTTCTCTGAATGGCGGGAGTATGAAAA | P22 |
| 276 | CCGCGAATGGTGAGATTGAGAA | P23 |
| 277 | ACGCAAAAAGGCCATCCGTCAG | P24 |
| 278 | ATTATTCTCGAGTTAATAGCCGGTGCCTAAGCCGCTACCACCA | P28 |
| | CGCCGACGCTGACGG | |
| 279 | AAATAAGCTTATGGATAGTAGTGCGGTGATCA | P31 |
| 280 | ATTATTCTCGAGCTAGTGGTGATGGTGATGGT | P32 |
| 281 | AACTAAGCTTTTCCTCCTGTTAGCCCAAAAAAC | P33 |
| 282 | AATACTCGAGGCTGTTTTGGCGGATGAGAGAA | P34 |
| 283 | AAATCCATGGATAGTAGTGCGGTGATCA | P35 |
| 284 | AAATCATATGGATAGTAGTGCGGTGATCA | P36 |
| 285 | AATACTCGAGCTAGTGGTGATGGTGATGGTGAG | P37 |
| 286 | AATACTCGAGAGAGCCAGAACCAGATCCCGGA | P38 |
| 287 | TAAGCCGCTACCACCACGCCGAC | P39 |
| 288 | GGTGCAATTCCTAAAACTCCAGTACAATACTTACTGC | P40 |
| 289 | GTATTGTACTGGAGTTTTAGGAATTGCACCATTTTCCTC | P41 |
| 290 | AATACTCGAGTTATTTTGGAGGATGGTCGCCACCA | P108 |
| 291 | AATAAAGCTTATGGGATCCGGTTCTGGCTCAGGTTCTTCC | P109 |
| 292 | NAANCANCANTGNCANAGNAANATTGTGGTTCTGGCTCTGGT | 11F |
| | CGTAAAA | |
| 293 | GNACNACNACNGCNAANGGNAANTTGTGGTTCTGGCTCTGGT | 12F |
| | CGTAAAA | |
| 294 | GNANCACNANTGCNANAGGNANATTGTGGTTCTGGCTCTGGT | 13F |
| | CGTAAAA | |
| 295 | NAACNANCACNGNCAANGNAAANTTGTGGTTCTGGCTCTGGT | 14F |
| | CGTAAAA | |
| 296 | GAANCANCANTGNCANAGNAANATTGTGGTTCTGGCTCTGGT | 15F |
| | CGTAAAA | |
| 297 | GAACNACNACNGCNAANGGNAANTTGTGGTTCTGGCTCTGGT | 16F |
| | CGTAAAA | |
| 298 | GAANCACNANTGCNANAGGNANATTGTGGTTCTGGCTCTGGT | 17F |
| | CGTAAAA | |
| 299 | GAACNANCACNGNCAANGNAAANTTGTGGTTCTGGCTCTGGT | 18F |
| | CGTAAAA | |
| 300 | GAANCANCACTGCCANAGNAAAATTGTGGTTCTGGCTCTGGTC | 19F |
| | GTAAAA | |
| 301 | NAACCACCANTGNCAAAGGAANATTGTGGTTCTGGCTCTGGTC | 20F |
| | GTAAAA | |
| 302 | GAANCACNACTGCCANAGGNAAATTGTGGTTCTGGCTCTGGTC | 21F |
| | GTAAAA | |
| 303 | GNACCACCANTGCNAAAGGAANATTGTGGTTCTGGCTCTGGTC | 22F |
| | GTAAAA | |
| 304 | TGNTGNTTNCANATNTTNGGNACACATTTAGCTGTCCTCCTTA | 11R |
| | CTAAAGTT | |
| 305 | TNGTNGTNCCNGANCTNCGNTACACATTTAGCTGTCCTCCTTA | 12R |
| | CTAAAGTT | |
| 306 | TGNTNGTTNCNGATNTNCGGNACACATTTAGCTGTCCTCCTTA | 13R |
| | CTAAAGTT | |
| 307 | TNGTGNTNCCANANCTTNGNTACACATTTAGCTGTCCTCCTTA | 14R |
| | CTAAAGTT | |
| 308 | TGNTGNTTCCANATNTTNGGNACACATTTAGCTGTCCTCCTTA | 15R |
| | CTAAAGTT | |
| 309 | TNGTNGTTCCNGANCTNCGNTACACATTTAGCTGTCCTCCTTA | 16R |
| | CTAAAGTT | |
| 310 | TGNTNGTTCCNGATNTNCGGNACACATTTAGCTGTCCTCCTTA | 17R |
| | CTAAAGTT | |
| 311 | TNGTGNTTCCANANCTTNGNTACACATTTAGCTGTCCTCCTTA | 18R |
| | CTAAAGTT | |
| 312 | TGGTGNTTNCAGATCTTNGGNACACATTTAGCTGTCCTCCTTA | 19R |
| | CTAAAGTT | |
| 313 | TGNTGGTTCCANATNTTCGGTACACATTTAGCTGTCCTCCTTAC | 20R |
| | TAAAGTT | |
| 314 | TGGTNGTTNCAGATCTNCGGNACACATTTAGCTGTCCTCCTTA | 21R |
| | CTAAAGTT | |
| 315 | TGNTGGTTCCNGATNTTCGGTACACATTTAGCTGTCCTCCTTAC | 22R |
| | TAAAGTT | |
| 316 | CCTGCAGGTAATACGACTCACTATAGGGTTAACTTTAGTAAGG | — |
| | AGGACAGCTAAAAGCTTATGTGTACCGAAGATCTGGAACCAC | |
| | CAGAACCACCACTGCCAAAGGAAAATTGTGGATCCGGCTCTG | |
| | GTCGTAAAAAGCGCCGTCAGCGTCGGCGTGGTGGCTCCGGTA | |
| | GCTTAGGTCACCATCACCATCACCACGGCACCGGCTATTAACT | |
| | CGAG | |
| 317 | ATGTGTACCGAAGATCTGGAACCACCAGAACCACCACTGCCA | — |
| | AAGGAAAATTGTGGATCCGGCTCTGGTCGTAAAAAGCGCCGT | |
| | CAGCGTCGGCGTGGTGGCTCCGGTAGCTTAGGTCACCATCACC | |
| | ATCACCACGGCACCGGCTATTAA | |
| 318 | AAATCCTGCAGGTAATACGACTCACTATAGGGTTAAC | — |
| 319 | ATTATTCTCGAGTTAATAGCCGGTGCCGTGGTGATGGTGATGG | — |
| | TGACCTA | |
| 320 | TATACCCTGCAGGTAATACGACTCACTATAGGGTTAACTTTAG | — |
| | TAAGGAGGACAGCTAAAAGCTTATGTGTGAAGACGCCAAAAA | |
| | CATAAAGAAAGGCCCGGCGCCATTCTATCCGTGTGGATCCGGC | |
| | TCTGGTCGTAAAAAGCGCCGTCAGCGTCGGCGTGGTGGCTCCG | |
| | GTAGCTTAGGTCACCATCACCATCACCACGGCACCGGCTATTA | |
| | ACTCGAGAGATC | |
| 321 | CCTGCAGGTAATACGACTCACTATAGGGTTAACTTTAGTAAGG | — |
| | AGGACAGCTAAAAGCTTATGTGTGAAGACGCCAAAAACATAA | |
| | AGAAAGGCCCGGCGCCATTCTATCCGTGTGGATCCGGCTCTGG | |
| | TCGTAAAAAGCGCCGTCAGCGTCGGCGTGGTGGCTCCGGTAG | |
| | CTTAGGTCACCATCACCATCACCACGGCACCGGCTATTAACTC | |
| | GAG | |
| 322 | ATGTGTGAAGACGCCAAAAACATAAAGAAAGGCCCGGCGCCA | — |
| | TTCTATCCGTGTGGATCCGGCTCTGGTCGTAAAAAGCGCCGTC | |
| | AGCGTCGGCGTGGTGGCTCCGGTAGCTTAGGTCACCATCACCA | |
| | TCACCACGGCACCGGCTATTAA | |
| 323 | AAATCCTGCAGGTAATACGACTCACTATAGGGTTAAC | — |
| 324 | ATTATTCTCGAGTTAATAGCCGGTGCCGTGGTGATGGTGATGG | — |
| | TGACCTA | |
| 2 | MCTEDLEPPEPPLPKENCGSGSGRKKRRQRRRGGSGSLGHHHHH | — |
| | HGTGY | |
| 3 | MCEDAKNIKKGPAPFYPCGSGSGRKKRRQRRRGGSGSLGHHHH | Control |
| | HHGTGY | |
| 4 | CAQKDYKDDDDKTEDLEPPEPPLPKENGRKKRRQRRRG | — |
| 5 | CTEDLEPPEPPLPKENSGDIMGEWGNEIFGAIAGFLGYGRKKRRQ | — |
| | RRRG^{cycli(head to tail)} | |
| 6 | TEDLEPPEPPLPKENRRWWRRWRRRRWWRr | — |
| 7 | EDLEPPEPPLPKRWWRRRRWRRWWRr | — |
| 8 | AGDLEQPEPPVAKKKKKNRRWWRRWRr | — |
| 9 | rrrrwrrwwrrwrrnekplppeppeldet | — |
| 10 | TEDLEPPEPPLPKENrrrrwrrwwrrwrr | — |
| 11 | TEDLEPPEPPLPKENRRRRRRC₁₄RRWWRRr | — |
| 12 | rwwrrC₁₄rrrrrkplppeppelde^{cyclic(head to tail)} | — |
| 13 | rrC₁₄rrC₁₄rrrrrrkplppeppelde^{cyclic(head to tail)} | — |
| 14 | rrC₁₄rrrkplppeppelde^{cyclic(head to tail)} | — |

Lower case = D amino acids; C₁₄ = 2-aminotetradecanoic acid; Φ is L-2-naphthylalanine; N = any nucleotide

Although certain presently preferred embodiments of the invention have been specifically described herein, it will be apparent to those skilled in the art to which the invention pertains that variations and modifications of the various embodiments shown and described herein may be made without departing from the spirit and scope of the invention.

### REFERENCES (FOR SPECIFICATION, EXCEPT EXAMPLES)

1 Bruce, A. W. et al. Genome-wide analysis of repressor element 1 silencing transcription factor/neuron-restrictive silencing factor (REST/NRSF) target genes. Proc Natl Acad Sci USA 101, 10458-10463, doi: 10.1073/pnas.04018271010401827101 [pii] (2004).
2 Nesti, E., Corson, G. M., McCleskey, M., Oyer, J. A. & Mandel, G. C-terminal domain small phosphatase 1 and MAP kinase reciprocally control REST stability and neuronal differentiation. Proc Natl Acad Sci USA 111, E3929-3936, doi: 1414770111 [pii]10.1073/pnas. 1414770111 (2014).
3 Nesti, E. METHODS AND COMPOSITIONS USEFUL IN MANIPULATING THE STABILITY OF RE1 SILENCING TRANSCRIPTION FACTOR. US, PTC patent (2014, 2015).
4 Nesti, E. Harnessing the master transcriptional repressor REST to reciprocally regulate neurogenesis. Neurogenesis 2, doi: 10.1080/23262133.2015.1055419 (2015).
5 Calderone, A. et al. Ischemic insults derepress the gene silencer REST in neurons destined to die. J Neurosci 23, 2112-2121, doi: 23/6/2112 [pii] (2003).
6 Kaneko, N., Hwang, J. Y., Gertner, M., Pontarelli, F. & Zukin, R. S. Casein kinase 1 suppresses activation of REST in insulted hippocampal neurons and halts ischemia-induced neuronal death. J Neurosci 34, 6030-6039, doi: 34/17/6030 [pii]10.1523/JNEUROSCI.4045-13.2014 (2014).
7 Noh, K. M. et al. Repressor element-1 silencing transcription factor (REST)-dependent epigenetic remodeling is critical to ischemia-induced neuronal death. Proc Natl Acad Sci USA 109, E962-971, doi: 1121568109 [pii]10.1073/pnas. 1121568109 (2012).
8 Abe, K. Therapeutic potential of neurotrophic factors and neural stem cells against ischemic brain injury. J Cereb Blood Flow Metab 20, 1393-1408, doi: 10.1097/00004647-200010000-00001 (2000).
9 Kan Ding, P. K. G., and Ramon Diaz-Arrastia. in Translational Research in Traumatic Brain Injury (ed Grant G Laskowitz D) Ch. 14, (Taylor and Francis, 2016).
10 Pasquina, P., Kirtley, R. & Ling, G. Moderate-to-severe traumatic brain injury. Semin Neurol 34, 572-583, doi: 10.1055/s-0034-1396010 (2014).
11 Hwang, J. Y., Kaneko, N., Noh, K. M., Pontarelli, F. & Zukin, R. S. The gene silencing transcription factor REST represses miR-132 expression in hippocampal neurons destined to die. J Mol Biol 426, 3454-3466, doi: 10.1016/j.jmb.2014.07.032 (2014).
12 McClelland, S. et al. The transcription factor NRSF contributes to epileptogenesis by selective repression of a subset of target genes. Elife 3, e01267 (2014).
13 Lu, T. et al. REST and stress resistance in ageing and Alzheimer's disease. Nature 507, 448-454, doi: nature13163 [pii]10.1038/nature13163 (2014).
14 Orta-Salazar, E. et al. REST/NRSF-induced changes of ChAT protein expression in the neocortex and hippocampus of the 3×Tg-AD mouse model for Alzheimer's disease. Life sciences 116, 83-89, doi: 10.1016/j.lfs.2014.09.013 (2014).
15 Sharp, D. J., Scott, G. & Leech, R. Network dysfunction after traumatic brain injury. Nat Rev Neuro/10, 156-166, doi: 10.1038/nrneurol.2014.15 (2014).
16 Roopra, A., Dingledine, R. & Hsieh, J. Epigenetics and epilepsy. Epilepsia 53 Suppl 9, 2-10, doi: 10.1111/epi. 12030 (2012).
17 Bergsland, M., Covacu, R., Perez Estrada, C., Svensson, M. & Brundin, L. Nitric oxide-induced neuronal to glial lineage fate-change depends on NRSF/REST function in neural progenitor cells. Stem Cells 32, 2539-2549, doi: 10.1002/stem. 1749 (2014).
18 Conaco, C., Otto, S., Han, J. J. & Mandel, G. Reciprocal actions of REST and a microRNA promote neuronal identity. Proc Natl Acad Sci USA 103, 2422-2427, doi: 0511041103 [pii]10.1073/pnas.0511041103 (2006).
19 Covey, M. V., Streb, J. W., Spektor, R. & Ballas, N. REST regulates the pool size of the different neural lineages by restricting the generation of neurons and oligodendrocytes from neural stem/progenitor cells. Development 139, 2878-2890, doi: dev.074765 [pii]10.1242/dev.074765 (2012).
20 Gao, Z. et al. The master negative regulator REST/NRSF controls adult neurogenesis by restraining the neurogenic program in quiescent stem cells. J Neurosci 31, 9772-9786, doi: 31/26/9772 [pii]10.1523/JNEUROSCI. 1604-11.2011 (2011).
21 Kohyama, J. et al. BMP-induced REST regulates the establishment and maintenance of astrocytic identity. J Cell Biol 189, 159-170, doi: jcb.200908048 [pii]10.1083/jcb.200908048 (2010).
22 Mandel, G. et al. Repressor element 1 silencing transcription factor (REST) controls radial migration and temporal neuronal specification during neocortical development. Proc Natl Acad Sci USA 108, 16789-16794, doi: 1113486108 [pii]10.1073/pnas. 1113486108 (2011).
23 Vincent, A. S., Roebuck-Spencer, T. M. & Cernich, A. Cognitive changes and dementia risk after traumatic brain injury: implications for aging military personnel. Alzheimers Dement 10, S174-187, doi: 10.1016/j.jalz.2014.04.006 (2014).
24 Kirn, J. R. The relationship of neurogenesis and growth of brain regions to song learning. Brain Lang 115, 29-44, doi: S0093-934X (09) 00135-7 [pii]10.1016/j.bandl.2009.09.006 (2010).
25 Oomen, C. A., Bekinschtein, P., Kent, B. A., Saksida, L. M. & Bussey, T. J. Adult hippocampal neurogenesis and its role in cognition. Wiley Interdiscip Rev Cogn Sci 5, 573-587, doi: 10.1002/wcs. 1304 (2014).
26 Samuels, I. S. et al. Deletion of ERK2 mitogen-activated protein kinase identifies its key roles in cortical neurogenesis and cognitive function. J Neurosci 28, 6983-6995, doi: 28/27/6983 [pii]10.1523/JNEUROSCI.0679-08.2008 (2008).
27 Shors, T. J. et al. Neurogenesis in the adult is involved in the formation of trace memories. Nature 410, 372-376, doi: 10.1038/35066584 (2001).
28 Villeda, S. A. et al. The ageing systemic milieu negatively regulates neurogenesis and cognitive function. Nature 477, 90-94, doi: 10.1038/nature10357 (2011).
29 Bird, C. M. & Burgess, N. The hippocampus supports recognition memory for familiar words but not unfamiliar faces. Curr Bio/18, 1932-1936, doi: 10.1016/j.cub.2008.10.046 (2008).
30 T. J. Shors, G. M., A. Beylin, M. Zhao, T. Rydel, E. Gould. Neurogenesis in the adult is involved in the formation of trace memories. Nature 410, 372-376 (2001).
31 VanElzakker, M., Fevurly, R. D., Breindel, T. & Spencer, R. L. Environmental novelty is associated with a selective increase in Fos expression in the output elements of the hippocampal formation and the perirhinal cortex. Learn Mem 15, 899-908, doi: 10.1101/1m. 1196508 (2008).
32 Thornton, G. K. & Woods, C. G. Primary microcephaly: do all roads lead to Rome? Trends Genet 25, 501-510, doi: 10.1016/j.tig.2009.09.011 (2009).
33 Ghaziuddin, M., Zaccagnini, J., Tsai, L. & Elardo, S. Is megalencephaly specific to autism? J Intellect Disabil Res 43 (Pt 4), 279-282 (1999).
34 Kuhn, H. G., Cooper-Kuhn, C. M., Boekhoorn, K. & Lucassen, P. J. Changes in neurogenesis in dementia and Alzheimer mouse models: are they functionally relevant? Eur Arch Psychiatry Clin Neurosci 257, 281-289, doi: 10.1007/s00406-007-0732-4 (2007).
35 Baker, M. Tumours spark stem-cell review. Nature 457, 941, doi: 10.1038/457941a (2009).
36 Brederlau, A. et al. Transplantation of human embryonic stem cell-derived cells to a rat model of Parkinson's disease: effect of in vitro differentiation on graft survival and teratoma formation. Stem Cells 24, 1433-1440, doi: 2005-0393 [pii]10.1634/stemcells.2005-0393 (2006).
37 Burns, T. C., Verfaillie, C. M. & Low, W. C. Stem cells for ischemic brain injury: a critical review. J Comp Neurol 515, 125-144, doi: 10.1002/cne.22038 (2009).
38 Carlson, A. P., Schermer, C. R. & Lu, S. W. Retrospective evaluation of anemia and transfusion in traumatic brain injury. J Trauma 61, 567-571, doi: 10.1097/01.ta.0000231768.44727.a200005373-200609000-00007 [pii] (2006).
39 Carmeliet, P. & Storkebaum, E. Vascular and neuronal effects of VEGF in the nervous system: implications for neurological disorders. Semin Cell Dev Biol 13, 39-53, doi: 10.1006/scdb.2001.0290S1084952101902903 [pii] (2002).
40 Choe, Y., Kozlova, A., Graf, D. & Pleasure, S. J. Bone morphogenic protein signaling is a major determinant of dentate development. J Neurosci 33, 6766-6775, doi: 33/16/6766 [pii]10.1523/JNEUROSCI.0128-13.2013 (2013).
41 Erdo, F. et al. Host-dependent tumorigenesis of embryonic stem cell transplantation in experimental stroke. J Cereb Blood Flow Metab 23, 780-785, doi: 10.1097/01.WCB.0000071886.63724.FB (2003).
42 Greig, N. H. et al. Incretin mimetics as pharmacologic tools to elucidate and as a new drug strategy to treat traumatic brain injury. Alzheimers Dement 10, S62-75, doi: S1552-5260 (13) 02925-7 [pii]10.1016/j.jalz.2013.12.011 (2014).
43 Kaplan, G. B., Vasterling, J. J. & Vedak, P. C. Brain-derived neurotrophic factor in traumatic brain injury, post-traumatic stress disorder, and their comorbid conditions: role in pathogenesis and treatment. Behav Pharmacol 21, 427-437, doi: 10.1097/FBP.0b013e32833d8bc9 (2010).
44 Knoepfler, P. S. Deconstructing stem cell tumorigenicity: a roadmap to safe regenerative medicine. Stem Cells 27, 1050-1056, doi: 10.1002/stem.37 (2009).
45 Luca Longhia, Elisa R. Zaniera, Nicolas Royob, Nino Stocchettia, Tracy K. McIntosha. Stem cell transplantation as a therapeutic strategy for traumatic brain injury. Transplant Immunology 15, 134-148 (2005).
46 Robertson, C. S. et al. Effect of erythropoietin and transfusion threshold on neurological recovery after traumatic brain injury: a randomized clinical trial. JAMA 312, 36-47, doi: 1884575 [pii]10.1001/jama.2014.6490 (2014).
47 Salim, A. et al. Role of anemia in traumatic brain injury. J Am Coll Surg 207, 398-406, doi: S1072-7515 (08) 00322-0 [pii]10.1016/j.jamcollsurg.2008.03.013 (2008).
48 Deng, W., Aimone, J. B. & Gage, F. H. New neurons and new memories: how does adult hippocampal neurogenesis affect learning and memory? Nat Rev Neurosci 11, 339-350, doi: nrn2822 [pii]10.1038/nrn2822 (2010).
49 Goldman, S. A. & Nottebohm, F. Neuronal production, migration, and differentiation in a vocal control nucleus of the adult female canary brain. Proc Natl Acad Sci USA 80, 2390-2394 (1983).
50 Gould, E., Beylin, A., Tanapat, P., Reeves, A. & Shors, T. J. Learning enhances adult neurogenesis in the hippocampal formation. Nat Neurosci 2, 260-265, doi: 10.1038/6365 (1999).
51 Kirn, J. R. & Nottebohm, F. Direct evidence for loss and replacement of projection neurons in adult canary brain. J Neurosci 13, 1654-1663 (1993).
52 Mu, Y. & Gage, F. H. Adult hippocampal neurogenesis and its role in Alzheimer's disease. Mol Neurodegener 6, 85, doi: 1750-1326-6-85 [pii]10.1186/1750-1326-6-85 (2011).
53 Pytte, C. L., Gerson, M., Miller, J. & Kirn, J. R. Increasing stereotypy in adult zebra finch song correlates with a declining rate of adult neurogenesis. Dev Neurobiol 67, 1699-1720, doi: 10.1002/dneu.20520 (2007).
54 Scharff, C., Kirn, J. R., Grossman, M., Macklis, J. D. & Nottebohm, F. Targeted neuronal death affects neuronal replacement and vocal behavior in adult songbirds. Neuron 25, 481-492, doi: S0896-6273 (00) 80910-1 [pii] (2000).
55 Seib, D. R. et al. Loss of Dickkopf-1 restores neurogenesis in old age and counteracts cognitive decline. Cell Stem Cell 12, 204-214, doi: S1934-5909 (12) 00644-3 [pii]10.1016/j.stem.2012.11.010 (2013).
56 Deister, C. & Schmidt, C. E. Optimizing neurotrophic factor combinations for neurite outgrowth. J Neural Eng 3, 172-179, doi: 10.1088/1741-2560/3/2/011 (2006).
57 Zhao, Y. et al. Brain REST/NRSF Is Not Only a Silent Repressor but Also an Active Protector. Mol Neurobiol 54, 541-550, doi: 10.1007/s12035-015-9658-4 (2017).
58 Uchida, H., Ma, L. & Ueda, H. Epigenetic Gene Silencing Underlies C-Fiber Dysfunctions in Neuropathic Pain. Journal of Neuroscience 30, 4806-4814, doi: 10.1523/JNEUROSCI.5541-09.2010 (2010).
59 Rose, K. et al. Transcriptional repression of the M channel subunit Kv7.2 in chronic nerve injury. Pain 152, 742-754, doi: 10.1016/j.pain.2010.12.028 (2011).
60 Costigan, M. et al. Multiple chronic pain states are associated with a common amino acid-changing allele in KCNS1. Brain 133, 2519-2527, doi: 10.1093/brain/awq195 (2010).
61 Ueda, H. et al. A mimetic of the mSin3-binding helix of NRSF/REST ameliorates abnormal pain behavior in chronic pain models. Bioorg Med Chem Lett 27, 4705-4709, doi: 10.1016/j.bmcl.2017.09.006 (2017).
62 Zhang, J., Chen, S. R., Chen, H. & Pan, H. L. RE1-silencing transcription factor controls the acute-to-chronic neuropathic pain transition and Chrm2 receptor gene expression in primary sensory neurons. J Biol Chem 293, 19078-19091, doi: 10.1074/jbc.RA118.005846 (2018).
63 Banerjee, P. N., Filippi, D. & Allen Hauser, W. The descriptive epidemiology of epilepsy—a review. Epilepsy Res 85, 31-45, doi: 10.1016/j.eplepsyres.2009.03.003 (2009).
64 Mucha, M. et al. Transcriptional control of KCNQ channel genes and the regulation of neuronal excitability. J Neurosci 30, 13235-13245, doi: 10.1523/JNEUROSCI.1981-10.2010 (2010).
65 Escayg, A. et al. Mutations of SCN1A, encoding a neuronal sodium channel, in two families with GEFS+2. Nat Genet 24, 343-345, doi: 10.1038/74159 (2000).
66 Martin, D. et al. REST represses a subset of the pancreatic endocrine differentiation program. Dev Biol 405, 316-327, doi: 10.1016/j.ydbio.2015.07.002 (2015).
67 Abderrahmani, A. et al. Neuronal traits are required for glucose-induced insulin secretion. FEBS Lett 565, 133-138, doi: 10.1016/j.febslet.2004.04.002 (2004).
68 Martin, D. et al. Functional significance of repressor element 1 silencing transcription factor (REST) target genes in pancreatic beta cells. Diabetologia 51, 1429-1439, doi: 10.1007/s00125-008-0984-1 (2008).
69 Rigamonti, D. et al. Loss of huntingtin function complemented by small molecules acting as repressor element 1/neuron restrictive silencer element silencer modulators. J Biol Chem 282, 24554-24562, doi: 10.1074/jbc. M609885200 (2007).
70 Sipione, S. et al. Early transcriptional profiles in huntingtin-inducible striatal cells by microarray analyses. Hum Mol Genet 25, 210, doi: 10.1093/hmg/ddv416 (2016).
71 Zuccato, C. et al. Huntingtin interacts with REST/NRSF to modulate the transcription of NRSE-controlled neuronal genes. Nat Genet 35, 76-83, doi: 10.1038/ng1219 (2003).
72 Bao, S. et al. Targeting cancer stem cells through L1CAM suppresses glioma growth. Cancer Res 68, 6043-6048, doi: 10.1158/0008-5472.CAN-08-1079 (2008).
73 Jackson, M., Hassiotou, F. & Nowak, A. Glioblastoma stem-like cells: at the root of tumor recurrence and a therapeutic target. Carcinogenesis 36, 177-185, doi: 10.1093/carcin/bgu243 (2015).
74 Liu, G. et al. Analysis of gene expression and chemoresistance of CD133+ cancer stem cells in glioblastoma. Mol Cancer 5, 67, doi: 10.1186/1476-4598-5-67 (2006).
75 Persano, L., Rampazzo, E., Basso, G. & Viola, G. Glioblastoma cancer stem cells: role of the microenvironment and therapeutic targeting. Biochem Pharmacol 85, 612-622, doi: 10.1016/j.bcp.2012.10.001 (2013).
76 Conti, L. et al. REST controls self-renewal and tumorigenic competence of human glioblastoma cells. PLOS One 7, e38486, doi: 10.1371/journal.pone.0038486 (2012).
77 Kamal, M. M. et al. REST regulates oncogenic properties of glioblastoma stem cells. Stem Cells 30, 405-414, doi: 10.1002/stem. 1020 (2012).
78 Wagoner, M. P. & Roopra, A. A REST derived gene signature stratifies glioblastomas into chemotherapy resistant and responsive disease. BMC Genomics 13, 686, doi: 10.1186/1471-2164-13-686 (2012).
79 Taylor, P. et al. REST is a novel prognostic factor and therapeutic target for medulloblastoma. Mol Cancer Ther 11, 1713-1723, doi: 10.1158/1535-7163.MCT-11-0990 (2012).
80 Jin, H. et al. Identification of RE1-Silencing Transcription Factor as a Promoter of Metastasis in Pancreatic Cancer. Front Oncol 9, 291, doi: 10.3389/fonc.2019.00291 (2019).

### REFERENCES (FOR EXAMPLES ONLY)

1 Bruce, A. W. et al. Genome-wide analysis of repressor element 1 silencing transcription factor/neuron-restrictive silencing factor (REST/NRSF) target genes. Proc Natl Acad Sci USA 101, 10458-10463, doi: 10.1073/pnas.04018271010401827101 [pii] (2004).
2 Nesti, E., Corson, G. M., McCleskey, M., Oyer, J. A. & Mandel, G. C-terminal domain small phosphatase 1 and MAP kinase reciprocally control REST stability and neuronal differentiation. Proc Natl Acad Sci USA 111, E3929-3936, doi: 1414770111 [pii]10.1073/pnas. 1414770111 (2014).
3 Nesti, E. METHODS AND COMPOSITIONS USEFUL IN MANIPULATING THE STABILITY OF REI SILENCING TRANSCRIPTION FACTOR. US, PTC patent (2014, 2015).
4 Nesti, E. Harnessing the master transcriptional repressor REST to reciprocally regulate neurogenesis. Neurogenesis 2, doi: 10.1080/23262133.2015.1055419 (2015).
5 Arranz-Gibert, P. et al. Immunosilencing peptides by stereochemical inversion and sequence reversal: retro-D-peptides. Sci Rep 8, 6446, doi: 10.1038/s41598-018-24517-6 (2018).
6 Hall, E. D., Bryant, Y. D., Cho, W. & Sullivan, P. G. Evolution of post-traumatic neurodegeneration after controlled cortical impact traumatic brain injury in mice and rats as assessed by the de Olmos silver and fluorojade staining methods. J Neurotrauma 25, 235-247, doi: 10.1089/neu.2007.0383 (2008).
7 Calderone, A. et al. Ischemic insults derepress the gene silencer REST in neurons destined to die. J Neurosci 23, 2112-2121, doi: 23/6/2112 [pii] (2003).
8 Kaneko, N., Hwang, J. Y., Gertner, M., Pontarelli, F. & Zukin, R. S. Casein kinase 1 suppresses activation of REST in insulted hippocampal neurons and halts ischemia-induced neuronal death. J Neurosci 34, 6030-6039, doi: 34/17/6030 [pii]10.1523/JNEUROSCI.4045-13.2014 (2014).
9 Noh, K. M. et al. Repressor element-1 silencing transcription factor (REST)-dependent epigenetic remodeling is critical to ischemia-induced neuronal death. Proc Natl Acad Sci USA 109, E962-971, doi: 1121568109 [pii]10.1073/pnas. 1121568109 (2012).
10 Abe, K. Therapeutic potential of neurotrophic factors and neural stem cells against ischemic brain injury. J Cereb Blood Flow Metab 20, 1393-1408, doi: 10.1097/00004647-200010000-00001 (2000).
11 Kan Ding, P. K. G., and Ramon Diaz-Arrastia. in Translational Research in Traumatic Brain Injury (ed Grant G Laskowitz D) Ch. 14, (Taylor and Francis, 2016).
12 Pasquina, P., Kirtley, R. & Ling, G. Moderate-to-severe traumatic brain injury. Semin Neurol 34, 572-583, doi: 10.1055/s-0034-1396010 (2014).
13 Hwang, J. Y., Kaneko, N., Noh, K. M., Pontarelli, F. & Zukin, R. S. The gene silencing transcription factor REST represses miR-132 expression in hippocampal neurons destined to die. J Mol Biol 426, 3454-3466, doi: 10.1016/j.jmb.2014.07.032 (2014).
14 McClelland, S. et al. The transcription factor NRSF contributes to epileptogenesis by selective repression of a subset of target genes. Elife 3, e01267 (2014).
15 Lu, T. et al. REST and stress resistance in ageing and Alzheimer's disease. Nature 507, 448-454, doi: nature13163 [pii]10.1038/nature13163 (2014).
16 Orta-Salazar, E. et al. REST/NRSF-induced changes of ChAT protein expression in the neocortex and hippocampus of the 3×Tg-AD mouse model for Alzheimer's disease. Life sciences 116, 83-89, doi: 10.1016/j.lfs.2014.09.013 (2014).
17 Sharp, D. J., Scott, G. & Leech, R. Network dysfunction after traumatic brain injury. Nat Rev Neurol 10, 156-166, doi: 10.1038/nrneurol.2014.15 (2014).
18 Roopra, A., Dingledine, R. & Hsieh, J. Epigenetics and epilepsy. Epilepsia 53 Suppl 9, 2-10, doi: 10.1111/epi.12030 (2012).
19 Bergsland, M., Covacu, R., Perez Estrada, C., Svensson, M. & Brundin, L. Nitric oxide-induced neuronal to glial lineage fate-change depends on NRSF/REST function in neural progenitor cells. Stem Cells 32, 2539-2549, doi: 10.1002/stem. 1749 (2014).
20 Conaco, C., Otto, S., Han, J. J. & Mandel, G. Reciprocal actions of REST and a microRNA promote neuronal identity. Proc Natl Acad Sci USA 103, 2422-2427, doi: 0511041103 [pii]10.1073/pnas.0511041103 (2006).
21 Covey, M. V., Streb, J. W., Spektor, R. & Ballas, N. REST regulates the pool size of the different neural lineages by restricting the generation of neurons and oligodendrocytes from neural stem/progenitor cells. Development 139, 2878-2890, doi: dev.074765 [pii]10.1242/dev.074765 (2012).
22 Gao, Z. et al. The master negative regulator REST/NRSF controls adult neurogenesis by restraining the neurogenic program in quiescent stem cells. J Neurosci 31, 9772-9786, doi: 31/26/9772 [pii]10.1523/JNEUROSCI.1604-11.2011 (2011).
23 Kohyama, J. et al. BMP-induced REST regulates the establishment and maintenance of astrocytic identity. J Cell Biol 189, 159-170, doi: jcb.200908048 [pii]10.1083/jcb.200908048 (2010).
24 Mandel, G. et al. Repressor element 1 silencing transcription factor (REST) controls radial migration and temporal neuronal specification during neocortical development. Proc Natl Acad Sci USA 108, 16789-16794, doi: 1113486108 [pii]10.1073/pnas. 1113486108 (2011).
25 Vincent, A. S., Roebuck-Spencer, T. M. & Cernich, A. Cognitive changes and dementia risk 25 after traumatic brain injury: implications for aging military personnel. Alzheimers Dement 10, S174-187, doi: 10.1016/j.jalz.2014.04.006 (2014).
26 Kirn, J. R. The relationship of neurogenesis and growth of brain regions to song learning. Brain Lang 115, 29-44, doi: S0093-934X (09) 00135-7 [pii]10.1016/j.bandl.2009.09.006 (2010).
27 Oomen, C. A., Bekinschtein, P., Kent, B. A., Saksida, L. M. & Bussey, T. J. Adult hippocampal neurogenesis and its role in cognition. Wiley Interdiscip Rev Cogn Sci 5, 573-587, doi: 10.1002/wcs. 1304 (2014).
28 Samuels, I. S. et al. Deletion of ERK2 mitogen-activated protein kinase identifies its key roles in cortical neurogenesis and cognitive function. J Neurosci 28, 6983-6995, doi: 28/27/6983 [pii]10.1523/JNEUROSCI.0679-08.2008 (2008).
29 Shors, T. J. et al. Neurogenesis in the adult is involved in the formation of trace memories. Nature 410, 372-376, doi: 10.1038/35066584 (2001).
30 Villeda, S. A. et al. The ageing systemic milieu negatively regulates neurogenesis and cognitive function. Nature 477, 90-94, doi: 10.1038/nature10357 (2011).
31 Bird, C. M. & Burgess, N. The hippocampus supports recognition memory for familiar words but not unfamiliar faces. Curr Biol 18, 1932-1936, doi: 10.1016/j.cub.2008.10.046 (2008).
32 T. J. Shors, G. M., A. Beylin, M. Zhao, T. Rydel, E. Gould. Neurogenesis in the adult is involved in the formation of trace memories. Nature 410, 372-376 (2001).
33 VanElzakker, M., Fevurly, R. D., Breindel, T. & Spencer, R. L. Environmental novelty is associated with a selective increase in Fos expression in the output elements of the hippocampal formation and the perirhinal cortex. Learn Mem 15, 899-908, doi: 10.1101/1m.1196508 (2008).
34 Thornton, G. K. & Woods, C. G. Primary microcephaly: do all roads lead to Rome? Trends Genet 25, 501-510, doi: 10.1016/j.tig.2009.09.011 (2009).
35 Ghaziuddin, M., Zaccagnini, J., Tsai, L. & Elardo, S. Is megalencephaly specific to autism? J Intellect Disabil Res 43 (Pt 4), 279-282 (1999).
36 Kuhn, H. G., Cooper-Kuhn, C. M., Boekhoorn, K. & Lucassen, P. J. Changes in neurogenesis in dementia and Alzheimer mouse models: are they functionally relevant? Eur Arch Psychiatry Clin Neurosci 257, 281-289, doi: 10.1007/s00406-007-0732-4 (2007).
37 Baker, M. Tumours spark stem-cell review. Nature 457, 941, doi: 10.1038/457941a (2009).
38 Brederlau, A. et al. Transplantation of human embryonic stem cell-derived cells to a rat model of Parkinson's disease: effect of in vitro differentiation on graft survival and teratoma formation. Stem Cells 24, 1433-1440, doi: 2005-0393 [pii]10.1634/stemcells.2005-0393 (2006).
39 Burns, T. C., Verfaillie, C. M. & Low, W. C. Stem cells for ischemic brain injury: a critical review. J Comp Neurol 515, 125-144, doi: 10.1002/cne.22038 (2009).
40 Carlson, A. P., Schermer, C. R. & Lu, S. W. Retrospective evaluation of anemia and transfusion in traumatic brain injury. J Trauma 61, 567-571, doi: 10.1097/01.ta.0000231768.44727.a200005373-200609000-00007 [pii] (2006).
41 Carmeliet, P. & Storkebaum, E. Vascular and neuronal effects of VEGF in the nervous system: implications for neurological disorders. Semin Cell Dev Biol 13, 39-53, doi: 10.1006/scdb.2001.0290S1084952101902903 [pii] (2002).
42 Choe, Y., Kozlova, A., Graf, D. & Pleasure, S. J. Bone morphogenic protein signaling is a major determinant of dentate development. J Neurosci 33, 6766-6775, doi: 33/16/6766 [pii]10.1523/JNEUROSCI.0128-13.2013 (2013).
43 Erdo, F. et al. Host-dependent tumorigenesis of embryonic stem cell transplantation in experimental stroke. J Cereb Blood Flow Metab 23, 780-785, doi: 10.1097/01.WCB.0000071886.63724.FB (2003).
44 Greig, N. H. et al. Incretin mimetics as pharmacologic tools to elucidate and as a new drug strategy to treat traumatic brain injury. Alzheimers Dement 10, S62-75, doi: S1552-5260 (13) 02925-7 [pii]10.1016/j.jalz.2013.12.011 (2014).
45 Kaplan, G. B., Vasterling, J. J. & Vedak, P. C. Brain-derived neurotrophic factor in traumatic brain injury, post-traumatic stress disorder, and their comorbid conditions: role in pathogenesis and treatment. Behav Pharmacol 21, 427-437, doi: 10.1097/FBP.0b013e32833d8bc9 (2010).
46 Knoepfler, P. S. Deconstructing stem cell tumorigenicity: a roadmap to safe regenerative medicine. Stem Cells 27, 1050-1056, doi: 10.1002/stem.37 (2009).
47 Luca Longhia, Elisa R. Zaniera, Nicolas Royob, Nino Stocchettia, Tracy K. McIntosha. Stem cell transplantation as a therapeutic strategy for traumatic brain injury. Transplant Immunology 15, 134-148 (2005).
48 Robertson, C. S. et al. Effect of erythropoietin and transfusion threshold on neurological recovery after traumatic brain injury: a randomized clinical trial. JAMA 312, 36-47, doi: 1884575 [pii]10.1001/jama.2014.6490 (2014).
49 Salim, A. et al. Role of anemia in traumatic brain injury. J Am Coll Surg 207, 398-406, doi: S1072-7515 (08) 00322-0 [pii]10.1016/j.jamcollsurg.2008.03.013 (2008).
50 Deng, W., Aimone, J. B. & Gage, F. H. New neurons and new memories: how does adult hippocampal neurogenesis affect learning and memory? Nat Rev Neurosci 11, 339-350, doi: nrn2822 [pii]10.1038/nrn2822 (2010).
51 Goldman, S. A. & Nottebohm, F. Neuronal production, migration, and differentiation in a vocal control nucleus of the adult female canary brain. Proc Natl Acad Sci USA 80, 2390-2394 (1983).
52 Gould, E., Beylin, A., Tanapat, P., Reeves, A. & Shors, T. J. Learning enhances adult neurogenesis in the hippocampal formation. Nat Neurosci 2, 260-265, doi: 10.1038/6365 (1999).
53 Kirn, J. R. & Nottebohm, F. Direct evidence for loss and replacement of projection neurons in adult canary brain. J Neurosci 13, 1654-1663 (1993).
54 Mu, Y. & Gage, F. H. Adult hippocampal neurogenesis and its role in Alzheimer's disease. Mol Neurodegener 6, 85, doi: 1750-1326-6-85 [pii]10.1186/1750-1326-6-85 (2011).
55 Pytte, C. L., Gerson, M., Miller, J. & Kirn, J. R. Increasing stereotypy in adult zebra finch song correlates with a declining rate of adult neurogenesis. Dev Neurobiol 67, 1699-1720, doi: 10.1002/dneu.20520 (2007).
56 Scharff, C., Kirn, J. R., Grossman, M., Macklis, J. D. & Nottebohm, F. Targeted neuronal death affects neuronal replacement and vocal behavior in adult songbirds. Neuron 25, 481-492, doi: S0896-6273 (00) 80910-1 [pii] (2000).
57 Seib, D. R. et al. Loss of Dickkopf-1 restores neurogenesis in old age and counteracts cognitive decline. Cell Stem Cell 12, 204-214, doi: S1934-5909 (12) 00644-3 [pii]10.1016/j.stem.2012.11.010 (2013).
58 Deister, C. & Schmidt, C. E. Optimizing neurotrophic factor combinations for neurite outgrowth. J Neural Eng 3, 172-179, doi: 10.1088/1741-2560/3/2/011 (2006).
59 Zhao, Y. et al. Brain REST/NRSF Is Not Only a Silent Repressor but Also an Active Protector. Mol Neurobiol 54, 541-550, doi: 10.1007/s12035-015-9658-4 (2017).
60 Uchida, H., Ma, L. & Ueda, H. Epigenetic Gene Silencing Underlies C-Fiber Dysfunctions in Neuropathic Pain. Journal of Neuroscience 30, 4806-4814, doi: 10.1523/JNEUROSCI.5541-09.2010 (2010).
61 Alvarado, S. et al. Peripheral nerve injury is accompanied by chronic transcriptome-wide changes in the mouse prefrontal cortex. Mol Pain 9, 21, doi: 10.1186/1744-8069-9-21 (2013).
62 Hammer, P. et al. mRNA-seq with agnostic splice site discovery for nervous system transcriptomics tested in chronic pain. Genome Res 20, 847-860, doi: 10.1101/gr.101204.109 (2010).
63 Willis, D. E., Wang, M., Brown, E., Fones, L. & Cave, J. W. Selective repression of gene expression in neuropathic pain by the neuron-restrictive silencing factor/repressor element-1 silencing transcription (NRSF/REST). Neuroscience Letters 625, 20-25, doi: 10.1016/j.neulet.2015.12.003 (2016).
64 Ballas, N., Grunseich, C., Lu, D. D., Speh, J. C. & Mandel, G. REST and its corepressors mediate plasticity of neuronal gene chromatin throughout neurogenesis. Cell 121, 645-657, doi: S0092-8674 (05) 00285-0 [pii]10.1016/j.cell.2005.03.013 (2005).
65 Chong, J. A. et al. REST: a mammalian silencer protein that restricts sodium channel gene expression to neurons. Cell 80, 949-957, doi: 0092-8674 (95) 90298-8 [pii] (1995).
66 Mortazavi, A., Leeper Thompson, E. C., Garcia, S. T., Myers, R. M. & Wold, B. Comparative genomics modeling of the NRSF/REST repressor network: from single conserved sites to genome-wide repertoire. Genome Res 16, 1208-1221, doi: gr.4997306 [pii]10.1101/gr.4997306 (2006).
67 Otto, S. J. et al. A new binding motif for the transcriptional repressor REST uncovers large gene networks devoted to neuronal functions. J Neurosci 27, 6729-6739, doi: 27/25/6729 [pii]10.1523/JNEUROSCI.0091-07.2007 (2007).
68 Schoenherr, C. J. & Anderson, D. J. Silencing is golden: negative regulation in the control of neuronal gene transcription. Curr Opin Neurobiol 5, 566-571, doi: 0959-4388 (95) 80060-3 [pii] (1995).
69 Schoenherr, C. J., Paquette, A. J. & Anderson, D. J. Identification of potential target genes for the neuron-restrictive silencer factor. Proc Natl Acad Sci USA 93, 9881-9886 (1996).
70 Rose, K. et al. Transcriptional repression of the M channel subunit Kv7.2 in chronic nerve injury. Pain 152, 742-754, doi: 10.1016/j.pain.2010.12.028 (2011).
71 Costigan, M. et al. Multiple chronic pain states are associated with a common amino acid-changing allele in KCNS1. Brain 133, 2519-2527, doi: 10.1093/brain/awq195 (2010).
72 Zhang, F. et al. Repressor element 1-silencing transcription factor drives the development of chronic pain states. Pain, doi: 10.1097/j.pain.0000000000001633 (2019).
73 Zhang, J., Chen, S. R., Chen, H. & Pan, H. L. RE1-silencing transcription factor controls the acute-to-chronic neuropathic pain transition and Chrm2 receptor gene expression in primary sensory neurons. J Biol Chem 293, 19078-19091, doi: 10.1074/jbc.RA118.005846 (2018).
74 Ueda, H. et al. A mimetic of the mSin3-binding helix of NRSF/REST ameliorates abnormal pain behavior in chronic pain models. Bioorg Med Chem Lett 27, 4705-4709, doi: 10.1016/j.bmcl.2017.09.006 (2017).
75 Uchida, H., Sasaki, K., Ma, L. & Ueda, H. Neuron-restrictive silencer factor causes epigenetic silencing of Kv4.3 gene after peripheral nerve injury. Neuroscience 166, 1-4, doi: 10.1016/j.neuroscience.2009.12.021 (2010).
76 Gervasi N M., D. A., Clark D M., Dingle M, Pisarchik A V., Nesti L J. C-terminal domain small phosphatase 1 (CTDSP1) regulates growth factor expression and axonal regeneration. Journal of Translational Medicine Manuscripted submitted for publication. (2020).
77 Nesti, E., Pisarchik, A. COMPOSITIONS AND METHODS FOR DEREPRESSING RE1 SILENCING TRANSCRIPTION FACTOR TARGET GENES. USA patent (2019).
78 Cole, L. A., Kurscheid, S., Nekrasov, M., Domaschenz, R., Dennis, J. H., Tremethick, D. J. Redefining the nucleosomal architecture of active and inactive promoters in the context of cellular plasticity and cancer. Nature Communications revision returned (2020).
79 Ren, Y., Vera, D. L., Hughes, K. A. & Dennis, J. H. Stimulation of the Drosophila immune system alters genome-wide nucleosome occupancy. Genom Data 3, 146-147, doi: 10.1016/j.gdata.2015.01.001 (2015).
80 Sexton, B. S. et al. The spring-loaded genome: nucleosome redistributions are widespread, transient, and DNA-directed. Genome Res 24, 251-259, doi: 10.1101/gr. 160150.113 (2014).
81 Sexton, B. S. et al. Hierarchical regulation of the genome: global changes in nucleosome organization potentiate genome response. Oncotarget 7, 6460-6475, doi: 10.18632/oncotarget.6841 (2016).
82 Lunyak, V. V. & Rosenfeld, M. G. No rest for REST: REST/NRSF regulation of neurogenesis. Cell 121, 499-501, doi: 10.1016/j.cell.2005.05.003 (2005).
83 Dirks, P. B. Making a commitment: neurons refuse cancer's advances. Nat Neurosci 22, 507-508, doi: 10.1038/s41593-019-0373-8 (2019).
84 Kaspar, A. A. & Reichert, J. M. Future directions for peptide therapeutics development. Drug Discov Today 18, 807-817, doi: 10.1016/j.drudis.2013.05.011 (2013).
85 Hamzeh-Mivehroud, M., Alizadeh, A. A., Morris, M. B., Bret Church, W. & Dastmalchi, S. Phage display as a technology delivering on the promise of peptide drug discovery. Drug Discovery Today 18, 1144-1157, doi: 10.1016/j.drudis.2013.09.001 (2013).
86 Hoekstra, E., Peppelenbosch, M. P. & Fuhler, G. M. Meeting report europhosphatase 2015: Phosphatases as drug targets in cancer. Cancer Research 76, 193-196, doi: 10.1158/0008-5472.CAN-15-2091 (2016).
87 Shi, Y. Serine/Threonine Phosphatases: Mechanism through Structure. Cell 139, 468-484, doi: 10.1016/j.cell.2009.10.006 (2009).
88 Sussman, F., Villaverde, M. C., L. Dominguez, J. & Danielson, U. H. On the Active Site Protonation State in Aspartic Proteases: Implications for Drug Design. Current Pharmaceutical Design, Volume 19, Number 23 19, 4257-4275, doi: 10.1002/(SICI) 1520-667X (1998) 10: 1<19::AID-MCS3>3.0.CO; 2-1 (2013).
89 Anobom, C. D. et al. From structure to catalysis: Recent developments in the biotechnological applications of lipases. BioMed Research International 2014, doi: 10.1155/2014/684506 (2014).
90 Blundell, T. L., Jhoti, H. & Abell, C. High-Throughput Crystallography for Lead Discovery in Drug Design. Nature Reviews Drug Discovery 1, 45-54, doi: 10.1038/nrd706 (2002).
91 Weir, M. R. Renin inhibitors: novel agents for renoprotection or a better angiotensin receptor blocker for blood pressure lowering? Current opinion in nephrology and hypertension 16, 416-421, doi: 10.1097/MNH.0b013e328209fe00 (2007).
92 Yeo, M. et al. Small CTD phosphatases function in silencing neuronal gene expression. Science 307, 596-600, doi: 307/5709/596 [pii]10.1126/science.1100801 (2005).
93 Fulda, S., Wick, W., Weller, M. & Debatin, K. M. Smac agonists sensitize for Apo2L/TRAIL- or anticancer drug-induced apoptosis and induce regression of malignant glioma in vivo. Nature Medicine 8, 808-815, doi: 10.1038/nm735 (2002).
94 Noguchi, H. et al. A new cell-permeable peptide allows successful allogeneic islet transplantation in mice. Nature Medicine 10, 305-309, doi: 10.1038/nm994 (2004).
95 Pooga, M. et al. Cellular translocation of proteins by transportan. The FASEB journal: official publication of the Federation of American Societies for Experimental Biology 15, 1451-1453, doi: 10.1096/fj.00-0780fje (2001).
96 Schwarze, S. R., Ho, A., Vocero-Akbani, A. & Dowdy, S. F. In vivo protein transduction: delivery of a biologically active protein into the mouse. Science (New York, N.Y.) 285, 1569-1572, doi: 10.1126/science.285.5433.1569 (1999).
97 Yeo, M., Lin, P. S., Dahmus, M. E. & Gill, G. N. A Novel RNA Polymerase II C-terminal Domain Phosphatase That Preferentially Dephosphorylates Serine 5. Journal of Biological Chemistry 278, 26078-26085, doi: 10.1074/jbc.M301791200 (2003).
98 Banerjee, P. N., Filippi, D. & Allen Hauser, W. The descriptive epidemiology of epilepsy—a review. Epilepsy Res 85, 31-45, doi: 10.1016/j.eplepsyres.2009.03.003 (2009).
99 Mucha, M. et al. Transcriptional control of KCNQ channel genes and the regulation of neuronal excitability. J Neurosci 30, 13235-13245, doi: 10.1523/JNEUROSCI.1981-10.2010 (2010).
100 Escayg, A. et al. Mutations of SCN1A, encoding a neuronal sodium channel, in two families with GEFS+2. Nat Genet 24, 343-345, doi: 10.1038/74159 (2000).
101 Martin, D. et al. REST represses a subset of the pancreatic endocrine differentiation program. Dev Biol 405, 316-327, doi: 10.1016/j.ydbio.2015.07.002 (2015).
102 Abderrahmani, A. et al. Neuronal traits are required for glucose-induced insulin secretion. FEBS Lett 565, 133-138, doi: 10.1016/j.febslet.2004.04.002 (2004).
103 Martin, D. et al. Functional significance of repressor element 1 silencing transcription factor (REST) target genes in pancreatic beta cells. Diabetologia 51, 1429-1439, doi: 10.1007/s00125-008-0984-1 (2008).
104 Rigamonti, D. et al. Loss of huntingtin function complemented by small molecules acting as repressor element 1/neuron restrictive silencer element silencer modulators. J Biol Chem 282, 24554-24562, doi: 10.1074/jbc.M609885200 (2007).
105 Sipione, S. et al. Early transcriptional profiles in huntingtin-inducible striatal cells by microarray analyses. Hum Mol Genet 25, 210, doi: 10.1093/hmg/ddv416 (2016).
106 Zuccato, C. et al. Huntingtin interacts with REST/NRSF to modulate the transcription of NRSE-controlled neuronal genes. Nat Genet 35, 76-83, doi: 10.1038/ng1219 (2003).
107 Bao, S. et al. Targeting cancer stem cells through LICAM suppresses glioma growth. Cancer Res 68, 6043-6048, doi: 10.1158/0008-5472.CAN-08-1079 (2008).
108 Jackson, M., Hassiotou, F. & Nowak, A. Glioblastoma stem-like cells: at the root of tumor recurrence and a therapeutic target. Carcinogenesis 36, 177-185, doi: 10.1093/carcin/bgu243 (2015).
109 Liu, G. et al. Analysis of gene expression and chemoresistance of CD133+ cancer stem cells in glioblastoma. Mol Cancer 5, 67, doi: 10.1186/1476-4598-5-67 (2006).
110 Persano, L., Rampazzo, E., Basso, G. & Viola, G. Glioblastoma cancer stem cells: role of the microenvironment and therapeutic targeting. Biochem Pharmacol 85, 612-622, doi: 10.1016/j.bcp.2012.10.001 (2013).
111 Conti, L. et al. REST controls self-renewal and tumorigenic competence of human glioblastoma cells. PLOS One 7, e38486, doi: 10.1371/journal.pone.0038486 (2012).
112 Kamal, M. M. et al. REST regulates oncogenic properties of glioblastoma stem cells. Stem Cells 30, 405-414, doi: 10.1002/stem.1020 (2012).
113 Wagoner, M. P. & Roopra, A. A REST derived gene signature stratifies glioblastomas into chemotherapy resistant and responsive disease. BMC Genomics 13, 686, doi: 10.1186/1471-2164-13-686 (2012).
114 Taylor, P. et al. REST is a novel prognostic factor and therapeutic target for medulloblastoma. Mol Cancer Ther 11, 1713-1723, doi: 10.1158/1535-7163.MCT-11-0990 (2012).
115 Jin, H. et al. Identification of RE1-Silencing Transcription Factor as a Promoter of Metastasis in Pancreatic Cancer. Front Oncol 9, 291, doi: 10.3389/fonc.2019.00291 (2019).
116 Jackson, W. M. et al. Mesenchymal progenitor cells derived from traumatized human muscle. J Tissue Eng Regen Med 3, 129-138, doi: 10.1002/term. 149 (2009).
117 Schmitz, H. C. & Beer, G. M. The toe-spreading reflex of the rabbit revisited—functional evaluation of complete peroneal nerve lesions. Lab Anim 35, 340-345, doi: 10.1258/0023677011911930 (2001).
118 Hems, T. E. & Glasby, M. A. The limit of graft length in the experimental use of muscle grafts for nerve repair. J Hand Surg Br 18, 165-170 (1993).
119 Ballas, N. et al. Regulation of neuronal traits by a novel transcriptional complex. Neuron 31, 353-365, doi: 10.1016/s0896-6273 (01) 00371-3 (2001).
120 Symes, A. J. et al. Ciliary neurotrophic factor coordinately activates transcription of neuropeptide genes in a neuroblastoma cell line. Proc Natl Acad Sci USA 90, 572-576, doi: 10.1073/pnas.90.2.572 (1993).
121 Chou, B. K. et al. Efficient human iPS cell derivation by a non-integrating plasmid from blood cells with unique epigenetic and gene expression signatures. Cell Res 21, 518-529, doi: 10.1038/cr.2011.12 (2011).
122 Swistowski, A. et al. Xeno-free defined conditions for culture of human embryonic stem cells, neural stem cells and dopaminergic neurons derived from them. PLOS One 4, e6233, doi: 10.1371/journal.pone.0006233 (2009).
123 Yan, Y. et al. Efficient and rapid derivation of primitive neural stem cells and generation of brain subtype neurons from human pluripotent stem cells. Stem Cells Transl Med 2, 862-870, doi: 10.5966/sctm.2013-0080 (2013).
124 Efthymiou, A. et al. Functional screening assays with neurons generated from pluripotent stem cell-derived neural stem cells. J Biomol Screen 19, 32-43, doi: 10.1177/1087057113501869 (2014).
125 Malik, N. et al. Compounds with species and cell type specific toxicity identified in a 2000 compound drug screen of neural stem cells and rat mixed cortical neurons. Neurotoxicology 45, 192-200, doi: 10.1016/j.neuro.2014.10.007 (2014).
126 Pei, Y. et al. Comparative neurotoxicity screening in human iPSC-derived neural stem cells, neurons and astrocytes. Brain Res 1638, 57-73, doi: 10.1016/j.brainres.2015.07.048 (2016).
127 Lischka, F. W. et al. Neonatal mouse cortical but not isogenic human astrocyte feeder layers enhance the functional maturation of induced pluripotent stem cell-derived neurons in culture. Glia 66, 725-748, doi: 10.1002/glia.23278 (2018).
128 Papapetrou, E. P. & Schambach, A. Gene Insertion Into Genomic Safe Harbors for Human Gene Therapy. Mol Ther 24, 678-684, doi: 10.1038/mt.2016.38 (2016).
129 He, R. et al. Recombinant luciferase-expressing human cytomegalovirus (CMV) for evaluation of CMV inhibitors. Virol J 8, 40, doi: 10.1186/1743-422X-8-40 (2011).
130 Jacqmain, J., Nudi, E. T., Fluharty, S. & Smith, J. S. Pre and post-injury environmental enrichment effects functional recovery following medial frontal cortical contusion injury in rats. Behav Brain Res 275, 201-211, doi: 10.1016/j.bbr.2014.08.056 (2014).
131 Monaco, C. M. et al. Environmental enrichment promotes robust functional and histological benefits in female rats after controlled cortical impact injury. Exp Neurol 247, 410-418, doi: 10.1016/j.expneurol.2013.01.007 (2013).
132 Shear, D. A. et al. Nicotinamide Treatment in Traumatic Brain Injury: Operation Brain Trauma Therapy. J Neurotrauma 33, 523-537, doi: 10.1089/neu.2015.4115 (2016).
133 Wagner, A. K., Postal, B. A., Darrah, S. D., Chen, X. & Khan, A. S. Deficits in novelty exploration after controlled cortical impact. J Neurotrauma 24, 1308-1320, doi: 10.1089/neu.2007.0274 (2007).
134 Zhang, J., Groff, R. F. & Dayawansa, S. Imipramine treatment increases cell proliferation following fluid percussion brain injury in rats. Neurol Res 35, 247-254, doi: 10.1179/1743132813Y.0000000164 (2013).
135 Xiong, Y. et al. Histological and functional outcomes after traumatic brain injury in mice null for the erythropoietin receptor in the central nervous system. Brain Res 1230, 247-257, doi: 10.1016/j.brainres.2008.06.127 (2008).
136 Susarla, B. T., Villapol, S., Yi, J. H., Geller, H. M. & Symes, A. J. Temporal patterns of cortical proliferation of glial cell populations after traumatic brain injury in mice. ASN Neuro 6, 159-170, doi: 10.1042/AN20130034 (2014).
137 Xiong, Y. et al. Role of gender in outcome after traumatic brain injury and therapeutic effect of erythropoietin in mice. Brain Res 1185, 301-312, doi: 10.1016/j.brainres.2007.09.052 (2007).
138 Choi, S. H., Woodlee, M. T., Hong, J. J. & Schallert, T. A simple modification of the water maze test to enhance daily detection of spatial memory in rats and mice. J Neurosci Methods 156, 182-193, doi: 10.1016/j.jneumeth.2006.03.002 (2006).
139 Omura, T. et al. Different expressions of BDNF, NT3, and NT4 in muscle and nerve after various types of peripheral nerve injuries. J Peripher Nerv Syst 10, 293-300, doi: 10.1111/j.1085-9489.2005.10307.x (2005).
140 Zhang, J. Y., Luo, X. G., Xian, C. J., Liu, Z. H. & Zhou, X. F. Endogenous BDNF is required for myelination and regeneration of injured sciatic nerve in rodents. Eur J Neurosci 12, 4171-4180 (2000).
141 Zhao, Y. et al. Brain REST/NRSF Is Not Only a Silent Repressor but Also an Active Protector. Molecular Neurobiology 54, 541-550, doi: 10.1007/s12035-015-9658-4 (2017).
142 Charbord, J. et al. High throughput screening for inhibitors of REST in neural derivatives of human embryonic stem cells reveals a chemical compound that promotes expression of neuronal genes. Stem Cells 31, 1816-1828, doi: 10.1002/stem.1430 (2013).
143 Nesti, L. J. et al. Differentiation potential of multipotent progenitor cells derived from war-traumatized muscle tissue. J Bone Joint Surg Am 90, 2390-2398, doi: 10.2106/JBJS.H.00049 (2008).
144 Bulken-Hoover, J. D. et al. Inducible expression of neurotrophic factors by mesenchymal progenitor cells derived from traumatically injured human muscle. Mol Biotechnol 51, 128-136, doi: 10.1007/s12033-011-9445-z (2012).
145 Mosmann, T. Rapid colorimetric assay for cellular growth and survival: application to proliferation and cytotoxicity assays. J Immunol Methods 65, 55-63, doi: 10.1016/0022-1759 (83) 90303-4 (1983).
146 Chan, J. R., Cosgaya, J. M., Wu, Y. J. & Shooter, E. M. Neurotrophins are key mediators of the myelination program in the peripheral nervous system. Proc Natl Acad Sci USA 98, 14661-14668, doi: 10.1073/pnas.251543398 (2001).
147 Leung, J. Y. et al. Metallothionein promotes regenerative axonal sprouting of dorsal root ganglion neurons after physical axotomy. Cell Mol Life Sci 69, 809-817, doi: 10.1007/s00018-011-0790-7 (2012).
148 Rothstein, J. D., Jin, L., Dykes-Hoberg, M. & Kuncl, R. W. Chronic inhibition of glutamate uptake produces a model of slow neurotoxicity. Proc Natl Acad Sci USA 90, 6591-6595 (1993).
149 Corse, A. M. et al. Preclinical testing of neuroprotective neurotrophic factors in a model of chronic motor neuron degeneration. Neurobiol Dis 6, 335-346, doi: 10.1006/nbdi. 1999.0253 (1999).
150 Mi, R., Chen, W. & Hoke, A. Pleiotrophin is a neurotrophic factor for spinal motor neurons. Proc Natl Acad Sci USA 104, 4664-4669, doi: 10.1073/pnas.0603243104 (2007).
151 Cargnin, F. et al. An RNA binding protein promotes axonal integrity in peripheral neurons by destabilizing REST. J Neurosci 34, 16650-16661, doi: 10.1523/JNEUROSCI.1650-14.2014 (2014).
152 Rueden, C. T. et al. ImageJ2: ImageJ for the next generation of scientific image data. BMC Bioinformatics 18, 529-529, doi: 10.1186/s12859-017-1934-z (2017).
153 Woolf, C. J. Phenotypic modification of primary sensory neurons: the role of nerve growth factor in the production of persistent pain. Philos Trans R Soc Lond B Biol Sci 351, 441-448, doi: 10.1098/rstb. 1996.0040 (1996).
154 Fremin, C. et al. ERK2 but not ERK1 plays a key role in hepatocyte replication: an RNAi-mediated ERK2 knockdown approach in wild-type and ERK1 null hepatocytes. Hepatology 45, 1035-1045, doi: 10.1002/hep.21551 (2007).
155 River, C. Sergical Services, <https://www.criver.com/sites/default/files/resources/VascularCatheterSurgeryOptionsInformationSheet.pdf> (2018).
156 Beer, S. a. The toe-spreading reflex of the rabbit revisited—functional evaluation of complete peroneal nerve lesions. Laboratory Animals 35, 340-345 (2001).
157 Glasby, M. A. & Hems, T. E. Repairing spinal roots after brachial plexus injuries. Paraplegia 33, 359-361, doi: 10.1038/sc.1995.80 (1995).
158 Jin, J. et al. Functional motor recovery after peripheral nerve repair with an aligned nanofiber tubular conduit in a rat model. Regen Med 7, 799-806, doi: 10.2217/rme. 12.87 (2012).
159 Han, D., Lu, J., Xu, L. & Xu, J. Comparison of two electrophysiological methods for the assessment of progress in a rat model of nerve repair. International Journal of Clinical and Experimental Medicine 8, 2392-2398 (2015).
160 Yektaş, A. et al. Perineural dexmedetomidine effects on sciatic nerve in rat. Brazilian Journal of Anesthesiology (English Edition) 67, 57-66, doi: 10.1016/j.bjane.2015.08.012 (2017).
161 Rigaud, M. et al. Species and strain differences in rodent sciatic nerve anatomy: implications for studies of neuropathic pain. Pain 136, 188-201, doi: 10.1016/j.pain.2008.01.016 (2008).
162 Sleigh, J. N., Weir, G. A. & Schiavo, G. A simple, step-by-step dissection protocol for the rapid isolation of mouse dorsal root ganglia. BMC Res Notes 9, 82, doi: 10.1186/s13104-016-1915-8 (2016).
163 Ronchi, G. et al. Discrepancies in quantitative assessment of normal and regenerated peripheral nerve fibers between light and electron microscopy. J Peripher Nerv Syst 19, 224-233, doi: 10.1111/jns. 12090 (2014).
164 Varejao, A. S. et al. Functional and morphological assessment of a standardized rat sciatic nerve crush injury with a non-serrated clamp. J Neurotrauma 21, 1652-1670, doi: 10.1089/neu.2004.21.1652 (2004).
165 Carriel, V., Garzon, I., Alaminos, M. & Cornelissen, M. Histological assessment in peripheral nerve tissue engineering. Neural Regen Res 9, 1657-1660, doi: 10.4103/1673-5374.141798 (2014).
166 Hazari, A., Wiberg, M., Johansson-Ruden, G., Green, C. & Terenghi, G. A resorbable nerve conduit as an alternative to nerve autograft in nerve gap repair. Br J Plast Surg 52, 653-657, doi: 10.1054/bjps. 1999.3184 (1999).
167 Shi, T. J. et al. Effect of peripheral nerve injury on dorsal root ganglion neurons in the C57 BL/6J mouse: marked changes both in cell numbers and neuropeptide expression. Neuroscience 105, 249-263 (2001).
168 Jackson, W. M. et al. Mesenchymal progenitor cells derived from traumatized muscle enhance neurite growth. J Tissue Eng Regen Med 7, 443-451, doi: 10.1002/term.539 (2013).
169 Kobiela Ketz A, B. K., Grunberg N E, Kasper C E, Osborne L, Pryor B, Tosini N L, Wu X, Anders J J. Characterization of macrophage/microglial activation and effect of photobiomodulation in the spared nerve injury model of neuropathic pain. Pain Medicine 5, 932-946, doi: 10.1093/pm/pnw144 (2016).
170 Whiteside, G. T., Adedoyin A, Leventhal L. Predictive validity of animal pain models? A comparison of the pharmacokinetic-pharmacodynamic relationship for pain drugs in rats and humans. Neuropharm, 767-775, doi: 10.1016/j.neuropharm.2008.01.001 (2008).
171 LaBuda, C. J. & Fuchs, P. N. A behavioral test paradigm to measure the aversive quality of inflammatory and neuropathic pain in rats. Exp Neurol 163, 490-494, doi: 10.1006/exnr.2000.7395 (2000).
172 Xu, Y. et al. Gait Assessment of Pain and Analgesics: Comparison of the DigiGait and CatWalk Gait Imaging Systems. Neurosci Bull 35, 401-418, doi: 10.1007/s12264-018-00331-y (2019).
173 Hargreaves, K., Dubner, R., Brown, F., Flores, C. & Joris, J. A new and sensitive method for measuring thermal nociception in cutaneous hyperalgesia. Pain 32, 77-88, doi: 10.1016/0304-3959 (88) 90026-7 (1988).
174 Baastrup, C., Jensen, T. S. & Finnerup, N. B. Pregabalin attenuates place escape/avoidance behavior in a rat model of spinal cord injury. Brain Res 1370, 129-135, doi: 10.1016/j.brainres.2010.11.008 (2011).
175 FDA. Guidance for industry (ed CDER; CBER) (FDA, https://www.fda.gov/downloads/Drugs/GuidanceComplianceRegulatoryInformation/Guidances/UCM074957.pdf, 1997).
176 FDA. S2B Genotoxicity: A Standard Battery for Genotoxicity Testing of Pharmaceuticals, <https://www.fda.gov/media/71971/download> (1997).
177 FDA. S7B Nonclinical Evaluation of the Potential for Delayed Ventricular Repolarization (QT Interval Prolongation) by Human Pharmaceuticals, <https://www.fda.gov/media/72043/download> (2005).
178 FDA. (ed CDER; CBER) (FDA, https://www.fda.gov/downloads/drugs/guidances/ucm073246.pdf, 2010).
179 FDA. (ed CDER; CBER) (FDA, https://www.fda.gov/downloads/drugs/guidances/ucm74959.pdf, July 2001).
180 Irwin, S. Comprehensive observational assessment: Ia. A systematic, quantitative procedure for assessing the behavioral and physiologic state of the mouse. Psychopharmacologia 13, 222-257 (1968).
181 Morrone, L., Scuteri, D., Rombola, L., Mizoguchi, H. & Bagetta, G. Opioids Resistance in Chronic Pain Management. Current Neuropharmacology 15, 444-456, doi: 10.2174/1570159X14666161101092822 (2017).
182 Ploj, K., Roman, E. & Nylander, I. Long-term effects of short and long periods of maternal separation on brain opioid peptide levels in male Wistar rats. Neuropeptides 37, 149-156, doi: 10.1016/S0143-4179 (03) 00043-X (2003).
183 Zhu, C. et al. Neuron-restrictive silencer factor-mediated downregulation of μ-opioid receptor contributes to the reduced morphine analgesia in bone cancer pain. PAIN 158, 879-890, doi: 10.1097/j.pain.0000000000000848 (2017).
184 Lu, C. e. et al. Neuron-restrictive silencer factor in periaqueductal gray contributes to remifentanil-induced postoperative hyperalgesia via repression of the mu-opioid receptor. Journal of the Neurological Sciences 352, 48-52, doi: 10.1016/j.jns.2015.03.018 (2015).
185 Schwartz, A. S. & Marchok, P. L. Depression of morphine-seeking behaviour by dopamine inhibition. Nature 248, 257-258 (1974).
186 Spyraki, C., Fibiger, H. C. & Phillips, A. G. Attenuation of heroin reward in rats by disruption of the mesolimbic dopamine system. Psychopharmacology 79, 278-283 (1983).
187 Phillips, A. G. & LePiane, F. G. Reinforcing effects of morphine microinjection into the ventral tegmental area. Pharmacology, biochemistry, and behavior 12, 965-968 (1980).
188 Phillips, A. G., LePiane, F. G. & Fibiger, H. C. Dopaminergic mediation of reward produced by direct injection of enkephalin into the ventral tegmental area of the rat. Life sciences 33, 2505-2511 (1983).
189 Prus A J, J. J., Rosecrans J A. in Methods of Behavior Analysis in Neuroscience (ed Buccafusco J J) Ch. 4, (CRC Press/Taylor & Francis, 2009).
190 Carr, K. D., Bak, T. H., Simon, E. J. & Portoghese, P. S. Effects of the selective kappa opioid antagonist, nor-binaltorphimine, on electrically-elicited feeding in the rat. Life sciences 45, 1787-1792 (1989).
191 Galli, R. et al. Isolation and characterization of tumorigenic, stem-like neural precursors from human glioblastoma. Cancer Res 64, 7011-7021, doi: 10.1158/0008-5472.CAN-04-1364 (2004).
192 Garzon-Muvdi, T. et al. Regulation of brain tumor dispersal by NKCC1 through a novel role in focal adhesion regulation. PLOS Biol 10, e1001320, doi: 10.1371/journal.pbio. 1001320 (2012).
193 Guerrero-Cazares, H., Chaichana, K. L. & Quinones-Hinojosa, A. Neurosphere culture and human organotypic model to evaluate brain tumor stem cells. Methods Mol Biol 568, 73-83, doi: 10.1007/978-1-59745-280-9_6 (2009).
194 Tilghman, J. et al. Regulation of Glioblastoma Tumor-Propagating Cells by the Integrin Partner Tetraspanin CD151. Neoplasia 18, 185-198, doi: 10.1016/j.neo.2016.02.003 (2016).
195 Lal, S. et al. An implantable guide-screw system for brain tumor studies in small animals. J Neurosurg 92, 326-333, doi: 10.3171/jns.2000.92.2.0326 (2000).
196 Manton, C. A. et al. Induction of cell death by the novel proteasome inhibitor marizomib in glioblastoma in vitro and in vivo. Sci Rep 6, 18953, doi: 10.1038/srep18953 (2016).
197 Kaplan, E. L. M., P. Nonparametric estimation from incomplete observations. Journal of the American Statistical Association 53, 457-481, doi: 10.2307/2281868 (1958).
198 Marubini E, V. M. C. Estimation of Survival Probabilities. Analysing survival data from clinical trials and observational studies. 41-81 (John Wiley and Sons, 1995).

What is claimed is:

1. A method for in vivo inhibition of REST activity, the method comprising the step of contacting an isolate peptide comprising an amino acid sequence, wherein the amino acid sequence is selected from the group consisting of SEQ ID NOS: 1, 15-17, and 18-117, with CTDSP1.

2. The method of claim 1, wherein the peptide binds to CTDSP1.

3. The method of claim 1, wherein the peptide is administered intravenously, subcutaneously, orally, or via a mucosal membrane.

4. The method of claim 1, wherein the amino acid sequence is a fusion peptide.

5. The method of claim 4, wherein the amino acid sequence is fused to a cell penetrating peptide or an endosomal release sequence.

6. The method of claim 5, wherein the cell penetrating peptide or the endosomal release sequence is selected from the group consisting of SEQ ID NOS: 118-137 or 140-159.

7. The method of claim 5, wherein a linker connects the amino acid sequence to the cell penetrating peptide or the endosomal release sequence.

8. The method of claim 7, wherein the linker is selected from the group consisting of SEQ ID NOS: 138, 139, 160, and 161.

9. The method of claim 5, wherein the cell penetrating peptide or the endosomal release sequence is fused to the amino acid sequence at an N- or a C-terminus.

10. The method of claim 9, wherein the fusion peptide is cyclized.

11. The method of claim 4, wherein the fusion peptide has an amino acid sequence selected from the group consisting of SEQ ID NOS: 2 and 4-14.

12. An isolate peptide comprising an amino acid sequence selected from the group consisting of SEQ ID NOS: 1, 15-17, and 18-117.

13. The use of the peptide of claim 12 to treat traumatic brain injury, chronic pain, peripheral nerve injury, epilepsy, diabetes, Alzheimer's disease, Huntington's disease, brain tumors (including glioblastoma multiforme), or pancreatic cancer in an animal.

14. A method for treating alleviating, or ameliorating a disease selected from the group consisting of traumatic brain injury, chronic pain, peripheral nerve injury, epilepsy, diabetes, Alzheimer's disease, Huntington's disease, brain tumors (including glioblastoma multiforme), or pancreatic cancer in an animal in need thereof, the method comprising the step of administering to the animal the peptide of claim 1 effective to treat, alleviate, or ameliorate the disease.

15. A pharmaceutical composition comprising the peptide of claim 12.


SEQUENCE LISTING (350 sequences)

SEQ ID NO: 1 - protein, 15 aa, synthetic construct
  REGION 1..15: note = Description of Artificial Sequence: Synthetic peptide
TEDLEPPEPPLPKEN

SEQ ID NO: 2 - protein, 49 aa, synthetic construct
  REGION 1..49: note = Description of Artificial Sequence: Synthetic polypeptide
MCTEDLEPPEPPLPKENCGSGSGRKKRRQRRRGGSGSLGHHHHHHGTGY

SEQ ID NO: 3 - protein, 49 aa, synthetic construct
  REGION 1..49: note = Description of Artificial Sequence: Synthetic polypeptide
MCEDAKNIKKGPAPFYPCGSGSGRKKRRQRRRGGSGSLGHHHHHHGTGY

SEQ ID NO: 4 - protein, 38 aa, synthetic construct
  REGION 1..38: note = Description of Artificial Sequence: Synthetic polypeptide
CAQKDYKDDDDKTEDLEPPEPPLPKENGRKKRRQRRRG

SEQ ID NO: 5 - protein, 49 aa, synthetic construct
  REGION 1..49: note = Description of Artificial Sequence: Synthetic polypeptide
CTEDLEPPEPPLPKENSGDIMGEWGNEIFGAIAGFLGYGRKKRRQRRRG

SEQ ID NO: 6 - protein, 30 aa, synthetic construct
  REGION 1..30: note = Description of Artificial Sequence: Synthetic polypeptide
  SITE 30: note = D-amino acid
TEDLEPPEPPLPKENRRWWRRWRRRRWWRR

SEQ ID NO: 7 - protein, 26 aa, synthetic construct
  REGION 1..26: note = Description of Artificial Sequence: Synthetic peptide
  SITE 26: note = D-amino acid
EDLEPPEPPLPKRWWRRRRWRRWWRR

SEQ ID NO: 8 - protein, 27 aa, synthetic construct
  REGION 1..27: note = Description of Artificial Sequence: Synthetic peptide
  SITE 27: note = D-amino acid
AGDLEQPEPPVAKKKKKNRRWWRRWRR

SEQ ID NO: 9 - protein, 29 aa, synthetic construct
  REGION 1..29: note = Description of Artificial Sequence: Synthetic peptide
  SITE 1..29: note = D-amino acid
RRRRWRRWWRRWRRNEKPLPPEPPELDET

SEQ ID NO: 10 - protein, 29 aa, synthetic construct
  REGION 1..29: note = Description of Artificial Sequence: Synthetic peptide
  SITE 16..29: note = D-amino acid
TEDLEPPEPPLPKENRRRRWRRWWRRWRR

SEQ ID NO: 11 - protein, 29 aa, synthetic construct
  REGION 1..29: note = Description of Artificial Sequence: Synthetic peptide
  SITE 22: note = 2-amino-tetradecanoic acid
  SITE 29: note = D-amino acid
TEDLEPPEPPLPKENRRRRRRXRRWWRRR

SEQ ID NO: 12 - protein, 24 aa, synthetic construct
  REGION 1..24: note = Description of Artificial Sequence: Synthetic peptide
  SITE 1..6: note = D-amino acid
  SITE 7: note = 2-amino-tetradecanoic acid
  SITE 8..24: note = D-amino acid
RRWWRRXRRRRRKPLPPEPPELDE

SEQ ID NO: 13 - protein, 23 aa, synthetic construct
  REGION 1..23: note = Description of Artificial Sequence: Synthetic peptide
  SITE 1..2: note = D-amino acid
  SITE 3: note = 2-amino-tetradecanoic acid
  SITE 4..5: note = D-amino acid
  SITE 6: note = 2-amino-tetradecanoic acid
  SITE 7..23: note = D-amino acid
RRXRRXRRRRRKPLPPEPPELDE

SEQ ID NO: 14 - protein, 18 aa, synthetic construct
  REGION 1..18: note = Description of Artificial Sequence: Synthetic peptide
  SITE 1..2: note = D-amino acid
  SITE 3: note = 2-amino-tetradecanoic acid
  SITE 4..18: note = D-amino acid
RRXRRRKPLPPEPPELDE

SEQ ID NO: 15 - protein, 12 aa, synthetic construct
  REGION 1..12: note = Description of Artificial Sequence: Synthetic peptide
EDLEPPEPPLPK

SEQ ID NO: 16 - protein, 15 aa, synthetic construct
  REGION 1..15: note = Description of Artificial Sequence: Synthetic peptide
  SITE 1..15: note = D-amino acid
NEKPLPPEPPELDET

SEQ ID NO: 17 - protein, 12 aa, synthetic construct
  REGION 1..12: note = Description of Artificial Sequence: Synthetic peptide
  SITE 1..12: note = D-amino acid
KPLPPEPPELDE

SEQ ID NO: 18 - protein, 18 aa, synthetic construct
  REGION 1..18: note = Description of Artificial Sequence: Synthetic peptide
MCTEDLEPPEPPLPKENC

SEQ ID NO: 19 - protein, 21 aa, synthetic construct
  REGION 1..21: note = Description of Artificial Sequence: Synthetic peptide
MCTEAPAPPEPALPKKKKKNC

SEQ ID NO: 20 - protein, 17 aa, synthetic construct
  REGION 1..17: note = Description of Artificial Sequence: Synthetic peptide
MCTEDLQPPTAVPQENC

SEQ ID NO: 21 - protein, 20 aa, synthetic construct
  REGION 1..20: note = Description of Artificial Sequence: Synthetic peptide
MCTEAPAPPEPALPKKKKNC

SEQ ID NO: 22 - protein, 20 aa, synthetic construct
  REGION 1..20: note = Description of Artificial Sequence: Synthetic peptide
MCTADLEPPEPRMEKKKVDC

SEQ ID NO: 23 - protein, 18 aa, synthetic construct
  REGION 1..18: note = Description of Artificial Sequence: Synthetic peptide
MCTGDLQPPKTTVSKKDC

SEQ ID NO: 24 - protein, 18 aa, synthetic construct
  REGION 1..18: note = Description of Artificial Sequence: Synthetic peptide
MCTEDLQSPKTTMTKENC

SEQ ID NO: 25 - protein, 18 aa, synthetic construct
  REGION 1..18: note = Description of Artificial Sequence: Synthetic peptide
MCTEDLEPPEPPLPKEDC

SEQ ID NO: 26 - protein, 18 aa, synthetic construct
  REGION 1..18: note = Description of Artificial Sequence: Synthetic peptide
MCTEDQEQQEEQLPEENC

SEQ ID NO: 27 - protein, 18 aa, synthetic construct
  REGION 1..18: note = Description of Artificial Sequence: Synthetic peptide
MCTADLKPPKTTMTKQNC

SEQ ID NO: 28 - protein, 18 aa, synthetic construct
  REGION 1..18: note = Description of Artificial Sequence: Synthetic peptide
MCPGDLKQPEPPMPKEYC

SEQ ID NO: 29 - protein, 18 aa, synthetic construct
  REGION 1..18: note = Description of Artificial Sequence: Synthetic peptide
MCTEDLEPPKATMTKKDC

SEQ ID NO: 30 - protein, 16 aa, synthetic construct
  REGION 1..16: note = Description of Artificial Sequence: Synthetic peptide
MCTEDQERPPVTKEDC

SEQ ID NO: 31 - protein, 18 aa, synthetic construct
  REGION 1..18: note = Description of Artificial Sequence: Synthetic peptide
MCIADPEPPEAQLPEGNC

SEQ ID NO: 32 - protein, 18 aa, synthetic construct
  REGION 1..18: note = Description of Artificial Sequence: Synthetic peptide
MCTGVQEPPEATLPKKNC

SEQ ID NO: 33 - protein, 18 aa, synthetic construct
  REGION 1..18: note = Description of Artificial Sequence: Synthetic peptide
MCSEAQEPPESRLPQVNC

SEQ ID NO: 34 - protein, 18 aa, synthetic construct
  REGION 1..18: note = Description of Artificial Sequence: Synthetic peptide
MCTKHLEPPGPPLPQENC

SEQ ID NO: 35 - protein, 18 aa, synthetic construct
  REGION 1..18: note = Description of Artificial Sequence: Synthetic peptide
MCTAAPEPPEPPVSKEYC

SEQ ID NO: 36 - protein, 18 aa, synthetic construct
  REGION 1..18: note = Description of Artificial Sequence: Synthetic peptide
MCTEDLQLPKTTMTKEYC

SEQ ID NO: 37 - protein, 18 aa, synthetic construct
  REGION 1..18: note = Description of Artificial Sequence: Synthetic peptide
MCSVDLQPPARLRPMVNC

SEQ ID NO: 38 - protein, 18 aa, synthetic construct
  REGION 1..18: note = Description of Artificial Sequence: Synthetic peptide
MCTGDLQPPESRQPQVNC

SEQ ID NO: 39 - protein, 18 aa, synthetic construct
  REGION 1..18: note = Description of Artificial Sequence: Synthetic peptide
MCTGDLQPPEAQVIEVNC

SEQ ID NO: 40 - protein, 18 aa, synthetic construct
  REGION 1..18: note = Description of Artificial Sequence: Synthetic peptide
MCTEDLQPPEPQLPEVNC

SEQ ID NO: 41 - protein, 18 aa, synthetic construct
  REGION 1..18: note = Description of Artificial Sequence: Synthetic peptide
MCTEDMEPRKTTMTKKYC

SEQ ID NO: 42 - protein, 22 aa, synthetic construct
  REGION 1..22: note = Description of Artificial Sequence: Synthetic peptide
MCTEAPAPPEPALPKKKKKKNC

SEQ ID NO: 43 - protein, 21 aa, synthetic construct
  REGION 1..21: note = Description of Artificial Sequence: Synthetic peptide
MCTEAPAPPEPALPKKKKKNC

SEQ ID NO: 44 - protein, 18 aa, synthetic construct
  REGION 1..18: note = Description of Artificial Sequence: Synthetic peptide
MCTEDLQSPKTTMTKENC

SEQ ID NO: 45 - protein, 21 aa, synthetic construct
  REGION 1..21: note = Description of Artificial Sequence: Synthetic peptide
MCTGDLKLPEPPMSKKKKKNC

SEQ ID NO: 46 - protein, 18 aa, synthetic construct
  REGION 1..18: note = Description of Artificial Sequence: Synthetic peptide
MCTEDLQPPKTTMAEKYC

SEQ ID NO: 47 - protein, 18 aa, synthetic construct
  REGION 1..18: note = Description of Artificial Sequence: Synthetic peptide
MCSEDPEPPKTTMTKKNC

SEQ ID NO: 48 - protein, 18 aa, synthetic construct
  REGION 1..18: note = Description of Artificial Sequence: Synthetic peptide
MCTEDLKPPEASLPEENC

SEQ ID NO: 49 - protein, 21 aa, synthetic construct
  REGION 1..21: note = Description of Artificial Sequence: Synthetic peptide
MCAGDLEQPEPPVAKKKKKNC

SEQ ID NO: 50 - protein, 18 aa, synthetic construct
  REGION 1..18: note = Description of Artificial Sequence: Synthetic peptide
MCNGDLERPEPPVAKEYC

SEQ ID NO: 51 - protein, 18 aa, synthetic construct
  REGION 1..18: note = Description of Artificial Sequence: Synthetic peptide
MCTEDLKPPEPPLPKENC

SEQ ID NO: 52 - protein, 16 aa, synthetic construct
  REGION 1..16: note = Description of Artificial Sequence: Synthetic peptide
MCNEALEPPPLRKEHC

SEQ ID NO: 53 - protein, 16 aa, synthetic construct
  REGION 1..16: note = Description of Artificial Sequence: Synthetic peptide
MCPEDLERPPLTKEHC

SEQ ID NO: 54 - protein, 18 aa, synthetic construct
  REGION 1..18: note = Description of Artificial Sequence: Synthetic peptide
MCTEDLEPPERPLPREIC

SEQ ID NO: 55 - protein, 18 aa, synthetic construct
  REGION 1..18: note = Description of Artificial Sequence: Synthetic peptide
MCAGDLKPPETTMSKKNC

SEQ ID NO: 56 - protein, 18 aa, synthetic construct
  REGION 1..18: note = Description of Artificial Sequence: Synthetic peptide
MCTEDLQQPERSQPMESC

SEQ ID NO: 57 - protein, 37 aa, synthetic construct
  REGION 1..37: note = Description of Artificial Sequence: Synthetic polypeptide
MCPEDLQPPEPALPEKKKKKIVVLALVVKSAVSVGVV

SEQ ID NO: 58 - protein, 35 aa, synthetic construct
  REGION 1..35: note = Description of Artificial Sequence: Synthetic polypeptide
MCTEVLVPRTTSGKGRLWFWLWSQKGHPSASACGS

SEQ ID NO: 59 - protein, 28 aa, synthetic construct
  REGION 1..28: note = Description of Artificial Sequence: Synthetic peptide
MCAEDLQPPPLLEAHCGSDSGRKKRRQC

SEQ ID NO: 60 - protein, 18 aa, synthetic construct
  REGION 1..18: note = Description of Artificial Sequence: Synthetic peptide
MCTAAPEPPEPQLPQANC

SEQ ID NO: 61 - protein, 18 aa, synthetic construct
  REGION 1..18: note = Description of Artificial Sequence: Synthetic peptide
MCPADLQQPETSLPEENC

SEQ ID NO: 62 - protein, 18 aa, synthetic construct
  REGION 1..18: note = Description of Artificial Sequence: Synthetic peptide
MCSVDLQPPARLRPMVNC

SEQ ID NO: 63 - protein, 18 aa, synthetic construct
  REGION 1..18: note = Description of Artificial Sequence: Synthetic peptide
MCTEALEPPEPPLTKENC

SEQ ID NO: 64 - protein, 18 aa, synthetic construct
  REGION 1..18: note = Description of Artificial Sequence: Synthetic peptide
MCTEAMEPPEPPLARESC

SEQ ID NO: 65 - protein, 18 aa, synthetic construct
  REGION 1..18: note = Description of Artificial Sequence: Synthetic peptide
MCTADLQPPEASLPQQNC

SEQ ID NO: 66 - protein, 18 aa, synthetic construct
  REGION 1..18: note = Description of Artificial Sequence: Synthetic peptide
MCTAAPEPPEPRLPEGNC

SEQ ID NO: 67 - protein, 18 aa, synthetic construct
  REGION 1..18: note = Description of Artificial Sequence: Synthetic peptide
MCTKDLAPQAPPLLKENC

SEQ ID NO: 68 - protein, 18 aa, synthetic construct
  REGION 1..18: note = Description of Artificial Sequence: Synthetic peptide
  SITE 1..18: note = D-amino acid
CNEKPLPPEPPELDETCM

SEQ ID NO: 69 - protein, 21 aa, synthetic construct
  REGION 1..21: note = Description of Artificial Sequence: Synthetic peptide
  SITE 1..21: note = D-amino acid
CNKKKKKPLAPEPPAPAETCM

SEQ ID NO: 70 - protein, 17 aa, synthetic construct
  REGION 1..17: note = Description of Artificial Sequence: Synthetic peptide
  SITE 1..17: note = D-amino acid
CNEQPVATPPQLDETCM

SEQ ID NO: 71 - protein, 20 aa, synthetic construct
  REGION 1..20: note = Description of Artificial Sequence: Synthetic peptide
  SITE 1..20: note = D-amino acid
CNKKKKPLAPEPPAPAETCM

SEQ ID NO: 72 - protein, 18 aa, synthetic construct
  REGION 1..18: note = Description of Artificial Sequence: Synthetic peptide
  SITE 1..18: note = D-amino acid
CDKKSVTTKPPQLDGTCM

SEQ ID NO: 73 - protein, 18 aa, synthetic construct
  REGION 1..18: note = Description of Artificial Sequence: Synthetic peptide
  SITE 1..18: note = D-amino acid
CDKKSVTTKPPQLDGTCM

SEQ ID NO: 74 - protein, 18 aa, synthetic construct
  REGION 1..18: note = Description of Artificial Sequence: Synthetic peptide
  SITE 1..18: note = D-amino acid
CNEKTMTTKPSQLDETCM

SEQ ID NO: 75 - protein, 18 aa, synthetic construct
  REGION 1..18: note = Description of Artificial Sequence: Synthetic peptide
  SITE 1..18: note = D-amino acid
CDEKPLPPEPPELDETCM

SEQ ID NO: 76 - protein, 18 aa, synthetic construct
  REGION 1..18: note = Description of Artificial Sequence: Synthetic peptide
  SITE 1..18: note = D-amino acid
CNEEPLQEELLELDETCM

SEQ ID NO: 77 - protein, 18 aa, synthetic construct
  REGION 1..18: note = Description of Artificial Sequence: Synthetic peptide
  SITE 1..18: note = D-amino acid
CNQKTMTTKPPKLDGPCM

SEQ ID NO: 78 - protein, 17 aa, synthetic construct
  REGION 1..17: note = Description of Artificial Sequence: Synthetic peptide
  SITE 1..17: note = D-amino acid
CYEKPMPPEPQLDGPCM

SEQ ID NO: 79 - protein, 19 aa, synthetic construct
  REGION 1..19: note = Description of Artificial Sequence: Synthetic peptide
  SITE 1..19: note = D-amino acid
CDEKKTMTAKPPELDETCM

SEQ ID NO: 80 - protein, 16 aa, synthetic construct
  REGION 1..16: note = Description of Artificial Sequence: Synthetic peptide
  SITE 1..16: note = D-amino acid
CDEKTVPPREQDETCM

SEQ ID NO: 81 - protein, 18 aa, synthetic construct
  REGION 1..18: note = Description of Artificial Sequence: Synthetic peptide
  SITE 1..18: note = D-amino acid
CNGEPLQAEPPEPDAICM

SEQ ID NO: 82 - protein, 18 aa, synthetic construct
  REGION 1..18: note = Description of Artificial Sequence: Synthetic peptide
  SITE 1..18: note = D-amino acid
CNKKPLTAEPPEQVGTCM

SEQ ID NO: 83 - protein, 18 aa, synthetic construct
  REGION 1..18: note = Description of Artificial Sequence: Synthetic peptide
  SITE 1..18: note = D-amino acid
CNVQPLPPGPPELHKTCM

SEQ ID NO: 84 - protein, 18 aa, synthetic construct
  REGION 1..18: note = Description of Artificial Sequence: Synthetic peptide
  SITE 1..18: note = D-amino acid
CNEQPLPPGPPEPAATCM

SEQ ID NO: 85 - protein, 18 aa, synthetic construct
  REGION 1..18: note = Description of Artificial Sequence: Synthetic peptide
  SITE 1..18: note = D-amino acid
CYEKSVPPEPPEPAATCM

SEQ ID NO: 86 - protein, 18 aa, synthetic construct
  REGION 1..18: note = Description of Artificial Sequence: Synthetic peptide
  SITE 1..18: note = D-amino acid
CYEKTMTTKPLQLDETCM

SEQ ID NO: 87 - protein, 18 aa, synthetic construct
  REGION 1..18: note = Description of Artificial Sequence: Synthetic peptide
  SITE 1..18: note = D-amino acid
CNVMPRLRAPPQLDVSCM

SEQ ID NO: 88 - protein, 18 aa, synthetic construct
  REGION 1..18: note = Description of Artificial Sequence: Synthetic peptide
  SITE 1..18: note = D-amino acid
CNVQPQRSEPPQLDGTCM

SEQ ID NO: 89 - protein, 18 aa, synthetic construct
  REGION 1..18: note = Description of Artificial Sequence: Synthetic peptide
  SITE 1..18: note = D-amino acid
CNVEIVQAEPPQLDGTCM

SEQ ID NO: 90 - protein, 18 aa, synthetic construct
  REGION 1..18: note = Description of Artificial Sequence: Synthetic peptide
  SITE 1..18: note = D-amino acid
CNVEPLQPEPPQLDETCM

SEQ ID NO: 91 - protein, 18 aa, synthetic construct
  REGION 1..18: note = Description of Artificial Sequence: Synthetic peptide
  SITE 1..18: note = D-amino acid
CYKKTMTTKPPEMDETCM

SEQ ID NO: 92 - protein, 23 aa, synthetic construct
  REGION 1..23: note = Description of Artificial Sequence: Synthetic peptide
  SITE 1..23: note = D-amino acid
CNKKKKKKPKLAPEPPAPAETCM

SEQ ID NO: 93 - protein, 21 aa, synthetic construct
  REGION 1..21: note = Description of Artificial Sequence: Synthetic peptide
  SITE 1..21: note = D-amino acid
CNKKKKKPLAPEPPAPAETCM

SEQ ID NO: 94 - protein, 18 aa, synthetic construct
  REGION 1..18: note = Description of Artificial Sequence: Synthetic peptide
  SITE 1..18: note = D-amino acid
CNEKTMTTKPSQLDETCM

SEQ ID NO: 95 - protein, 22 aa, synthetic construct
  REGION 1..22: note = Description of Artificial Sequence: Synthetic peptide
  SITE 1..22: note = D-amino acid
CNKKKKKSMPPEPPLKLDGTCM

SEQ ID NO: 96 - protein, 18 aa, synthetic construct
  REGION 1..18: note = Description of Artificial Sequence: Synthetic peptide
  SITE 1..18: note = D-amino acid
CYKEAMTTKPPQLDETCM

SEQ ID NO: 97 - protein, 18 aa, synthetic construct
  REGION 1..18: note = Description of Artificial Sequence: Synthetic peptide
  SITE 1..18: note = D-amino acid
CNKKTMTTKPPEPDESCM

SEQ ID NO: 98 - protein, 18 aa, synthetic construct
  REGION 1..18: note = Description of Artificial Sequence: Synthetic peptide
  SITE 1..18: note = D-amino acid
CNEEPLSAEPPKLDETCM

SEQ ID NO: 99 - protein, 21 aa, synthetic construct
  REGION 1..21: note = Description of Artificial Sequence: Synthetic peptide
  SITE 1..21: note = D-amino acid
CNKKKKKAVPPEPQELDGACM

SEQ ID NO: 100 - protein, 18 aa, synthetic construct
  REGION 1..18: note = Description of Artificial Sequence: Synthetic peptide
  SITE 1..18: note = D-amino acid
CYEKAVPPEPRELDGNCM

SEQ ID NO: 101 - protein, 18 aa, synthetic construct
  REGION 1..18: note = Description of Artificial Sequence: Synthetic peptide
  SITE 1..18: note = D-amino acid
CNEKPLPPEPPKLDETCM

SEQ ID NO: 102 - protein, 16 aa, synthetic construct
  REGION 1..16: note = Description of Artificial Sequence: Synthetic peptide
  SITE 1..16: note = D-amino acid
CHEKRLPPPELAENCM

SEQ ID NO: 103 - protein, 16 aa, synthetic construct
  REGION 1..16: note = Description of Artificial Sequence: Synthetic peptide
  SITE 1..16: note = D-amino acid
CHEKTLPPRELDEPCM

SEQ ID NO: 104 - protein, 18 aa, synthetic construct
  REGION 1..18: note = Description of Artificial Sequence: Synthetic peptide
  SITE 1..18: note = D-amino acid
CIERPLPREPPELDETCM

SEQ ID NO: 105 - protein, 18 aa, synthetic construct
  REGION 1..18: note = Description of Artificial Sequence: Synthetic peptide
  SITE 1..18: note = D-amino acid
CNKKSMTTEPPKLDGACM

SEQ ID NO: 106 - protein, 18 aa, synthetic construct
  REGION 1..18: note = Description of Artificial Sequence: Synthetic peptide
  SITE 1..18: note = D-amino acid
CSMPQSREPQQLEDETCM

SEQ ID NO: 107 - protein, 38 aa, synthetic construct
  REGION 1..38: note = Description of Artificial Sequence: Synthetic polypeptide
  SITE 1..38: note = D-amino acid
CVVGVSVASKVVLALVVIKKKKKEPLAPEPPQLDEPCM

SEQ ID NO: 108 - protein, 36 aa, synthetic construct
  REGION 1..36: note = Description of Artificial Sequence: Synthetic polypeptide
  SITE 1..36: note = D-amino acid
CSGCASASPHGKQSWLWFWLRGKGSTTRPVLVETCM

SEQ ID NO: 109 - protein, 28 aa, synthetic construct
  REGION 1..28: note = Description of Artificial Sequence: Synthetic peptide
  SITE 1..28: note = D-amino acid
CQRRKKRGSDSGCHAELLPPPQLDEACM

SEQ ID NO: 110 - protein, 18 aa, synthetic construct
  REGION 1..18: note = Description of Artificial Sequence: Synthetic peptide
  SITE 1..18: note = D-amino acid
CNAQPLQPEPPEPAATCM

SEQ ID NO: 111 - protein, 18 aa, synthetic construct
  REGION 1..18: note = Description of Artificial Sequence: Synthetic peptide
  SITE 1..18: note = D-amino acid
CNEEPLSTEPQQLDAPCM

SEQ ID NO: 112 - protein, 18 aa, synthetic construct
  REGION 1..18: note = Description of Artificial Sequence: Synthetic peptide
  SITE 1..18: note = D-amino acid
CNVMPRLRAPPQLDVSCM

SEQ ID NO: 113 - protein, 18 aa, synthetic construct
  REGION 1..18: note = Description of Artificial Sequence: Synthetic peptide
  SITE 1..18: note = D-amino acid
CNEKTLPPEPPELAETCM

SEQ ID NO: 114 - protein, 18 aa, synthetic construct
  REGION 1..18: note = Description of Artificial Sequence: Synthetic peptide
  SITE 1..18: note = D-amino acid
CSERALPPEPPEMAETCM

SEQ ID NO: 115 - protein, 18 aa, synthetic construct
  REGION 1..18: note = Description of Artificial Sequence: Synthetic peptide
  SITE 1..18: note = D-amino acid
CNQQPLSAEPPQLDATCM

SEQ ID NO: 116 - protein, 18 aa, synthetic construct
  REGION 1..18: note = Description of Artificial Sequence: Synthetic peptide
  SITE 1..18: note = D-amino acid
CNGEPLRPEPPEPAATCM

SEQ ID NO: 117 - protein, 18 aa, synthetic construct
  REGION 1..18: note = Description of Artificial Sequence: Synthetic peptide
  SITE 1..18: note = D-amino acid
CNEKLLPPAQPALDKTCM

SEQ ID NO: 118 - protein, 10 aa, synthetic construct
  REGION 1..10: note = Description of Artificial Sequence: Synthetic peptide
GRKKRRQRRR

SEQ ID NO: 119 - protein, 31 aa, synthetic construct
  REGION 1..31: note = Description of Artificial Sequence: Synthetic polypeptide
GDIMGEWGNEIFGAIAGFLGYGRKKRRQRRR

SEQ ID NO: 120 - protein, 8 aa, synthetic construct
  REGION 1..8: note = Description of Artificial Sequence: Synthetic peptide
RRRRRRRR

SEQ ID NO: 121 - protein, 8 aa, synthetic construct
  REGION 1..8: note = Description of Artificial Sequence: Synthetic peptide
KKKKKKKK

SEQ ID NO: 122 - protein, 19 aa, synthetic construct
  REGION 1..19: note = Description of Artificial Sequence: Synthetic peptide
DIMGEWGNEIFGAIAGFLG

SEQ ID NO: 123 - protein, 22 aa, synthetic construct
  REGION 1..22: note = Description of Artificial Sequence: Synthetic peptide
CHHHHHRKKRRQRRRRHHHHHC

SEQ ID NO: 124 - protein, 21 aa, synthetic construct
  REGION 1..21: note = Description of Artificial Sequence: Synthetic peptide
CHHHHHRRRRRRRRRHHHHHC

SEQ ID NO: 125 - protein, 17 aa, synthetic construct
  REGION 1..17: note = Description of Artificial Sequence: Synthetic peptide
FFLIPKGRRRRRRRRGC

SEQ ID NO: 126 - protein, 6 aa, synthetic construct
  REGION 1..6: note = Description of Artificial Sequence: Synthetic peptide
  SITE 2: note = L-2-naphthylalanine
FXRRRR

SEQ ID NO: 127 - protein, 15 aa, synthetic construct
  REGION 1..15: note = Description of Artificial Sequence: Synthetic peptide
  SITE 15: note = D-amino acid
RRWWRRWRRRRWWRR

SEQ ID NO: 128 - protein, 14 aa, synthetic construct
  REGION 1..14: note = Description of Artificial Sequence: Synthetic peptide
  SITE 14: note = D-amino acid
RWWRRRRWRRWWRR

SEQ ID NO: 129 - protein, 14 aa, synthetic construct
  REGION 1..14: note = Description of Artificial Sequence: Synthetic peptide
  SITE 14: note = D-amino acid
RRWWRRWRRRRWWR

SEQ ID NO: 130 - protein, 11 aa, synthetic construct
  REGION 1..11: note = Description of Artificial Sequence: Synthetic peptide
  SITE 11: note = D-amino acid
RRWWRRWRRRR

SEQ ID NO: 131 - protein, 9 aa, synthetic construct
  REGION 1..9: note = Description of Artificial Sequence: Synthetic peptide
  SITE 9: note = D-amino acid
RRWWRRWRR

SEQ ID NO: 132 - protein, 10 aa, synthetic construct
  REGION 1..10: note = Description of Artificial Sequence: Synthetic peptide
RRWWRRWRRR

SEQ ID NO: 133 - protein, 14 aa, synthetic construct
  REGION 1..14: note = Description of Artificial Sequence: Synthetic peptide
  SITE 7: note = 2-amino-tetradecanoic acid
  SITE 14: note = D-amino acid
RRRRRRXRRWWRRR

SEQ ID NO: 134 - protein, 18 aa, synthetic construct
  REGION 1..18: note = Description of Artificial Sequence: Synthetic peptide
YALTSAISRIITHHHHHH

SEQ ID NO: 135 - protein, 12 aa, synthetic construct
  REGION 1..12: note = Description of Artificial Sequence: Synthetic peptide
  SITE 6: note = 2-amino-tetradecanoic acid
RRRRRXRRWWRR

SEQ ID NO: 136 - protein, 11 aa, synthetic construct
  REGION 1..11: note = Description of Artificial Sequence: Synthetic peptide
  SITE 3: note = 2-amino-tetradecanoic acid
  SITE 6: note = 2-amino-tetradecanoic acid
RRXRRXRRRRR

SEQ ID NO: 137 - protein, 6 aa, synthetic construct
  REGION 1..6: note = Description of Artificial Sequence: Synthetic peptide
  SITE 3: note = 2-amino-tetradecanoic acid
RRXRRR

SEQ ID NO: 138 - skipped

SEQ ID NO: 139 - protein, 4 aa, synthetic construct
  REGION 1..4: note = Description of Artificial Sequence: Synthetic peptide
GSGS

SEQ ID NO: 140 - protein, 10 aa, synthetic construct
  REGION 1..10: note = Description of Artificial Sequence: Synthetic peptide
  SITE 1..10: note = D-amino acid
RRRQRRKKRG

SEQ ID NO: 141 - protein, 33 aa, synthetic construct
  REGION 1..33: note = Description of Artificial Sequence: Synthetic polypeptide
  SITE 1..33: note = D-amino acid
RRRQRRKKRGYGLFGAIAGFIENGWEGMGMIDG

SEQ ID NO: 142 - protein, 8 aa, synthetic construct
  REGION 1..8: note = Description of Artificial Sequence: Synthetic peptide
  SITE 1..8: note = D-amino acid
RRRRRRRR

SEQ ID NO: 143 - protein, 8 aa, synthetic construct
  REGION 1..8: note = Description of Artificial Sequence: Synthetic peptide
  SITE 1..8: note = D-amino acid
KKKKKKKK

SEQ ID NO: 144 - protein, 19 aa, synthetic construct
  REGION 1..19: note = Description of Artificial Sequence: Synthetic peptide
  SITE 1..19: note = D-amino acid
GLFGAIAGFIENGWEGMID

SEQ ID NO: 145 - protein, 22 aa, synthetic construct
  REGION 1..22: note = Description of Artificial Sequence: Synthetic peptide
  SITE 1..22: note = D-amino acid
CHHHHHRRRRQRRKKRHHHHHC

SEQ ID NO: 146 - protein, 21 aa, synthetic construct
  REGION 1..21: note = Description of Artificial Sequence: Synthetic peptide
  SITE 1..21: note = D-amino acid
CHHHHHRRRRRRRRRHHHHHC

SEQ ID NO: 147 - protein, 18 aa, synthetic construct
  REGION 1..18: note = Description of Artificial Sequence: Synthetic peptide
  SITE 1..18: note = D-amino acid
CGRRRRRRRRGKPILFFC

SEQ ID NO: 148 - protein, 6 aa, synthetic construct
  REGION 1..6: note = Description of Artificial Sequence: Synthetic peptide
  SITE 1..4: note = D-amino acid
  SITE 5: note = L-2-naphthylalanine
  SITE 6: note = D-amino acid
RRRRXF

SEQ ID NO: 149 - protein, 15 aa, synthetic construct
  REGION 1..15: note = Description of Artificial Sequence: Synthetic peptide
  SITE 1..15: note = D-amino acid
RRWWRRRRWRRWWRR

SEQ ID NO: 150 - protein, 14 aa, synthetic construct
  REGION 1..14: note = Description of Artificial Sequence: Synthetic peptide
  SITE 1..14: note = D-amino acid
RRWWRRWRRRRWWR

SEQ ID NO: 151 - protein, 14 aa, synthetic construct
  REGION 1..14: note = Description of Artificial Sequence: Synthetic peptide
  SITE 1..14: note = D-amino acid
RWWRRRRWRRWWRR

SEQ ID NO: 152 - protein, 11 aa, synthetic construct
  REGION 1..11: note = Description of Artificial Sequence: Synthetic peptide
  SITE 1..11: note = D-amino acid
RRRRWRRWWRR

SEQ ID NO: 153 - protein, 9 aa, synthetic construct
  REGION 1..9: note = Description of Artificial Sequence: Synthetic peptide
  SITE 1..9: note = D-amino acid
RRWRRWWRR

SEQ ID NO: 154 - protein, 10 aa, synthetic construct
  REGION 1..10: note = Description of Artificial Sequence: Synthetic peptide
  SITE 1..10: note = D-amino acid
RRRWRRWWRR

SEQ ID NO: 155 - protein, 14 aa, synthetic construct
  REGION 1..14: note = Description of Artificial Sequence: Synthetic peptide
  SITE 1..7: note = D-amino acid
  SITE 8: note = 2-amino-tetradecanoic acid
  SITE 9..14: note = D-amino acid
RRRWWRRXRRRRRR

SEQ ID NO: 156 - protein, 18 aa, synthetic construct
  REGION 1..18: note = Description of Artificial Sequence: Synthetic peptide
  SITE 1..18: note = D-amino acid
HHHHHHTIIRSIASTLAY

SEQ ID NO: 157 - protein, 12 aa, synthetic construct
  REGION 1..12: note = Description of Artificial Sequence: Synthetic peptide
  SITE 1..6: note = D-amino acid
  SITE 7: note = 2-amino-tetradecanoic acid
  SITE 8..12: note = D-amino acid
RRWWRRXRRRRR

SEQ ID NO: 158 - protein, 11 aa, synthetic construct
  REGION 1..11: note = Description of Artificial Sequence: Synthetic peptide
  SITE 1..2: note = D-amino acid
  SITE 3: note = 2-amino-tetradecanoic acid
  SITE 4..5: note = D-amino acid
  SITE 6: note = 2-amino-tetradecanoic acid
  SITE 7..11: note = D-amino acid
RRXRRXRRRRR

SEQ ID NO: 159 - protein, 6 aa, synthetic construct
  REGION 1..6: note = Description of Artificial Sequence: Synthetic peptide
  SITE 1..2: note = D-amino acid
  SITE 3: note = 2-amino-tetradecanoic acid
  SITE 4..6: note = D-amino acid
RRXRRR

SEQ ID NO: 160 - skipped

SEQ ID NO: 161 - protein, 4 aa, synthetic construct
  REGION 1..4: note = Description of Artificial Sequence: Synthetic peptide
  SITE 1..4: note = D-amino acid
SGSG

SEQ ID NO: 162 - DNA, 816 bp, synthetic construct
  misc_feature 1..816: note = Description of Artificial Sequence: Synthetic polynucleotide
  source 1..816: mol_type = other DNA
atggatagtagtgcggtgatcacacaaatctccaaggaggaagcccgtgggccgctgcgggggaagggtgatcaaaaatcggcagctagtcaaaaacctcgctctcgtgggatacttcattcgctgttttgctgcgtctgccgcgatgacggagaagcattgcctgcgcattcaggggcgcctttacttgttgaggaaaatggtgcaattcctaaacaaactccagtacaatacttactgccggaggcaaaggcacaagacagtgataagatatgtgtagtaatagacttagatgaaacactggtacattcgtcattcaaacctgttaataatgcggatttcatcatacctgtagaaatcgacggggttgtccatcaggtttacgtcctgaagcggcctcatgtagatgaatttttacagcggatgggcgagttatttgaatgtgtgctgtttacagctagtcttgccaagtacgcggatcctgtcgcggatttgcttgataagtggggtgcgtttcgggcgagattatttcgcgaatcttgcgtttttcacagaggtaactacgtgaaggaccttagtcgtctgggtagagatcttagaagagtgctgatccttgacaacagcccagccagctatgtctttcatccggataacgcagtacccgtggcgtcttggttcgacaatatgtcggacacggagctgcatgacctgttgccgttctttgagcagttgagtcgcgttgatgacgtttactcggttttgcgtcaaccccgtccgggatctggttctggctctcaccatcaccatcaccactag

SEQ ID NO: 163 - protein, 271 aa, synthetic construct
  REGION 1..271: note = Description of Artificial Sequence: Synthetic polypeptide
MDSSAVITQISKEEARGPLRGKGDQKSAASQKPRSRGILHSLFCCVCRDDGEALPAHSGAPLLVEENGAIPKQTPVQYLLPEAKAQDSDKICVVIDLDETLVHSSFKPVNNADFIIPVEIDGVVHQVYVLKRPHVDEFLQRMGELFECVLFTASLAKYADPVADLLDKWGAFRARLFRESCVFHRGNYVKDLSRLGRDLRRVLILDNSPASYVFHPDNAVPVASWFDNMSDTELHDLLPFFEQLSRVDDVYSVLRQPRPGSGSGSHHHHHH

SEQ ID NO: 164 - DNA, 732 bp, synthetic construct
  misc_feature 1..732: note = Description of Artificial Sequence: Synthetic polynucleotide
  source 1..732: mol_type = other DNA
atgtgtaccgaagatctggaaccaccagaaccaccactgccaaaggaaaattgtggatccggttctggctcaggttcttcccctatactaggttattggaaaattaagggccttgtgcaacccactcgacttcttttggaatatcttgaagaaaaatatgaagagcatttgtatgagcgcgatgaaggtgataaatggcgaaacaaaaagtttgaattgggtttggagtttcccaatcttccttattatattgatggtgatgttaaattaacacagtctatggccatcatacgttatatagctgacaagcacaacatgttgggtggttgtccaaaagagcgtgcagagatttcaatgcttgaaggagcggttttggatattagatacggtgtttcgagaattgcatatagtaaagactttgaaactctcaaagttgattttcttagcaagctacctgaaatgctgaaaatgttcgaagatcgtttatgtcataaaacatatttaaatggtgatcatgtaacccatcctgacttcatgttgtatgacgctcttgatgttgttttatacatggacccaatgtgcctggatgcgttcccaaaattagtttgttttaaaaaacgtattgaagctatcccacaaattgataagtacttgaaatccagcaagtatatagcatggcctttgcagggctggcaagccacgtttggtggtggcgaccatcctccaaaataa

SEQ ID NO: 165 - protein, 243 aa, synthetic construct
  REGION 1..243: note = Description of Artificial Sequence: Synthetic polypeptide
MCTEDLEPPEPPLPKENCGSGSGSGSSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLNGDHVTHPDFMLYDALDVVLYMDPMCLDAFPKLVCFKKRIEAIPQIDKYLKSSKYIAWPLQGWQATFGGGDHPPK

SEQ ID NO: 166 - DNA, 53 bp, synthetic construct
  misc_feature 1..53: note = Description of Artificial Sequence: Synthetic primer
  source 1..53: mol_type = other DNA
aataaagcttatgtgtaccgaagatctggaaccaccagaaccaccactgccaa

SEQ ID NO: 167 - DNA, 48 bp, synthetic construct
  misc_feature 1..48: note = Description of Artificial Sequence: Synthetic primer
  source 1..48: mol_type = other DNA
aataggatccacaattttcctttggcagtggtggttctggtggttcca

SEQ ID NO: 168 - DNA, 55 bp, synthetic construct
  misc_feature 1..55: note = Description of Artificial Sequence: Synthetic primer
  source 1..55: mol_type = other DNA
aataaagcttatgtgtaccgaagctccggcaccaccagaaccagcactgccaaag

SEQ ID NO: 169 - DNA, 55 bp, synthetic construct
  misc_feature 1..55: note = Description of Artificial Sequence: Synthetic primer
  source 1..55: mol_type = other DNA
aataggatccacaattttttttttttttctttggcagtgctggttctggtggtgc

SEQ ID NO: 170 - DNA, 50 bp, synthetic construct
  misc_feature 1..50: note = Description of Artificial Sequence: Synthetic primer
  source 1..50: mol_type = other DNA
aataaagcttatgtgtaccgaagatctgcaaccaccaacagcagtgccac

SEQ ID NO: 171 - DNA, 52 bp, synthetic construct
  misc_feature 1..52: note = Description of Artificial Sequence: Synthetic primer
  source 1..52: mol_type = other DNA
aataggatccacaattttcctgtggcactgctgttggtggttgcagatcttc

SEQ ID NO: 172 - DNA, 55 bp, synthetic construct; identical to SEQ ID NO: 168
  misc_feature 1..55: note = Description of Artificial Sequence: Synthetic primer
  source 1..55: mol_type = other DNA

SEQ ID NO: 173 - DNA, 52 bp, synthetic construct
  misc_feature 1..52: note = Description of Artificial Sequence: Synthetic primer
  source 1..52: mol_type = other DNA
aataggatccacaatttttttttttctttggcagtgctggttctggtggtgc

SEQ ID NO: 174 - DNA, 42 bp, synthetic construct
  misc_feature 1..42: note = Description of Artificial Sequence: Synthetic primer
  source 1..42: mol_type = other DNA
atgtgtaccgcagatctggaaccaccagaaccacgaatggaa

SEQ ID NO: 175 - DNA, 54 bp, synthetic construct
  misc_feature 1..54: note = Description of Artificial Sequence: Synthetic primer
  source 1..54: mol_type = other DNA
aataggatccacaatctaccttttttttttccattcgtggttctggtggttcca

SEQ ID NO: 176 - DNA, 53 bp, synthetic construct
  misc_feature 1..53: note = Description of Artificial Sequence: Synthetic primer
  source 1..53: mol_type = other DNA
aataaagcttatgtgtaccggagatctgcaaccaccaaaaacaacagtgtcaa

SEQ ID NO: 177 - DNA, 50 bp, synthetic construct
  misc_feature 1..50: note = Description of Artificial Sequence: Synthetic primer
  source 1..50: mol_type = other DNA
aataggatccacaatctttctttgacactgttgtttttggtggttgcaga

SEQ ID NO: 178 - DNA, 53 bp, synthetic construct
  misc_feature 1..53: note = Description of Artificial Sequence: Synthetic primer
  source 1..53: mol_type = other DNA
aataaagcttatgtgtaccgaagatctgcaatcaccaaaaacaacaatgacaa

SEQ ID NO: 179 - DNA, 50 bp, synthetic construct
  misc_feature 1..50: note = Description of Artificial Sequence: Synthetic primer
  source 1..50: mol_type = other DNA
aataggatccacaattttcctttgtcattgttgtttttggtgattgcaga

SEQ ID NO: 180 - DNA, 53 bp, synthetic construct; identical to SEQ ID NO: 166
  misc_feature 1..53: note = Description of Artificial Sequence: Synthetic primer
  source 1..53: mol_type = other DNA

SEQ ID NO: 181 - DNA, 51 bp, synthetic construct
  misc_feature 1..51: note = Description of Artificial Sequence: Synthetic primer
  source 1..51: mol_type = other DNA
aataggatcccacaatcttcctttggcagtggtggttctggtggttccaga

SEQ ID NO: 182 - DNA, 49 bp, synthetic construct
  misc_feature 1..49: note = Description of Artificial Sequence: Synthetic primer
  source 1..49: mol_type = other DNA
aataaagcttatgtgtaccgaagatcaggaacaacaagaagaacaactg

SEQ ID NO: 183 - DNA, 52 bp, synthetic construct
  misc_feature 1..52: note = Description of Artificial Sequence: Synthetic primer
  source 1..52: mol_type = other DNA
aataggatccacaattttcctctggcagttgttcttcttgttgttcctgatc

SEQ ID NO: 184 - DNA, 53 bp, synthetic construct
  misc_feature 1..53: note = Description of Artificial Sequence: Synthetic primer
  source 1..53: mol_type = other DNA
aataaagcttatgtgtaccgcagatctgaaaccaccaaaaacaacaatgacaa

SEQ ID NO: 185 - DNA, 50 bp, synthetic construct
  misc_feature 1..50: note = Description of Artificial Sequence: Synthetic primer
  source 1..50: mol_type = other DNA
aataggatccacaattttgctttgtcattgttgtttttggtggtttcaga

SEQ ID NO: 186 - DNA, 53 bp, synthetic construct
  misc_feature 1..53: note = Description of Artificial Sequence: Synthetic primer
  source 1..53: mol_type = other DNA
aataaagcttatgtgtcccggagatctgaaacaaccagaaccaccaatgccaa

SEQ ID NO: 187 - DNA, 50 bp, synthetic construct
  misc_feature 1..50: note = Description of Artificial Sequence: Synthetic primer
  source 1..50: mol_type = other DNA
aataggatccacaatattcctttggcattggtggttctggttgtttcaga

SEQ ID NO: 188 - DNA, 53 bp, synthetic construct
  misc_feature 1..53: note = Description of Artificial Sequence: Synthetic primer
  source 1..53: mol_type = other DNA
aataaagcttatgtgtaccgaagatctggaaccaccaaaagcaacaatgacaa

SEQ ID NO: 189 - DNA, 50 bp, synthetic construct
  misc_feature 1..50: note = Description of Artificial Sequence: Synthetic primer
  source 1..50: mol_type = other DNA
aataggatccacaatctttctttgtcattgttgcttttggtggttccaga

SEQ ID NO: 190 - DNA, 49 bp, synthetic construct
  misc_feature 1..49: note = Description of Artificial Sequence: Synthetic primer
  source 1..49: mol_type = other DNA
aataaagcttatgtgtaccgaagatcaggaacgaccaccagtgacaaag

SEQ ID NO: 191 - DNA, 49 bp, synthetic construct
  misc_feature 1..49: note = Description of Artificial Sequence: Synthetic primer
  source 1..49: mol_type = other DNA
aataggatccacaatcttcctttgtcactggtggtcgttcctgatcttc

SEQ ID NO: 192 - DNA, 49 bp, synthetic construct
  misc_feature 1..49: note = Description of Artificial Sequence: Synthetic primer
  source 1..49: mol_type = other DNA
aataaagcttatgtgtatcgcagatccggaaccaccagaagcacaactg

SEQ ID NO: 193 - DNA, 54 bp, synthetic construct
  misc_feature 1..54: note = Description of Artificial Sequence: Synthetic primer
  source 1..54: mol_type = other DNA
aataggatccacaatttccctctggcagttgtgcttctggtggttccggatctg

SEQ ID NO: 194 - DNA, 49 bp, synthetic construct
  misc_feature 1..49: note = Description of Artificial Sequence: Synthetic primer
  source 1..49: mol_type = other DNA
aataaagcttatgtgtaccggagttcaggaaccaccagaagcaacactg

SEQ ID NO: 195 - DNA, 52 bp, synthetic construct
  misc_feature 1..52: note = Description of Artificial Sequence: Synthetic primer
  source 1..52: mol_type = other DNA
aataggatccacaatttttctttggcagtgttgcttctggtggttcctgaac

SEQ ID NO: 196 - DNA, 49 bp, synthetic construct
  misc_feature 1..49: note = Description of Artificial Sequence: Synthetic primer
  source 1..49: mol_type = other DNA
aataaagcttatgtgtagcgaagctcaggaaccaccagaatcacgactg

SEQ ID NO: 197 - DNA, 51 bp, synthetic construct
  misc_feature 1..51: note = Description of Artificial Sequence: Synthetic primer
  source 1..51: mol_type = other DNA
aataggatccacaatttacctgtggcagtcgtgattctggtggttcctgag

SEQ ID NO: 198 - DNA, 50 bp, synthetic construct
  misc_feature 1..50: note = Description of Artificial Sequence: Synthetic primer
  source 1..50: mol_type = other DNA
aataaagcttatgtgtaccaaacatctggaaccaccaggaccaccattgc

SEQ ID NO: 199 - DNA, 52 bp, synthetic construct
  misc_feature 1..52: note = Description of Artificial Sequence: Synthetic primer
  source 1..52: mol_type = other DNA
aataggatccacaattttcctgtggcaatggtggtcctggtggttccagatg

SEQ ID NO: 200 - DNA, 50 bp, synthetic construct
  misc_feature 1..50: note = Description of Artificial Sequence: Synthetic primer
  source 1..50: mol_type = other DNA
aataaagcttatgtgtaccgcagctccggaaccaccagaaccaccagtgt

SEQ ID NO: 201 - DNA, 51 bp, synthetic construct
  misc_feature 1..51: note = Description of Artificial Sequence: Synthetic primer
  source 1..51: mol_type = other DNA
aataggatccacaatattcctttgacactggtggttctggtggttccggag

SEQ ID NO: 202 - DNA, 52 bp, synthetic construct
  misc_feature 1..52: note = Description of Artificial Sequence: Synthetic primer
  source 1..52: mol_type = other DNA
aataaagcttatgtgtaccgaagatctgcaactaccaaaaacaacaatgaca

SEQ ID NO: 203 - DNA, 50 bp, synthetic construct
  misc_feature 1..50: note = Description of Artificial Sequence: Synthetic primer
  source 1..50: mol_type = other DNA
aataggatccacaatattcctttgtcattgttgtttttggtagttgcaga

SEQ ID NO: 204 - DNA, 53 bp, synthetic construct
  misc_feature 1..53: note = Description of Artificial Sequence: Synthetic primer
  source 1..53: mol_type = other DNA
aataaagcttatgtgttccgtagatctgcaaccaccagcacgactacggccaa

SEQ ID NO: 205 - DNA, 51 bp, synthetic construct
  misc_feature 1..51: note = Description of Artificial Sequence: Synthetic primer
  source 1..51: mol_type = other DNA
aataggatccacaatttaccattggccgtagtcgtgctggtggttgcagat

SEQ ID NO: 206 - DNA, 52 bp, synthetic construct
  misc_feature 1..52: note = Description of Artificial Sequence: Synthetic primer
  source 1..52: mol_type = other DNA
aataaagcttatgtgtaccggagatctgcaaccaccagaatcacgacagcca

SEQ ID NO: 207 - DNA, 51 bp, synthetic construct
  misc_feature 1..51: note = Description of Artificial Sequence: Synthetic primer
  source 1..51: mol_type = other DNA
aataggatccacaatttacctgtggctgtcgtgattctggtggttgcagat

SEQ ID NO: 208 - DNA, 50 bp, synthetic construct
  misc_feature 1..50: note = Description of Artificial Sequence: Synthetic primer
  source 1..50: mol_type = other DNA
aataaagcttatgtgtaccggagatctgcaaccaccagaagcacaagtga

SEQ ID NO: 209 - DNA, 51 bp, synthetic construct
  misc_feature 1..51: note = Description of Artificial Sequence: Synthetic primer
  source 1..51: mol_type = other DNA
aataggatccacaatttacctctatcacttgtgcttctggtggttgcagat

SEQ ID NO: 210 - DNA, 52 bp, synthetic construct
  misc_feature 1..52: note = Description of Artificial Sequence: Synthetic primer
  source 1..52: mol_type = other DNA
aataaagcttatgtgtaccgaagatctgcaaccaccagaaccacaactgcca

SEQ ID NO: 211 - DNA, 51 bp, synthetic construct
  misc_feature 1..51: note = Description of Artificial Sequence: Synthetic primer
  source 1..51: mol_type = other DNA
aataggatccacaatttacctctggcagttgtggttctggtggttgcagat

SEQ ID NO: 212 - DNA, 50 bp, synthetic construct
  misc_feature 1..50: note = Description of Artificial Sequence: Synthetic primer
  source 1..50: mol_type = other DNA
aataaagcttatgtgtaccgaagatatggaaccacgaaaaacaacaatga

SEQ ID NO: 213 - DNA, 51 bp, synthetic construct
  misc_feature 1..51: note = Description of Artificial Sequence: Synthetic primer
  source 1..51: mol_type = other DNA
aataggatccacaatatttctttgtcattgttgtttttcgtggttccatat

SEQ ID NO: 214 - DNA, 55 bp, synthetic construct; identical to SEQ ID NO: 168
  misc_feature 1..55: note = Description of Artificial Sequence: Synthetic primer
  source 1..55: mol_type = other DNA

SEQ ID NO: 215 - DNA, 56 bp, synthetic construct
  misc_feature 1..56: note = Description of Artificial Sequence: Synthetic primer
  source 1..56: mol_type = other DNA
aataggatccacaatttttttttttttttttctttggcagtgctggttctggtggt

SEQ ID NO: 216 - DNA, 55 bp, synthetic construct; identical to SEQ ID NO: 168
  misc_feature 1..55: note = Description of Artificial Sequence: Synthetic primer
  source 1..55: mol_type = other DNA

SEQ ID NO: 217 - DNA, 54 bp, synthetic construct
  misc_feature 1..54: note = Description of Artificial Sequence: Synthetic primer
  source 1..54: mol_type = other DNA
aataggatccacaattttttttttttttctttggcagtgctggttctggtggtg

SEQ ID NO: 218 - DNA, 49 bp, synthetic construct
  misc_feature 1..49: note = Description of Artificial Sequence: Synthetic primer
  source 1..49: mol_type = other DNA
aataaagcttatgtgtaccgaagatctgcaatcaccaaaaacaacaatg

SEQ ID NO: 219 - DNA, 50 bp, synthetic construct; identical to SEQ ID NO: 179
  misc_feature 1..50: note = Description of Artificial Sequence: Synthetic primer
  source 1..50: mol_type = other DNA

SEQ ID NO: 220 - DNA, 55 bp, synthetic construct
  misc_feature 1..55: note = Description of Artificial Sequence: Synthetic primer
  source 1..55: mol_type = other DNA
aataaagcttatgtgtaccggagatctgaaactaccagaaccaccaatgtcaaag

SEQ ID NO: 221 - DNA, 52 bp, synthetic construct
  misc_feature 1..52: note = Description of Artificial Sequence: Synthetic primer
  source 1..52: mol_type = other DNA
aataggatccacaattttttttttttttctttgacattggtggttctggtag

SEQ ID NO: 222 - DNA, 49 bp, synthetic construct
  misc_feature 1..49: note = Description of Artificial Sequence: Synthetic primer
  source 1..49: mol_type = other DNA
aataaagcttatgtgtaccgaagatctgcaaccaccaaaaacaacaatg

SEQ ID NO: 223 - DNA, 51 bp, synthetic construct
  misc_feature 1..51: note = Description of Artificial Sequence: Synthetic primer
  source 1..51: mol_type = other DNA
aataggatccacaatatttctctgccattgttgtttttggtggttgcagat

SEQ ID NO: 224 - DNA, 51 bp, synthetic construct
  misc_feature 1..51: note = Description of Artificial Sequence: Synthetic primer
  source 1..51: mol_type = other DNA
aataaagcttatgtgtagcgaagatccggaaccaccaaaaacaacaatgac

SEQ ID NO: 225 - DNA, 51 bp, synthetic construct
  misc_feature 1..51: note = Description of Artificial Sequence: Synthetic primer
  source 1..51: mol_type = other DNA
aataggatccacaatttttctttgtcattgttgtttttggtggttccggat

SEQ ID NO: 226 - DNA, 50 bp, synthetic construct
  misc_feature 1..50: note = Description of Artificial Sequence: Synthetic primer
  source 1..50: mol_type = other DNA
aataaagcttatgtgtaccgaagatctgaaaccaccagaagcatcactgc

SEQ ID NO: 227 - DNA, 51 bp, synthetic construct
  misc_feature 1..51: note = Description of Artificial Sequence: Synthetic primer
  source 1..51: mol_type = other DNA
aataggatccacaattttcctctggcagtgatgcttctggtggtttcagat

SEQ ID NO: 228 - DNA, 54 bp, synthetic construct
  misc_feature 1..54: note = Description of Artificial Sequence: Synthetic primer
  source 1..54: mol_type = other DNA
aataaagcttatgtgtgccggagatctggaacaaccagaaccaccagtggcaaa

SEQ ID NO: 229 - DNA, 55 bp, synthetic construct
  misc_feature 1..55: note = Description of Artificial Sequence: Synthetic primer
  source 1..55: mol_type = other DNA
aataggatccacaattttttttttttttctttgccactggtggttctggttgttc

SEQ ID NO: 230 - DNA, 49 bp, synthetic construct
  misc_feature 1..49: note = Description of Artificial Sequence: Synthetic primer
  source 1..49: mol_type = other DNA
aataaagcttatgtgtaacggagatctggaacgaccagaaccaccagtg

SEQ ID NO: 231 - DNA, 51 bp, synthetic construct
  misc_feature 1..51: note = Description of Artificial Sequence: Synthetic primer
  source 1..51: mol_type = other DNA
aataggatccacaatattcctttgccactggtggttctggtcgttccagat

SEQ ID NO: 232 - DNA, 50 bp, synthetic construct
  misc_feature 1..50: note = Description of Artificial Sequence: Synthetic primer
  source 1..50: mol_type = other DNA
aataaagcttatgtgtaccgaagatctgaaaccaccagaaccaccactgc

SEQ ID NO: 233 - DNA, 51 bp, synthetic construct
  misc_feature 1..51: note = Description of Artificial Sequence: Synthetic primer
  source 1..51: mol_type = other DNA
aataggatccacaattttcctttggcagtggtggttctggtggtttcagat

SEQ ID NO: 234 - DNA, 49 bp, synthetic construct
  misc_feature 1..49: note = Description of Artificial Sequence: Synthetic primer
  source 1..49: mol_type = other DNA
aataaagcttatgtgtaacgaagctctggaaccaccaccactgcgaaag

SEQ ID NO: 235 - DNA, 49 bp, synthetic construct
  misc_feature 1..49: note = Description of Artificial Sequence: Synthetic primer
  source 1..49: mol_type = other DNA
aataggatccacaatgttcctttcgcagtggtggtggttccagagcttc

SEQ ID NO: 236 - DNA, 49 bp, synthetic construct
  misc_feature 1..49: note = Description of Artificial Sequence: Synthetic primer
  source 1..49: mol_type = other DNA
aataaagcttatgtgtcccgaagatctggaacgaccaccattgacaaag

SEQ ID NO: 237 - DNA, 45 bp, synthetic construct
  misc_feature 1..45: note = Description of Artificial Sequence: Synthetic primer
  source 1..45: mol_type = other DNA
aataggatccacaatgttcctttgtcaatggtggtcgttccagat

SEQ ID NO: 238 - DNA, 50 bp, synthetic construct
  misc_feature 1..50: note = Description of Artificial Sequence: Synthetic primer
  source 1..50: mol_type = other DNA
aataaagcttatgtgtaccgaagatctggaaccaccagaacgaccactgc

SEQ ID NO: 239 - DNA, 50 bp, synthetic construct
  misc_feature 1..50: note = Description of Artificial Sequence: Synthetic primer
  source 1..50: mol_type = other DNA
aataggatccacaaatttcccttggcagtggtcgttctggtggttccaga

SEQ ID NO: 240 - DNA, 51 bp, synthetic construct
  misc_feature 1..51: note = Description of Artificial Sequence: Synthetic primer
  source 1..51: mol_type = other DNA
aataaagcttatgtgtgccggagatctgaaaccaccagaaacaacaatgtc

SEQ ID NO: 241 - DNA, 50 bp, synthetic construct
  misc_feature 1..50: note = Description of Artificial Sequence: Synthetic primer
  source 1..50: mol_type = other DNA
aataggatccacaatttttctttgacattgttgtttctggtggtttcaga

SEQ ID NO: 242 - DNA, 50 bp, synthetic construct
  misc_feature 1..50: note = Description of Artificial Sequence: Synthetic primer
  source 1..50: mol_type = other DNA
aataaagcttatgtgcaccgaagatctgcaacaaccagaacgatcacagc

SEQ ID NO: 243 - DNA, 50 bp, synthetic construct
  misc_feature 1..50: note = Description of Artificial Sequence: Synthetic primer
  source 1..50: mol_type = other DNA
aataggatccacaactttccattggctgtgatcgttctggttgttgcaga

SEQ ID NO: 244 - DNA, 60 bp, synthetic construct
  misc_feature 1..60: note = Description of Artificial Sequence: Synthetic primer
  source 1..60: mol_type = other DNA
aataaagcttatgtgtcccgaagatctgcaaccaccagaaccagcactgccagagaaaaa

SEQ ID NO: 245 - DNA, 60 bp, synthetic construct
  misc_feature 1..60: note = Description of Artificial Sequence: Synthetic primer
  source 1..60: mol_type = other DNA
aataggatccgaccacgccgacgctgacggcgctttttacgaccagagccagaaccacaa

SEQ ID NO: 246 - DNA, 60 bp, synthetic construct
  misc_feature 1..60: note = Description of Artificial Sequence: Synthetic primer
  source 1..60: mol_type = other DNA
accagaaccagcactgccagagaaaaaaaaaaaaaaaattgtggttctggctctggtcgt

SEQ ID NO: 247 - DNA, 57 bp, synthetic construct
  misc_feature 1..57: note = Description of Artificial Sequence: Synthetic primer
  source 1..57: mol_type = other DNA
aataaagcttatgtgtaccgaagttctggtaccacgaaccaccagtggcaaaggaag

SEQ ID NO: 248 - DNA, 61 bp, synthetic construct
  misc_feature 1..61: note = Description of Artificial Sequence: Synthetic primer
  source 1..61: mol_type = other DNA
aataggatccggagccacacgccgatgctgacggatggcctttttgcgaccagagccagaa

SEQ ID NO: 249 - DNA, 60 bp, synthetic construct
  misc_feature 1..60: note = Description of Artificial Sequence: Synthetic primer
  source 1..60: mol_type = other DNA
accacgaaccaccagtggcaaaggaagattgtggttctggctctggtcgcaaaaaggcca

SEQ ID NO: 250 - DNA, 52 bp, synthetic construct
  misc_feature 1..52: note = Description of Artificial Sequence: Synthetic primer
  source 1..52: mol_type = other DNA
aataaagcttatgtgtgccgaagatctgcaaccaccaccactgctagaggca

SEQ ID NO: 251 - DNA, 59 bp, synthetic construct
  misc_feature 1..59: note = Description of Artificial Sequence: Synthetic primer
  source 1..59: mol_type = other DNA
aataggatccacactgacggcgctttttacgaccagagtcagaaccacaatgtgcctct

SEQ ID NO: 252 - DNA, 59 bp, synthetic construct
  misc_feature 1..59: note = Description of Artificial Sequence: Synthetic primer
  source 1..59: mol_type = other DNA
atctgcaaccaccaccactgctagaggcacattgtggttctgactctggtcgtaaaaag

SEQ ID NO: 253 - DNA, 49 bp, synthetic construct
  misc_feature 1..49: note = Description of Artificial Sequence: Synthetic primer
  source 1..49: mol_type = other DNA
aataaagcttatgtgtaccgcagctccggaaccaccagaaccacaactg

SEQ ID NO: 254 - DNA, 51 bp, synthetic construct
  misc_feature 1..51: note = Description of Artificial Sequence: Synthetic primer
  source 1..51: mol_type = other DNA
aataggatccacaatttgcctgtggcagttgtggttctggtggttccggag

SEQ ID NO: 255 - DNA, 49 bp, synthetic construct
  misc_feature 1..49: note = Description of Artificial Sequence: Synthetic primer
  source 1..49: mol_type = other DNA
aataaagcttatgtgtcccgcagatctgcaacaaccagaaacatcactg

SEQ ID NO: 256 - DNA, 51 bp, synthetic construct
  misc_feature 1..51: note = Description of Artificial Sequence: Synthetic primer
  source 1..51: mol_type = other DNA
aataggatccacaattttcctctggcagtgatgtttctggttgttgcagat

SEQ ID NO: 257 - DNA, 48 bp, synthetic construct
  misc_feature 1..48: note = Description of Artificial Sequence: Synthetic primer
  source 1..48: mol_type = other DNA
aataaagcttatgtgttccgtagatctgcaaccaccagcacgactacg

SEQ ID NO: 258 - DNA, 51 bp, synthetic construct; identical to SEQ ID NO: 205
  misc_feature 1..51: note = Description of Artificial Sequence: Synthetic primer
  source 1..51: mol_type = other DNA

SEQ ID NO: 259 - DNA, 49 bp, synthetic construct
  misc_feature 1..49: note = Description of Artificial Sequence: Synthetic primer
  source 1..49: mol_type = other DNA
aataaagcttatgtgcaccgaggctttggaaccaccagaaccaccactg

SEQ ID NO: 260 - DNA, 51 bp, synthetic construct
  misc_feature 1..51: note = Description of Artificial Sequence: Synthetic primer
  source 1..51: mol_type = other DNA
aataggatccacaattttcctttgtcagtggtggttctggtggttccaaag

SEQ ID NO: 261 - DNA, 49 bp, synthetic construct
  misc_feature 1..49: note = Description of Artificial Sequence: Synthetic primer
  source 1..49: mol_type = other DNA
aataaagcttatgtgtaccgaagctatggaaccaccagaaccaccactg

SEQ ID NO: 262 - DNA, 51 bp, synthetic construct
  misc_feature 1..51: note = Description of Artificial Sequence: Synthetic primer
  source 1..51: mol_type = other DNA
aataggatccacaactttcccttgccagtggtggttctggtggttccatag

SEQ ID NO: 263 - DNA, 49 bp, synthetic construct
  misc_feature 1..49: note = Description of Artificial Sequence: Synthetic primer
  source 1..49: mol_type = other DNA
aataaagcttatgtgtaccgcagatctgcaaccaccagaagcatcactg

SEQ ID NO: 264 - DNA, 51 bp, synthetic construct
  misc_feature 1..51: note = Description of Artificial Sequence: Synthetic primer
  source 1..51: mol_type = other DNA
aataggatccacaattttgctgtggcagtgatgcttctggtggttgcagat

SEQ ID NO: 265 - DNA, 52 bp, synthetic construct
  misc_feature 1..52: note = Description of Artificial Sequence: Synthetic primer
  source 1..52: mol_type = other DNA
aataaagcttatgtgtaccgcagctccggaaccaccagaaccacgactgcca

SEQ ID NO: 266 - DNA, 47 bp, synthetic construct
  misc_feature 1..47: note = Description of Artificial Sequence: Synthetic primer
  source 1..47: mol_type = other DNA
aataggatccacaatttccctctggcagtcgtggttctggtggttcc

SEQ ID NO: 267 - DNA, 51 bp, synthetic construct
  misc_feature 1..51: note = Description of Artificial Sequence: Synthetic primer
  source 1..51: mol_type = other DNA
aataaagcttatgtgtaccaaagatttggcaccacaagcaccaccattgct

SEQ ID NO: 268 - DNA, 50 bp, synthetic construct
  misc_feature 1..50: note = Description of Artificial Sequence: Synthetic primer
  source 1..50: mol_type = other DNA
aataggatccacaattttcctttagcaatggtggtgcttgtggtgccaaa

SEQ ID NO: 269 - DNA, 36 bp, synthetic construct
  misc_feature 1..36: note = Description of Artificial Sequence: Synthetic primer
  source 1..36: mol_type = other DNA
aaataagcttgtatatctccttcttaaagttaaaca

SEQ ID NO: 270 - DNA, 32 bp, synthetic construct
  misc_feature 1..32: note = Description of Artificial Sequence: Synthetic primer
  source 1..32: mol_type = other DNA
aataactcgagagatccggctgctaacaaagc

SEQ ID NO: 271 - DNA, 54 bp, synthetic construct
  misc_feature 1..54: note = Description of Artificial Sequence: Synthetic primer
  source 1..54: mol_type = other DNA
ataagctttaatacgactcactatagggttaactttagtaaggaggacagctaa

SEQ ID NO: 272 - DNA, 23 bp, synthetic construct
  misc_feature 1..23: note = Description of Artificial Sequence: Synthetic primer
  source 1..23: mol_type = other DNA
gtggtgatggtgatggtgaccta

SEQ ID NO: 273 - DNA, 50 bp, synthetic construct
  misc_feature 1..50: note = Description of Artificial Sequence: Synthetic primer
  source 1..50: mol_type = other DNA
attattctcgagttaatagccggtgccgtggtgatggtgatggtgaccta

SEQ ID NO: 274 - DNA, 35 bp, synthetic construct
  misc_feature 1..35: note = Description of Artificial Sequence: Synthetic primer
  source 1..35: mol_type = other DNA
aaatactcgagtcgttttatctgttgtttgtcggt

SEQ ID NO: 275 - DNA, 35 bp, synthetic construct
  misc_feature 1..35: note = Description of Artificial Sequence: Synthetic primer
  source 1..35: mol_type = other DNA
aaataaagcttctctgaatggcgggagtatgaaaa

SEQ ID NO: 276 - DNA, 22 bp, synthetic construct
  misc_feature 1..22: note = Description of Artificial Sequence: Synthetic primer
  source 1..22: mol_type = other DNA
ccgcgaatggtgagattgagaa

SEQ ID NO: 277 - DNA, 22 bp, synthetic construct
  misc_feature 1..22: note = Description of Artificial Sequence: Synthetic primer
  source 1..22: mol_type = other DNA
acgcaaaaaggccatccgtcag

SEQ ID NO: 278 - DNA, 58 bp, synthetic construct
  misc_feature 1..58: note = Description of Artificial Sequence: Synthetic primer
  source 1..58: mol_type = other DNA
attattctcgagttaatagccggtgcctaagccgctaccaccacgccgacgctgacgg

SEQ ID NO: 279 - DNA, 32 bp, synthetic construct
  misc_feature 1..32: note = Description of Artificial Sequence: Synthetic primer
  source 1..32: mol_type = other DNA
aaataagcttatggatagtagtgcggtgatca

SEQ ID NO: 280 - DNA, 32 bp, synthetic construct
  misc_feature 1..32: note = Description of Artificial Sequence: Synthetic primer
  source 1..32: mol_type = other DNA
attattctcgagctagtggtgatggtgatggt

SEQ ID NO: 281 - DNA, 33 bp, synthetic construct
  misc_feature 1..33: note = Description of Artificial Sequence: Synthetic primer
  source 1..33: mol_type = other DNA
aactaagcttttcctcctgttagcccaaaaaac

SEQ ID NO: 282 - DNA, 32 bp, synthetic construct
  misc_feature 1..32: note = Description of Artificial Sequence: Synthetic primer
  source 1..32: mol_type = other DNA
aatactcgaggctgttttggcggatgagagaa

SEQ ID NO: 283 - DNA, 28 bp, synthetic construct
  misc_feature 1..28: note = Description of Artificial Sequence: Synthetic primer
  source 1..28: mol_type = other DNA
aaatccatggatagtagtgcggtgatca

SEQ ID NO: 284 - DNA, 29 bp, synthetic construct
  misc_feature 1..29: note = Description of Artificial Sequence: Synthetic primer
  source 1..29: mol_type = other DNA
aaatcatatggatagtagtgcggtgatca

SEQ ID NO: 285 - DNA, 33 bp, synthetic construct
  misc_feature 1..33: note = Description of Artificial Sequence: Synthetic primer
  source 1..33: mol_type = other DNA
aatactcgagctagtggtgatggtgatggtgag

SEQ ID NO: 286 - DNA, 32 bp, synthetic construct
  misc_feature 1..32: note = Description of Artificial Sequence: Synthetic primer
  source 1..32: mol_type = other DNA
aatactcgagagagccagaaccagatcccgga

SEQ ID NO: 287 - DNA, 23 bp, synthetic construct
  misc_feature 1..23: note = Description of Artificial Sequence: Synthetic primer
  source 1..23: mol_type = other DNA
taagccgctaccaccacgccgac

SEQ ID NO: 288 - DNA, 37 bp, synthetic construct
  misc_feature 1..37: note = Description of Artificial Sequence: Synthetic primer
  source 1..37: mol_type = other DNA
ggtgcaattcctaaaactccagtacaatacttactgc

SEQ ID NO: 289 - DNA, 39 bp, synthetic construct
  misc_feature 1..39: note = Description of Artificial Sequence: Synthetic primer
  source 1..39: mol_type = other DNA
gtattgtactggagttttaggaattgcaccattttcctc

SEQ ID NO: 290 - DNA, 35 bp, synthetic construct
  misc_feature 1..35: note = Description of Artificial Sequence: Synthetic primer
  source 1..35: mol_type = other DNA
aatactcgagttattttggaggatggtcgccacca

SEQ ID NO: 291 - DNA, 40 bp, synthetic construct
  misc_feature 1..40: note = Description of Artificial Sequence: Synthetic primer
  source 1..40: mol_type = other DNA
aataaagcttatgggatccggttctggctcaggttcttcc

SEQ ID NO: 292 - DNA, 49 bp, synthetic construct
  misc_feature 1..49: note = Description of Artificial Sequence: Synthetic primer
  misc_difference 1: note = a, c, t, g, unknown or other
  misc_difference 4: note = a, c, t, g, unknown or other
  misc_difference 7: note = a, c, t, g, unknown or other
  misc_difference 10: note = a, c, t, g, unknown or other
  misc_difference 13: note = a, c, t, g, unknown or other
  misc_difference 16: note = a, c, t, g, unknown or other
  misc_difference 19: note = a, c, t, g, unknown or other
  misc_difference 22: note = a, c, t, g, unknown or other
  source 1..49: mol_type = other DNA
naancancantgncanagnaanattgtggttctggctctggtcgtaaaa

SEQ ID NO: 293 - DNA, 49 bp, synthetic construct
  misc_feature 1..49: note = Description of Artificial Sequence: Synthetic primer
  misc_difference 2: note = a, c, t, g, unknown or other
  misc_difference 5: note = a, c, t, g, unknown or other
  misc_difference 8: note = a, c, t, g, unknown or other
  misc_difference 11: note = a, c, t, g, unknown or other
  misc_difference 14: note = a, c, t, g, unknown or other
  misc_difference 17: note = a, c, t, g, unknown or other
  misc_difference 20: note = a, c, t, g, unknown or other
  misc_difference 23: note = a, c, t, g, unknown or other
  source 1..49: mol_type = other DNA
gnacnacnacngcnaanggnaanttgtggttctggctctggtcgtaaaa

SEQ ID NO: 294 - DNA, 49 bp, synthetic construct
  misc_feature 1..49: note = Description of Artificial Sequence: Synthetic primer
  misc_difference 2: note = a, c, t, g, unknown or other
  misc_difference 4: note = a, c, t, g, unknown or other
  misc_difference 8: note = a, c, t, g, unknown or other
  misc_difference 10: note = a, c, t, g, unknown or other
  misc_difference 14: note = a, c, t, g, unknown or other
  misc_difference 16: note = a, c, t, g, unknown or other
  misc_difference 20: note = a, c, t, g, unknown or other
  misc_difference 22: note = a, c, t, g, unknown or other
  source 1..49: mol_type = other DNA
gnancacnantgcnanaggnanattgtggttctggctctggtcgtaaaa

SEQ ID NO: 295 - DNA, 49 bp, synthetic construct
  misc_feature 1..49: note = Description of Artificial Sequence: Synthetic primer
  misc_difference 1: note = a, c, t, g, unknown or other
  misc_difference 5: note = a, c, t, g, unknown or other
  misc_difference 7: note = a, c, t, g, unknown or other
  misc_difference 11: note = a, c, t, g, unknown or other
  misc_difference 13: note = a, c, t, g, unknown or other
  misc_difference 17: note = a, c, t, g, unknown or other
  misc_difference 19: note = a, c, t, g, unknown or other
  misc_difference 23: note = a, c, t, g, unknown or other
  source 1..49: mol_type = other DNA
naacnancacngncaangnaaanttgtggttctggctctggtcgtaaaa

SEQ ID NO: 296 - DNA, 49 bp, synthetic construct
  misc_feature 1..49: note = Description of Artificial Sequence: Synthetic primer
  misc_difference 4: note = a, c, t, g, unknown or other
  misc_difference 7: note = a, c, t, g, unknown or other
  misc_difference 10: note = a, c, t, g, unknown or other
  misc_difference 13: note = a, c, t, g, unknown or other
  misc_difference 16: note = a, c, t, g, unknown or other
  misc_difference 19: note = a, c, t, g, unknown or other
  misc_difference 22: note = a, c, t, g, unknown or other
  source 1..49: mol_type = other DNA
gaancancantgncanagnaanattgtggttctggctctggtcgtaaaa

SEQ ID NO: 297 - DNA, 49 bp, synthetic construct
  misc_feature 1..49: note = Description of Artificial Sequence: Synthetic primer
  misc_difference 5: note = a, c, t, g, unknown or other
  misc_difference 8: note = a, c, t, g, unknown or other
  misc_difference 11: note = a, c, t, g, unknown or other
  misc_difference 14: note = a, c, t, g, unknown or other
  misc_difference 17: note = a, c, t, g, unknown or other
  misc_difference 20: note = a, c, t, g, unknown or other
  misc_difference 23: note = a, c, t, g, unknown or other
  source 1..49: mol_type = other DNA
gaacnacnacngcnaanggnaanttgtggttctggctctggtcgtaaaa

SEQ ID NO: 298 - DNA, 49 bp, synthetic construct
  misc_feature 1..49: note = Description of Artificial Sequence: Synthetic primer
  misc_difference 4: note = a, c, t, g, unknown or other
  misc_difference 8: note = a, c, t, g, unknown or other
  misc_difference 10: note = a, c, t, g, unknown or other
  misc_difference 14: note = a, c, t, g, unknown or other
  misc_difference 16: note = a, c, t, g, unknown or other
  misc_difference 20: note = a, c, t, g, unknown or other
  misc_difference 22: note = a, c, t, g, unknown or other
  source 1..49: mol_type = other DNA
gaancacnantgcnanaggnanattgtggttctggctctggtcgtaaaa

SEQ ID NO: 299 - DNA, 49 bp, synthetic construct
  misc_feature 1..49: note = Description of Artificial Sequence: Synthetic primer
  misc_difference 5: note = a, c, t, g, unknown or other
  misc_difference 7: note = a, c, t, g, unknown or other
  misc_difference 11: note = a, c, t, g, unknown or other
  misc_difference 13: note = a, c, t, g, unknown or other
  misc_difference 17: note = a, c, t, g, unknown or other
  misc_difference 19: note = a, c, t, g, unknown or other
  misc_difference 23: note = a, c, t, g, unknown or other
  source 1..49: mol_type = other DNA
gaacnancacngncaangnaaanttgtggttctggctctggtcgtaaaa

SEQ ID NO: 300 - DNA, 49 bp, synthetic construct
  misc_feature 1..49: note = Description of Artificial Sequence: Synthetic primer
  misc_difference 4: note = a, c, t, g, unknown or other
  misc_difference 7: note = a, c, t, g, unknown or other
  misc_difference 16: note = a, c, t, g, unknown or other
  misc_difference 19: note = a, c, t, g, unknown or other
  source 1..49: mol_type = other DNA
gaancancactgccanagnaaaattgtggttctggctctggtcgtaaaa

SEQ ID NO: 301 - DNA, 49 bp, synthetic construct
  misc_feature 1..49: note = Description of Artificial Sequence: Synthetic primer
  misc_difference 1: note = a, c, t, g, unknown or other
  misc_difference 10: note = a, c, t, g, unknown or other
  misc_difference 13: note = a, c, t, g, unknown or other
  misc_difference 22: note = a, c, t, g, unknown or other
  source 1..49: mol_type = other DNA
naaccaccantgncaaaggaanattgtggttctggctctggtcgtaaaa

SEQ ID NO: 302 - DNA, 49 bp, synthetic construct
  misc_feature 1..49: note = Description of Artificial Sequence: Synthetic primer
  misc_difference 4: note = a, c, t, g, unknown or other
  misc_difference 8: note = a, c, t, g, unknown or other
  misc_difference 16: note = a, c, t, g, unknown or other
  misc_difference 20: note = a, c, t, g, unknown or other
  source 1..49: mol_type = other DNA
gaancacnactgccanaggnaaattgtggttctggctctggtcgtaaaa

SEQ ID NO: 303 - DNA, 49 bp, synthetic construct
  misc_feature 1..49: note = Description of Artificial Sequence: Synthetic primer
  misc_difference 2: note = a, c, t, g, unknown or other
  misc_difference 10: note = a, c, t, g, unknown or other
  misc_difference 14: note = a, c, t, g, unknown or other
  misc_difference 22: note = a, c, t, g, unknown or other
  source 1..49: mol_type = other DNA
gnaccaccantgcnaaaggaanattgtggttctggctctggtcgtaaaa

SEQ ID NO: 304 - DNA, 51 bp, synthetic construct
  misc_feature 1..51: note = Description of Artificial Sequence: Synthetic primer
  misc_difference 3: note = a, c, t, g, unknown or other
  misc_difference 6: note = a, c, t, g, unknown or other
  misc_difference 9: note = a, c, t, g, unknown or other
  misc_difference 12: note = a, c, t, g, unknown or other
  misc_difference 15: note = a, c, t, g, unknown or other
  misc_difference 18: note = a, c, t, g, unknown or other
  misc_difference 21: note = a, c, t, g, unknown or other
  source 1..51: mol_type = other DNA
tgntgnttncanatnttnggnacacatttagctgtcctccttactaaagtt

SEQ ID NO: 305 - DNA, 51 bp, synthetic construct
  misc_feature 1..51: note = Description of Artificial Sequence: Synthetic primer
  misc_difference 2: note = a, c, t, g, unknown or other
  misc_difference 5: note = a, c, t, g, unknown or other
  misc_difference 8: note = a, c, t, g, unknown or other
  misc_difference 11: note = a, c, t, g, unknown or other
  misc_difference 14: note = a, c, t, g, unknown or other
  misc_difference 17: note = a, c, t, g, unknown or other
  misc_difference 20: note = a, c, t, g, unknown or other
  source 1..51: mol_type = other DNA
tngtngtnccnganctncgntacacatttagctgtcctccttactaaagtt

SEQ ID NO: 306 - DNA, 51 bp, synthetic construct
  misc_feature 1..51: note = Description of Artificial Sequence: Synthetic primer
  misc_difference 3: note = a, c, t, g, unknown or other
  misc_difference 5: note = a, c, t, g, unknown or other
  misc_difference 9: note = a, c, t, g, unknown or other
  misc_difference 11: note = a, c, t, g, unknown or other
  misc_difference 15: note = a, c, t, g, unknown or other
  misc_difference 17: note = a, c, t, g, unknown or other
  misc_difference 21: note = a, c, t, g, unknown or other
  source 1..51: mol_type = other DNA
tgntngttncngatntncggnacacatttagctgtcctccttactaaagtt

SEQ ID NO: 307 - DNA, 51 bp, synthetic construct
  misc_feature 1..51: note = Description of Artificial Sequence: Synthetic primer
  misc_difference 2: note = a, c, t, g, unknown or other
  misc_difference 6: note = a, c, t, g, unknown or other
  misc_difference 8: note = a, c, t, g, unknown or other
  misc_difference 12: note = a, c, t, g, unknown or other
  misc_difference 14: note = a, c, t, g, unknown or other
  misc_difference 18: note = a, c, t, g, unknown or other
  misc_difference 20: note = a, c, t, g, unknown or other
  source 1..51: mol_type = other DNA
tngtgntnccanancttngntacacatttagctgtcctccttactaaagtt

SEQ ID NO: 308 - DNA, 51 bp, synthetic construct
  misc_feature 1..51: note = Description of Artificial Sequence: Synthetic primer
  misc_difference 3: note = a, c, t, g, unknown or other
  misc_difference 6: note = a, c, t, g, unknown or other
  misc_difference 12: note = a, c, t, g, unknown or other
  misc_difference 15: note = a, c, t, g, unknown or other
  misc_difference 18: note = a, c, t, g, unknown or other
  misc_difference 21: note = a, c, t, g, unknown or other
  source 1..51: mol_type = other DNA
tgntgnttccanatnttnggnacacatttagctgtcctccttactaaagtt

SEQ ID NO: 309 - DNA, 51 bp, synthetic construct
  misc_feature 1..51: note = Description of Artificial Sequence: Synthetic primer
  misc_difference 2: note = a, c, t, g, unknown or other
  misc_difference 5: note = a, c, t, g, unknown or other
  misc_difference 11: note = a, c, t, g, unknown or other
  misc_difference 14: note = a, c, t, g, unknown or other
  misc_difference 17: note = a, c, t, g, unknown or other
  misc_difference 20: note = a, c, t, g, unknown or other
  source 1..51: mol_type = other DNA
tngtngttccnganctncgntacacatttagctgtcctccttactaaagtt

SEQ ID NO: 310 - DNA, 51 bp, synthetic construct
  misc_feature 1..51: note = Description of Artificial Sequence: Synthetic primer
  misc_difference 3: note = a, c, t, g, unknown or other
  misc_difference 5: note = a, c, t, g, unknown or other
  misc_difference 11: note = a, c, t, g, unknown or other
  misc_difference 15: note = a, c, t, g, unknown or other
  misc_difference 17: note = a, c, t, g, unknown or other
  misc_difference 21: note = a, c, t, g, unknown or other
  source 1..51: mol_type = other DNA
tgntngttccngatntncggnacacatttagctgtcctccttactaaagtt

SEQ ID NO: 311 - DNA, 51 bp, synthetic construct
  misc_feature 1..51: note = Description of Artificial Sequence: Synthetic primer
  misc_difference 2: note = a, c, t, g, unknown or other
  misc_difference 6: note = a, c, t, g, unknown or other
  misc_difference 12: note = a, c, t, g, unknown or other
  misc_difference 14: note = a, c, t, g, unknown or other
  misc_difference 18: note = a, c, t, g, unknown or other
  misc_difference 20: note = a, c, t, g, unknown or other
  source 1..51: mol_type = other DNA
tngtgnttccanancttngntacacatttagctgtcctccttactaaagtt

SEQ ID NO: 312 - DNA, 51 bp, synthetic construct
  misc_feature 1..51: note = Description of Artificial Sequence: Synthetic primer
  misc_difference 6: note = a, c, t, g, unknown or other
  misc_difference 9: note = a, c, t, g, unknown or other
  misc_difference 18: note = a, c, t, g, unknown or other
  misc_difference 21: note = a, c, t, g, unknown or other
  source 1..51: mol_type = other DNA
tggtgnttncagatcttnggnacacatttagctgtcctccttactaaagtt

SEQ ID NO: 313 - DNA, 51 bp, synthetic construct
  misc_feature 1..51: note = Description of Artificial Sequence: Synthetic primer
  misc_difference 3: note = a, c, t, g, unknown or other
  misc_difference 12: note = a, c, t, g, unknown or other
  misc_difference 15: note = a, c, t, g, unknown or other
  source 1..51: mol_type = other DNA
tgntggttccanatnttcggtacacatttagctgtcctccttactaaagtt

SEQ ID NO: 314 - DNA, 51 bp, synthetic construct
  misc_feature 1..51: note = Description of Artificial Sequence: Synthetic primer
  misc_difference 5: note = a, c, t, g, unknown or other
  misc_difference 9: note = a, c, t, g, unknown or other
  misc_difference 17: note = a, c, t, g, unknown or other
  misc_difference 21: note = a, c, t, g, unknown or other
  source 1..51: mol_type = other DNA
tggtngttncagatctncggnacacatttagctgtcctccttactaaagtt

SEQ ID NO: 315 - DNA, 51 bp, synthetic construct
  misc_feature 1..51: note = Description of Artificial Sequence: Synthetic primer
  misc_difference 3: note = a, c, t, g, unknown or other
  misc_difference 11: note = a, c, t, g, unknown or other
  misc_difference 15: note = a, c, t, g, unknown or other
  source 1..51: mol_type = other DNA
tgntggttccngatnttcggtacacatttagctgtcctccttactaaagtt

SEQ ID NO: 316 - DNA, 216 bp, synthetic construct
  misc_feature 1..216: note = Description of Artificial Sequence: Synthetic polynucleotide
  source 1..216: mol_type = other DNA
cctgcaggtaatacgactcactatagggttaactttagtaaggaggacagctaaaagcttatgtgtaccgaagatctggaaccaccagaaccaccactgccaaaggaaaattgtggatccggctctggtcgtaaaaagcgccgtcagcgtcggcgtggtggctccggtagcttaggtcaccatcaccatcaccacggcaccggctattaactcgag

SEQ ID NO: 317 - DNA, 150 bp, synthetic construct
  misc_feature 1..150: note = Description of Artificial Sequence: Synthetic polynucleotide
  source 1..150: mol_type = other DNA
atgtgtaccgaagatctggaaccaccagaaccaccactgccaaaggaaaattgtggatccggctctggtcgtaaaaagcgccgtcagcgtcggcgtggtggctccggtagcttaggtcaccatcaccatcaccacggcaccggctattaa

SEQ ID NO: 318 - DNA, 37 bp, synthetic construct
  misc_feature 1..37: note = Description of Artificial Sequence: Synthetic primer
  source 1..37: mol_type = other DNA
aaatcctgcaggtaatacgactcactatagggttaac

SEQ ID NO: 319 - DNA, 50 bp, synthetic construct; identical to SEQ ID NO: 273
  misc_feature 1..50: note = Description of Artificial Sequence: Synthetic primer
  source 1..50: mol_type = other DNA

SEQ ID NO: 320 - DNA, 226 bp, synthetic construct
  misc_feature 1..226: note = Description of Artificial Sequence: Synthetic polynucleotide
  source 1..226: mol_type = other DNA
tataccctgcaggtaatacgactcactatagggttaactttagtaaggaggacagctaaaagcttatgtgtgaagacgccaaaaacataaagaaaggcccggcgccattctatccgtgtggatccggctctggtcgtaaaaagcgccgtcagcgtcggcgtggtggctccggtagcttaggtcaccatcaccatcaccacggcaccggctattaactcgagagatc

SEQ ID NO: 321 - DNA, 216 bp, synthetic construct
  misc_feature 1..216: note = Description of Artificial Sequence: Synthetic polynucleotide
  source 1..216: mol_type = other DNA
cctgcaggtaatacgactcactatagggttaactttagtaaggaggacagctaaaagcttatgtgtgaagacgccaaaaacataaagaaaggcccggcgccattctatccgtgtggatccggctctggtcgtaaaaagcgccgtcagcgtcggcgtggtggctccggtagcttaggtcaccatcaccatcaccacggcaccggctattaactcgag

SEQ ID NO: 322 - DNA, 150 bp, synthetic construct
  misc_feature 1..150: note = Description of Artificial Sequence: Synthetic polynucleotide
  source 1..150: mol_type = other DNA
atgtgtgaagacgccaaaaacataaagaaaggcccggcgccattctatccgtgtggatccggctctggtcgtaaaaagcgccgtcagcgtcggcgtggtggctccggtagcttaggtcaccatcaccatcaccacggcaccggctattaa

SEQ ID NO: 323 - DNA, 37 bp, synthetic construct; identical to SEQ ID NO: 318
  misc_feature 1..37: note = Description of Artificial Sequence: Synthetic primer
  source 1..37: mol_type = other DNA

SEQ ID NO: 324 - DNA, 50 bp, synthetic construct; identical to SEQ ID NO: 273
  misc_feature 1..50: note = Description of Artificial Sequence: Synthetic primer
  source 1..50: mol_type = other DNA

SEQ ID NO: 325 - DNA, 51 bp, synthetic construct; identical to SEQ ID NO: 207
  misc_feature 1..51: note = Description of Artificial Sequence: Synthetic primer
  source 1..51: mol_type = other DNA

SEQ ID NO: 326 - DNA, 41 bp, synthetic construct
  misc_feature 1..41: note = Description of Artificial Sequence: Synthetic primer
  source 1..41: mol_type = other DNA
acaatttaccattggccgtagtcgtgctggtggttgcagat

SEQ ID NO: 327 - DNA, 48 bp, synthetic construct
  misc_feature 1..48: note = Description of Artificial Sequence: Synthetic primer
  source 1..48: mol_type = other DNA
aataaagcttatgtgcaccgagctttggaaccaccagaaccaccactg

SEQ ID NO: 328 - DNA, 50 bp, synthetic construct
  misc_feature 1..50: note = Description of Artificial Sequence: Synthetic primer
  source 1..50: mol_type = other DNA
aataggatccacaattttgctgtggcagtgatgcttctggtggttgcaga

SEQ ID NO: 329 - protein, 14 aa, unidentified
  REGION 1..14: note = Description of Unknown: REST peptide
  MOD_RES 4: note = Phosphorylated Ser
  MOD_RES 7: note = Phosphorylated Ser
TEDSPPSPPLPKEN

SEQ ID NO: 330 - RNA, 24 nt, synthetic construct
  misc_feature 1..24: note = Description of Artificial Sequence: Synthetic oligonucleotide
  source 1..24: mol_type = other RNA
tagccggtgaaaaaaaaaaaaaaa

SEQ ID NO: 331 - DNA, 25 bp, synthetic construct
  misc_feature 1..25: note = Description of Artificial Sequence: Synthetic oligonucleotide
  source 1..25: mol_type = other DNA
ttttttttttttttttttttttttt

SEQ ID NO: 332 - DNA, 19 bp, synthetic construct
  misc_feature 1..19: note = Description of Artificial Sequence: Synthetic primer
  source 1..19: mol_type = other DNA
agagcacgagctgcctgac

SEQ ID NO: 333 - DNA, 18 bp, synthetic construct
  misc_feature 1..18: note = Description of Artificial Sequence: Synthetic primer
  source 1..18: mol_type = other DNA
ggatgccacaggactcca

SEQ ID NO: 334 - DNA, 22 bp, synthetic construct
  misc_feature 1..22: note = Description of Artificial Sequence: Synthetic primer
  source 1..22: mol_type = other DNA
tattagtgagtgggtaacggcg

SEQ ID NO: 335 - DNA, 23 bp, synthetic construct
  misc_feature 1..23: note = Description of Artificial Sequence: Synthetic primer
  source 1..23: mol_type = other DNA
gaagtattgcttcagttggcctt

SEQ ID NO: 336 - DNA, 20 bp, synthetic construct
  misc_feature 1..20: note = Description of Artificial Sequence: Synthetic primer
  source 1..20: mol_type = other DNA
ctcactaccacctgctgctc

SEQ ID NO: 337 - DNA, 20 bp, synthetic construct
  misc_feature 1..20: note = Description of Artificial Sequence: Synthetic primer
  source 1..20: mol_type = other DNA
tcagtccgtcgtctgctttc

SEQ ID NO: 338 - DNA, 20 bp, synthetic construct
  misc_feature 1..20: note = Description of Artificial Sequence: Synthetic primer
  source 1..20: mol_type = other DNA
tatcctggccacactgaggt

SEQ ID NO: 339 - DNA, 19 bp, synthetic construct
  misc_feature 1..19: note = Description of Artificial Sequence: Synthetic primer
  source 1..19: mol_type = other DNA
tcctgcagggacattgctc

SEQ ID NO: 340 - DNA, 20 bp, synthetic construct
  misc_feature 1..20: note = Description of Artificial Sequence: Synthetic primer
  source 1..20: mol_type = other DNA
agagctatgagctgcctgac

SEQ ID NO: 341 - DNA, 24 bp, synthetic construct
  misc_feature 1..24: note = Description of Artificial Sequence: Synthetic primer
  source 1..24: mol_type = other DNA
agctgtgcctcagaactaggcttt

SEQ ID NO: 342 - DNA, 24 bp, synthetic construct
  misc_feature 1..24: note = Description of Artificial Sequence: Synthetic primer
  source 1..24: mol_type = other DNA
taccagaaagacgcagggatgctt

SEQ ID NO: 343 - DNA, 20 bp, synthetic construct
  misc_feature 1..20: note = Description of Artificial Sequence: Synthetic primer
  source 1..20: mol_type = other DNA
ccggcagaactcagaagaag

SEQ ID NO: 344 - DNA, 20 bp, synthetic construct
  misc_feature 1..20: note = Description of Artificial Sequence: Synthetic primer
  source 1..20: mol_type = other DNA
tttgaggccaggggtaagat

SEQ ID NO: 345 - DNA, 24 bp, synthetic construct
  misc_feature 1..24: note = Description of Artificial Sequence: Synthetic primer
  source 1..24: mol_type = other DNA
ttcctggtcatgtatgtgattgta

SEQ ID NO: 346 - DNA, 21 bp, synthetic construct
  misc_feature 1..21: note = Description of Artificial Sequence: Synthetic primer
  source 1..21: mol_type = other DNA
gggcagtgtactggtcgctaa

SEQ ID NO: 347 - protein, 5 aa, unidentified
  REGION 1..5: note = Description of Unknown: REST peptide
SPPSP

SEQ ID NO: 348 - protein, 5 aa, synthetic construct
  REGION 1..5: note = Description of Artificial Sequence: Synthetic peptide
  SITE 1..5: note = D-amino acid
PEPPE

SEQ ID NO: 349 - DNA, 223 bp, synthetic construct
  misc_feature 1..223: note = Description of Artificial Sequence: Synthetic polynucleotide
  source 1..223: mol_type = other DNA
aataataagctttaatacgactcactatagggttaactttagtaaggaggacagctaaatgtgtaccgaagatctggaaccaccagaaccaccactgccaaaggaaaattgtggttctggctctggtcgtaaaaagcgccgtcagcgtcggcgtggtggctccggtagcggcttaggtcaccatcaccatcaccgacgcaccggctattaactcgagaataat

SEQ ID NO: 350 - protein, 50 aa, synthetic construct
  REGION 1..50: note = Description of Artificial Sequence: Synthetic polypeptide
MCTEDLEPPEPPLPKENCGSGSGRKKRRQRRRGGSGSGLGHHHHHHGTGY